(19) United States
(12) Patent Application Publication
(10) Pub. No.: US 2014/0087362 A1
(43) Pub. Date: Mar. 27, 2014

(54) Methods for assessing effectiveness and monitoring oncolytic virus treatment

(71) Applicants: Aladar A. Szalay, Highland, CA (US); Nanhai G. Chen, San Diego, CA (US); Huiqiang Wang, San Diego, CA (US); Melody Fells, San Diego, CA (US); Albert Roeder, Bernried (DE); Qian Zhang, San Diego, CA (US); Boris Minev, San Diego, CA (US)

(72) Inventors: Aladar A. Szalay, Highland, CA (US); Nanhai G. Chen, San Diego, CA (US); Huiqiang Wang, San Diego, CA (US); Melody Fells, San Diego, CA (US); Albert Roeder, Bernried (DE); Qian Zhang, San Diego, CA (US); Boris Minev, San Diego, CA (US)

(21) Appl. No.: 13/815,727

(22) Filed: Mar. 13, 2013

Related U.S. Application Data
(60) Provisional application No. 61/690,468, filed on Jun. 26, 2012.
(60) Provisional application No. 61/685,367, filed on Mar. 16, 2012.

(51) Int. Cl. C12Q 1/70

(52) U.S. Cl. CPC C12Q 1/701
USPC 435/5; 530/389.4; 530/327; 530/387.9

(57) ABSTRACT

Diagnostic methods for in vivo and ex vivo detection of circulating tumor cells (CTCs) for the diagnosis and treatment of cancer are provided. The diagnostic methods employ oncolytic viruses alone or in combination with one or more tumor cell enrichment and/or detection methods. Combinations and kits for use in the practicing the methods also are provided.

### RELATED APPLICATIONS

Benefit of priority is claimed to U.S. Provisional Application Ser. No. 61/690,468, filed Jun. 26, 2012, to Aladar A. Szalay, Nanhai G. Chen, Huiqiang Wang, Melody Fells, Albert Roeder, Qian Zhang and Boris Minev entitled “METHODS FOR ASSESSING EFFECTIVENESS AND MONITORING ONCOLYTIC VIRUS TREATMENT,” and to U.S. Provisional Application Ser. No. 61/685,367, filed Mar. 16, 2012, to Aladar A. Szalay, Nanhai G. Chen, Huiqiang Wang and Melody Fells, entitled “METHODS FOR ASSESSING EFFECTIVENESS AND MONITORING ONCOLYTIC VIRUS TREATMENT.”

This application is related to International PCT Application No. (Attorney Dkt. No. 33316-4833PC), filed Mar. 13, 2013, entitled “METHODS FOR ASSESSING EFFECTIVENESS AND MONITORING ONCOLYTIC VIRUS TREATMENT,” which also claims priority to U.S. Provisional Application Ser. Nos. 61/685,367 and 61/690,468. The subject matter of each of the above-noted applications is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy 1 and Copy 2), the contents of which are incorporated by reference in their entirety. The computer-readable file on each of the aforementioned compact discs, created on Mar. 11, 2013, is identical, 3.93 MB in size, and titled 4833SEQ.001.txt.

### FIELD OF INVENTION

Diagnostic methods for in vivo and ex vivo detection of circulating tumor cells for the diagnosis and treatment of cancer are provided. The diagnostic methods employ oncolytic viruses alone or in combination with one or more tumor cell enrichment methods. Combinations and kits for use in the practicing the methods also are provided.

### BACKGROUND

Most cancer deaths result from the metastatic spread of cancer in which tumor cells escape from the primary tumor and relocate to distant sites (Talmadge et al. (2010) AACR Cancer Res 70(14):5649-5669). Metastatic tumor cells found in body fluids, such as blood, lymphatic, cerebrospinal and ascitic fluids are biomarkers for evaluating cancer prognosis and for monitoring therapeutic response. Also, prevention and elimination of such metastatic tumor cells can increase survival rates and time. Metastatic tumor cells in the peripheral blood (i.e. circulating tumor cells (CTCs)), are prognostic biomarkers for solid tumors, including non-small cell lung cancer, breast cancer, colorectal cancer and prostate cancer (see, e.g., Balic et al. (2012) Expert Rev Mol Diagn 12(3):303-312; and van de Stolpe et al. (2011) Cancer Res 71(18):5955-5960). There are few methods for effective detection of CTCs.

Oncolytic viral therapy is effected by administering a virus that accumulates in tumor cells and replicates in the tumor cells. By virtue of replication in the cells, and optional delivery of therapeutic agents, treatment is effected because tumor cells are lysed resulting in shrinkage of the tumor, the optional therapeutic protein is expressed, which can treat the tumor, and other effects, such as antibody responses to released tumor antigens effect treatment.

It, however, can take months to observe results of treatment, and a plurality of treatments may be needed. Thus, it may be months, to know whether the oncolytic therapy is effective. If it is not effective, alternative therapies can be tried, whose effectiveness can be defeated by any delay in treatment. If it can be determined within a few weeks of initiating oncolytic therapy whether the therapy is not likely to be effective, an alternative therapy can be initiated earlier.

Hence, there is a need for a method or protocol to monitor oncolytic therapy, including determining whether a particular oncolytic therapy is effective. In addition, there is a need for diagnostic methods to stratify patients for responsiveness to cancer treatments in order to avoid delays in treatment and provide necessary modifications to ineffective therapeutic regimens. In addition, there is a need for the development of comprehensive, sensitive, and specific methods for detecting CTC detection.

### SUMMARY

Oncolytic viruses effect treatment by colonizing or accumulating in tumor cells and replicating. They provide an effective weapon in the tumor treatment arsenal. In some instances, a particular virus may not be effective for treating a particular tumor. It, however, is difficult to assess or early in treatment whether a virus is effective. A change in tumor or size or a decrease in metastasis may not be detectable for months after treatment; valuable time can be lost waiting to assess whether a virus is effective or whether a different virus could be more effective.

Provided herein are methods for detecting tumor cells in a body fluid. The methods herein employ oncolytic viruses that encode reporters for detection of the viruses to detect the tumor cells. In some embodiments, the oncolytic virus is administered to a subject, a body fluid sample is obtained at a pre-determined time after administration or at intervals thereafter, and virus is detected in cells in the sample. Since oncolytic viruses accumulate in tumor cells, the detected cells will be tumor cells. The timing of sampling and detection depends upon the application. Also, the tumor cells in the sample can be enriched by methods known in the art.

The ability to detect tumor cells, particular viable, not dead or dying tumor cells, in a body fluid can be employed in a variety of applications, particularly those that provide an indication of the status or stage of a tumor, regression of a tumor, remission, recurrence, effectiveness of treatment and other such parameters. The applications include methods for assessing the potential efficacy of treatment of a tumor with a particular oncolytic virus in which detection of infected tumor cells in body fluids following systemic administration is indicative that the viral therapy will infect and replicate in tumor cells; methods for monitoring progression of treatment, where an effective treatment results in a decrease in infected tumor cells over time, detection of metastatic disease, and other such methods, particularly any methods in which detection of circulating tumor cells is employed. It is shown herein that the tumor cells, such as circulating tumor cells (CTCs) that are detected appear to be live (viable) tumor cells; whereas methods that rely on other properties of CTCs, such as tumor markers, detect CTCs, but detect dead or dying cells as well as living, viable cells. Such methods will not provide an accurate picture of the status of tumor development, metastasis, and/or treatment.

Hence, provided herein are methods for assessing or predicting whether a particular treatment or treatment regimen is having an effect relatively soon, typically within a week or two after initiating treating. In addition, provided are methods for detection and/or enumeration of live tumor cells in preclinical and clinical liquid biopsies The methods herein also have other applications as described herein. In particular, shortly after administration of an oncolytic virus, such as within a day and before about 24 or at 24 days, the presence of virus in tumor cells in a body fluid indicates that the virus has infected tumors and tumor cells and/or is present in tumor cells released from tumors. The presence of virus thus indicates that the treatment should be continued. The absence of virus indicates that virus likely is not effectively infecting tumors or replicating, and treatment should be discontinued. After treatment has been ongoing, then the methods herein can be used to monitor treatment. Once viral infection of tumor cells and replication therein has been established, then, the numbers of tumor cells detected should decrease over time as the treatment eliminates tumor cells.

Provided herein are methods for monitoring efficacy of treatment with an oncolytic virus by testing a body fluid sample, such as but not limited to, blood, plasma, urine and cerebral spinal fluid, from a subject to whom an oncolytic virus has been administered. The virus includes, or is modified, such as by including a reporter protein or protein that induces a detectable signal, so that the virus is detectable (i.e., is an oncolytic reporter virus). If the virus has colonized or infected and is replicating in tumors in the subject, it is shown herein tumor cells that are released into circulation from the tumors will contain virus and are detectable within a short time, such as 1, 2, 3, 4, 5, 6, 7, 8, 9, 10, 12, 14, 16, 18, 20, 22, 24 days (before tumor shrinkage or disease remission or stabilization can be reliably detected) after administration. Detection of the oncolytic reporter virus in the sample indicates that tumor cells in the sample contain the virus, which indicates that the virus is likely or will or is effective against the tumor in that it has infected tumor cells and has replicated sufficiently to be detectable. Suitable controls can be employed for comparison. Also, samples can be obtained/monitored over time, such as daily or other suitable periods, to detect virus. If virus is detected, particularly at a level above a control, such as the level immediately after treatment or compared to an established standard, it can be concluded the virus is going to have an ameliorative effect. The virus that is administered to a subject, typically is administered at a therapeutically effective dosage, but a lower dosage can be administered in order to assess whether the virus is suitable for treatment of a particular tumor or particular subject, before administering a therapeutic dosage. Subjects include any mammal, particularly humans, but also include other mammals, including but not limited to, domesticated animals and wild animals, such as pets and zoo animals. Hence, the methods herein have veterinary applications.

Hence provided are methods in which a body fluid sample is tested to detect virus. Testing typically is performed at a pre-determined time or periodically following administration of the virus. Detection of virus, indicates that the tumor cells are infected, which is indicative that the treatment is or will be efficacious. Testing typically is performed on a body fluid sample in vitro after obtaining or providing the body fluid sample from a subject. Also, testing can be effected by obtaining a body fluid sample from a subject and contacting the sample with virus to assess whether the virus infects any tumor cells in the sample. Generally, prior to testing the body fluid sample, administering the oncolytic reporter virus to the subject. As noted, the oncolytic virus can be administered at therapeutic dosages or it at a dosage sufficient to be detected that is lower than a treatment dosage. Exemplary dosage ranges are selected from among, 1×10² pfu to 1×10⁸ pfu, or is administered in an amount that is at least or at least about or is or is about 1×10² pfu, 1×10³ pfu, 1×10⁴ pfu, 1×10⁵ pfu, 1×10⁶ pfu, 1×10⁷ pfu, 1×10⁸ pfu, 1×10⁹ pfu, 1×10¹° pfu, 1×10¹¹ pfu, 1×10¹² pfu or higher. Other exemplary ranges can be selected from among 1×10⁶ pfu to 1×10¹⁴ pfu, or is administered in an amount that is at least or at least about or is or is about 1×10⁸ pfu, 1×10⁹ pfu, 1×10¹° pfu, 1×10¹¹ pfu, 1×10¹² pfu, 1×10¹³ pfu, or 1×10¹⁴ pfu. Dosage depends upon the particular oncolytic virus employed and protocols therefor, and can be determined by a skilled practitioner as needed. If it is determined that a particular virus is likely to be effective or is effective, the reporter gene can be inactivated, removed or replaced or the virus can be used with the reporter. Thus, if the treatment is efficacious as evidenced by the presence of detectable virus in the sample or presence at a level determined to be so-indicative, such as at level greater than within 24 hours after initiating treatment, continuing treatment of the subject by administering an oncolytic virus for treatment, wherein the oncolytic virus is the same oncolytic reporter virus or is an oncolytic virus where the reporter gene is not present or is replaced with a different heterologous nucleic acid. It is understood, that early on in treatment levels of detectable virus in a body fluid should increase. As treatment proceeds, levels of virus, particularly virus in viable cells, ultimately should decrease. Monitoring can be performed throughout the course of treatment to assess effectiveness and/or to monitor the progress of treatment. As treatment progresses, detectable virus should decrease in body fluid samples.

For practicing any of the methods provided herein, the sample can be treated to enrich the concentration or amount of tumor cells to produce an enriched sample prior to testing the sample. Tumor cells also can be isolated for detection. Methods for enriching and/or isolating are well known to those of skill in the art.

Methods for detecting a tumor cell in a body fluid sample are provided. These methods include: a) enriching tumor cells in a body fluid sample from a subject administered with an oncolytic reporter virus to produce an enriched sample; and b) testing the enriched sample for tumor cells that are infected with the oncolytic virus by detecting the oncolytic reporter virus in the sample, thereby detecting tumor cells in the sample. In the methods, wherein enriching tumor cells in a body fluid sample can be effected after obtaining or providing the body fluid sample from a subject. Generally the oncolytic reporter virus is administered prior to enriching tumor cells in a body fluid sample or obtaining the body fluid sample, administering an oncolytic reporter virus to the subject. Embodiments are provided in which the virus is contacted with the sample, typically, after enriching, rather than administering it to the subject.

For all methods herein, as noted above, the oncolytic reporter virus can be administered at a therapeutic dosage or at a lower dosage sufficient to be detected if the virus colonizes and replicates in tumors that is lower than a treatment dosage. Such dosages, include, for example, 1×10² pfu to 1×10⁸ pfu, or is or is about 1×10² pfu, 1×10³ pfu, 1×10⁴ pfu, 1×10⁵ pfu, 1×10⁶ pfu, 1×10⁷ pfu or 1×10⁸ pfu. The oncolytic reporter virus can be administered to the subject in an amount for treatment of a tumor or cancer, such as, for example, but not limited to, 1×10⁶ pfu to 1×10¹⁴ pfu, or is administered in an amount that is at least or at least about or is or is about 1×10⁶ pfu, 1×10⁷ pfu or 1×10⁸ pfu, 1×10⁹ pfu, 1×10¹⁰ pfu, 1×10¹¹ pfu, 1×10¹² pfu, 1×10¹³ pfu, or 1×10¹⁴ pfu. As noted, dosage depends upon the particular oncolytic virus, the subject, the type of tumor(s) and other parameters. If necessary, the skilled practitioner can determine an appropriate dosage.

In these methods, detecting tumor cells can be performed to monitor treatment (or to assess continued efficacy of treatment) of the subject. The amount or level of detected tumor cells is compared to a control sample, such as a predetermined standard, or compared to samples from the subject earlier in time or over time, as an indicator of the progress of treatment. Generally, initially there will be an increase in virus detected, indicating colonization and replication of virus, and, then as treatment progresses, the level or amount can level off or decrease as the virus stabilizes or eliminates tumors or tumor cells.

In practicing the methods, treatment can be modified in accord with the results achieved. For example, early on in treatment, if infected tumor cells in the sample are substantially the same or increased compared to a control, then the treatment can be continued or accelerated; if infected tumor cells in the sample are reduced compared to a control, then the treatment is reduced or discontinued; and if no infected tumor cells are detected, then the treatment is discontinued. As noted, but after treatment has been shown to be effective, then the goal is to eliminate detectable tumor cells in a sample. Controls for this method as well as the other methods provided herein, for example include, but are not limited to, predetermined standards, a sample from a healthy subject, a baseline sample from the subject prior to treatment or immediately following with the oncolytic virus, is a sample from a subject after a previous dose, or is a sample from a subject prior to the last dose of oncolytic virus. Alternatively, for monitoring treatment, samples can be tested over time to assess the levels or to detect virus in cells. As noted, the level of cells initially should increase as the virus infects/colonizes tumors and/or tumor and replicates, but then the levels should decrease or level off as the virus eradicates tumor cells. Typically, the control sample is the same type of bodily fluid sample as the tested sample. In practicing the methods the body fluid sample is tested at a pre-determined time following administration of the virus. The predetermined time should be sufficient for the virus to infect a tumor cell and replicate in the tumor or tumor cell in the subject. The predetermined time can be long enough for free virus, such as virus administered intravenously, to clear from non-tumor tissues. It is not necessary for such to occur, since comparison with an appropriate control can eliminate inclusion of such background or baseline levels of virus. The predetermined time for assessing efficacy or monitoring therapy can be at 6 more hours after administration of the initial dosage of the virus. Generally for monitoring efficacy, it is less than one month or less than about a month following administration of the virus. For monitoring therapy, it can be performed throughout the course of therapy and subsequent to therapy, since the presence of any tumor cells in body fluids can be an indicator that the tumor is disseminating or metastasizing. In addition, the presence of tumor cells in the body fluid can be indicative of the recurrence of a tumor. These cells can be detected early in the progress of such recurrence permitting early detection. In addition, the methods provided herein, also can be used to detect or diagnose cancer or a tumor.

For practicing the methods, the predetermined time can be at least or no more than 6 hours, 12 hours, 18 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days or 24 days following administration of the virus. A body fluid sample is obtained a plurality of times at successive time points following administration of the virus, whereby a plurality of samples are obtained from the subject. A body fluid sample can be assessed at a predetermined time or times after each successive administration of the virus in a cycle of administration.

Provided are methods for detecting a tumor cell in a body fluid sample in which a body fluid sample from a subject is tested by: a) enriching tumor cells from the sample to produce an enriched sample; b) contacting tumor cells from the sample with an oncolytic reporter virus; and c) detecting the oncolytic reporter virus, thereby detecting tumor cells in the sample. Detecting tumor cells in a sample indicates that the oncolytic virus is a candidate for treatment of the tumor and/or indicates that the subject is a candidate for treatment with the oncolytic virus.

The methods herein also can be adapted or employed for prognosing a cancer. The stage of a cancer can be determined. Also, the presence of and/or level of cancer stem cells, which cells have been associated with a poorer prognosis can be detected/determined. Exemplary of such methods is method in which a body fluid sample from a subject by is obtained. The sample can be contacted with an oncolytic reporter virus, or the oncolytic virus can be administered to the subject and then the body fluid sample obtained. The presence of cancer stem cells can be identified by: i) detecting the oncolytic reporter virus to identify cells infected with the virus and from the identified cells identifying stem cells; and/or ii) identifying stem cells and from among the identified stem cells identifying cells infected with virus, whereby the presence of cancer stem cells is indicative of the presence of an aggressive cancer. Stem cells can be identified by methods known to those of skill in the art. For example, stem cells can be identified by detecting a stem cell marker, such as, for example, expression of aldehyde dehydrogenase (ALDH1). The method optionally includes enriching tumor cells in the sample to produce an enriched sample. Contacting cells with an oncolytic reporter virus in embodiments in which virus is contacted with the sample in vivo, can be performed before or after enriching tumor cells from the sample. When it is performed prior to enriching the tumor cells from the sample, the sample can be contacted with the virus at least or at 0.5, 1, 2, 3, 4, 5, 6, 7, 8, 9, 10, 12, 14, 16, 18, 20, 22, or 24 hours prior to enriching the tumor cells. Virus is contacted with cells at a suitable multiplicity of infection (moi), such as at least about or at 0.00001 to 10.0, 0.01 to 10, and 0.0001 to 1.0, or any suitable or empirically determined moi. The methods for detecting tumor cells can include treatment of the subject from whom a sample is obtained by administering an oncolytic virus for treatment of the subject. This includes oncolytic virus that is the same oncolytic reporter virus or is an oncolytic virus where the reporter gene is not present or is replaced by a different heterologous nucleic acid. Dosages are as noted above.

In connection with all methods provided herein, the oncolytic virus can be administered at least one time over a cycle of administration or several times and for a single cycle and a plurality of cycles. For example, in some instances, such as administration of LIVP, including the exemplary virus GLV-1h68 (having a genome set forth in SEQ ID NO:1), the oncolytic virus is administered in an amount that is at least 1×10⁹ pfu at least one time over a cycle of administration. A cycle of administration can be at least or is two days, three days, four days, five days, six days, seven days, 14 days, 21 days or 28 days. In each cycle, the amount of virus is administered two times, three times, four times, five times, six times or seven times over the cycle of administration. Exemplary of cycles, the virus can be administered on the first day of the cycle, the first and second day of the cycle, each of the first three consecutive days of the cycle, each of the first four consecutive days of the cycle, each of the first five consecutive days of the cycle, each of the first six consecutive days of the cycle, or each of the first seven consecutive days of the cycle.

For the methods herein, enriching tumor cells from the sample include selecting tumor cells from the sample or removing non-tumor cells from the fluid sample. Exemplary enrichment can remove about 10%, 20%, 30%, 40%, 50%, 60%, 70%, 80%, 90%, 95%, 96%, 97%, 98%, 99% of non-tumor cells from the sample or can retain at least 50%, 60%, 70%, 80%, 90%, 95%, 96%, 97%, 98%, 99% of tumor cells from the sample. For example, in an embodiment, the sample can be a blood sample, and enriching tumor cells is effected by a method that includes lysis of erythrocytes in the sample. Enriching tumors can be effected by any method known to those of skill in the art. These methods include, but are not limited to, capturing or selecting cells based upon larger size, shear modulus, increased stiffness, reduced deformability, increased density, expression of a surface moiety or moieties or other markers.

Methods of enriching tumor cells include, for example, separating tumor cells from non-tumor cells with a device adapted to sort or separate cells based on physical properties. These include, for example, microfluidic devices, microfilters, density gradients for separation, immunomagnetic separation methods and acoustophoresis. A plurality of methods can be employed. Microfluidic devices can include isolation wells or loci, such as in an array. Each well or locus can include: a cell trap that prevents the passage of tumor cells and permits the passage of non-tumor cells and other components of the fluid sample; or a cell trap that prevents the passage of non-tumor cells and permits the passage of tumor cell in the fluid sample. Separation in a microfluidic device or other suitable device or medium can separate tumor cells based on deformability, size or stiffness. An exemplary microfluidic device contains one or more linear channels, where: each linear channel has a length and a cross-section of a height and a width defining an aspect ratio adapted to isolate tumor cells along at least one portion of the cross-section of the channel based on reduced deformability or larger size of tumor cells as compared to non-tumor cells; and tumor cells flow along a first portion of the channel to a first outlet and non-tumor cells flow along a second portion of the channel to a second outlet.

Enriching can be effected by separation from non-tumor cells based on expression of a moiety on the tumor cell surface, such as chip or bead that contains an immobilized capturing agent that binds to a moiety on a tumor cell surface moiety, such as, but are not limited to, cytokeratin, epithelial cell adhesion molecule (designated EpCAM) or other tumor antigen or marker. Capturing agents include, but are not limited to, an antibody, an antibody fragment, a receptor or a ligand binding domain. Exemplary of such capturing agents are anti-tumor antibodies, such as an anti-EpCAM antibody and antigen binding fragments thereof. Enrichment can be effected by processing the sample through a microfilter, such as a microfilter that contains a plurality, such as an array, of pores of a predetermined shape and size.

In some examples, the capturing agent is immobilized, such as on a solid support, such as a solid support described herein, including a magnetic bead, and enrichment is effected by separating the solid support from the sample. Capturing agents also include, for example, antibodies and antigen-binding fragments thereof that immunospecifically bind to a protein expressed on the surface of the tumor cell. In some examples, the capturing agent binds to a protein encoded by the oncolytic virus and expressed on the surface of a cell infected by the virus.

In some examples, the protein encoded by the oncolytic virus is a cell surface protein, including but not limited to, transporter proteins. Exemplary transporter proteins that can be encoded by the viruses provided herein are listed elsewhere herein and include, for example, a norepinephrine transporter (NET) and a sodium iodide symporter (NIS). Exemplary viruses that encode the human norepinephrine transporter (hNET) include, but are not limited to, GLV-1h99, GLV-1h100, GLV-1h101, GLV-1h139, GLV-1h146 and GLV-1h150 (see, e.g., U.S. Patent Publication No. US-2009-0117034). Exemplary viruses provided herein that encode the human sodium iodide symporter (hNIS) include, but are not limited to, GLV-1h151, GLV-1h152 and GLV-1h153 (see, e.g., U.S. Patent Publication No. US-2009-0117034). All are derivatives of GLV-1h68.

GLV-1h151, GLV-1h151 and GLV-1h153 encode hNIS under the control of a vaccinia synthetic early promoter, vaccinia synthetic early/late promoter or vaccinia synthetic late promoter, respectively, in place of the gusA expression cassette at the HA locus in GLV-1h68. For example, the capturing agent, including, for example antibodies and antigen-binding fragments thereof provided herein, binds to the extracellular domain of NIS.

Provided are antibodies and antigen-binding fragments thereof that immunospecifically bind to the extracellular domain of NIS. In some examples, an antibody provided herein that binds to the extracellular domain of NIS binds to an amino acid sequence within a region of NIS having the sequence RGVMLVGGPRQVLTLAQNHSRINLMDFNPDPRSR (SEQ ID NOS: 50), YPPSEQTMRVLPSSAARCVALSVNASGLLDPALLPANDSSRAPSSGMDASRPALADS FYA (SEQ ID NO: 51), NHSRINLMDFNPDP (SEQ ID NO: 52) or PSEQTMRVLPSSAA (SEQ ID NO: 54); or to

an amino acid sequence corresponding to the sequence RGVMLVGGPRQVLTLAQNHSRINLMDFNPDPRSR (SEQ ID NOS: 50), YPPSEQTMRVLPSSAARCVALSVNASGLLDPALLPANDSSRAPSSGMDASRPALADS FYA (SEQ ID NO: 51), NHSRINLMDFNPDP (SEQ ID NO: 52) or PSEQTMRVLPSSAA (SEQ ID NO: 54) in a NIS polypeptide set forth in SEQ ID NO: 46. In some examples, an antibody provided herein that binds to the extracellular domain of NIS can bind to amino acids 225-238, 468-481 or 502-515 of NIS or a region corresponding to amino acids 225-238, 468-481 or 502-515 of a polypeptide set forth in SEQ ID NO: 46.

Also provided herein are methods for preparing antibodies that bind to the extracellular domain, particular, the portion thereof that can be captured by an antibody of a transporter protein, such as a NIS protein. Methods for preparing antibodies that bind to the extracellular domain of NIS include any methods for preparing antibodies known in the art or described herein. For example, antibodies that bind to the extracellular domain of NIS can be prepared as polyclonal antibodies or monoclonal antibodies.

Provided are antibodies that specifically binds to the extracellular domain of NIS. In particular, the antibodies bind to cells infected with an oncolytic virus that expresses the NIS protein. Also provided are isolated polypeptides that contain the sequence 238, 468-481 and/or 502-515 of hNIS (SEQ ID NO: 46) but do not comprise the complete extracellular domain of NIS. In particular, provided are such polypeptides that contain residues 225-238, 468-481 or 502-515 of hNIS (SEQ ID NO:46) or a corresponding region in a non-human NIS are provided. Immunizing polypeptides that contain residues 225-238, 468-481 or 502-515 of hNIS or a corresponding region in a non-human NIS conjugated to a hapten for immunization are provided. Antibodies that specifically bind to these polypeptides also are provided.

Detection of virus in a sample can be effected by any suitable method. The method depends upon the particular reporter selected. Methods, include, but are not limited to those that detect light or electromagnetic radiation, such as, flow cytometry, fluorescence microscopy, fluorescence spectroscopy, magnetic resonance spectroscopy and luminescence spectroscopy.

For the methods herein, fluid samples the body fluid sample is a sample from blood, lymph, bone marrow fluid, pleural fluid, peritoneal fluid, spinal fluid, abdominal fluid, pancreatic fluid, cerebrospinal fluid, brain fluid, ascites, urine, saliva, bronchial lavage, bile, sweat, tears, ear flow, sputum, semen, vaginal flow, milk, amniotic fluid, or secretions of respiratory, intestinal or genitourinary tract. Exemplary of such samples, is a peripheral blood sample, and a body fluid sample that contains dissociated bone marrow cells from a bone marrow biopsy. The volume of sample is any that is convenient for testing, such as at least about 0.01 mL to about 50 mL or 100 ml.

For the methods herein, subjects include human and non-human animals, such as an ape, monkey, mouse, rat, rabbit, ferret, chicken, goat, cow, deer, zebra, giraffe, sheep, horse, pig, dog and cat. The subjects to be tested include those known to have cancer and, particularly for methods of detecting tumors, those who are screened for cancer and those suspected of having cancer. Cancers include, but are not limited to, cancer of the lung, breast, colon, brain, prostate, liver, pancreas, esophagus, kidney, stomach, thyroid, bladder, uterus, cervix or ovary. Also included are blood and bone marrow cancers, such leukemias, and solid tumors and both. Included are metastatic cancers.

Oncolytic virus and oncolytic reporter viruses include any oncolytic virus, such as vaccinia viruses and other pox viruses, vesticular stomatitis virus (VSV), oncolytic adenoviruses and herpes viruses. Exemplary of vaccinia viruses are Lister strain viruses and Wyeth strain viruses and derivatives thereof, such as GLV-1 h68 and derivatives thereof (Genelux Corporation) and JX-594 (Jennerex Biotherapeutics). Lister strain viruses include LIVP and derivatives thereof, such as derivatives that contain nucleic acid encoding a heterologous gene product. The heterologous gene product can be inserted into or in place of a non-essential gene or region in the genome of the virus or in other locus in which it can be expressed without eliminating replication of the virus. For the LIVP strain, loci for insertion, include, but are not limited to, at or in or in place of the hemagglutinin (HA), thymidine kinase (TK), F14.5L, vaccinia growth factor (VGF), A35R, N1L, E2L/E3L, K1L/K2L, superoxide dismutase locus, 7.5K, C7-K1 L, B13R+B14R, A26L or 14L gene locus in the genome of the virus.

Exemplary LIVP virus is one that includes a sequence of nucleotides set forth in SEQ ID NO:2, or a sequence of nucleotides that has at least 95% sequence identity to SEQ ID NO:2 and derivatives thereof that contain insertions and deletions to modulate toxicity and/or to introduce encoded reporters and/or therapeutic products. Viruses include, but are not limited to, clonal strains of LIVP and modified forms that contain insertions or deletions. Exemplary of such clonal strains, are viruses that contain a sequence of nucleotides selected from among: a) nucleotides 2,256-180,095 of SEQ ID NO: 36, nucleotides 11,243-182,721 of SEQ ID NO: 37, nucleotides 6,264-181,390 of SEQ ID NO: 38, nucleotides 7,044-181,820 of SEQ ID NO: 39, nucleotides 6,674-181,409 of SEQ ID NO:40, nucleotides 6,716-181,367 of SEQ ID NO:41 or nucleotides 6,899-181,870 of SEQ ID NO: 42; and b) a sequence of nucleotides that has at least 97% sequence identity to a sequence of nucleotides 2,256-180,095 of SEQ ID NO: 36, nucleotides 11,243-182,721 of SEQ ID NO: 37, nucleotides 6,264-181,390 of SEQ ID NO: 38, nucleotides 7,044-181,820 of SEQ ID NO: 39, nucleotides 6,674-181,409 of SEQ ID NO: 40, nucleotides 6,716-181,367 of SEQ ID NO: 41 or nucleotides 6,899-181,870 of SEQ ID NO: 42. The clonal strain can include a left and/or right inverted terminal repeat. Particular exemplary viruses, include, but are not limited to, vaccinia virus and modified forms that contain a sequence of nucleotides set forth in any of SEQ ID NOS: 36-42, a sequence of nucleotides that has at least 97% sequence identity to a sequence of nucleotides set forth in any of SEQ ID NO: 36-42. The viruses can be modified, if necessary to encode a reporter gene product.

Other oncolytic viruses include, but are not limited to, the LIVP viruses and derivatives whose sequence includes sequence of nucleotides selected from among any of SEQ ID NOS:1 and 3-7, or a sequence of nucleotides that exhibits at least 99% sequence identity to any of SEQ ID NOS: 1 and 3-7. As described herein, practice of the methods are exemplified with the virus GLV-1h68 (also referred to as GL-ONC1), which is an LIVP virus. Such virus is exemplary only because the methods herein detect infection/colonization by a virus and replication in a tumor. Such properties are not unique to the exemplified virus, but are properties shared by oncolytic viruses. Hence, demonstration of the methods with the virus designated GLV-1h68, evidences and shows practice of any of the methods with an oncolytic virus. Whether the virus is efficacious or not can be determined by the methods herein; it is not necessary that such virus have been proven effective.

Typically, a reporter gene product is inserted into or in place of a non-essential gene or region in the genome of the virus. Exemplary reporters include any known to those of skill in the art, such as, but are not limited to, a fluorescent protein, a bioluminescent protein, a receptor and an enzyme. The fluorescent protein can be selected, for example, from among a green fluorescent protein, an enhanced green fluorescent protein, a blue fluorescent protein, a cyan fluorescent protein, a yellow fluorescent protein, a red fluorescent protein and a far-red fluorescent protein. Exemplary of a fluorescent protein green or red fluorescent proteins, and mutant forms thereof, is the protein designated TurboFP635 (Katushka, available from Evrogen, Moscow, RU; see, also, e.g., Shcherbo et al. (2007) Nat Methods 4:741-746), which is a readily detectable in vivo far-red mutant of the red fluorescent protein from sea anemone Entacmaea quadricolor. Other exemplary reporter enzymes, include, but are not limited to, for example, a luciferase, β-glucuronidase, β-galactosidase, chloramphenicol acetyl tranferase (CAT), alkaline phosphatase, and horseradish peroxidase. Enzymes can be detected by detecting of the product of a substrate whose reaction is catalyzed by the enzyme. Other reporters include, but are not limited to, a receptor that binds to detectable moiety or a ligand attached to a detectable moiety, such as, for example, radiolabel, a chromogen or a fluorescent moiety. Reporter genes typically are operatively linked to a promoter, including a constitutive or inducible promoter. As noted, the viruses that are administered are oncolytic viruses. Generally oncolytic viruses effect treatment by replicating in tumors or tumor cells resulting in lysis. Other activities can be introduced and/or anti-tumor activity can be enhanced by including nucleic acid encoding a heterologous gene product that is a therapeutic and/or diagnostic agent or agents. Exemplary thereof are gene products selected from among an anticancer agent, an anti-metastatic agent, an antiangiogenic agent, an immunomodulatory molecule, an antigen, a cell matrix degradative gene, genes for tissue regeneration and reprogramming human somatic cells to pluripotency, enzymes that modify a substrate to produce a detectable product or signal or are detectable by antibodies, proteins that can bind a contrasting agent, genes for optical imaging or detection, genes for positron emission tomography (PET) imaging and genes encoding products that are detectable by magnetic resonance imaging (MM).

Provided are methods for detecting infected tumor cells, such as in a body fluid, or monitoring treatment or any of the other methods provided herein in which infected cells are identified, where the oncolytic virus encodes a protein that is expressed on the surface of the infected cell; and detection of the virus is effected by detecting the protein expressed on the surface of the infected cell. Cell surface proteins include any cell surface receptors, such as but are not limited to, transporter proteins, such as norepinephrine transporter (NET) or the sodium iodide symporter (NIS), including human NIS or NET protein. Detection can be effected by contacting the cells or a cell sample, such as fluid sample or biopsy, with an antibody that specifically binds to an epitope on the extracellular domain of the protein expressed on the cell surface. The antibody includes polyclonal antibody preparations and also monoclonal antibodies or antigen binding fragments thereof. The antibodies or fragments thereof can be immobilized on a solid support, such as a magnetic bead. This permits separating cells that express the cell surface protein from other cells in a sample to thereby isolate or enrich for virus-infected cells.

Also provided are antibodies that specifically bind to the extracellular domain of NIS as expressed in cell, where the NIS protein is encoded by an oncolytic virus that has infected the cells that express the NIS protein. Also provided are isolated polypeptides that include sequence NDSSRAPSSGMDAS (SEQ ID NO: 53) or an epitope contained therein (or a sequence corresponding to that set forth in SEQ ID NO: 53 from different NIS protein, where corresponding sequences are identified by alignment), where the polypeptide does not comprise the complete extracellular domain of NIS. Thus provided are antibodies that specifically bind to these polypeptides and also binds to an epitope on the extracellular domain of NIS when expressed on the surface of a cell.

Provided are antibodies (monoclonal, polyclonal, and antigen-binding fragments of antibodies) that specifically bind to an epitope within a region corresponding to amino acids 502-515 of the NIS polypeptide of SEQ ID NO: 46. Also provided are conjugates containing amino acids 502-515 of hNIS or a corresponding region from a non-human NIS conjugated directly or indirectly to a hapten, such as via a polypeptide linker. Haptens include any known to those of skill in the art, such as the hapten keyhole limpet hemocyanin.

Methods are provided herein for detection of leptomeningeal metastases (LM), which result from the spread of metastatic tumor cells to the cerebrospinal fluid (CSF) and leptomeninges. Methods are provided herein to detect and diagnose LM, and also to effect treatment thereof. Methods are provided herein to detect peritoneal carcinomatosis (PC), which is the locoregional progression of cancers of gastrointestinal and gynecological origins. As exemplified herein, oncolytic viruses and the methods provided herein effect detection of LM and PC. In addition, the oncolytic virus infects and eliminates tumor cells in LM and PC.

### DETAILED DESCRIPTION

```
Outline
  A. DEFINITIONS
  B. OVERVIEW
    1. Circulating Tumor Cells As Cancer Prognostic and Diagnostic
    Indicators
    2. Existing Methods For Detection of CTCs
    3. Infection of Metastatic Cells and Cancer Stem Cells by Oncolytic
    Viruses
  C. METHODS FOR DETECTING CIRCULATING TUMOR CELLS
  USING ONCOLYTIC REPORTER VIRUSES
    1. Exemplary Methods for Detection of CTCs with an Oncolytic
    Reporter Virus
      a. Ex vivo Detection of CTCs in Samples Treated with an
      Oncolytic Reporter Virus
      b. Ex vivo Detection of CTCs in Samples from Subjects
      Treated with an Oncolytic Reporter Virus
      c. In vivo Detection of CTCs in Subjects Treated with an
      Oncolytic Reporter Virus
    2. Methods for Enrichment of CTCs for Use in Combination with
    an Oncolytic Reporter Virus
      a. Microfiltration
      b. Microfluidic Devices
      c. Immunomagnetic Separation
      d. Acoustophoresis
      e. Dielectrophoresis
      f. Density Gradient Separation
      g. Selective Cell Lysis (RBC lysis of blood cells)
      h. Combinations of Tumor Cell Enrichment Methods
    3. Detection Methods
    4. Samples for Use in the Methods
      a. Sources
      b. Methods of Obtaining Samples
      c. Control Samples
    5. Viruses for Use in the Method
      a. General Characteristics for Virus Selection
      b. Expression of a Reporter Gene Product
        i. Exemplary Reporter Proteins
          (1) Fluorescent proteins
          (2) Bioluminescent proteins
          (3) Other enzymes
          (4) Proteins that bind to detectable ligands
          (5) Transporter Proteins—
            (a) sodium iodide symporter
            (b) norepinephrine transporter
          (6) Proteins detectable by antibodies
          (7) Fusion proteins
          (8) Proteins that interact with host cell
          proteins
        ii. Operable linkage to promoter
          (1) Promoter characteristics
            (a) Viral and host factors
            (b) Exemplary promoters
        iii. Expression of multiple reporter proteins
      c. Further Modifications of the Viruses
        i. Expression of a Therapeutic and other Gene
        Products exemplary products
        ii. Metastatic Genes
      d. Exemplary Oncolytic Reporter Viruses For Use in the
      Methods
        i. Poxviruses
          (1) Vaccinia Viruses
            (a) Modified Vaccinia Viruses
            (b) Exemplary Modified Vaccinia
            Viruses
        ii. Other Oncolytic Viruses
      e. Production and Preparation of Virus
      Methods for Generating Recombinant Virus
    6. Antibodies for capture of tumor cells
      a. General structure of antibodies
        i. Structural and Functional Domains of Antibodies
        ii. Antibody Fragments
      b. Additional modifications of antibodies
        i. Pegylation
      c. Methods for Producing Antibodies
        i. Nucleic Acids
        ii. Purification
    7. Applications of the Method
    8. Additional Analysis of Identified CTCs and Validation of Results
D. THERAPEUTIC METHODS
E. COMBINATIONS, KITS, AND ARTICLES OF MANUFACTURE
F. EXAMPLES
```

### A. DEFINITIONS

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, a circulating tumor cell or CTC refers to a tumor cell derived from a primary cancer site that has detached from the primary tumor mass. CTCs include cancer cells, malignant tumor cells and cancer stem cells. CTCs include any cancer cell or cluster of cancer cells that is found in a fluid sample obtained from a subject. CTCs are often epithelial cells shed from solid tumors. CTCs also can be mesothelial cells from carcinomas or melanocytes from melanomas. A CTC is typically a cell originating from a primary tumor, but also can be a cell shed from a metastatic tumor (e.g., a secondary or tertiary tumor).

As used herein, the term “CTC” is intended to encompass any tumor cell that has detached from a tumor. Thus, as used herein, a CTC encompasses tumor cells found in circulation, such as in the blood or lymph, or in other fluid samples, such as, but not limited to, pleural fluid, peritoneal fluid, central spinal fluid, abdominal fluid, pancreatic fluid, cerebrospinal fluid, brain fluid, ascites, urine, saliva, bronchial lavage, bile, sweat, tears, ear flow, sputum, semen, vaginal flow, milk, amniotic fluid, and secretions of respiratory, intestinal or genitourinary tract. The term CTC as used herein also includes disseminated tumor cells (DTCs) found in the bone marrow.

As used herein, a “tumor cell” is any cell that is part of a tumor or that is shed from a tumor (e.g. a circulating tumor cell). Tumor cells typically are cells undergoing early, intermediate, or advanced stages of neoplastic progression, including a pre-neoplastic cells (i.e. hyperplastic cells and dysplastic cells) and neoplastic cells.

As used herein, a disseminated tumor cell (DTC) typically refers to a tumor cell derived from a primary cancer site that has detached from the primary tumor mass and is found in the bone marrow. For purposes herein, a DTC is defined as a type of CTC. Thus, the methods provided herein for the detection of CTCs encompass detection of DTCs found in the bone marrow.

As used herein, a cancer stem cell (CSC), refers to a sub-population of cancer cells that possesses characteristics normally associated with stem cells, such as self-renewal, the ability to differentiate into multiple cell types and give rise to multiple cancer cell types, indefinite life span due to telomerase activity and abbreviated cell cycle regulation. CSCs are tumorigenic and are capable of forming tumors from very small number of cells in animal tumor models. CSCs can persist in tumors as a distinct sub-population and cause relapse and metastasis by giving rise to new tumors. CSCs also are found in sub-populations in the bone marrow and among subsets of CTCs.

As used herein, a “tumor cell enrichment method” refers to any method that increases the proportion of tumor cells in a sample relative to non-tumor cells. The tumor cell enrichment method can involve separation of tumor cells from non-tumor cells based on a difference in one or more properties of the tumor cells compared to non-tumor cells. For example, a tumor cell enrichment method can involve positive selection and/or negative selection methods. For example, the tumor cell enrichment method can involve positive selection and separation of tumor cells from non-tumor cells and other components of the sample based on one or more properties exhibited by the tumor cell and/or can involve negative selection and removal of non-tumor cells or other components from the sample based on one or more properties exhibited by the non-tumor cells.

As used herein, “enriching tumor cells” in a sample means increasing the proportion of tumor cells in a sample relative to non-tumor cells in the sample including, for example, selection of one or more tumor cells or removal of one or more non-tumor cells to produce an enriched sample. Where one or more tumor cells are selected, the selected tumor cells represent the enriched sample. The enriched sample can include, for example, cells selected in a solution, column, or gradient, cells captured on a microfluidic device or a microfilter, or selected cells on a column, gradient, microfluidic device or a microfilter that have been transferred to a new container or medium.

As used herein, a physical property of a cell refers to any mechanical property of a cell including, but not limited to, size, stiffness, density, shear modulus, deformability and electrical charge.

As used herein a biological property of a cell refers to any property of the cell that relates to the biological activity of the cell including, but not limited to, surface protein expression, viability, and invasiveness.

As used herein, a microfilter refers to any type of filtration device containing an array of pores of a sufficient size to reduce or inhibit the passage of tumor cells through the pore and permit the passage of non-tumor cells through the pore.

As used herein, the term “microfluidic device” refers to a device for handling, processing, ejecting and/or analyzing a fluid sample including at least one channel or chamber having microscale dimensions. For example the typical channels of chambers have at least one cross sectional dimension in the range of about 0.1 microns (μm) to about 1500 μm, such as for example in the range of 0.2 μm to 1000 μm, such as for example in the range of 0.4 μm to 500 μm. Typically, microfluidic chambers of channel hold small quantities of fluid, such as, for example, 10 nanoliters (nL) to 5 milliliters (mL), such as, for example, 200 mL to 500 microliters (μL) such as for example, 500 nL to 200 μL.

As used herein, level or amount of tumor cells in a sample refers to concentration of tumor cells in any given sample (i.e. the number of tumor cells per volume of a fluid sample).

As used herein, cytosine refers to the well known technique by which a single layer of cells is deposited onto defined area of a surface, such as a glass slide. As used herein, a sample refers to a sample containing at least one cell from a subject.

A sample encompasses a body fluid or tissue sample from a subject. A sample can include, for example, buffer solutions, saline solutions, cell culture media; or other components added to the sample for use in the methods.

As used herein, a fluid sample refers to any liquid sample that contains one or more cells from a subject. The fluid sample can be a sample that is a bodily fluid from a subject or can be a liquid cell suspension generated by dispersion of cells from a tissue sample from a subject in a suitable liquid medium.

As used herein, contacting a sample containing cells with a virus means co-incubation of a virus with the sample such that the virus infects one or more tumor cells contained in the sample.

As used herein, a biopsy refers to a tissue sample that is removed from a subject for the purpose of determining if the sample contains cancer cells.

As used herein, morphological analysis refers to visually observable characteristics of a cell, such as size, shape, or the presence or absence of certain features of the cell.

As used herein, a control sample refers to any sample that serves as a reference in the methods provided. For example, the control sample can be a sample with a known level of CTCs, from a subject with a known cancer prognosis, from a subject with a particular cancer, from a subject with a particular stage of cancer, or from a subject without any detectable cancer. The control sample can be a sample from a subject that has not received an anti-cancer therapy. The control sample can be from an individual or from a population pool.

As used herein, a “cycle of administration” refers to the dosing schedule of an oncolytic virus or oncolytic reporter virus, including the duration of the cycle, the number of times of administration of the virus and the timing of administration of the virus. For example, the duration of a cycle of administration can be days, weeks or months, such as two days, three days, four days, five days, six days, seven days, 14 days, 21 days or 28 days. The number of times of administration refers to the number of times the virus is administered over the duration of the cycle. For example, in each cycle, the virus can be administered one time or several times, for example, two times, three times, four times, five times, six times or seven times. The timing of administration refers to when the virus is administered over the duration of the cycle. For example, the virus can be administered on the first day of the cycle, the first and second day of the cycle, each of the first three consecutive days of the cycle, each of the first four consecutive days of the cycle, each of the first five consecutive days of the cycle, each of the first six consecutive days of the cycle, or each of the first seven consecutive days of the cycle. A virus can be administered for one cycle of administration or for a plurality of cycles.

As used herein, “prognosis” refers to a prediction of how a patient will progress, and whether there is a chance of recovery. “Cancer prognosis” as used herein refers to a prediction of the probable course or outcome of the cancer. As used herein, cancer prognosis includes the prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. Prognosis includes prediction of favorable responses to cancer treatments, such as a conventional cancer therapy.

A favorable or poor prognosis can, for example, be assessed in terms of patient survival, likelihood of disease recurrence or disease metastasis. Patient survival, disease recurrence and metastasis can for example be assessed in relation to a defined time point, e.g. at a given number of years after a cancer treatment (e.g. surgery to remove one or more tumors) or after initial diagnosis. In one example, a favorable or poor prognosis can be assessed in terms of overall survival or disease free survival.

As used herein, cancer progression refers to the process by which a cancer develops, for example, from abnormal cell growth to the growth of a tumor to the advancement of the tumor into a malignant and aggressive phenotype. Generally, tumor growth is characterized in stages, or the extent of cancer in the body. Staging is typically based on the size of the tumor, the number of tumors present, whether lymph nodes contain cancer, biological and/or morphological characteristics of the tumor cells (e.g., gene expression profile, gene mutation, chromosomal abnormality, cell size or shape), and whether the cancer has spread from the original site to other parts of the body. Stages of cancer include stage I, stage II, stage III and stage IV. Higher stage numbers generally indicate more extensive disease (e.g. larger tumor size and/or spread of the cancer beyond the organ in which it first developed to nearby lymph nodes and/or organs adjacent to the location of the primary tumor). Staging of cancers is dependent on the cancer type. Guidelines for staging particular cancers are well-known in the art. Early stage cancer, generally Stage I or Stage II cancer, refers to cancers that have been clinically determined to be detected by conventional methods such as, for example, mammography for breast cancer patients or X-ray. Late stage cancer, or Stage IV cancer, typically refers to cancer that has metastasized to surrounding and/or distant organs or other parts of the body.

As used herein, reciting that a treatment is efficacious means that the treatment as assessed by the methods herein at the time of assessment exhibits properties indicative of treatments that are efficacious. Thus, for example, detection of reporter virus in a CTC in a body fluid sample, following, such as within a day or two, systemic administration of the virus to a subject indicates that the virus has infected cells in a tumor and is replicating there such that it appears in tumor cells in circulation. When such is observed, it indicates that the oncolytic virus has infected and begun replicating in tumor cells, and, thus is behaving as an effective treatment.

As used herein, cancer remission refers to the period of time after treatment of a cancer in a subject, where the subject does not exhibit any symptoms of the cancer and the cancer is not detectable (complete remission) or where the subject exhibits a reduction in one or more symptoms of the cancer and a decrease in the number of cancer cells (partial remission).

As used herein, the term “circulating tumor cell marker,” “CTC cell marker” or “CTC specific marker” refers to a nucleic acid or peptide expressed by a gene whose expression level, alone or in combination with other genes, is correlated with the presence of CTCs in a sample. The correlation can relate to either an increased or decreased expression of the gene (e.g. increased or decreased levels of mRNA or the peptide encoded by the gene).

As used herein, the term “cancer stem cell marker” refers to a nucleic acid or peptide expressed by a gene whose expression level, alone or in combination with other genes, is correlated with the presence of cancer stem cells (i.e. tumorigenic cancer cells). The correlation can relate to either an increased or decreased expression of the gene (e.g. increased or decreased levels of mRNA or the peptide encoded by the gene).

As used herein, epithelial to mesenchymal transition, or EMT, refers to the process whereby epithelial-type cells, which are normally immobile, undergo transition into a mesenchymal-type cell characterized by a proliferative and mobile phenotype. In cancer, EMT is involved in tumor invasion and metastasis of epithelial type tumors. During metastasis of a tumor, tumor cells at the invasive front of the primary tumor typically lose expression of one or more cell adhesion molecules, such as E-cadherin, EpCAM and cytokeratin (CK), dissociate from the neighboring epithelial cells, and become single motile cells. Hence, EMT as used herein with respect to tumor cells refers to the metastatic process by which tumor cells acquire the capacity to detach from the primary tumor and invade surrounding tissues and/or enter circulation.

As used herein, the term “EMT marker” refers to a nucleic acid or peptide expressed by a gene whose expression level, alone or in combination with other genes, is correlated with the presence of cells that have undergone epithelial-mesenchymal transition. The correlation can relate to either an increased or decreased expression of the gene (e.g. increased or decreased levels of mRNA or the peptide encoded by the gene).

As used herein, a subject includes any organism, including an animal, for whom diagnosis, screening, monitoring or treatment is contemplated. Animals include mammals, such as, for example, primates, domesticated animals and livestock. An exemplary primate is a human.

A patient refers to a subject, such as a mammal, primate, human, domesticated animal or livestock, or other animal subject afflicted with a disease condition or for which a disease condition is to be determined or risk of a disease condition is to be determined. Typically, a patient refers to a human subject exhibiting symptoms of a disease or disorder.

As used herein, animals include any animal, such as, but are not limited to, primates, including humans, apes and monkeys; rodents, such as mice, rats, rabbits, and ferrets; fowl, such as chickens; ruminants, such as goats, cows, deer, and sheep; horses, pigs, dogs, cats, fish, and other animals. Non-human animals exclude humans as the contemplated animal.

As used herein, cancer recurrence or relapse refers to the return of cancer after treatment and after a period of time during which the cancer cannot be detected. The length of time between when the cancer is undetectable and recurrence can vary. The same cancer can recur at the same site of original tumor growth or at a different location in the body. For example, prostate cancer can return in the area of the prostate gland, even if the gland was removed, or it can recur in the bone marrow.

As used herein, the term “subject suspected of having cancer” refers to a mammal, typically a human, who is being tested or screened for cancer. Generally such subjects, present a symptoms indicative of a cancer (e.g., a noticeable lump or mass) or is being screened for a cancer (e.g., during a routine physical). A subject suspected of having cancer also can have one or more risk factors, such as the presence of a genetic marker indicative of risk of a cancer. A “subject suspected of having cancer” encompasses an individual who has received an initial diagnosis but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, and previous incidents of cancer, preexisting non-cancer diseases, and lifestyle.

As used herein, the term “suffering from disease” refers to a subject (e.g., a human) that is experiencing a particular disease. It is not intended that the methods provided be limited to any particular signs or symptoms, nor disease. Thus, it is intended that the methods provided encompass subjects that are experiencing any range of disease, from sub-clinical to full-blown disease, wherein the subject exhibits at least some of the indicia (e.g., signs and symptoms) associated with the particular disease.

As used herein, the term “subject diagnosed with a cancer” refers to a subject who has been tested and found to have cancerous cells. The cancer can be diagnosed using any suitable method, including but not limited to, biopsy, x-ray, MRI, PET, blood test, and the diagnostic methods provided herein.

As used herein, a “metastatic cell” is a cell that has the potential for metastasis. Metastatic cells have the ability to metastasize from a first tumor in a subject and can colonize tissue at a different site in the subject to form a second tumor at the site.

As used herein, “metastasis” refers to the spread of cancer from one part of the body to another. For example, in the metastatic process, malignant cells can spread from the site of the primary tumor in which the malignant cells arose and move into lymphatic and blood vessels, which transport the cells to normal tissues elsewhere in an organism where the cells continue to proliferate. A tumor formed by cells that have spread by metastasis is called a “metastatic tumor,” a “secondary tumor” or a “metastasis.”

As used herein, “tumorigenic cell,” is a cell that, when introduced into a suitable site in a subject, can form a tumor. The cell can be non-metastatic or metastatic.

As used herein, a “normal cell” or “non-tumor cell” are used interchangeably and refer to a cell that is not derived from a tumor.

As used herein, the term “cell” refers to the basic unit of structure and function of a living organism as is commonly understood in the biological sciences. A cell can be a unicellular organism that is self-sufficient and that can exist as a functional whole independently of other cells. A cell also can be one that, when not isolated from the environment in which it occurs in nature, is part of a multicellular organism made up of more than one type of cell. Such a cell, which can be thought of as a “non-organism” or “non-organismal” cell, generally is specialized in that it performs only a subset of the functions performed by the multicellular organism as whole. Thus, this type of cell is not a unicellular organism. Such a cell can be a prokaryotic or eukaryotic cell, including animal cells, such as mammalian cells, human cells and non-human animal cells or non-human mammalian cells. Animal cells include any cell of animal origin that can be found in an animal. Thus, animal cells include, for example, cells that make up the various organs, tissues and systems of an animal.

As used herein an “isolated cell” is a cell that exists in vitro and is separate from the organism from which it was originally derived.

As used herein, a “cell line” is a population of cells derived from a primary cell that is capable of stable growth in vitro for many generations. Cell lines are commonly referred to as “immortalized” cell lines to describe their ability to continuously propagate in vitro.

As used herein a “tumor cell line” is a population of cells that is initially derived from a tumor. Such cells typically have undergone some change in vivo such that they theoretically have indefinite growth in culture; unlike primary cells, which can be cultured only for a finite period of time. Such cells can form tumors after they are injected into susceptible animals.

As used herein, a “primary cell” is a cell that has been isolated from a subject.

As used herein, a “host cell” or “target cell” are used interchangeably to mean a cell that can be infected by a virus.

As used herein, the term “tissue” refers to a group, collection or aggregate of similar cells generally acting to perform a specific function within an organism.

As used herein, “virus” refers to any of a large group of infectious entities that cannot grow or replicate without a host cell. Viruses typically contain a protein coat surrounding an RNA or DNA core of genetic material, but no semipermeable membrane, and are capable of growth and multiplication only in living cells. Viruses include, but are not limited to, poxviruses, herpesviruses, adenoviruses, adeno-associated viruses, lentiviruses, retroviruses, rhabdoviruses, papillomaviruses, vesicular stomatitis virus, measles virus, Newcastle disease virus, picornavirus, Sindbis virus, papillomavirus, parvovirus, reovirus, coxsackievirus, influenza virus, mumps virus, poliovirus, and semliki forest virus.

As used herein, oncolytic viruses refer to viruses that replicate selectively in tumor cells in tumorous subjects. Some oncolytic viruses can kill a tumor cell following infection of the tumor cell. For example, an oncolytic virus can cause death of the tumor cell by lysing the tumor cell or inducing cell death of the tumor cell.

As used herein the term “vaccinia virus” or “VACV” denotes a large, complex, enveloped virus belonging to the poxvirus family. It has a linear, double-stranded DNA genome approximately 190 kbp in length, and which encodes approximately 200 proteins. Vaccinia virus strains include, but are not limited to, strains of, derived from, or modified forms of Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health vaccinia virus strains.

As used herein, Lister Strain of the Institute of Viral Preparations (LIVP) or LIVP virus strain refers to a virus strain that is the attenuated Lister strain (ATCC Catalog No. VR-1549) that was produced by adaption to calf skin at the Institute of Viral Preparations, Moscow, Russia (Al'tshtein et al. (1985) Dokl. Akad. Nauk USSR 285:696-699). The LIVP strain can be obtained, for example, from the Institute of Viral Preparations, Moscow, Russia (see. e.g., Kutinova et al. (1995) Vaccine 13:487-493); the Microorganism Collection of FSRI SRC VB Vector (Kozlova et al. (2010) Environ. Sci. Technol. 44:5121-5126); or can be obtained from the Moscow Ivanovsky Institute of Virology (C0355 K0602; Agranovski et al. (2006) Atmospheric Environment 40:3924-3929). It also is well known to those of skill in the art; it was the vaccine strain used for vaccination in the USSR and throughout Asia and India. The strain is used by researchers and is well known (see e.g., Altshteyn et al. (1985) Dokl. Akad. Nauk USSR 285:696-699; Kutinova et al. (1994) Arch. Virol. 134:1-9; Kutinova et al. (1995) Vaccine 13:487-493; Shchelkunov et al. (1993) Virus Research 28:273-283; Sroller et al. (1998) Archives Virology 143:1311-1320; Zinoviev et al. (1994) Gene 147:209-214; and Chkheidze et al. (1993) FEBS 336:340-342). Among the LIVP strains is one that contains a genome having a sequence of nucleotides set forth in SEQ ID NO: 2, or a sequence that is at least or at least about 99% identical to the sequence of nucleotides set forth in SEQ ID NO: 2.

As used herein, an LIVP clonal strain or LIVP clonal isolate refers to a virus that is derived from the LIVP virus strain by plaque isolation, or other method in which a single clone is propagated, and that has a genome that is homogenous in sequence. Hence, an LIVP clonal strain includes a virus whose genome is present in a virus preparation propagated from LIVP. An LIVP clonal strain does not include a recombinant LIVP virus that is genetically engineered by recombinant means using recombinant DNA methods to introduce heterologous nucleic acid. In particular, an LIVP clonal strain has a genome that does not contain heterologous nucleic acid that contains an open reading frame encoding a heterologous protein. For example, an LIVP clonal strain has a genome that does not contain non-viral heterologous nucleic acid that contains an open reading frame encoding a non-viral heterologous protein. As described herein, however, it is understood that any of the LIVP clonal strains provided herein can be modified in its genome by recombinant means to generate a recombinant virus. For example, an LIVP clonal strain can be modified to generate a recombinant LIVP virus that contains insertion of nucleotides that contain an open reading frame encoding a heterologous protein.

As used herein, LIVP 1.1.1 is an LIVP clonal strain that has a genome having a sequence of nucleotides set forth in SEQ ID NO: 36 or a genome having a sequence of nucleotides that has at least 99% sequence identity to the sequence of nucleotides set forth in SEQ ID NO: 36.

As used herein, LIVP 2.1.1 is an LIVP clonal strain that has a genome having a sequence of nucleotides set forth in SEQ ID NO: 37, or a genome having a sequence of nucleotides that has at least 99% sequence identity to the sequence of nucleotides set forth in SEQ ID NO: 37.

As used herein, LIVP 4.1.1 is an LIVP clonal strain that has a genome having a sequence of nucleotides set forth in SEQ ID NO: 38, or a genome having a sequence of nucleotides that has at least 99% sequence identity to the sequence of nucleotides set forth in SEQ ID NO: 38.

As used herein, LIVP 5.1.1 is an LIVP clonal strain that has a genome having a sequence of nucleotides set forth in SEQ ID NO: 39, or a genome having a sequence of nucleotides that has at least 99% sequence identity to the sequence of nucleotides set forth in SEQ ID NO: 39.

As used herein, LIVP 6.1.1 is an LIVP clonal strain that has a genome having a sequence of nucleotides set forth in SEQ ID NO: 40, or a genome having a sequence of nucleotides that has at least 99% sequence identity to the sequence of nucleotides set forth in SEQ ID NO: 40.

As used herein, LIVP 7.1.1 is an LIVP clonal strain that has a genome having a sequence of nucleotides set forth in SEQ ID NO: 41, or a genome having a sequence of nucleotides that has at least 99% sequence identity to the sequence of nucleotides set forth in SEQ ID NO: 41.

As used herein, LIVP 8.1.1 is an LIVP clonal strain that has a genome having a sequence of nucleotides set forth in SEQ ID NO: 42, or a genome having a sequence of nucleotides that has at least 99% sequence identity to the sequence of nucleotides set forth in SEQ ID NO: 42.

As used herein, multiplicity of infection (MOI) refers to the ratio of viral particles to cells used for infection. For example, infection at a MOI of 1 mean that virus is added to a sample of cells at a ratio of 1 virus particle to one cell.

As used herein, the term “modified virus” refers to a virus that is altered compared to a parental strain of the virus. Typically modified viruses have one or more truncations, mutations, insertions or deletions in the genome of virus. A modified virus can have one or more endogenous viral genes modified and/or one or more intergenic regions modified. Exemplary modified viruses can have one or more heterologous nucleic acid sequences inserted into the genome of the virus. Modified viruses can contain one or more heterologous nucleic acid sequences in the form of a gene expression cassette for the expression of a heterologous gene.

As used herein, a modified LIVP virus strain refers to an LIVP virus that has a genome that is not contained in LIVP, but is a virus that is produced by modification of a genome of a strain derived from LIVP. Typically, the genome of the virus is modified by substitution (replacement), insertion (addition) or deletion (truncation) of nucleotides. Modifications can be made using any method known to one of skill in the art such as genetic engineering and recombinant DNA methods. Hence, a modified virus is a virus that is altered in its genome compared to the genome of a parental virus. Exemplary modified viruses have one or more heterologous nucleic acid sequences inserted into the genome of the virus. Typically, the heterologous nucleic acid contains an open reading frame encoding a heterologous protein. For example, modified viruses herein can contain one or more heterologous nucleic acid sequences in the form of a gene expression cassette for the expression of a heterologous gene.

As used herein a “gene expression cassette” or “expression cassette” is a nucleic acid construct, containing nucleic acid elements that are capable of effecting expression of a gene in hosts that are compatible with such sequences. Expression cassettes include at least promoters and optionally, transcription termination signals. Typically, the expression cassette includes a nucleic acid to be transcribed operably linked to a promoter. Expression cassettes can contain genes that encode, for example, a therapeutic gene product, or a detectable protein or a selectable marker gene.

As used herein, a heterologous nucleic acid (also referred to as exogenous nucleic acid or foreign nucleic acid) refers to a nucleic acid that is not normally produced in vivo by an organism or virus from which it is expressed or that is produced by an organism or a virus but is at a different locus, or that mediates or encodes mediators that alter expression of endogenous nucleic acid, such as DNA, by affecting transcription, translation, or other regulatable biochemical processes. Hence, heterologous nucleic acid is often not normally endogenous to a virus into which it is introduced. Heterologous nucleic acid can refer to a nucleic acid molecule from another virus in the same organism or another organism, including the same species or another species. Heterologous nucleic acid, however, can be endogenous, but is nucleic acid that is expressed from a different locus or altered in its expression or sequence (e.g., a plasmid). Thus, heterologous nucleic acid includes a nucleic acid molecule not present in the exact orientation or position as the counterpart nucleic acid molecule, such as DNA, is found in a genome. Generally, although not necessarily, such nucleic acid encodes RNA and proteins that are not normally produced by the virus or in the same way in the virus in which it is expressed. Any nucleic acid, such as DNA, that one of skill in the art recognizes or considers as heterologous, exogenous or foreign to the virus in which the nucleic acid is expressed is herein encompassed by heterologous nucleic acid. Examples of heterologous nucleic acid include, but are not limited to, nucleic acid that encodes exogenous peptides/proteins, including diagnostic and/or therapeutic agents. Proteins that are encoded by heterologous nucleic acid can be expressed within the virus, secreted, or expressed on the surface of the virus in which the heterologous nucleic acid has been introduced.

As used herein, a heterologous protein or heterologous polypeptide (also referred to as exogenous protein, exogenous polypeptide, foreign protein or foreign polypeptide) refers to a protein that is not normally produced by a virus.

As used herein, operative linkage of heterologous nucleic acids to regulatory and effector sequences of nucleotides, such as promoters, enhancers, transcriptional and translational stop sites, and other signal sequences refers to the relationship between such nucleic acid, such as DNA, and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA. Thus, operatively linked or operationally associated refers to the functional relationship of a nucleic acid, such as DNA, with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or transcription, it can be necessary to remove, add or alter 5′ untranslated portions of the clones to eliminate extra, potentially inappropriate, alternative translation initiation (i.e., start) codons or other sequences that can interfere with or reduce expression, either at the level of transcription or translation. In addition, consensus ribosome binding sites can be inserted immediately 5′ of the start codon and can enhance expression (see, e.g., Kozak J. Biol. Chem. 266: 19867-19870 (1991) and Shine and Delgarno, Nature 254(5495):34-38 (1975)). The desirability of (or need for) such modification can be empirically determined.

As used herein, a heterologous promoter refers to a promoter that is not normally found in the wild-type organism or virus or that is at a different locus as compared to a wild-type organism or virus. A heterologous promoter is often not endogenous to a virus into which it is introduced, but has been obtained from another virus or prepared synthetically. A heterologous promoter can refer to a promoter from another virus in the same organism or another organism, including the same species or another species. A heterologous promoter, however, can be endogenous, but is a promoter that is altered in its sequence or occurs at a different locus (e.g., at a different location in the genome or on a plasmid). Thus, a heterologous promoter includes a promoter not present in the exact orientation or position as the counterpart promoter is found in a genome.

A synthetic promoter is a heterologous promoter that has a nucleotide sequence that does not occur in nature. A synthetic promoter can be a nucleic acid molecule that has a synthetic sequence or a sequence derived from a native promoter or portion thereof. A synthetic promoter also can be a hybrid promoter composed of different elements derived from different native promoters.

As used herein, a “reporter gene” is a gene that encodes a reporter molecule that can be detected when expressed by a virus provided herein or encodes a molecule that modulates expression of a detectable molecule, such as nucleic acid molecule or a protein, or modulates an activity or event that is detectable. Hence reporter molecules include, nucleic acid molecules, such as expressed RNA molecules, and proteins.

As used herein, a “heterologous reporter gene” is a reporter gene that is not natively present in a virus or is a gene that is present at a different locus than in its native locus in a virus. Heterologous reporter genes can contain nucleic acid that is not endogenous to the virus into which it is introduced, but has been obtained from another virus or cell or prepared synthetically. Heterologous reporter genes, however, can be endogenous, but contain nucleic acid that is expressed from a different locus or altered in its expression or sequence. Generally, such reporter genes encode RNA and proteins that are not normally produced by the virus or that are not produced under the same regulatory schema, such as the promoter.

As used herein, a “reporter protein” or “reporter gene product” refers to any detectable protein or product expressed by a reporter gene. Reporter proteins can be expressed from endogenous or heterologous genes. Exemplary reporter proteins are provided herein and include, for example, receptors or other proteins that can specifically bind to a detectable compound, proteins that can emit a detectable signal such as a fluorescence signal, and enzymes that can catalyze a detectable reaction or catalyze formation of a detectable product. Reporter gene products also can include detectable nucleic acids.

As used herein, a “reporter virus” is a virus that expresses or encodes a reporter gene or a reporter protein or a detectable protein or moiety. It is a virus that is detectable in a cell. As used herein, an oncolytic reporter virus is an oncolytic virus that expresses or encodes a reporter gene or a reporter protein or a detectable protein or moiety.

As used herein, “detecting an oncolytic reporter virus” means detecting tumor cells infected by the virus by one or more methods that detect a reporter gene product encoded by the virus that is expressed during infection of the tumor cell. Such methods include, but are not limited to detection of proteins such fluorescent proteins, luminescent proteins or proteins that bind to detectable ligands or antibodies.

As used herein, a fluorescent protein (FP) refers to a protein that possesses the ability to fluoresce (i.e., to absorb energy at one wavelength and emit it at another wavelength). For example, a green fluorescent protein (GFP) refers to a polypeptide that has a peak excitation spectrum at 490 nm or about 490 nm and peak emission spectrum at 510 nm or about 510 nm (expressed herein as excitation/emission 490 nm/510 nm). A variety of FPs that emit at various wavelengths are known in the art. Exemplary FPs include, but are not limited to, a violet fluorescent protein (VFP; peak excitation/emission at or about 355 nm/424 nm), a blue fluorescent protein (BFP; peak excitation/emission at or about 380-400 nm/450 nm), cyan fluorescent protein (CFP; peak excitation/emission at or about 430-460 nm/480-490 nm), green fluorescent protein (GFP; peak excitation/emission at or about 490 nm/510 nm), yellow fluorescent protein (YFP; peak excitation/emission at or about 515 nm/530 nm), orange fluorescent protein (OFP; peak excitation/emission at or about 550 nm/560 nm), red fluorescent protein (RFP; peak excitation/emission at or about 560-590 nm/580-610 nm), far-red fluorescent protein (peak excitation/emission at or about 590 nm/630-650 nm), or near-infrared fluorescent protein (peak excitation/emission at or about 690 nm/713 nm). Extending the spectrum of available colors of fluorescent proteins to blue, cyan, orange, yellow and red variants provides a method for multicolor tracking of proteins.

Examples of fluorescent proteins and their variants include, but are not limited to, GFPs, such as Emerald (EmGFP; Invitrogen, Carlsbad, Calif.), EGFP (Clontech, Palo Alto, Calif.), Azami-Green (MBL International, Woburn, Mass.), Kaede (MBL International, Woburn, Mass.), ZsGreen1 (Clontech, Palo Alto, Calif.) and CopGFP (Evrogen/Axxora, LLC, San Diego, Calif.); CFPs, such as Cerulean (Rizzo, Nat. Biotechnol. 22(4):445-9 (2004)), mCFP (Wang et al. (2004) Proc. Natl. Acad. Sci. USA 101(48):16745-9), AmCyan1 (Clontech, Palo Alto, Calif.), MiCy (MBL International, Woburn, Mass.), and CyPet (Nguyen and Daugherty, Nat. Biotechnol. 23(3):355-60 (2005)); BFPs, such as EBFP (Clontech, Palo Alto, Calif.); YFPs, such as EYFP (Clontech, Palo Alto, Calif.), YPet (Nguyen and Daugherty, Nat. Biotechnol. 23(3):355-60 (2005)), Venus (Nagai et al. Nat. Biotechnol. 20(1):87-90 (2002)), ZsYellow (Clontech, Palo Alto, Calif.), and mCitrine (Wang et al., Proc. Natl. Acad. Sci. USA 101(48):16745-9 (2004)); OFPs, such as cOFP (Strategene, La Jolla, Calif.), mKO (MBL International, Woburn, Mass.), and mOrange; RFPs, such as Discosoma RFP (DsRed) isolated from the corallimorph Discosoma (Matz et al. (1999) Nature Biotechnology 17: 969-973) and Discosoma variants, such as monomeric red fluorescent protein 1 (mRFP1), mCherry, tdTomato, mStrawberry, mTangerine (Wang et al. (2004) Proc. Natl. Acad. Sci. USA 101(48):16745-9), DsRed2 (Clontech, Palo Alto, Calif.), and DsRed-T1 (Bevis and Glick, Nat. Biotechnol., 20: 83-87 (2002)), Anthomedusa J-Red (Evrogen) and Anemonia AsRed2 (Clontech, Palo Alto, Calif.); far-red FPs, such as Actinia AQ143 (Shkrob et al. (2005) Biochem J. 392(Pt 3):649-54), Entacmaea eqFP611 (Wiedenmann et al. (2002) Proc. Natl. Acad. Sci. USA. 99(18):11646-51), Discosoma variants, such as mPlum and mRasberry (Wang et al. (2004) Proc. Natl. Acad. Sci. USA 101(48):16745-9), Heteractis HcRed1 and t-HcRed (Clontech, Palo Alto, Calif.), TurboFP635 (Katushka), mKate, and mNeptune; near-infrared FPs, such as and IFP1.4 (Scherbo et al. (2007) Nat Methods 4:741-746), eqFP650 and eqFP670; and others (see, e.g., Shaner N C, Steinbach P A, and Tsien R Y. (2005) Nat Methods. 2(12):905-9 and Chudakov et al. (2010) Physil Rev 90:1103-1163 for description of additional exemplary FPs of various excitation/emission spectra)

As used herein, Aequorea GFP refers to GFPs from the genus Aequorea and to mutants or variants thereof. Such variants and GFPs from other species, such as Anthozoa reef coral, Anemonia sea anemone, Renilla sea pansy, Galaxea coral, Acropora brown coral, Trachyphyllia and Pectimidae stony coral and other species are well known and are available and known to those of skill in the art.

As used herein, luminescence refers to the detectable electromagnetic (EM) radiation, generally, ultraviolet (UV), infrared (IR) or visible EM radiation that is produced when the excited product of an exergonic chemical process reverts to its ground state with the emission of light. Chemiluminescence is luminescence that results from a chemical reaction. Bioluminescence is chemiluminescence that results from a chemical reaction using biological molecules (or synthetic versions or analogs thereof) as substrates and/or enzymes. Fluorescence is luminescence in which light of a visible color is emitted from a substance under stimulation or excitation by light or other forms radiation such as ultraviolet (UV), infrared (IR) or visible EM radiation.

As used herein, chemiluminescence refers to a chemical reaction in which energy is specifically channeled to a molecule causing it to become electronically excited and subsequently to release a photon, thereby emitting visible light. Temperature does not contribute to this channeled energy. Thus, chemiluminescence involves the direct conversion of chemical energy to light energy.

As used herein, bioluminescence, which is a type of chemiluminescence, refers to the emission of light by biological molecules, particularly proteins. The essential condition for bioluminescence is molecular oxygen, either bound or free in the presence of an oxygenase, a luciferase, which acts on a substrate, a luciferin. Bioluminescence is generated by an enzyme or other protein (luciferase) that is an oxygenase that acts on a substrate luciferin (a bioluminescence substrate) in the presence of molecular oxygen and transforms the substrate to an excited state, which, upon return to a lower energy level releases the energy in the form of light.

As used herein, the substrates and enzymes for producing bioluminescence are generically referred to as luciferin and luciferase, respectively. When reference is made to a particular species thereof, for clarity, each generic term is used with the name of the organism from which it derives such as, for example, click beetle luciferase or firefly luciferase.

As used herein, luciferase refers to oxygenases that catalyze a light emitting reaction. For instance, bacterial luciferases catalyze the oxidation of flavin mononucleotide (FMN) and aliphatic aldehydes, which reaction produces light. Another class of luciferases, found among marine arthropods, catalyzes the oxidation of Cypridina (Vargula) luciferin and another class of luciferases catalyzes the oxidation of Coleoptera luciferin. Thus, luciferase refers to an enzyme or photoprotein that catalyzes a bioluminescent reaction (a reaction that produces bioluminescence). The luciferases, such as firefly and Gaussia and Renilla luciferases, are enzymes which act catalytically and are unchanged during the bioluminescence generating reaction. The luciferase photoproteins, such as the aequorin photoprotein to which luciferin is non-covalently bound, are changed, such as by release of the luciferin, during bioluminescence generating reaction. The luciferase is a protein, or a mixture of proteins (e.g., bacterial luciferase), that occurs naturally in an organism or a variant or mutant thereof, such as a variant produced by mutagenesis that has one or more properties, such as thermal stability; that differ from the naturally-occurring protein. Luciferases and modified mutant or variant forms thereof are well known. For purposes herein, reference to luciferase refers to either the photoproteins or luciferases.

Reference, for example, to Renilla luciferase refers to an enzyme isolated from member of the genus Renilla or an equivalent molecule obtained from any other source, such as from another related copepod, or that has been prepared synthetically. It is intended to encompass Renilla luciferases with conservative amino acid substitutions that do not substantially alter activity. Conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

As used herein, bioluminescence substrate refers to the compound that is oxidized in the presence of a luciferase and any necessary activators and generates light. These substrates are referred to as luciferins herein, are substrates that undergo oxidation in a bioluminescence reaction. These bioluminescence substrates include any luciferin or analog thereof or any synthetic compound with which a luciferase interacts to generate light. Typical substrates include those that are oxidized in the presence of a luciferase or protein in a light-generating reaction. Bioluminescence substrates, thus, include those compounds that those of skill in the art recognize as luciferins. Luciferins, for example, include firefly luciferin, Cypridina (also known as Vargula) luciferin (coelenterazine), bacterial luciferin, as well as synthetic analogs of these substrates or other compounds that are oxidized in the presence of a luciferase in a reaction the produces bioluminescence.

As used herein, capable of conversion into a bioluminescence substrate refers to being susceptible to chemical reaction, such as oxidation or reduction, which yields a bioluminescence substrate. For example, the luminescence producing reaction of bioluminescent bacteria involves the reduction of a flavin mononucleotide group (FMN) to reduced flavin mononucleotide (FMNH₂) by a flavin reductase enzyme. The reduced flavin mononucleotide (substrate) then reacts with oxygen (an activator) and bacterial luciferase to form an intermediate peroxy flavin that undergoes further reaction, in the presence of a long-chain aldehyde, to generate light. With respect to this reaction, the reduced flavin and the long chain aldehyde are bioluminescence substrates.

As used herein, a bioluminescence generating system refers to the set of reagents required to conduct a bioluminescent reaction. Thus, the specific luciferase, luciferin and other substrates, solvents and other reagents that can be required to complete a bioluminescent reaction form a bioluminescence system. Thus a bioluminescence generating system refers to any set of reagents that, under appropriate reaction conditions, yield bioluminescence. Appropriate reaction conditions refer to the conditions necessary for a bioluminescence reaction to occur, such as pH, salt concentrations and temperature. In general, bioluminescence systems include a bioluminescence substrate, luciferin, a luciferase, which includes enzymes luciferases and photoproteins and one or more activators. A specific bioluminescence system can be identified by reference to the specific organism from which the luciferase derives; for example, the Renilla bioluminescence system includes a Renilla luciferase, such as a luciferase isolated from Renilla or produced using recombinant methods or modifications of these luciferases. This system also includes the particular activators necessary to complete the bioluminescence reaction, such as oxygen and a substrate with which the luciferase reacts in the presence of the oxygen to produce light.

As used herein, the term “modified” with reference to a gene refers to a gene encoding a gene product, having one or more truncations, mutations, insertions or deletions; to a deleted gene; or to a gene encoding a gene product that is inserted (e.g., into the chromosome or on a plasmid, phagemid, cosmid, and phage), typically accompanied by at least a change in function of the modified gene product or virus.

As used herein, a “non-essential gene or region” of a virus genome is a location or region that can be modified by insertion, deletion, or mutation without inhibiting the infection life cycle of the virus. Modification of a “non-essential gene or region” is intended to encompass modifications that have no effect on the virus life cycle and modifications that attenuate or reduce the toxicity of the virus, but do not completely inhibit infection, replication and production of new virus.

As used herein, an “attenuated virus” refers to a virus that has been modified to alter one or more properties of the virus that affect, for example, virulence, toxicity, or pathogenicity of the virus compared to a virus without such modification. Typically, the viruses for use in the methods provided herein are attenuated viruses with respect to the wild-type form of the virus.

As used herein, an “attenuated LIVP virus” with reference to LIVP refers to a virus that exhibits reduced or less virulence, toxicity or pathogenicity compared to LIVP.

As used herein, “toxicity” (also referred to as virulence or pathogenicity herein) with reference to a virus refers to the deleterious or toxic effects to a host upon administration of the virus. For an oncolytic virus, such as LIVP, the toxicity of a virus is associated with its accumulation in non-tumorous organs or tissues, which can impact the survival of the host or result in deleterious or toxic effects. Toxicity can be measured by assessing one or more parameters indicative of toxicity. These include accumulation in non-tumorous tissues and effects on viability or health of the subject to whom it has been administered, such as effects on weight.

As used herein, “reduced toxicity” means that the toxic or deleterious effects upon administration of the virus to a host are attenuated or lessened compared to a host that is administered with another reference or control virus. For purposes herein, exemplary of a reference or control virus with respect to toxicity is the LIVP virus designated GLV-1h68 (described, for example, in U.S. Pat. No. 7,588,767) or a virus that is the same as the virus administered except not including a particular modification that reduces toxicity. Whether toxicity is reduced or lessened can be determined by assessing the effect of a virus and, if necessary, a control or reference virus, on a parameter indicative of toxicity. It is understood that when comparing the activity of two or more different viruses, the amount of virus (e.g. pfu) used in an in vitro assay or administered in vivo is the same or similar and the conditions (e.g. in vivo dosage regime) of the in vitro assay or in vivo assessment are the same or similar. For example, when comparing effects upon in vivo administration of a virus and a control or reference virus the subjects are the same species, size, gender and the virus is administered in the same or similar amount under the same or similar dosage regime. In particular, a virus with reduced toxicity can mean that upon administration of the virus to a host, such as for the treatment of a disease, the virus does not accumulate in non-tumorous organs and tissues in the host to an extent that results in damage or harm to the host, or that impacts survival of the host to a greater extent than the disease being treated does or to a greater extent than a control or reference virus does. For example, a virus with reduced toxicity includes a virus that does not result in death of the subject over the course of treatment.

As used herein, accumulation of a virus in a particular tissue refers to the distribution of the virus in particular tissues of a host organism after a time period following administration of the virus to the host, long enough for the virus to infect the host's organs or tissues. As one skilled in the art will recognize, the time period for infection of a virus will vary depending on the virus, the organ(s) or tissue(s), the immunocompetence of the host and dosage of the virus. Generally, accumulation can be determined at time points from about less than 1 day, about 1 day to about 2, 3, 4, 5, 6 or 7 days, about 1 week to about 2, 3 or 4 weeks, about 1 month to about 2, 3, 4, 5, 6 months or longer after infection with the virus. For purposes herein, the viruses preferentially accumulate in immunoprivileged tissue, such as inflamed tissue or tumor tissue, but are cleared from other tissues and organs, such as non-tumor tissues, in the host to the extent that toxicity of the virus is mild or tolerable and at most, not fatal.

As used herein, “preferential accumulation” refers to accumulation of a virus at a first location at a higher level than accumulation at a second location (i.e., the concentration of viral particles, or titer, at the first location is higher than the concentration of viral particles at the second location). Thus, a virus that preferentially accumulates in immunoprivileged tissue (tissue that is sheltered from the immune system), such as inflamed tissue, and tumor tissue, relative to normal tissues or organs, refers to a virus that accumulates in immunoprivileged tissue, such as tumor, at a higher level (i.e., concentration or viral titer) than the virus accumulates in normal tissues or organs.

As used herein, the terms immunoprivileged cells and immunoprivileged tissues refer to cells and tissues, such as solid tumors, which are sequestered from the immune system. Generally, administration of a virus to a subject elicits an immune response that clears the virus from the subject. Immunoprivileged sites, however, are shielded or sequestered from the immune response, permitting the virus to survive and generally to replicate. Immunoprivileged tissues include proliferating tissues, such as tumor tissues.

As used herein, “anti-tumor activity” or “anti-tumorigenic” refers to virus strains that prevent or inhibit the formation or growth of tumors in vitro or in vivo in a subject. Anti-tumor activity can be determined by assessing a parameter or parameters indicative of anti-tumor activity.

As used herein, “greater” or “improved” activity with reference to anti-tumor activity or anti-tumorigenicity means that a virus strain is capable of preventing or inhibiting the formation or growth of tumors in vitro or in vivo in a subject to a greater extent than a reference or control virus or to a greater extent than absence of treatment with the virus. Whether anti-tumor activity is “greater” or “improved” can be determined by assessing the effect of a virus and, if necessary, a control or reference virus, on a parameter indicative of anti-tumor activity. It is understood that when comparing the activity of two or more different viruses, the amount of virus (e.g. pfu) used in an in vitro assay or administered in vivo is the same or similar, and the conditions (e.g. in vivo dosage regime) of the in vitro assay or in vivo assessment are the same or similar.

As used herein, “genetic therapy” or “gene therapy” involves the transfer of heterologous nucleic acid, such as DNA, into certain cells, target cells, of a mammal, particularly a human, with a disorder or conditions for which such therapy is sought. The nucleic acid, such as DNA, is introduced into the selected target cells, such as directly or in a vector or other delivery vehicle, in a manner such that the heterologous nucleic acid, such as DNA, is expressed and a therapeutic product encoded thereby is produced. Alternatively, the heterologous nucleic acid, such as DNA, can in some manner mediate expression of DNA that encodes the therapeutic product, or it can encode a product, such as a peptide or RNA that in some manner mediates, directly or indirectly, expression of a therapeutic product. Genetic therapy also can be used to deliver nucleic acid encoding a gene product that replaces a defective gene or supplements a gene product produced by the mammalian or the cell in which it is introduced. The introduced nucleic acid can encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous nucleic acid, such as DNA, encoding the therapeutic product can be modified prior to introduction into the cells of the afflicted host in order to enhance or otherwise alter the product or expression thereof. Genetic therapy also can involve delivery of an inhibitor or repressor or other modulator of gene expression.

As used herein, the terms overproduce or overexpress when used in reference to a substance, molecule, compound or composition made in a cell refers to production or expression at a level that is greater than a baseline, normal or usual level of production or expression of the substance, molecule, compound or composition by the cell. A baseline, normal or usual level of production or expression includes no production/expression or limited, restricted or regulated production/expression. Such overproduction or overexpression is typically achieved by modification of cell.

As used herein, a tumor, also known as a neoplasm, is an abnormal mass of tissue that results when cells proliferate at an abnormally high rate. Tumors can show partial or total lack of structural organization and functional coordination with normal tissue. Tumors can be benign (not cancerous), or malignant (cancerous). As used herein, a tumor is intended to encompass hematopoietic tumors as well as solid tumors.

Malignant tumors can be broadly classified into three major types. Carcinomas are malignant tumors arising from epithelial structures (e.g. breast, prostate, lung, colon, pancreas). Sarcomas are malignant tumors that originate from connective tissues, or mesenchymal cells, such as muscle, cartilage, fat or bone. Leukemias and lymphomas are malignant tumors affecting hematopoietic structures (structures pertaining to the formation of blood cells) including components of the immune system. Other malignant tumors include, but are not limited to, tumors of the nervous system (e.g. neurofibromatomas), germ cell tumors, and blastic tumors.

As used herein, a disease or disorder refers to a pathological condition in an organism resulting from, for example, infection or genetic defect, and characterized by identifiable symptoms. An exemplary disease as described herein is a neoplastic disease, such as cancer.

As used herein, proliferative disorders include any disorders involving abnormal proliferation of cells (i.e. cells proliferate more rapidly compared to normal tissue growth), such as, but not limited to, neoplastic diseases.

As used herein, neoplastic disease refers to any disorder involving cancer, including tumor development, growth, metastasis and progression.

As used herein, cancer is a term for diseases caused by or characterized by any type of malignant tumor, including metastatic cancers, lymphatic tumors, and blood cancers. Exemplary cancers include, but are not limited to, acute lymphoblastic leukemia, acute lymphoblastic leukemia, acute myeloid leukemia, acute promyelocytic leukemia, adenocarcinoma, adenoma, adrenal cancer, adrenocortical carcinoma, AIDS-related cancer, AIDS-related lymphoma, anal cancer, appendix cancer, astrocytoma, basal cell carcinoma, bile duct cancer, bladder cancer, bone cancer, osteosarcoma/malignant fibrous histiocytoma, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, Burkitt lymphoma, carcinoid tumor, carcinoma, central nervous system lymphoma, cervical cancer, chronic lymphocytic leukemia, chronic myelogenous leukemia, chronic myeloproliferative disorder, colon cancer, cutaneous T-cell lymphoma, desmoplastic small round cell tumor, endometrial cancer, ependymoma, epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, Hodgkin lymphoma, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, lymphomas, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenstrom's macroglobulinemia or Wilm's tumor. Exemplary cancers commonly diagnosed in humans include, but are not limited to, cancers of the bladder, brain, breast, bone marrow, cervix, colon/rectum, kidney, liver, lung/bronchus, ovary, pancreas, prostate, skin, stomach, thyroid, or uterus. Exemplary cancers commonly diagnosed in dogs, cats, and other pets include, but are not limited to, lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma (e.g., granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma, follicular lymphoma, intestinal lymphosarcoma, fibrosarcoma and pulmonary squamous cell carcinoma. Exemplary cancers diagnosed in rodents, such as a ferret, include, but are not limited to, insulinoma, lymphoma, sarcoma, neuroma, pancreatic islet cell tumor, gastric MALT lymphoma and gastric adenocarcinoma. Exemplary neoplasias affecting agricultural livestock include, but are not limited to, leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lymphadenitis (CLA): chronic, infectious, contagious disease of sheep and goats caused by the bacterium Corynebacterium pseudotuberculosis, and contagious lung tumor of sheep caused by jaagsiekte.

As used herein, an aggressive cancer refers to a cancer characterized by a rapidly growing tumor or tumors. Typically the tumor(s) is actively metastasizing or is at risk of metastasizing. Aggressive cancer typically refer to metastatic cancers that spread to multiple locations in the body.

As used herein, an in vivo method refers to any method that is performed within the living body of a subject. As used herein, an in vitro method refers to any method that is performed outside the living body of a subject.

As used herein, an ex vivo method refers to a method performed on a sample obtained from a subject.

As used herein, the term “therapeutic virus” refers to a virus that is administered for the treatment of a disease or disorder, such as a neoplastic disease, such as cancer, a tumor and/or a metastasis or inflammation or wound or diagnosis thereof and or both. Generally, a therapeutic virus herein is one that exhibits anti-tumor activity and minimal toxicity.

As used herein, treatment means ameliorating a disease or a symptom thereof.

As used herein, treatment of a subject that has a neoplastic disease, including a tumor or metastasis, means any manner of treatment in which the symptoms of having the neoplastic disease are ameliorated or otherwise beneficially altered. Typically, treatment of a tumor or metastasis in a subject encompasses any manner of treatment that results in slowing of tumor growth, lysis of tumor cells, reduction in the size of the tumor, prevention of new tumor growth, or prevention of metastasis of a primary tumor, including inhibition vascularization of the tumor, tumor cell division, tumor cell migration or degradation of the basement membrane or extracellular matrix.

As used herein, therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, amelioration or alleviation of the symptoms of a particular disorder, such as by administration of a particular pharmaceutical composition or therapeutic, refers to any lessening, whether permanent or temporary, lasting or transient that can be attributed to or associated with administration of the composition or therapeutic.

As used herein, efficacy means that upon systemic administration of an oncolytic virus, the virus will colonize tumor cells and replicate. In particular, it will replicate sufficiently so that tumor cells released into circulation will contain virus. Colonization and replication in tumor cells is indicative that the treatment is or will be an effective treatment.

As used herein, effective treatment with a virus is one that can increase survival compared to the absence of treatment therewith. For example, a virus is an effective treatment if it stabilizes disease, causes tumor regression, decreases severity of disease or slows down or reduces metastasizing of the tumor.

As used herein, therapeutic agents are agents that ameliorate the symptoms of a disease or disorder or ameliorate the disease or disorder. Therapeutic agents can be any molecule, such as a small molecule, a peptide, a polypeptide, a protein, an antibody, an antibody fragment, a DNA, or a RNA. Therapeutic agent, therapeutic compound, or therapeutic regimens include conventional drugs and drug therapies, including vaccines for treatment or prevention (i.e., reducing the risk of getting a particular disease or disorder), which are known to those skilled in the art and described elsewhere herein. Therapeutic agents for the treatment of neoplastic disease include, but are not limited to, moieties that inhibit cell growth or promote cell death, that can be activated to inhibit cell growth or promote cell death, or that activate another agent to inhibit cell growth or promote cell death. Therapeutic agents for use in the methods provided herein can be, for example, an anticancer agent. Exemplary therapeutic agents include, for example, therapeutic microorganisms, such as therapeutic viruses and bacteria, chemotherapeutic compounds, cytokines, growth factors, hormones, photosensitizing agents, radionuclides, toxins, antimetabolites, signaling modulators, anticancer antibiotics, anticancer antibodies, anti-cancer oligopeptides, anti-cancer oligonucleotide (e.g., antisense RNA and siRNA), angiogenesis inhibitors, radiation therapy, or a combination thereof.

As used herein, an anti-cancer agent or compound (used interchangeably with “anti-tumor or anti-neoplastic agent”) refers to any agents, or compounds, used in anti-cancer treatment. These include any agents, when used alone or in combination with other compounds or treatments, that can alleviate, reduce, ameliorate, prevent, or place or maintain in a state of remission of clinical symptoms or diagnostic markers associated with neoplastic disease, tumors and cancer, and can be used in methods, combinations and compositions provided herein.

As used herein, a “chemotherapeutic agent” is any drug or compound that is used in anti-cancer treatment. Exemplary of such agents are alkylating agents, nitrosoureas, antitumor antibiotics, antimetabolites, antimitotics, topoisomerase inhibitors, monoclonal antibodies, and signaling inhibitors. Exemplary chemotherapeutic agent include, but are not limited to, chemotherapeutic agents, such as Ara-C, cisplatin, carboplatin, paclitaxel, doxorubicin, gemcitabine, camptothecin, irinotecan, cyclophosphamide, 6-mercaptopurine, vincristine, 5-fluorouracil, and methotrexate. The term “chemotherapeutic agent” can be used interchangeably with the term “anti-cancer agent” when referring to drugs or compounds for the treatment of cancer. As used herein, reference to a chemotherapeutic agent includes combinations or a plurality of chemotherapeutic agents unless otherwise indicated.

As used herein, an anti-metastatic agent is an agent that ameliorates the symptoms of metastasis or ameliorates metastasis. Generally, anti-metastatic agents directly or indirectly inhibit one or more steps of metastasis, including but not limited to, degradation of the basement membrane and proximal extracellular matrix, which leads to tumor cell detachment from the primary tumor, tumor cell migration, tumor cell invasion of local tissue, tumor cell division and colonization at the secondary site, organization of endothelial cells into new functioning capillaries in a tumor, and the persistence of such functioning capillaries in a tumor. Anti-metastatic agents include agents that inhibit the metastasis of a cell from a primary tumor, including release of the cell from the primary tumor and establishment of a secondary tumor, or that inhibits further metastasis of a cell from a site of metastasis. Treatment of a tumor bearing subject with anti-metastatic agents can result in, for example, the delayed appearance of secondary (i.e. metastatic) tumors, slowed development of primary or secondary tumors, decreased occurrence of secondary tumors, slowed or decreased severity of secondary effects of neoplastic disease, arrested tumor growth and regression.

As used herein, an effective amount of a virus or compound for treating a particular disease is an amount that is sufficient to ameliorate, or in some manner reduce the symptoms associated with the disease. Such an amount can be administered as a single dosage or can be administered in multiple dosages according to a regimen, whereby it is effective. The amount can cure the disease but, typically, is administered in order to ameliorate the symptoms of the disease. Repeated administration can be required to achieve the desired amelioration of symptoms.

As used herein, a compound produced in a tumor refers to any compound that is produced in the tumor or tumor environment by virtue of the presence of an introduced virus, generally a recombinant virus, expressing one or more gene products. For example, a compound produced in a tumor can be, for example, an encoded polypeptide or RNA, a metabolite, or compound that is generated by a recombinant polypeptide and the cellular machinery of the tumor.

As used herein, the term “ELISA” refers to enzyme-linked immunosorbent assay. Numerous methods and applications for carrying out an ELISA are well known in the art, and provided in many sources (See, e.g., Crowther, “Enzyme-Linked Immunosorbent Assay (ELISA),” in Molecular Biomethods Handbook, Rapley et al. [eds.], pp. 595-617, Hzumana Press, Inc., Totowa, N.J. [1998]; Harlow and Lane (eds.), Antibodies: A Laboratory Manual, Cold Spring Harbor Laboratory Press [1988]; and Ausubel et al. (eds.), Current Protocols in Molecular Biology, Ch. 11, John Wiley & Sons, Inc., New York [1994]; and Newton, et al. (2006) Neoplasia. 8:772-780). A “direct ELISA” protocol involves a target-binding molecule, such as a cell, cell lysate, or isolated protein, first bound and immobilized to a microtiter plate well. A “sandwich ELISA” involves a target-binding molecule attached to the substrate by capturing it with an antibody that has been previously bound to the microtiter plate well. The ELISA method detects an immobilized ligand-receptor complex (binding) by use of fluorescent detection of fluorescently labeled ligands or an antibody-enzyme conjugate, where the antibody is specific for the antigen of interest, such as a phage virion, while the enzyme portion allows visualization and quantitation by the generation of a colored or fluorescent reaction product. The conjugated enzymes commonly used in the ELISA include horseradish peroxidase, urease, alkaline phosphatase, glucoamylase or O-galactosidase. The intensity of color development is proportional to the amount of antigen present in the reaction well.

As used herein, a delivery vehicle for administration refers to a lipid-based or other polymer-based composition, such as liposome, micelle or reverse micelle, that associates with an agent, such as a virus provided herein, for delivery into a host subject.

As used herein, a “diagnostic agent” refer to any agent that can be applied in the diagnosis or monitoring of a disease or health-related condition. The diagnostic agent can be any molecule, such as a small molecule, a peptide, a polypeptide, a protein, an antibody, an antibody fragment, a DNA, or a RNA.

As used herein, a detectable label or detectable moiety or diagnostic moiety (also imaging label, imaging agent, or imaging moiety) refers to an atom, molecule or composition, wherein the presence of the atom, molecule or composition can be directly or indirectly measured. Detectable labels can be used to image one or more of any of the viruses provided herein. Detectable labels can be used in any of the methods provided herein. Detectable labels include, for example, chemiluminescent moieties, bioluminescent moieties, fluorescent moieties, radionuclides, and metals. Methods for detecting labels are well known in the art. Such a label can be detected, for example, by visual inspection, by fluorescence spectroscopy, by reflectance measurement, by flow cytometry, by X-rays, by a variety of magnetic resonance methods such as magnetic resonance imaging (MRI) and magnetic resonance spectroscopy (MRS). Methods of detection also include any of a variety of tomographic methods including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography, and ultrasonic tomography. Direct detection of a detectable label refers to, for example, measurement of a physical phenomenon of the detectable label itself, such as energy or particle emission or absorption of the label itself, such as by X-ray or MRI. Indirect detection refers to measurement of a physical phenomenon of an atom, molecule or composition that binds directly or indirectly to the detectable label, such as energy or particle emission or absorption, of an atom, molecule or composition that binds directly or indirectly to the detectable label. In a non-limiting example of indirect detection, a detectable label can be biotin, which can be detected by binding to avidin. Non-labeled avidin can be administered systemically to block non-specific binding, followed by systemic administration of labeled avidin. Thus, included within the scope of a detectable label or detectable moiety is a bindable label or bindable moiety, which refers to an atom, molecule or composition, wherein the presence of the atom, molecule or composition can be detected as a result of the label or moiety binding to another atom, molecule or composition. Exemplary detectable labels include, for example, metals such as colloidal gold, iron, gadolinium, and gallium-67, fluorescent moieties, and radionuclides. Exemplary fluorescent moieties and radionuclides are provided elsewhere herein.

As used herein, a radionuclide, a radioisotope or radioactive isotope is used interchangeably to refer to an atom with an unstable nucleus. The nucleus is characterized by excess energy which is available to be imparted either to a newly-created radiation particle within the nucleus, or else to an atomic electron. The radionuclide, in this process, undergoes radioactive decay, and emits a gamma ray and/or subatomic particles. Such emissions can be detected in vivo by method such as, but not limited to, positron emission tomography (PET), single-photon emission computed tomography (SPECT) or planar gamma imaging. Radioisotopes can occur naturally, but also can be artificially produced. Exemplary radionuclides for use in in vivo imaging include, but are not limited to, ¹¹C, ¹¹F, ¹³C, ¹³N, ¹⁵N, ¹⁵0, 18F, ¹⁹F, ³²P, ⁵²Fe, ⁵¹Cr, ⁵⁵Co, ⁵⁵Fe, ⁵⁷Co, ⁵⁸Co, ⁵⁷Ni, ⁵⁹Fe ⁶⁰Co, ⁶⁴Cu, ⁶⁷Ga, ⁶⁸Ga, ⁶⁰Cu(II), ⁶⁷Cu(II), ⁹⁹Tc, ⁹⁰Y, ⁹⁹Tc, ¹⁰³Pd, ¹⁰⁶Ru, ¹¹¹In. ¹¹⁷Lu, ¹²³I, ¹²⁵I, ¹²⁴I, ¹³¹I, ¹³⁷Cs, ¹⁵³Gd, ¹⁵³Sm, ¹⁸⁶Re, ¹⁸⁸Re, ¹⁹²Ir, ¹⁹⁸Au, ²¹¹At, ²¹²Bi, ²¹³Bi and ²⁴¹Am. Radioisotopes can be incorporated into or attached to a compound, such as a metabolic compound. Exemplary radionuclides that can be incorporated or linked to a metabolic compound, such as nucleoside analog, include, but are not limited to, ¹²³I, ¹²⁴I, ¹²⁵I, ¹³¹I, ¹⁸F, ¹⁹F, ¹¹C, ¹³C, ¹⁴C, ⁷⁵Br, ⁷⁶Br, and ³H.

As used herein, magnetic resonance imaging (MRI) refers to the use of a nuclear magnetic resonance spectrometer to produce electronic images of specific atoms and molecular structures in solids, especially human cells, tissues, and organs. MRI is non-invasive diagnostic technique that uses nuclear magnetic resonance to produce cross-sectional images of organs and other internal body structures. The subject lies inside a large, hollow cylinder containing a strong electromagnet, which causes the nuclei of certain atoms in the body (such as, for example, ¹H, ¹³C and ¹⁹F) to align magnetically. The subject is then subjected to radio waves, which cause the aligned nuclei to flip; when the radio waves are withdrawn the nuclei return to their original positions, emitting radio waves that are then detected by a receiver and translated into a two-dimensional picture by computer. For some MRI procedures, contrast agents such as gadolinium are used to increase the accuracy of the images.

As used herein, an X-ray refers to a relatively high-energy photon, or a stream of such photons, having a wavelength in the approximate range from 0.01 to 10 nanometers. X-rays also refer to photographs taken with x-rays.

As used herein, a compound conjugated to a moiety refers to a complex that includes a compound bound to a moiety, where the binding between the compound and the moiety can arise from one or more covalent bonds or non-covalent interactions such as hydrogen bonds, or electrostatic interactions. A conjugate also can include a linker that connects the compound to the moiety. Exemplary compounds include, but are not limited to, nanoparticles and siderophores. Exemplary moieties, include, but are not limited to, detectable moieties and therapeutic agents.

As used herein, “modulate” and “modulation” or “alter” refer to a change of an activity of a molecule, such as a protein. Exemplary activities include, but are not limited to, biological activities, such as signal transduction. Modulation can include an increase in the activity (i.e., up-regulation or agonist activity), a decrease in activity (i.e., down-regulation or inhibition) or any other alteration in an activity (such as a change in periodicity, frequency, duration, kinetics or other parameter). Modulation can be context dependent and typically modulation is compared to a designated state, for example, the wildtype protein, the protein in a constitutive state, or the protein as expressed in a designated cell type or condition.

As used herein, an agent or compound that modulates the activity of a protein or expression of a gene or nucleic acid either decreases or increases or otherwise alters the activity of the protein or, in some manner, up- or down-regulates or otherwise alters expression of the nucleic acid in a cell.

As used herein, “nucleic acids” include DNA, RNA and analogs thereof, including peptide nucleic acids (PNA) and mixtures thereof. Nucleic acids can be single or double-stranded. Nucleic acids can encode gene products, such as, for example, polypeptides, regulatory RNAs, microRNAs, siRNAs and functional RNAs.

As used herein, a sequence complementary to at least a portion of an RNA, with reference to antisense oligonucleotides, means a sequence of nucleotides having sufficient complementarity to be able to hybridize with the RNA, generally under moderate or high stringency conditions, forming a stable duplex; in the case of double-stranded antisense nucleic acids, a single strand of the duplex DNA (i.e., dsRNA) can thus be assayed, or triplex formation can be assayed. The ability to hybridize depends on the degree of complementarity and the length of the antisense nucleic acid. Generally, the longer the hybridizing nucleic acid, the more base mismatches with an encoding RNA it can contain and still form a stable duplex (or triplex, as the case can be). One skilled in the art can ascertain a tolerable degree of mismatch by use of standard procedures to determine the melting point of the hybridized complex.

As used herein, a peptide refers to a polypeptide that is greater than or equal to 2 amino acids in length, and less than or equal to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “amino acid residue” refers to an amino acid formed upon chemical digestion (hydrolysis) of a polypeptide at its peptide linkages; The amino acid residues described herein are presumed to be in the “L” isomeric form. Residues in the “D” isomeric form, which are so designated, can be substituted for any L-amino acid residue as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243: 3557-3559 (1968), and adopted 37 C.F.R. §§1.821-1.822, abbreviations for amino acid residues are shown in Table 1:

TABLE 1
Table of Amino Acid Correspondence

| SYMBOL | | |
|---|---|---|
| 1-Letter | 3-Letter | AMINO ACID |
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glu and/or Gln |
| W | Trp | Tryptophan |
| R | Arg | Arginine |
| D | Asp | Aspartic acid |
| N | Asn | Asparagine |
| B | Asx | Asn and/or Asp |
| C | Cys | Cysteine |
| X | Xaa | Unknown or other |

All amino acid residue sequences represented herein by formulae have a left to right orientation in the conventional direction of amino-terminus to carboxyl-terminus. In addition, the phrase “amino acid residue” is defined to include the amino acids listed in the Table of Correspondence (Table 1) and modified and unusual amino acids, such as those referred to in 37C.F.R. §§1.821-1.822, and incorporated herein by reference. Furthermore, a dash at the beginning or end of an amino acid residue sequence indicates a peptide bond to a further sequence of one or more amino acid residues, to an amino-terminal group such as NH₂ or to a carboxyl-terminal group such as COOH.

As used herein, the “naturally occurring α-amino acids” are the residues of those 20 α-amino acids found in nature which are incorporated into protein by the specific recognition of the charged tRNA molecule with its cognate mRNA codon in humans. Non-naturally occurring amino acids thus include, for example, amino acids or analogs of amino acids other than the 20 naturally-occurring amino acids and include, but are not limited to, the D-isostereomers of amino acids. Exemplary non-natural amino acids are described herein and are known to those of skill in the art.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule may not be paired. Such unpaired ends will, in general, not exceed 20 nucleotides in length.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g. Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exists a number of methods to measure identity between two polynucleotide or polypeptides, the term “identity” is well known to skilled artisans (Carrillo, H. & Lipton, D., SIAM J Applied Math 48:1073 (1988)).

As used herein, homologous (with respect to nucleic acid and/or amino acid sequences) means about greater than or equal to 25% sequence homology, typically greater than or equal to 25%, 40%, 50%, 60%, 70%, 80%, 85%, 90% or 95% sequence homology; the precise percentage can be specified if necessary. For purposes herein the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073). By sequence homology, the number of conserved amino acids is determined by standard alignment algorithms programs, and can be used with default gap penalties established by each supplier. Substantially homologous nucleic acid molecules hybridize typically at moderate stringency or at high stringency all along the length of the nucleic acid of interest. Also contemplated are nucleic acid molecules that contain degenerate codons in place of codons in the hybridizing nucleic acid molecule.

Whether any two molecules have nucleotide sequences or amino acid sequences that are at least 60%, 70%, 80%, 85%, 90%, 95%, 96%, 97%, 98% or 99% “identical” or “homologous” can be determined using known computer algorithms such as the “FASTA” program, using for example, the default parameters as in Pearson et al. (1988) Proc. Natl. Acad. Sci. USA 85:2444 (other programs include the GCG program package (Devereux, J., et al. Nucleic Acids Research 12(I):387 (1984)), BLASTP, BLASTN, FASTA (Altschul, S. F., et al. J Mol Biol 215:403 (1990)); Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. Such identify is assessed by comparing a sequence of interest to reference sequence.

As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes a test and reference polypeptide length of 100 amino acids are compared. No more than 10% (i.e., 10 out of 100) of the amino acids in the test polypeptide differs from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result is independent of the program and gap parameters set; such high levels of identity can be assessed readily, often by manual alignment without relying on software. As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically the two molecules are selected such that they will hybridize under conditions of high stringency.

As used herein, substantially identical to a product means sufficiently similar so that the property of interest is sufficiently unchanged so that the substantially identical product can be used in place of the product.

As used herein, it also is understood that the terms “substantially identical” or “similar” varies with the context as understood by those skilled in the relevant art.

As used herein, an allelic variant or allelic variation references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide. Reference to an allelic variant herein generally refers to variations n proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include modifications such as substitutions, deletions and insertions of nucleotides. An allele of a gene also can be a form of a gene containing a mutation.

As used herein, species variants refer to variants in polypeptides among different species, including different mammalian species, such as mouse and human. Generally, species variants have 70%, 75%, 80%, 85%, 90%, 91%, 92%, 93%, 94%, 95%, 96%, 97%, 98% or sequence identity. Corresponding residues between and among species variants can be determined by comparing and aligning sequences to maximize the number of matching nucleotides or residues, for example, such that identity between the sequences is equal to or greater than 95%, equal to or greater than 96%, equal to or greater than 97%, equal to or greater than 98% or equal to greater than 99%. The position of interest is then given the number assigned in the reference nucleic acid molecule. Alignment can be effected manually or by eye, particularly, where sequence identity is greater than 80%.

As used herein, a human protein is one encoded by a nucleic acid molecule, such as DNA, present in the genome of a human, including all allelic variants and conservative variations thereof. A variant or modification of a protein is a human protein if the modification is based on the wildtype or prominent sequence of a human protein.

As used herein, a splice variant refers to a variant produced by differential processing of a primary transcript of genomic DNA that results in more than one type of mRNA.

As used herein, modification is in reference to modification of a sequence of amino acids of a polypeptide or a sequence of nucleotides in a nucleic acid molecule and includes deletions, insertions, and replacements (e.g. substitutions) of amino acids and nucleotides, respectively. Exemplary of modifications are amino acid substitutions. An amino-acid substituted polypeptide can exhibit 65%, 70%, 80%, 85%, 90%, 91%, 92%; 93%, 94%, 95%, 96%, 97%, 98% or more sequence identity to a polypeptide not containing the amino acid substitutions. Amino acid substitutions can be conservative or non-conservative. Generally, any modification to a polypeptide retains an activity of the polypeptide. Methods of modifying a polypeptide are routine to those of skill in the art, such as by using recombinant DNA methodologies.

As used herein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made in accordance with those set forth in Table 2 as follows:

TABLE 2
Table of Exemplary Conservative Amino Acid Substitutions

| Original residue | Exemplary Conservative Substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, the term promoter means a portion of a gene containing DNA sequences that provide for the binding of RNA polymerase and initiation of transcription. Promoter sequences are commonly, but not always, found in the 5′ non-coding region of genes.

As used herein, isolated or purified polypeptide or protein or biologically-active portion thereof is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

Hence, reference to a substantially purified polypeptide, refers to preparations of proteins that are substantially free of cellular material includes preparations of proteins in which the protein is separated from cellular components of the cells from which it is isolated or recombinantly-produced. In one example, the term substantially free of cellular material includes preparations of enzyme proteins having less that about 30% (by dry weight) of non-enzyme proteins (also referred to herein as a contaminating protein), generally less than about 20% of non-enzyme proteins or 10% of non-enzyme proteins or less that about 5% of non-enzyme proteins. When the enzyme protein is recombinantly produced, it also is substantially free of culture medium, i.e., culture medium represents less than about or at 20%, 10% or 5% of the volume of the enzyme protein preparation.

As used herein, the term substantially free of chemical precursors or other chemicals includes preparations of enzyme proteins in which the protein is separated from chemical precursors or other chemicals that are involved in the synthesis of the protein. The term includes preparations of enzyme proteins having less than about 30% (by dry weight), 20%, 10%, 5% or less of chemical precursors or non-enzyme chemicals or components.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant means or using recombinant DNA methods means the use of the well known methods of molecular biology for expressing proteins encoded by cloned DNA.

As used herein, a DNA construct is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, vector (or plasmid) refers to a nucleic acid construct that contains discrete elements that are used to introduce heterologous nucleic acid into cells for either expression of the nucleic acid or replication thereof. The vectors typically remain episomal, but can be designed to effect stable integration of a gene or portion thereof into a chromosome of the genome. Selection and use of such vectors are well known to those of skill in the art.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the term “viral vector” is used according to its art-recognized meaning. It refers to a nucleic acid vector that includes at least one element of viral origin and can be packaged into a viral vector particle. The viral vector particles can be used for the purpose of transferring DNA, RNA or other nucleic acids into cells either in vitro or in vivo. Viral vectors include, but are not limited to, poxvirus vectors (e.g., vaccinia vectors), retroviral vectors, lentivirus vectors, herpes virus vectors (e.g., HSV), baculovirus vectors, cytomegalovirus (CMV) vectors, papillomavirus vectors, simian virus (SV40) vectors, semliki forest virus vectors, phage vectors, adenoviral vectors and adeno-associated viral (AAV) vectors.

As used herein equivalent, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions that do not substantially alter the activity or function of the protein or peptide. When equivalent refers to a property, the property does not need to be present to the same extent (e.g., two peptides can exhibit different rates of the same type of enzymatic activity), but the activities are usually substantially the same.

As used herein, a composition refers to any mixture. It can be a solution, suspension, liquid, powder, paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a kit is a packaged combination, optionally, including instructions for use of the combination and/or other reactions and components for such use.

As used herein, the singular forms “a,” “an” and “the” include plural referents unless the context clearly dictates otherwise.

As used herein, ranges and amounts can be expressed as “about” or “approximately” a particular value or range. “About” or “approximately” also includes the exact amount. Hence, “about 5 milliliters” means “about 5 milliliters” and also “5 milliliters.” Generally “about” includes an amount that would be expected to be within experimental error.

As used herein, “about the same” means within an amount that one of skill in the art would consider to be the same or to be within an acceptable range of error. For example, typically, for pharmaceutical compositions, within at least 1%, 2%, 3%, 4%, 5% or 10% is considered about the same. Such amount can vary depending upon the tolerance for variation in the particular composition by subjects.

As used herein, “optional” or “optionally” means that the subsequently described event or circumstance does or does not occur, and that the description includes instances where said event or circumstance occurs and instances where it does not.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

### B. OVERVIEW

Metastasis involves the formation of progressively growing tumor foci at sites secondary to a primary lesion (Yoshida et al. (2000) J. Natl. Cancer Inst. 92(21):1717-1730; Welch et al. (1999) J. Natl. Cancer Inst. 91:1351-1353) and is a major cause of morbidity and mortality in human malignancies (Nathoo et al. J. Clin. Pathol. 58:237-242 (2005); Fidler et al. Cell 79:185-188 (1994)). In vivo metastasis follows a series of steps known as the metastatic cascade, in which tumor cells invade local tissue, intravasate through the bloodstream or lymphatics as emboli or single tumor cells (i.e. circulating tumor cells (CTCs)), and are transported to secondary sites, where they can lodge into the microvasculature and form metastatic lesions (Kauffman et al. (2003) J. Urology 169:1122-1133).

Methods for detecting metastasis include histological examination of tissue biopsies of the lymph nodes and other organs for evidence of tumor cell invasion and tumor biopsies for evaluation and grading of tumor differentiation. Such methods include morphological evaluation of tumor cells and immunostaining with tumor cell markers. While such information is useful in diagnosis and prescribing treatment, tissue biopsies are invasive procedures that can be painful, risky, and costly to the patient. In addition, in order to determine changes in the cancer over time and over the course of treatment, multiple biopsies are required, subjecting patients multiple painful and inconvenient procedures. MRI, CT and PET scanning procedures also are routinely used for monitoring location of tumors and tumor size, but these procedures also can be costly and are limited to detection of tumors that are greater than 2-3 mm in size. Thus, a metastatic tumor may not be detected until well after widespread metastasis of the primary tumor has occurred which decreases the chances of successful treatment of the cancer.

Provided herein are methods to detect tumor cells in body fluid samples and the use of such methods in various applications. Included among the applications are methods for diagnosis and treatment metastasis based on the detection and enumeration of circulating tumor cells (CTCs). The methods are exploit the ability of oncolytic viruses, such as the LIVP vaccinia virus, to preferentially infect metastatic tumor cells in vivo in a subject and ex vivo in a bodily sample from a subject. Using modified oncolytic viruses that encode a detectable reporter protein to detect CTCs provides superior prognostic and treatment selection information compared to other methods of detecting metastasis, including other available methods of detecting CTCs. As described herein, the oncolytic reporter viruses also can be used in combination with available tumor cell enrichment methods to provide convenient and reliable detection of CTCs without the need for additional processing steps which can damage samples obtained for analysis.

The methods provided herein are useful for, but not limited to, diagnosis of a cancer and/or metastases, staging of cancers, providing a cancer prognosis, predicting or diagnosing cancer recurrence, classification of patients for selection of an anti-cancer therapy, such as an oncolytic virus therapy, and monitoring therapy of a cancer. Among the methods provided are point of care diagnostic methods that can easily be performed in the clinic for detection of CTCs in a sample.

As described herein, it is found herein that subjects administered oncolytic reporter viruses produced CTCs detectable in the peripheral blood that were infected with the virus. Accordingly, the oncolytic reporter viruses can be employed for ex vivo detection and enumeration of CTCs in a sample, such as a tissue or body fluid sample, from a subject treated with the oncolytic reporter virus. In addition, the oncolytic reporter viruses also can be employed for in vivo detection and enumeration of CTCs in a subject treated with the oncolytic reporter virus.

As described herein, it also is found herein that an oncolytic reporter virus can provide high-throughput, specific and sensitive detection of CTCs in a sample when used in combination with one or more in vitro tumor cell enrichment methods for detection and enumeration of CTCs. Accordingly, the oncolytic reporter viruses can be employed for ex vivo detection and enumeration of CTCs in a sample, such as a tissue or body fluid sample, where the sample is processed by a tumor cell enrichment method in combination with infection with the oncolytic reporter virus for detection.

In exemplary methods described herein, the oncolytic reporter viruses can be used for the detection of cancer or detection of metastasis of a cancer. In some examples the oncolytic reporter virus is an oncolytic vaccinia virus, such as an LIVP vaccinia virus. The viruses can be used to infect a sample from a subject that has cancer, is suspected of having cancer, or is at risk of having cancer. Detection of infected cells in the sample indicates that the subject has cancer and/or active metastasis. In exemplary methods, the sample can be processed by a tumor cell enrichment method prior to, following, or concurrent with virus infection of the sample.

In other exemplary methods, a subject that has cancer, is suspected of having cancer, or is at risk of having cancer can be administered an oncolytic reporter virus and detection of the tumor cells is performed. Detection of infected cancer cells can be performed in vivo in the subject or ex vivo in a sample from the subject. In some examples, an ex vivo sample can be processed by a tumor cell enrichment method prior to detection of the infected tumor cells.

As described herein, vaccinia virus treatment of a subject with a metastasizing tumor also results in a significant reduction in the number and size of secondary metastases reduces the number of CTCs found in the blood (see, e.g., Examples 9 and 12 provided herein). Accordingly, oncolytic viruses, such as vaccinia virus, provide a means for detecting and enumerating CTCs in a subject and also can provide simultaneous treatment of the metastatic disease.

Also provided herein are combinations and kits that contain an oncolytic reporter virus (for example, any provided herein below in Section C), and optionally, other accompanying materials and reagents for use in practicing the methods, including materials and reagents for performing a tumor cell enrichment method, and selecting, monitoring and/or treating cancer.

1. Circulating Tumor Cells (CTCs) as Cancer Prognostic and Diagnostic Indicators

Circulating tumor cells were first observed in blood samples of deceased patients with advanced cancers as early as 1869 (Ashworth (1869) Aust Med J 14:146-149). More recently, studies on clinical samples, particularly in breast, colon and prostate cancer patients, have shown a correlation between the presence of CTCs in the peripheral blood and cancer prognosis. Detection of CTCs is predictive of metastatic disease, and the quantity of CTCs detected correlates with the severity of metastatic disease. The presence of CTCs in patient samples after therapy also has been associated with tumor progression and spread, poor response to therapy, relapse of disease, and/or decreased survival over a period of several years. Detection of CTCs can provide a means for early detection and treatment of metastatic disease and monitoring of disease therapy.

Detection and enumeration of CTCs in fluid samples from a patient, i.e. a “liquid biopsy”, such as a lymph or blood sample, is much less invasive than a tissue biopsy, and can be repeated frequently, allowing real-time monitoring of cancer progression and response to treatment. In addition, detection and enumeration of CTCs offers a convenient means to stratify patients for baseline characteristics that predict initial risk and subsequent risk based upon response to therapy.

Because circulating tumor cells (CTCs) have the potential to form tumors and their quantity in circulation correlates with metastatic disease, the ability to accurately identify and quantify CTCs in patient samples would aid in the early diagnosis and prognosis of many types of cancers and the monitoring of cancer treatments. Effective detection of CTCs in bodily samples, such as in the blood, lymph or other bodily fluids, also will aid in staging of particular tumors and evaluating metastatic activity.

Leptomeningeal metastases (LM) result from the spread of metastatic tumor cells to the cerebrospinal fluid (CSF) and leptomeninges. The incidence of LM in cancer patients ranges between 5 and 15% and is on the rise as the survival of cancer patients increases. LM are underdiagnosed since some metastases may remain asymptomatic. The prognosis for patients with LM is extremely poor with the median survival measured in months. Treatment of LM is mainly palliative. Early diagnosis and effective treatment are critical to prevent important neurological deficits, improve quality of life and prolong survival. Methods for the diagnosis of LM include clinical examination, neuroimaging, and CSF analysis. LM is diagnosed by cytological examination of the CSF, a method with limited sensitivity and specificity. Methods are provided herein to detect and diagnose LM, and also to effect treatment thereof.

Peritoneal carcinomatosis (PC) is the locoregional progression of cancers of gastrointestinal and gynecological origins. At the time of diagnosis, about 10 to 15% of patients with gastrointestinal and gynecological cancers have already developed PC, a terminal condition and a consequence of the underlying systemic nature of the disease (see, e.g., Spiliotis (2010) Hepatogastroenterology 57:1173-1177). Treatment with cytoreductive surgery (CRS), followed by hyperthermic intraperitoneal chemotherapy (HIPEC) has demonstrated a survival benefit, but this treatment is expensive and is associated with a very high postoperative morbidity rate, ranging from 25 to 56% (Spiliotis (2010) Hepatogastroenterology 57:1173-1177). As exemplified herein, oncolytic viruses and the methods provided herein effect detection of LM and PC. In addition, the oncolytic virus infects and eliminates tumor cells in LM and PC.

2. Existing Methods for Detection of CTCs

In patients with metastatic cancer, an estimated 1 million tumor cells per day per gram weight of a primary or secondary tumor are shed into the circulation; however, the half-life of most CTCs in circulation is short in vivo (˜1.0-2.4 hours). Thus, the effective levels of viable CTCs in circulation is low. In addition, detection of CTCs in patient blood samples is difficult due to the low concentration of CTCs relative to other blood components such as erythrocytes and leukocytes. It is estimated that ˜1-3 CTCs among a background of approximately 1×10⁹ erythrocytes and 1×10⁶ leukocytes are present in the blood of cancer patients with metastatic cancers.

In order to be effective, methods to identify CTCs require high throughput, high specificity, and high sensitivity. Because CTCs are present in low concentrations in bodily samples, such as blood, high throughput methods that can process larger samples in a reasonable amount of time following collection increase the chances of viable CTCs being present and detected in a particular sample; high specificity for CTC detection prevents or significantly decreases the detection of false positives (i.e. categorization of cells as CTCs that are not actually CTCs); and high sensitivity increases the probability that CTCs present in a sample will be detected.

Various methods to detect CTCs in patients have been developed. These methods include indirect and direct methods of measuring levels of CTCs in a sample.

Indirect methods of detecting CTCs include detection of CTC specific markers in patient fluid samples by methods such as reverse transcription-polymerase chain reaction (RT-PCR), quantitative RT-PCR (qRT-PCR), and nested RT-PCR. Because these methods rely on pooled samples of cells for detection of marker expression, they do not detect CTCs individually; morphological and quantitive analysis of the cells and confirmation of tumor cell identity cannot be performed.

Direct methods involve positive or negative selection of CTCs based on physical or biological properties of the CTCs. Such methods include selection for expression of CTC-specific cell surface markers and/or removal of non-tumor cells (e.g. normal blood cells) from samples. A majority of metastatic tumors are epithelial in origin which allows CTCs to be distinguished from other non-CTC cell types, such as, for example, blood cells. Some available methods of CTC isolation employ immuno-mediated enrichment based on expression of epithelial cell specific markers, such as epithelial cell adhesion molecule (EpCAM/CD326) and cytokeratin (CK), which are expressed on the cell surface of many epithelial malignancies. For example, the CellSearch (Veridex, Raritan, N.J.) system and the Magnetic Activated Cell Sorting (MACS) EpCAM-MicroBeads system (Miltenyi Biotech) use immunomagnetic capture of CTCs using magnetic beads coated with anti-EpCAM antibodies. CTCs that bind to the antibodies are captured under a magnetic field. Other methods of positive selection based on cell surface markers include laser scanning cytometry and micro-fluidic chips with surfaces coated with EpCAM. Additional characterization of the captured cells is required to confirm identity of the cells and generally involves staining with 4′,6-diamidino-2-phenylindole (DAPI) to show that the cell is nucleated, immunofluorescence with antibodies against cytokeratin to confirm that the captured cell is an epithelial cell, and negative CD45 staining to demonstrate that the captured cell is not a leukocyte. Magnetic bead-based systems require multiple preparatory steps, including centrifugation, washing, and incubation steps that often result in loss, induction of cell death, or destruction of a significant proportion of cells. Such aggressive multistep batch purification isolation procedures tend to generate low yield, purity and viability of CTCs.

Methods that use antibodies to capture CTCs also are prone to bias due to selection of only those circulating tumor cells bearing the surface markers for which the antibodies are specific. Not all circulating tumor cells express EpCAM. During induction of epithelial to mesenchymal transition (EMT) which facilitates cell migration during metastasis, EpCAM and cytokeratin (CK) are downregulated. Thus, tumor cells that have that entered circulation following extravasation may express low or no EpCAM or CK and may not be identified in such immunocapture methods. The method is thus subject to large range in recovery rates due to variable expression of the cell surface markers. In order to increase the overall capture of CTCs, such methods can be used in conjunction with other CTC enrichment methods, such as size-based capture.

Additional examples of methods to identify CTCs include removal of non-tumor cells from the sample. For example, some methods employ immunocapture of leukocytes from a sample using anti-CD45 antibodies and/or targeted lysis of red blood cells, which leaves nucleated cells in the sample. These procedures enrich the proportion of CTCs in the sample relative to non-tumor cells, thus allowing for easier analysis of the remaining cells. Following removal of non-tumor cells, the CTCs are typically detected by immunostaining.

Other direct methods of CTC isolation include methods that separate tumor cells based on physical properties of CTCs, such as by size, stiffness, and deformability of CTCs. Such methods include, for example, cell microfiltration systems. Examples of microfiltration methods include using microfilters with arrays of openings of a predetermined shape and size (˜8-14 μm) to prevent passage of tumor cells through the microfilter while allowing the smaller cells, for example, red and white blood cells in a blood sample, to pass through (e.g. Isolation by Size of Epithelial Tumor cells, ISET; CellSieve™ microfilters (Creatv Microtech)). These methods offer high throughput capabilities and low cost. Because these methods rely solely on cell size or other physical properties they can often lack sensitivity and specificity for CTCs. Membrane microfilters, for example, can process large volumes of blood (˜9-18 ml) with about 85% recovery of CTCs in the sample, though large number of leukocytes are often retained as well. Thus, additional CTC specific detection procedures are required to detect the CTCs in the pool of retained cells.

Additional filtration-type methods employ microfluidic chips that contain arrays of cell traps that inhibit passage of tumor cells based on properties unique to or characteristic of CTCs, such as, but not limited to, shear modulus, stiffness, size and/or deformability. Exemplary of such chips is the CTChip® chip (Clearbridge Biomedics Pte Ltd., Singapore; see also, Tan S. J. et al. (2009) Biomedical Microdevices 11(4): 883-892 and Tan et al. (2010) Biosens and Bioelect 26:1701-1705; see, also International PCT application No WO 2011/109762). CTCs, which are larger and stiffer are retained in the traps on the chips while the more deformable non-tumor cells, e.g. blood cells, pass through.

Density gradient methods, such as Ficoll density gradient separation and OncoQuick (Hexyl Gentech/Geiner Bio-One), enrich CTCs based on their lower buoyant density (<1.077 g/ml). The Ficoll density gradient method includes the steps od passing blood samples through a Ficoll gradient in a one step centrifugation. The upper mononucleocyte (MNC) fraction contains mononuclear blood cells as well as the CTCs. Following isolation of this layer, subsequent immunostaining with epithelial cell markers is generally required to positively identify CTCs. The OncoQuick method employs discontinuous gradient cell separation medium overlayed with a porous barrier. During centrifugation, the medium moves up through the porous barrier, while mononuclear cells move downward through the barrier and become trapped below the barrier. The OncoQuick provides a more enriched sample of CTCs compared to traditional Ficoll density gradient separation because contaminating mononuclear cells are depleted from the CTC fraction. The OncoQuick density gradient separation can produce a CTC fraction containing about 9.5×10⁴ mononuclear cells compared to 1.8×10⁷ mononuclear cells in the Ficoll density gradient separation for a 10 mL blood sample (Gertler et al. (2003) Recent Results Cancer Res. 162:149-55). As with Ficoll density gradient separation, the OncoQuick enriched sample still requires detection of CTCs by immunostaining.

CTCs that have been isolated by available direct isolation methods, such as those described herein, all generally require some method of detection to confirm that the isolated cells are CTCs. Such methods typically involve immunostaining for epithelial cell and other tumor cell markers, fluorescence in situ hybridization (FISH) and/or morphological analysis. Analysis of individual cells can be time consuming and difficult to automate. In addition, antibody staining procedures often involve multiple binding and washing steps which can damage the cells or cause loss of viable cells. Immunostaining with fluorophore-conjugated antibodies can be used to fluorescently label cells, and detection of a fluorescent signal can be automated. There, however, are problems associated with cell loss and variable detection.

In vivo methods of detecting CTCs also are available, including quantitation by intravital flow cytometry (see, e.g., He et al. (2007) Proc. Natl. Acad. Sci. USA 104(28):11760-11765). Effective in vivo methods for quantification of CTCs are highly desirable because it allows scanning of larger volumes of body fluids for the rare circulating cells. Scanning of larger volumes of blood can increase the statistical significance of the method and provide more accurate quantitation of rare events (<1 CTC per ml). For example, the entire blood volume content circulating in a subject could be scanned by scanning CTCs as they pass through the peripheral vasculature. Currently available in vivo methods rely on the administration of labeled antibodies and other detectable ligands or substrates (e.g., folate-FITC, folate-AlexaFluor 488, and folate rhodamine) that either specifically bind to or are taken up by the tumor cells. Detection is accomplished by fluorescence or radiographic scanning of surface blood vessels to detect the labeled agent bound to the circulating tumor cells. Such methods require high specificity binding of the reagent in vivo, and administration of high doses of the agent in order to ensure that the rare tumor cells are exposed to the reagent. Such high doses of detectable agents, for example, conjugated fluorescent dyes, can cause toxicity in the subject.

In general, there is considerable variability in the numbers of CTCs that are detected among the different currently available CTC detection methods, which is likely due to the variability in the nature of the methods for detection, differences in the sensitivity and specificity for CTCs, and reproducibility of the methods. Because of the lack of standardization in the field, implementing CTC detection into clinical practice in making treatment decisions has yet not been achieved. Several existing tumor enrichment methods described above are effective for capturing CTCs, but are ineffective in detecting CTCs due to the disadvantages of immunostaining and/or time consuming cell analysis. As described herein, oncolytic reporter viruses can obviate these problems by providing a means to detect CTCs without the need for additional staining procedures and extensive washing steps. The methods provided herein exploit the property of oncolytic viruses, such as vaccinia virus, to preferentially infect CTCs versus non-tumor cells. Infection of CTCs by oncolytic viruses does not rely on expression of a CTC specific marker and thus is not susceptible to the variable expression of these genes during metastasis.

Among the methods provided herein are improved methods for detecting CTCs in a sample using a combination of a tumor cell enrichment method with an oncolytic reporter virus for detection. Also among the methods provided herein are improved for detecting CTCs in vivo by administering oncolytic reporter viruses which eliminate the need for CTC-specific antibodies or other ligands which can be difficult to generate and/or are toxic.

3. Infection of Metastatic Cells and Cancer Stem Cells by Oncolytic Viruses

As described herein and in the examples provided herein, oncolytic viruses, such as LIVP vaccinia viruses, exhibit a preference for infecting metastasizing cells and metastatic tumors (see, e.g., Examples 5-10). In a mouse xenograft model of prostate cancer metastasis, vaccinia virus that was administered systemically to the tumor-bearing mouse infected and replicated in the primary tumor and also infected and replicated in migrating metastatic cells in lymphatic vessels and secondary lymph node metastases. Infection of the primary tumor and metastases was detectable via expression of a reporter gene encoded by the virus. Analysis of the excised metastatic lymph node tumors indicated that higher virus titer was present in the metastatic lymph node tumors that arose at later time points, indicating a preference for infection and/or replication of metastasizing tumor cells. Higher blood vessel density was observed at the sites of metastasis which can contribute to increased access of the virus to the metastasizing cells. Accordingly, such viruses can be used to monitor the real-time metastatic spread of a tumor.

In addition to the colonizing migrating metastatic cells in the lymphatic vessels, vaccinia virus also was found in over 78% of CTCs isolated from the peripheral blood of the tumor-bearing mice at one week following virus infection, as detected by expression of the reporter gene encoded by the virus in purified CTCs, isolated on a size-based CTC chip (e.g., the CTChip® chip (Clearbridge Biomedics Pte Ltd., Singapore; see, also, Tan S. J. et al. (2009) Biomedical Microdevices 11(4): 883-892 and Tan et al. (2010) Biosens and Bioelect 26:1701-1705; see, also International PCT application No WO 2011/109762). Vaccinia virus normally is rapidly cleared from the blood stream and non-tumor tissues following intravenous infection. Circulating CTCs also have a short half life in circulation. Thus, detection of infected CTCs at one week following infection indicates that the detected CTCs are likely tumor cells shed from the infected tumor. Oncolytic viruses, such as vaccinia virus, can thus be employed for the detection of CTCs that are shed from a metastasizing tumor.

In preclinical models, cancer stem cells are highly invasive and exhibit metastatic properties. As described herein, oncolytic viruses such as LIVP vaccinia virus exhibit increased infection and/or replication in subpopulations of tumor cells displaying cancer stem cell properties (e.g. expression of cancer stem cell markers, such as aldehyde dehydrogenase (ALDH1) and CD44) and higher tumorigenic potential and in tumor cells that have undergone epithelial mesenchymal transition (EMT) (see, e.g. Examples 28, 29, 33 and 36). For example, in GI-101A breast cancer cell lines, ALDH1⁺ cells display properties of cancer stem cells, including higher invasiveness, tumorigenic potential and chemotherapeutic and ionizing radiation resistance compared to ALDH1⁻ cells. It is shown herein that oncolytic viruses, such as vaccinia viruses, exhibit enhanced replication in ALDH¹⁺ cells and selective targeting and tumor regression in ALDH1⁺ cell derived tumors. These data indicate that LIVP vaccinia viruses exhibit preferential infection and/or replication in tumor cell populations that have higher potential for forming tumors in vivo. Thus, oncolytic viruses such as LIVP vaccinia viruses provide a means for more specific identification of tumorigenic CTCs over other methods. Thus, the number of CTCs identified by oncolytic viruses such as LIVP vaccinia viruses can have higher clinical relevance compared to numbers of CTCs selected by other methods in the art.

Current methods for detection of CTCs lack specificity, sensitivity or involve labor intensive processing steps that result in the loss of CTCs. As described herein, oncolytic reporter viruses exhibit preferential infection and/replication in tumor cells, including metastatic tumor cells, in vivo in a subject and ex vivo in a sample, and can be employed in methods of detecting and enumerating CTCs that are shed from primary tumors. The oncolytic virus effectively labels the metastatic cells, and labeled cells can be detected upon shedding into the circulatory system, other bodily fluids, or disseminated into the bone marrow.

Accordingly, provided herein are methods of detecting CTCs that use oncolytic reporter viruses for infection and detection of CTCs in vivo in a subject and ex vivo in a sample from a subject. The oncolytic viruses can be used alone or in combination with one or more methods of enrichment of CTCs. By combining tumor cell enrichment methods that maximize the level of CTCs retained in a sample with the tumor cell detection capabilities of oncolytic reporter viruses, high throughput of samples and high specificity and high sensitivity CTC detection can be achieved. Further, the specificity and ability of oncolytic viruses, such as LIVP vaccinia virus, to infect metastasizing cells in vivo demonstrates that such viruses can be administered for in vivo detection and ex vivo detection in samples, such as from subjects undergoing oncolytic virus therapy.

### C. METHODS FOR DETECTING CIRCULATING TUMOR CELLS USING ONCOLYTIC REPORTER VIRUSES

The methods provided herein for the detection of circulating tumor cells (CTCs) are based on the ability of oncolytic viruses to preferentially infect tumor cells, including CTCs, in vitro and in vivo, compared to non-tumor cells. In particular, it is described herein that oncolytic viruses, including, for example, vaccinia viruses, such as LIVP vaccinia viruses, exhibit preferential replication in tumor cell subpopulations with high tumorigenic potential, including cancer stem cells, EMT-induced tumor cells, and in vivo metastasizing cells.

According to the methods provided herein, the oncolytic reporter viruses can infect CTCs, and the infected CTCs can be easily detected via expression of a reporter gene encoded by the virus. In some examples, the oncolytic reporter virus infects the tumor cells of a primary tumor in vivo, and the CTCs that are shed from the tumor are infected CTCs that can be detected. Methods of detection of reporter genes are known in the art and can be performed in vivo in a subject or ex vivo with a sample. Accordingly, the methods provided herein for detecting one or more CTCs using oncolytic reporter viruses can be performed in vivo or ex vivo. The methods provided herein for detecting one or more CTCs in vivo in a subject or ex vivo in a sample involve evaluating the preferential infection of CTCs by the oncolytic virus via detection expression of a reporter gene encoded by the virus, thereby identifying the CTCs. In particular examples, the oncolytic reporter virus is an oncolytic vaccinia virus, such as an LIVP vaccinia virus.

The methods provided herein for detection of a circulating tumor cell (CTC) encompass ex vivo detection of CTCs in a sample from a subject or in vivo detection of CTCs in a subject using an oncolytic reporter virus encoding a reporter gene. In some examples, a method for detection of CTCs includes infection of a sample from a subject with an oncolytic reporter virus, such as an oncolytic vaccinia virus encoding a reporter gene, and then detecting the expressed reporter protein by the infected cells in the sample, thereby detecting the CTCs. In some examples, a method for detection of CTCs includes detecting CTCs in a sample, where the sample is from a subject treated with an oncolytic reporter virus, such as an oncolytic vaccinia virus encoding a reporter gene, and detection involves detection of the expressed reporter protein by the infected cells in the sample, thereby detecting the CTCs. In some examples, a method for detection of CTCs includes administering an oncolytic reporter virus, such as an oncolytic vaccinia virus encoding a reporter gene, to a subject and then detecting the reporter protein expressed by the infected cells in vivo, thereby detecting the CTCs in the subject.

Among the methods provided herein are methods that increase the sensitivity and specificity of CTC detection in a sample. As described herein, using oncolytic reporter viruses for CTC detection obviates the need for staining procedures that can cause loss of CTCs in a sample, produce false positives or lack sensitivity for detecting tumorigenic CTCs. The use of oncolytic viruses for CTC detection can improve the detection capabilities of existing tumor cell enrichment methods. In some examples, the CTCs are detected using a combination of a tumor cell enrichment method and infection with an oncolytic reporter virus, such as an oncolytic vaccinia virus encoding a reporter gene. For example, in some examples, the sample is first processed using a tumor cell enrichment method to enrich or concentrate the CTCs in the sample, and then the CTC enriched sample is infected with the vaccinia virus for detection of CTCs by detection of the expressed reporter protein. In other examples, the sample is first infected with an oncolytic reporter virus, such as an oncolytic vaccinia virus encoding a reporter gene, and then the infected sample is processed using a tumor cell enrichment method, where the CTCs are detected by detection of the expressed reporter protein. In some examples, one tumor cell enrichment method is employed. In some examples, two or more tumor cell enrichment methods are employed. The sample can be infected with the oncolytic reporter virus before or during or subsequent to performing one or more tumor cell enrichment methods on the sample.

A tumor cell enrichment method can involve positive selection and/or negative selection methods to enrich for CTCs in the sample. For example, the tumor cell enrichment method can involve selection and separation of tumor cells from non-tumor cells and other components of the sample (i.e. positive selection) and/or can involve selection and removal of non-tumor cells or other components from the sample (i.e. negative selection). Positive selection of tumor cells can be based on any property of the cells including, but not limited, physical properties, such as, for example, size, stiffness, density, shear modulus, or deformability, or biological properties, such as the expression of a tumor cell specific marker or cell invasiveness. Using an oncolytic reporter virus for detection of CTCs enriched in a sample using a tumor cell enrichment method avoids need for additional cell manipulations such as immunostaining because CTCs that are infected with the reporter virus express a detectable reporter gene product, such as, for example, a fluorescent protein (e.g. GFP or TurboFP635), a luminescent protein, an enzyme that produces a detectable product, or a protein that binds to a detectable substrate (e.g. a receptor). Additional exemplary detectable gene products are provided elsewhere herein.

In some examples, positive selection of tumor cells can be based on expression of a virally encoded protein. Infection of cells with a virus that encodes for a protein results in expression of the protein in the tumor cells. Cells that express the protein can be isolated. For example, if the virally encoded protein is a membrane protein, such as a receptor or transporter, cells that encode the protein can be isolated by immunocapture using an antibody specific for the protein.

Detection and/or enumeration of CTCs can be used, for example, for diagnosis of cancer, staging a cancer, determining the prognosis of a cancer, predicting the responsiveness of a subject to therapy with an oncolytic virus and/or monitoring effectiveness of an anti-cancer therapy, including therapy with an oncolytic virus alone or in combination with one or more additional anti-cancer agents. This can be effected by comparison to a control or reference sample or reference number of classifications of known levels of CTCs. For example, as described herein, it is found that oncolytic reporter viruses such as LIVP vaccinia viruses, preferentially infect metastasizing cells and cancer stem cells and decrease metastasis. Thus, detection of metastasis by detection of CTCs as provided herein, also can be used to stratify patients for treatment with an oncolytic virus to treat the metastasis.

In some examples, the oncolytic reporter viruses are employed to detect one or more CTCs in a fluid sample from a subject. Exemplary fluid samples are provided elsewhere herein and include, for example, blood, lymph, cerebrospinal fluid, pleural fluid, and peritoneal fluid. Typically, the sample contains one or more non-tumor cells in the sample. In some examples, such as a blood sample, the sample contains non-tumor cells including but not limited to red blood cells (RBCs, erythrocytes) and white blood cells, including leukocytes and platelets. In some examples, a CTC is detected among 1, 10, 100, 1×10³, 1×10⁴, 1×10⁵, 1×10⁶, 1×10⁷, ×10⁸, 1×10⁹, 1×10¹⁰, 1×10¹¹, 1×10¹², 1×10¹³, 1×10¹⁴, 1×10¹⁵, or more non-tumor cells.

In particular examples, the methods provided herein can detect 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1000, 1500, 2000 or more tumor cells in a body fluid sample, such as 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1000, 1500, 2000 or more tumor cells per 1 mL of a body fluid sample. In particular examples, the methods provided herein can detect 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1000, 1500, 2000 or more tumor cells in a blood sample, such as 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1000, 1500, 2000 or more tumor cells per 1 mL of a blood sample.

Cancer progression or effectiveness of cancer treatment can be determined using the methods provided herein. In particular examples, the level of CTCs is measured at a first time point using the methods provided and then compared to the level of CTCs measured at a second later time point by the same method. In some examples, the first time point is at a predetermined time prior to administration of a therapy, such as an anti-cancer therapy, and the second time point is at a predetermined time following administration of the therapy, during the administration of the therapy, or between successive administrations of the therapy. In exemplary methods, the sample can be obtained from the subject, for example, at least, at about or at 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or later following administration of the anti-cancer therapy to the subject. In some examples, samples are collected at a plurality of time points, such as at more than one time point, including, for example, at 1, 2, 3, 4, 5, 6, 7, 8, 9, 10, 15, 20 or more time points following administration of the anti-cancer therapy to the subject. In some examples, samples are collected at regular intervals following administration of the anti-cancer therapy to the subject.

In particular examples, the level of CTCs is measured at a first time point and then compared to the level of CTCs measured at a second later time point to determine cancer progression over time, where if the level of CTCs at the second time point is greater than the level of CTCs at the first time point, then the cancer has advanced in progression. In particular examples, if the level of CTCs at a second time point is 2, 3, 4, 5, 6, 7, 8, 8, 9, 10, 20, 30, 40, 50, 60, 70, 80, 90, 100, 200, 300, 400, 500, 600, 700, 800, 900, 1000 or more times greater than the level of CTCs at a first time point, then the cancer has advanced in progression.

In particular examples, the level of CTCs is measured at a first time and then compared to the level of CTCs measured at a second later time point to determine cancer regression over time, where if the level of CTCs at the second time point is less than the levels of CTCs at the first time point, then the cancer has regressed. In particular examples, if the level of CTCs at a first time point is 2, 3, 4, 5, 6, 7, 8, 8, 9, 10, 20, 30, 40, 50, 60, 70, 80, 90, 100, 200, 300, 400, 500, 600, 700, 800, 900, 1000 or more times greater than the level of CTCs at a second time point, then the cancer has regressed.

In particular examples, the level of CTCs is measured at a first time and then compared to the level of CTCs measured at a second later time point to determine stabilization of cancer over time, where if the level of CTCs at the second time point is equal to or about the same as the levels of CTCs at the first time point, then the cancer has stabilized.

In particular examples, the level of CTCs is measured at a first time point and then compared to the level of CTCs measured at a second later time point to determine the effectiveness of therapy in inhibiting cancer progression, where if the level of CTCs at the second time point is less than or equal to the levels of CTCs at the first time point, then the therapy is effective at inhibiting cancer progression. In particular examples, if the level of CTCs at a first time point is equal to or 2, 3, 4, 5, 6, 7, 8, 8, 9, 10, 20, 30, 40, 50, 60, 70, 80, 90, 100, 200, 300, 400, 500, 600, 700, 800, 900, 1000 or more times greater than the level of CTCs at a second time point, then the therapy is effective at inhibiting cancer progression.

In particular examples, the level of CTCs is measured at a first time point and then compared to the level of CTCs measured at a second later time point to determine the effectiveness of therapy in inhibiting cancer progression, where if the level of CTCs at the second time point is greater than the levels of CTCs at the first time point, then the therapy is not effective at inhibiting cancer progression. In particular examples, if the level of CTCs at a second time point is 2, 3, 4, 5, 6, 7, 8, 8, 9, 10, 20, 30, 40, 50, 60, 70, 80, 90, 100, 200, 300, 400, 500, 600, 700, 800, 900, 1000 or more times greater than the level of CTCs at a first time point, then the therapy is not effective at inhibiting cancer progression.

In some examples, the methods provided herein can detect at or about a 2-fold, 3-fold, 4-fold, 5-fold, 6-fold, 7-fold, 8-fold, 9-fold, 10-fold, 200-fold, 300-fold, 400-fold, 500-fold, 600-fold, 700-fold, 800-fold, 900-fold, 1000-fold or higher increase in the level of CTCs over time relative to a control sample. In particular examples, the methods provided herein can detect at or about a 2-fold, 3-fold, 4-fold, 5-fold, 6-fold, 7-fold, 8-fold, 9-fold, 10-fold, 200-fold, 300-fold, 400-fold, 500-fold, 600-fold, 700-fold, 800-fold, 900-fold, 1000-fold or higher decrease in the level of CTCs over time relative to a control sample. In some examples, the control sample is a sample obtained from a subject at a first time point and compared to a sample obtained from the subject at a second time point. In some examples, the control sample is a sample with a known amount of CTCs. In some examples, the control sample is a sample obtained from a subject with a particular cancer, a known stage of cancer, or a known cancer prognosis.

In some examples, a single body fluid sample is obtained from the subject at a particular time point. In some examples, a plurality of body fluid samples are obtained from the subject at a particular time. In some examples, body fluid samples of two or more different types are obtained, such as for example, a blood sample and a lymph sample. Exemplary types of fluid samples are provided herein.

In some examples, the oncolytic reporter virus is administered to a subject for the diagnosis and therapy. As is known in the art and described herein, oncolytic viruses, such as the LIVP vaccinia virus, can accumulate in tumors and metastases and are able to treat to the metastases without the expression of any additional gene products. Accordingly, the oncolytic reporter virus can be administered to a subject for detection of CTCs in vivo or ex vivo according to the methods provided herein and additionally treat the primary tumor, secondary metastases and/or CTCs.

Expression of one or more additional therapeutic gene products can enhance the therapy of the cancer. Accordingly, in some examples, the oncolytic reporter virus encodes one or more genes for therapy, such as a therapeutic gene for the treatment of cancer. Exemplary therapeutic gene products are provided elsewhere herein. In particular examples, the therapeutic gene encodes an anti-metastatic gene product.

1. Exemplary Methods for Detection of CTCs with an Oncolytic Reporter Virus

a. Ex Vivo Detection of CTCs in Samples Treated with an Oncolytic Reporter Virus

In some examples, the method involves ex vivo detection and/or enumeration of CTCs in a sample obtained from a subject. For example, the method for detection and/or enumeration of CTCs in a sample involves contacting a sample from a subject with an oncolytic reporter virus and detecting infected cells by expression of a reporter protein. Because the oncolytic reporter viruses preferentially infect the tumor cells in the sample compared to non-tumor cells, detection of the expressed reporter gene product in infected cells thereby detects the CTCs in the sample. In some examples, the sample is obtained from a subject who has a cancer or metastasis or is suspected of having a cancer or metastasis.

In exemplary methods for ex vivo detection of tumor cells in a body fluid sample from a subject, the method involves the steps of: 1) providing a body fluid sample from a subject; 2) contacting the sample with an oncolytic reporter virus; and 3) detecting one or more cells infected by the oncolytic virus in the sample, thereby detecting one or more tumor cells. In some examples, the method includes the step of collecting the sample from the subject. In some examples, cells infected by the oncolytic reporter virus are detected by detecting expression of a reporter gene product encoded by the virus.

In some examples, the sample is infected with the oncolytic reporter virus immediately following collection of the sample from the subject. In other examples, the sample is infected with the oncolytic virus at about 1, 2, 4, 6, 12, 24, 48 or 72 hours or more after collection of the sample. In some examples, the cells in the sample are first concentrated by centrifugation, and then resuspended in an appropriate medium prior to infection with the virus.

In some examples, a method for ex vivo detection of CTCs in a sample from a subject involves performing a tumor cell enrichment method on the sample in combination with infection with an oncolytic reporter virus. Exemplary tumor cell enrichment methods are provided elsewhere herein, and include, for example, the passage of the sample through a microfilter or microfluidic device, immunomagnetic separation and/or removal of non-tumor cells from the sample. In some examples, the sample can be infected with the oncolytic reporter virus prior to performing of the tumor cell enrichment method. For example, the sample can be infected with the oncolytic reporter virus 1, 2, 3, 4, 5, 6, 7, 8, 9, 10, 11, 12, 13, 14, 15, 16, 17, 18, 19, 20, 21, 22, 23, 24 or more hours prior to performing of the tumor cell enrichment method. In other examples, the sample can be infected with the oncolytic reporter virus during performance of the tumor cell enrichment method. In other examples, the enriched sample can be infected with the oncolytic reporter virus following performance of the tumor cell enrichment method (i.e. the virus is used to infect the enriched sample). For example, the enriched sample can be infected with the oncolytic reporter virus 1, 2, 3, 4, 5, 6, 7, 8, 9, 10, 11, 12, 13, 14, 15, 16, 17, 18, 19, 20, 21, 22, 23, 24 or more hours after performing of the tumor cell enrichment method.

In some exemplary methods for ex vivo detection tumor cells in a sample from a subject, the method involves the steps of: 1) providing a body fluid sample from a subject; 2) performing a tumor cell enrichment method on the sample; 3) contacting the sample with an oncolytic reporter virus; and 4) detecting one or more cells infected by the oncolytic virus in the sample, thereby detecting one or more tumor cells. In some examples, step 2 is performed prior to step 3. In some examples, step 2 is performed following step 3. In some examples, steps 2 and 3 are performed simultaneously. In some examples, the method includes the step of collecting the sample from the subject. In some examples, cells infected by the oncolytic reporter virus are detected by detecting expression of a reporter gene product encoded by the virus.

For virus infection, the oncolytic reporter virus is added to the sample at a sufficient concentration, or multiplicity of infection (MOI) as to effect an appropriate level of infection that enables detection of CTCs by a particular method. The level of infection required can be determined by one of skill in the art. For example, if the level of expression of a reporter protein is to be assessed within hours of infection of the CTCs, then a sufficiently high level of infection can be achieved immediately to rapidly produce a detectable amount of the reporter protein. The type of reporter protein, the strength of the promoter, and the sensitivity of the detection methods also can influence the level of infection required. In some examples, the MOI is about 0.00001 to about 10, such as for example, about 0.0001 to about 1.0. Exemplary MOI include, for example, at or about 0.00001, 0.0001, 0.001, 0.01, 0.1, 1.0, 10 or more.

Determination of a multiplicity of infection to use in the assay for a particular reporter virus can be determined using well-known methods to assess infectivity, such as by a plaque-forming unit (pfu) assay. For an assay to measure the level of CTCs in a sample, typically a multiplicity of infection is selected to ensure all CTCs are infected while non-CTCs are not infected. The precise conditions for infection of cells with an oncolytic reporter virus are selected according to the sample, the particular reporter virus and the detection method. Such conditions can be readily determined and modified by one of skill in the art. Exemplary conditions for infection of samples are provided in the Examples provided herein. In non-limiting examples, 10 pfu, 100 pfu, 1×10³ pfu, 1×10⁴ pfu, 1×10⁵ pfu, 1×10⁶ pfu, 1×10⁷ pfu, 1×10⁸ pfu, 1×10⁹ pfu, 1×10 pfu or more of an oncolytic reporter virus, such as a vaccinia virus, is used to infect 1 mL of a fluid sample, such as a blood sample, from a subject.

Detection of the expressed reporter gene product in the infected CTCs can be performed at a predetermined time following infection or at multiple time points following infection. A detectable level of reporter protein can accumulate in, for example, 2 hours or more, 4 hours or more, 6 hours or more, 8 hours or more, 12 hours or more, 24 hours or more, or 48 hours or more following viral infection. The type of reporter protein and the sensitivity of the detection methods can influence the incubation time required. Determination of the optimal time for detection of the expressed reporter gene is well within the capabilities of one of skill in the art and can be determined empirically in a sample that contains a known level of CTCs.

Exemplary methods of detecting expressed reporter gene products are provided elsewhere herein and include, but are not limited to fluorescent, luminescent, spectrophotometric, chromogenic assays, or radioactive detection methods.

b. Ex Vivo Detection of CTCs in Samples from Subjects Treated with an Oncolytic Reporter Virus

In some examples, the method for detection and/or enumeration of CTCs in a sample involves detecting a reporter gene expressed in a sample from a subject to whom a oncolytic reporter virus was administered. As described herein, tumor cells, in particular, metastasizing cells and cells exhibiting stem cell like properties, are preferentially infected by oncolytic viruses, such as vaccinia virus, in vivo following administration to a subject with a metastasizing tumor. The CTCs that are shed from the tumors also are infected with the oncolytic virus, thus permitting their detection in fluid samples from the subject. In some examples, the sample is obtained from a subject who has a cancer or metastasis or is suspected of having a cancer or metastasis.

In some exemplary methods for ex vivo detection tumor cells in a body fluid sample from a subject, the method involves the steps of: 1) providing a sample from a subject that has been administered an oncolytic reporter virus; and 2) detecting one or more cells infected by the oncolytic virus in the sample, thereby detecting one or more tumor cells. In some examples, the method includes the step of collecting the sample from the subject. In some examples, cells infected by the oncolytic reporter virus are detected by detecting expression of a reporter gene product encoded by the virus.

In some examples, the method includes a step of administering an oncolytic virus encoding a reporter gene to a subject that has cancer or is suspected of having cancer for the detection of CTCs. For example, in some exemplary methods for ex vivo detection tumor cells in a body fluid sample from a subject, the method involves the steps of: 1) administering an oncolytic reporter virus to a subject; 2) obtaining a body fluid sample from the subject; and 3) detecting one or more cells infected by the oncolytic virus in the sample, thereby detecting one or more tumor cells. In some examples, cells infected by the oncolytic reporter virus are detected by detecting expression of a reporter gene product encoded by the virus.

The oncolytic viruses encoding a reporter gene can be administered to the subject by any suitable method for administering a diagnostic or therapeutic oncolytic virus. Administration of oncolytic viruses to a subject, including a human subject or non-human mammalian subject, is well-known in the art. The oncolytic reporter virus can be administered by any suitable route. For example, the oncolytic viruses encoding a reporter gene can be administered to the subject systemically or locally to the tumor. Exemplary routes of administration include, but are not limited to intravenous, intraarterial, intratumoral, endoscopic, intralesional, intramuscular, intradermal, intraperitoneal, intravesicular, intraarticular, intrapleural, percutaneous, subcutaneous, oral, parenteral, intranasal, intratracheal, inhalation, intracranial, intraprostatic, intravitreal, topical, ocular, vaginal, or rectal routes of administration. In particular examples, the oncolytic viruses encoding a reporter gene are administered intraperitoneally or intravenously.

The dosage regimen can be any of a variety of methods and amounts, and can be determined by one skilled in the art according to known clinical factors. As is known in the medical arts, dosages for any one subject can depend on many factors, including the subject's species, size, body surface area, age, sex, immunocompetence, and general health, the particular virus to be administered, duration and route of administration, the kind and stage of the disease, for example, tumor size, and other treatments or compounds, such as chemotherapeutic drugs, being administered concurrently. In addition to the above factors, such levels can be affected by the infectivity of the virus, and the nature of the virus, as can be determined by one skilled in the art. In the present methods, appropriate minimum dosage levels of viruses can be levels sufficient for the virus to survive, grow and replicate in a tumor or metastasis. Exemplary minimum levels for administering a virus to a 65 kg human can include at least or about 1×10⁵ plaque forming units (PFU), at least about 5×10⁵ PFU, at least about 1×10⁶ PFU, at least about 5×10⁶ PFU, at least about 1×10⁷ PFU, at least about 1×10⁸ PFU, at least about 1×10⁹ PFU, or at least about 1×10¹⁰ PFU. In the present methods, appropriate maximum dosage levels of viruses can be levels that are not toxic to the host, levels that do not cause splenomegaly of 3 times or more, levels that do not result in colonies or plaques in normal tissues or organs after about 1 day or after about 3 days or after about 7 days. Exemplary maximum levels for administering a virus to a 65 kg human can include no more than about 1×10¹¹ PFU, no more than about 5×10¹⁰ PFU, no more than about 1×10¹⁰ PFU, no more than about 5×10⁹ PFU, no more than about 1×10⁹ PFU, or no more than about 1×10⁸ PFU.

Typically, the body fluid sample is obtained at a predetermined time following administration of the virus. In some examples, the predetermined time is sufficient for the virus to infect a tumor cell in the subject. In some example the predetermined time is sufficient for the free virus to be cleared from the subject. As one skilled in the art will recognize, the time period for oncolytic virus infection of the tumor and appearance of infected CTCs in a fluid sample from the subject will vary. For example, the time period for infection of a virus will vary depending on factors, such as the infectivity of the virus, the route of administration, the immunocompetence of the host and dosage of the virus. Such times can be empirically determined if necessary.

Generally, expression of reporter protein in CTCs infected with an oncolytic reporter virus can be determined at time points from about less than 1 day, about or 1 day to about 2, 3, 4, 5, 6 or 7 days, about or 1 week to about 2, 3 or 4 weeks, about or 1 month to about 2, 3, 4, 5, 6 months or longer after administration of the virus. In exemplary methods, the sample can be obtained from the subject, for example, at least, at about or at 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or later following administration of the oncolytic reporter virus to the subject. In some examples, samples are collected from the subject at multiple time points, such as at more than one time point, including, for example, at 2, 3, 4, 5, 6, 7, 8, 9, 10, 15, 20 or more time points.

As shown in the examples provided, oncolytic reporter viruses, such as the oncolytic reporter vaccinia viruses, are therapeutic and are able to treat metastases and CTCs in the subject. This leads to a decrease in the number of tumor cells that are metastasizing and are shed from the tumor. Thus, for use in initial detection of metastasis in a subject, a body fluid sample generally is obtained from the subject within a time period prior to significant reduction of metastasis due to oncolytic activity of the virus. In exemplary methods for the initial detection of the metastasis using an oncolytic reporter virus, a body fluid sample typically is obtained a predetermined time within a few weeks following administration of the virus. In particular examples, the body fluid sample is obtained from the subject 6 hours, 12 hours, 18 hours, I day, 2 days, 3 days, 4 days, 5 days, 6 days, 7 days, 8 days, 9 days, 10 days, 11 days, 12 days, 13 days, or 14 days following administration of the virus to the subject.

In some examples, a method for ex vivo detection of CTCs in a sample from a subject involves performing a tumor cell enrichment method on the sample. For example, in some exemplary methods for ex vivo detection tumor cells in a sample from a subject, the method involves the steps of: 1) providing a body fluid sample from a subject that that has been administered an oncolytic reporter virus; 2) performing a tumor cell enrichment method on the sample; and 3) detecting one or more cells infected by the oncolytic virus in the sample, thereby detecting one or more tumor cells. In some examples, cells infected by the oncolytic reporter virus are detected by detecting expression of a reporter gene product encoded by the virus. In some examples, the method includes the step of collecting the body fluid sample from the subject.

In some examples, the method includes a step of administering an oncolytic virus encoding a reporter gene to a subject for the detection of tumor cells and also involves performing a tumor cell enrichment method on the sample. In some exemplary methods for ex vivo detection tumor cells in a sample from a subject, the method involves the steps of: 1) administering an oncolytic reporter virus to a subject; 2) obtaining a body fluid sample from the subject; and 3) detecting one or more cells infected by the oncolytic virus in the sample, thereby detecting one or more tumor cells. In some examples, cells infected by the oncolytic reporter virus are detected by detecting expression of a reporter gene product encoded by the virus. In some examples, the method also involves performing a tumor cell enrichment method on the sample.

Exemplary methods of detecting expressed reporter proteins are provided elsewhere herein and include, but are not limited to fluorescent, luminescent, spectrophotometric, chromogenic assays, or radioactive detection methods.

c. In Vivo Detection of CTCs in Subjects Treated with an Oncolytic Reporter Virus

In exemplary methods, real time detection and quantification of CTCs can be performed in vivo as the CTCs circulate through a live subject. Such methods can be performed without extraction of a body fluid sample from the subject. For example, CTCs expressing a detectable protein can be detected as the cells pass through peripheral blood vessels close to the surface of the skin (e.g., intravital flow cytometry; see, e.g., He et al. (2007) Proc. Natl. Acad. Sci. USA 104(28):11760-11765). In some examples, CTCs expressing a fluorescent protein can be irradiated to excite the expressed fluorescent protein, and the labeled cells can be quantified by detecting the fluorescent radiation emitted by the excited cells by an in vivo flow cytometry method. In some examples, the cells are detected as they circulate pass near an external detector. In some examples, an implantable device is employed for detection. Examples of such methods for in vivo detection of circulating cells, including labeled cancer cells, are described in, for example, in Georgakoudi et al. (2004) Cancer Research 64: 5044, Boutrus et al. (2007) J. Biomed. Opt. 12(2): 020507, Gal et al. (2005) Arthritis and Rheumatism 52: 3269, Novak et al. (2004) Optics Letters 29(1): 77, and Wie et al. (2005) Mol Imaging 4(4): 415-416.

As described herein, oncolytic reporter viruses administered to a tumor bearing subject result in CTCs that are infected with the oncolytic reporter virus. Such cells can be detected in vivo using an in vivo flow cytometry method which detects expression of the reporter protein by the infected CTCs.

Accordingly, a subject having cancer or metastasis or is suspected of having a cancer or metastasis can be administered an oncolytic reporter virus, such as a vaccinia virus, encoding a detectable protein, such as a fluorescent protein, and detected in vivo using an in vivo detection method such as an in vivo flow cytometry method. In an exemplary method for in vivo detection of circulating tumor cells in a subject, the method involves the steps of: 1) administering an oncolytic reporter virus to a subject; and 2) detecting one or more cells infected by the oncolytic virus in vivo, thereby detecting one or more tumor cells. In some examples, cells infected by the oncolytic reporter virus are detected by detecting expression of a reporter gene product encoded by the virus.

In some examples, where the reporter protein is a receptor, a detectable ligand, such as a fluorescent or radiolabeled ligand, that binds to the receptor can be administered to the subject for detection of the CTCs in vivo. In other examples, where the reporter protein is an enzyme, a detectable substrate can be administered to the subject for detection of the CTCs in vivo.

Exemplary methods of detecting expressed reporter proteins are provided elsewhere herein and include, but are not limited to fluorescent, luminescent, spectrophotometric, chromogenic assays, or radioactive detection methods.

2. Methods for Enrichment of CTCs for Use in Combination with an Oncolytic Reporter Virus

Among the methods provided herein are methods of detecting one or more CTCs in a sample where the method involves performing one or more tumor cell enrichment methods in combination with detection of CTCs using an oncolytic virus. Any method that increases the amount of tumor cells in a sample relative to non-tumor cells or other non-cellular components in the sample can be employed to enrich the CTCs and can be used in combination with an oncolytic reporter virus for detection of CTCs. Such methods include, but are not limited to, positive selection of tumor cells based on one or more properties of a tumor cell or negative selection where non-tumor cells, such as, for example, blood cells, are removed from the sample. As described herein, use of an oncolytic virus in combination with a tumor cell enrichment method improves detection and enumeration of CTCs in a sample by providing a simple, easy and highly sensitive and specific method of identifying CTCs in the enriched sample without additional processing steps. Detection of CTCs with oncolytic reporter viruses does not require multistep staining procedures and reagents that are typically required for immunostaining procedures.

Tumor cell enrichment methods for use in combination with an oncolytic virus can be selected based on the specificity and/or sensitivity of the method. For example, a tumor cell enrichment method can be selected based on the ability of the method to decrease the amount of tumor cells in the sample with minimal or no loss of CTCs in the sample. In some examples, the tumor cell enrichment method results in the removal of at least about 10%, 20%, 30%, 40%, 50%, 60%, 70%, 80%, 90%, 95%, 96%, 97%, 98%, 99% of non-tumor cells from the sample. In some examples, the tumor cell enrichment method results in retention of at least about 50%, 60%, 70%, 80%, 90%, 95%, 96%, 97%, 98%, 99% of CTCs in the sample.

In some examples, the tumor cell enrichment method for use in combination with an oncolytic reporter virus involves selection of CTCs based on physical properties of CTCs. Exemplary physical properties include, for example, size, density, stiffness, deformability, and electrical charge compared to a non-tumor cell. In some examples, the tumor cell enrichment method for use in combination with an oncolytic reporter virus involves selection of CTCs based on biological properties of CTCs. Exemplary biological properties include, for example, expression of a cell surface marker or cell invasiveness. In some examples the tumor cell enrichment method for use in combination with an oncolytic reporter virus involves selection of CTCs based on a combination of one or more physical and/or one or more biological properties of a CTC. In particular examples, the tumor cell enrichment method uses a microfilter or a microfluidic device for the capture or retention of CTCs.

In some examples, the tumor cell enrichment method for use in combination with an oncolytic reporter virus involves positive and/or negative selection of CTCs in a sample based on the expression of one or more cell surface markers. For example, cell surface markers can be employed to select CTCs in a sample (i.e. positive selection) or to remove non-CTCs from a sample (i.e. negative selection). Exemplary markers for positive selection of CTCs include epithelial specific markers, markers of epithelial mesenchymal transition (EMT), cancer cell markers and cancer stem cell markers. Exemplary epithelial specific markers include, but are not limited to, EpCAM and cytokeratin (CK). Exemplary markers for negative selection of CTCs includes, but is not limited to, CD45 for selection of leukocytes.

Exemplary methods for tumor cell enrichment include, but are not limited to, microfiltration, microfluidic chip capture, immunomagnetic separation, density gradient separation, acoustophoresis, dielectrophoresis and selective lysis of particular cell types, for example, red blood cells in a blood sample (see also, Pantel and Alix-Panabieres (2010) Trends Mol Med 16(9):398-406).

a. Microfiltration

In some examples, the tumor enrichment method for use in combination with an oncolytic reporter virus involves capture of tumor cells by size segregation on a microfilter. For example, a microfilter that allows the passage of non-tumor cells but not tumor cells based on the larger size of the tumor cells can be employed to enrich CTCs in a sample. The CTCs in the enriched sample can be detected by infection with the oncolytic reporter virus and detection of the expressed reporter gene product encoded by the virus.

In exemplary methods, CTCs are enriched in a body fluid sample by applying the fluid sample to a microfilter. The enriched sample is then infected with the oncolytic reporter virus, and expression of the reporter gene is detected, thereby detecting the CTCs in the enriched sample.

Use of an oncolytic reporter virus for detection of CTCs allows CTCs to be detected on the microfilter without additional staining procedures. In some examples, infection of the captured CTCs is performed directly on the microfilter. For example, infection with the oncolytic reporter virus can be performed by adding the virus to the captured cells that have not passed through the microfilter. Exemplary methods for infecting cells on a microfilter are provided herein. In some examples, the microfilter is incubated in a suitable medium containing the virus for infection. In some examples, the infected CTCs are detected directly on the microfilter. In some example, the infected CTCs are removed from the microfilter and then detected.

In some examples, infection of the captured CTCs is performed after recovery of the captured cells from the microfilter. For example, the captured cells that have not passed through the microfilter can be gently removed from the microfilter using an suitable buffer to remove the cells from the surface of the filter and then contacted with the oncolytic reporter virus in a suitable medium for infection.

In some examples, the sample is first infected with oncolytic reporter virus and then the infected sample is passed through the microfilter. The cells that have not passed through the filter then can be detected directly on the filter.

In other exemplary methods, a microfilter is employed to enrich CTCs in a sample from a subject previously administered an oncolytic reporter virus. In such methods, the sample from the subject is passed through the microfilter and then expression of the reporter gene is detected, thereby detecting the captured CTCs in the enriched sample.

Microfilters for the enrichment of CTCs in a sample are available in the art for use in combination with an oncolytic reporter virus for detection. Exemplary microfilters include, but are not limited to, parylene slot filters (see e.g., Xu et al. (2010) Cancer Res 70(16):6420-6426 and U.S. Pat. Pub. No. 2011/0053152), track-etched filters (e.g. Nucleopore track-etched polycarbonate membrane filter (Whatman)), and CellSieve™ micropore filters (Creatv MicroTech). In some examples, the microfilter employed is part of an extracorporeal filtration device for the removal of CTCs from the subject's blood stream (see, e.g. US Pat. Pub. No. 2011/024443). In such examples, blood is directed from the subject through the filtration device, where CTCs are retained by the microfilter, and the filtered blood is administered back into the subject.

In some examples, the microfilter contains a plurality of pores. The pores can be any suitable geometric shape, provided the pores prevent passage of CTCs through the microfilter. For example, the pores can be circular, elliptical, oval, rectangular, square, symmetrical polygonal, unsymmetrical polygonal, or irregular shaped, or can comprise a combination of pores of different shapes. In some examples, the pores are arranged in an array on the microfilter. In some examples, the pores are spaced at regular intervals from each other (i.e. equidistant). In some examples, the pores are irregularly spaced. In some examples, the pores are arrayed in rows. In some examples, the pores in consecutive rows are offset from one another.

In some examples, where the microfilter contains circular pores, the pores are uniform in diameter. In some examples, where the microfilter contains circular pores, the pores are not uniform in diameter. In some examples, where the microfilter contains circular pores, the diameter of the pores is about 6 μm, 6.5 μm, 7 μm, 7.5 μm, 8 μm, 8.5 μm, 9 μm or 9.5 μm in diameter. Typically, the diameter of the pores is about 8 μm.

In particular examples, the filter contains rectangular slots. In some examples, the rectangular slots comprise a shape generally having a length and width where the length is longer than the width. In some examples the width of the rectangular slots is less than about 9.5 μm, 9 μm, 8.5 μm, 8 μm, 7.5 μm, 7 μm, 6.5 μm, or 5 μm. In some examples, the ratio of length to width of the rectangular slot is about 2:1, 3:1, 4:1, 5:1, 6:1, 7:1, 8:1, 9:1, 10:1 or greater. In particular examples, the rectangular slot size of the microfilter is about 6 μm in width and about 40 μm in length.

In some examples, the thickness of the microfilter membrane is at least about 0.1 μm, 0.5 μm, 1 μm, 1.5 μm, 2 μm, 2.5 μm, 3 μm, 3.5 μm, 4 μm, 4.5 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm or thicker. In particular examples, the thickness of the microfilter membrane is about 10 μm. In some examples, the thickness of the microfilter is about 1%, 2%, 3%, 4%, 5%, 6%, 7%, 8%, 9%, 10%, 15%, 20%, 25%, 30% or greater than the width or diameter of the pore. In particular examples, the thickness of the microfilter is between about 5% to about 25% the width or diameter of the pore.

In some examples, the microfilter has a pore density of from about 1 to 40,000, 1,000 to 40,000, 5,000 to 40,000; 6,000 to 40,000, 7000 to 40,000, 10,000 to 40,000; 10,000 to 30,000; 20,000 to 30,000; 20,000 to 40,000; or 30,000 to 40,000 pores per square millimeter. In some examples, the microfilter has a pore density at least about 1, 10, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000 or more pores per square millimeter.

In some examples, a constant pressure can be applied to the sample to facilitate the filtration process, such as a constant low-pressure is applied to the sample. In some examples, the pressure can range from about 0.01 to about 0.5 psi, such as, for example, from 0.05 to 0.4 psi, such as, for example, 0.1 to 0.3 psi or from 0.1 to 0.25 psi.

In some examples, the microfilter contains a single porous membrane. In some examples, the microfilter contains two or more porous membranes (see, e.g. Pat. Pub. No. 2009/0188864) arranged in layers. In some examples, where the microfilter contains two or more porous membranes, adjacent membranes are typically arranged such that the pores of one membrane are horizontally offset from the pores of an adjacent membrane. In such examples, two adjacent membranes typically are separated by a gap that is smaller than the diameter of a CTC (e.g. less than about 8 μm). In some examples, where the microfilter contains two or more porous membranes, the pores of adjacent membranes can be the same size or a different size. In a particular example, the microfilter contains a first top membrane having an array of pores ˜9 μm in diameter and a bottom membrane having an array of pores ˜8 μm in diameter, where the top membrane and the bottom membrane are separated by a gap ˜6.5 μm in width.

In particular examples, the sample is passed through the filter using a constant low pressure delivery system. In some examples, the sample is passed through the microfilter at a rate of about 0.01 ml/min, 0.05 ml/min, 0.1 ml/min, 0.5 ml/min, 1 ml/min, 2 ml/min, 3 ml/min, 4 ml/min, 5 ml/min, 6 ml/min, 7 ml/min, 8 ml/min, 9 ml/min, 10 ml/min, 11 ml/min, 12 ml/min, 13 ml/min, 14 ml/min, 15 ml/min or faster. In some examples, a vaccuum manifold is employed to draw the sample through the filter.

In some examples, the microfilter is a parylene microfilter. In some examples, the microfilter is a parylene-C slot microfilter (see e.g., Xu et al. (2010) Cancer Res 70(16):6420-6426 and U.S. Pat. Pub. No. 2011/0053152).

b. Microfluidic Devices

In some examples, the tumor cell enrichment method for use in combination with an oncolytic reporter virus involves capture of tumor cells using a microfluidic device. A variety of microfluidic devices are available in the art for the selection of CTCs in a fluid sample. Such microfluidic devices include for example, microfluidic devices that select tumor cells based on physical properties such as, for example, size, stiffness, and deformability, or based on biological properties such as, for example, the expression of a cell surface marker. Use of an oncolytic reporter virus for detection of CTCs allows CTCs to be detected on the microfluidic device without additional staining procedures since the infected CTCs can be detected by expression of a reporter gene product encoded by the virus.

In some examples, passage of a sample through the microfluidic device captures CTCs based on physical properties of the CTC but other non-tumor cells pass through the device and are not captured. In exemplary methods, the microfluidic device contains a microfluidic channel having a plurality of obstacles for the capture of CTCs where the obstacles are arranged to trap CTCs based on physical properties of the CTCs. An exemplary microfluidic device that captures CTCs based on physical properties of the CTC includes, but is not limited to the CTC Microfiltration Biochip (ClearCell™ System and CTChip®, Clearbridge Biomedics Pte Ltd., Singapore; see e.g. Tan et al. (2009) Biomedical Microdevices 11(4): 883-892 and Tan et al. (2010) Biosens Bioelectron 26:1701-1705; see, also International PCT application No. WO 2011/109762).

In exemplary methods, microfluid device contains a plurality of cell traps. Exemplary cell traps contain gaps of a sufficient size to allow for passage of non-tumor cells, but retain tumor cells. For example, cell traps can contain 1, 2, 3, 4 or more gaps. In some examples, the gaps are about 4 μm to about 5 μm. In some example, the cell traps from a crescent shape, such as, for example, “U” shape, “V” shape or “C” shaped structure. The cell traps can be arranged in the microfluidic device as a plurality of rows, sufficiently spaced apart to minimize clogging of the device, such as, for example about 10 μm to about 100 μm, such, for example about 10 μm, 20 μm, 30 μm, 40 μm, 50 μm, 60 μm, 70 μm, 80 μm, 90 μm or 100 μm. The cell traps in a particular row can be offset from the cell traps in a successive row, such as, for example, about 20 μm to about 50 μm. In some examples, the rows contain alternating left and right titled orientations of the crescent shaped cell traps in successive row of the cell traps.

In some examples, passage of a sample through the microfluidic device captures CTCs based on expression of one or more CTC-specific cell surface proteins on the CTC but other non-tumor cells that do not express the protein pass through the device and are not captured. In some exemplary methods, the microfluidic device contains a microfluidic channel having a plurality of obstacles (e.g. micropost) for the capture of CTCs (i.e. cell capture surface) where the obstacles are bound to a tumor specific binding agent. In other exemplary methods, the microfluidic device contains a plurality of microfluidic channels having a plurality of surfaces bound to a tumor specific binding agent. In some examples, the plurality of surfaces from one or more ridges. In some examples, the one or more ridges are arranged sequentially to form a herringbone shape.

In some examples, a single tumor specific binding agent is employed. In some examples, two or more binding agents are employed. Exemplary tumor specific binding agents include but are not limited to an antibody, antibody fragment, a receptor or a peptide. Exemplary tumor specific binding agents include but are not limited to anti-epithelial cell adhesion molecule (EpCAM) or anti-cytokeratin antibodies or antigen binding fragments thereof. In some examples, the tumor specific binding agent is an RGD peptide.

In some examples, the microfluidic device also contains a cell rolling-inducing agent is immobilized to a cell capture surface of the microfluidic device (see, e.g. International PCT Publication No. WO 2010/124227). A cell rolling-inducing agent can aid in the capture of the CTCs by the tumor specific binding agent. In some examples, the cell rolling-inducing agent is a selectin, such as, for example E-selectin, P-selectin or L-selectin.

In some examples, the tumor specific binding agent and/or cell rolling-inducing agent is immobilized to the cell capture surface (e.g. micropost or other surface of the microfluidic device) by the attachment of the tumor specific binding agent directly to the cell capture surface. In some examples, the tumor specific binding agent and/or cell rolling-inducing agent is covalently attached to the cell capture surface through a chemical moiety, including, but not limited to, an epoxy group, a carboxyl group, a thiol group, an alkyne group, an azide group, a maleimide group, a hydroxyl group, an amine group, an aldehyde group, and combinations thereof. In some examples, the tumor specific binding agent and/or cell rolling-inducing agent is immobilized to the cell capture surface using a peptide or chemical linker. Exemplary linkers include, but are not limited to, dextran, a dendrimer, polyethylene glycol, poly(L-lysine), poly(L-glutamic acid), polyvinyl alcohol, polyethyleneimine, poly(lactic acid), poly(glycolic acid) and combinations thereof.

An exemplary microfluidic device that captures CTCs based on expression of one or more CTC-specific cell surface proteins is the CTC-chip, which contains anti-EpCAM antibodies coupled to microposts (see, e.g. Nagrath et al. (2007) Nature 450:1235-1239). Another exemplary microfluidic device that contains a plurality of microfluidic channels having a plurality of surfaces bound to a tumor specific binding agent that binds to a CTC includes, but is not limited to the Herringbone CTC Chip (see, e.g. Stott et al. (2010) Proc. Natl. Acad. Sci. U.S.A. 107(43):18392-19397; see also International PCT Publication No. WO 2010/124227).

In exemplary methods, CTCs are enriched in a sample by applying the sample to a microfluidic device. The enriched sample (i.e. the cell population that is retained by the microfluidic device which is enriched for tumor cells) is then infected with the oncolytic reporter virus and expression of the reporter gene product is detected, thereby detecting the tumor cells in the enriched sample. In some examples, infection with the oncolytic reporter virus is performed by adding the virus to the captured cells on the microfluidic device. In some examples, the captured cells are removed from the microfluidic device and then contacted with the oncolytic reporter virus.

In exemplary methods, the microfluidic device has a channel volume of 10 μl-20 ml, for example 100 μl-15 ml, 100 μl-10 ml, 100 μl-5 ml, 100 μl-1 ml, or 100 μl-0.5 ml. In some examples, the channel of the microfluidic device can be connected to a reservoir that holds the fluid sample prior to capture and feeds the fluid sample into the microfluidic channel. The reservoir can have a volume, for example, of about 10 μl, 25 μl, 50 μl, 100 μl, 250 μl, 500 μl, 1 ml, 2.5 ml, 5 ml, 10 ml, 25 ml, 50 mL or more. The microfluidic devices can be combined with pumps for the delivery of samples to the device, delivery of the oncolytic report virus for infection of the retained cell and/or wash buffers or other labeling reagents.

In other exemplary methods, CTCs are enriched in a sample from a subject to whom an oncolytic reporter virus is administered. Enrichment can be effected by applying the sample to a microfluidic device that captures CTCs, and then detecting the expression of the reporter gene product, thereby detecting the CTCs in the enriched sample. In some examples, the infected CTCs are detected on the microfluidic device. In some examples, the infected CTCs are removed from the microfluidic device and then detected.

c. Immunomagnetic Separation

In some examples, the tumor enrichment method for use in combination with an oncolytic reporter virus involves immunomagnetic separation based on positive selection of CTCs or negative selection and removal of non-CTCs from the sample (i.e. immunodepletion). Such methods employ magnetic beads coupled to antibodies. In examples where positive selection is employed, the magnetic beads can be coupled to an antibody specific for a protein specifically expressed by the CTCs.

Exemplary methods for selection of CTCs based on immunomagnetic separation include but are not limited to purification based on expression of EpCam and/or cytokeratin. Such methods are known in the art and include, for example, the CellSearch® platform (Veridex, Warren, N.J., USA; see e.g. Pantel et al. (2009) Nat. Rev. Clin Oncol. 6:339-351), CTC-chip Ephesia method (see, e.g. Saliba et al. (2010) Proc. Natl. Acad. Sci. USA 107:14524-14529), MagSweeper system (see, e.g. Talasaz et al. (2009) Proc. Natl. Acad. Sci. USA 106:3970-3975), and Ariol® system (see, e.g. Deng et al. (2008) Breast Cancer Res 10:R69.).

In examples where positive selection is employed, the magnetic beads can be coupled to an antibody specific for a protein expressed by one or more non-tumor cell types in the sample. For example, lymphocytes can be removed from a sample by immunodepletion of CD45 positive cells by immunomagnetic separation using magnetic beads coupled to anti-CD45 antibodies.

In some examples, magnetic beads can be coupled to an antibody specific for a virally encoded protein, including any antibody described herein, that is expressed on the surface of a tumor cell, particularly a CTC. For example, the protein can be a membrane protein expressed on the surface of CTC. Examples of virally encoded proteins include any described herein, such as, for example, cell surface receptor, including transporter proteins. In some examples, the virally encoded protein is NIS or NET, and the magnetic beads are coupled to an antibody specific for an epitope on the extracellular domain of NIS that permits capture of such cells.

d. Acoustophoresis

In some examples, the tumor enrichment method for use in combination with an oncolytic reporter virus involves selection of CTCs in a sample based on the differential response of CTCs to sound waves due to their larger size (see, e.g., Augustsson et al. (2010) 14th International Conference on Miniaturized Systems for Chemistry and Life Sciences, 3-7 Oct. 2010, Groningen, The Netherlands 1592-1594; Lenshof and Laurell (2011) J Lab Autom. 16(6):443-449 and Wiklund and Onfelt (2012) Methods Mol. Biol. 853:177-196). For example, fluid samples, such as a blood fluid sample, can be processed through a microfluidic chamber, where an acoustic force is applied to stream of cells flowing through the chamber creating an ultrasonic standing wave field. Cells are separated in to bifurcating channels based on deflection of the cells through the acoustic field. Tumor cells are able to be separated from normal blood based on their differential deflection through the wave field. The CTCs in the enriched sample can be detected by infection with the oncolytic reporter virus and detection of the expressed reporter gene product encoded by the virus.

e. Dielectrophoresis

In some examples, the tumor enrichment method for use in combination with an oncolytic reporter virus involves selection of CTCs in a sample based on the dielectric properties of CTCs. Dielectric properties (polarisability) of cells are dependant upon factors, such as cell diameter, membrane area, density, conductivity and volume. Exemplary methods for enrichment of CTCs in a sample include, but are not limited to, dielectrophoretic field-flow fractionation (depFFF) (e.g., ApoStream™ (ApoCell); see, e.g. Gascoyne P R et al. (2009) Electrophoresis 30:1388-1398 and Wang et al. (2000) Anal Chem. 72(4):832-839). For example, fluid samples, such blood fluid sample, can be processed through a microfluidic chamber containing an electrode array that attracts or repels cells depending on their dielectric properties. In a blood sample, for example, tumor cells are pull towards the electrode array, while blood cells are repelled. This results in retardation of the flow of tumor cells through the chamber, while the blood cell flow more quickly. Thus, normal blood cells are separated from the slower moving tumor cells allowing for enrichment of a tumor cell fraction. The CTCs in the enriched sample can be detected by infection with the oncolytic reporter virus and detection of the expressed reporter gene product encoded by the virus.

f. Density Gradient Separation

In some examples, the tumor enrichment method for use in combination with an oncolytic reporter virus involves selection of CTCs in a sample based on the cellular density of the CTCs relative to other cells in a sample using a cell separation medium. Mononuclear cells (e.g. monocytes and lymphocytes) and CTCs have a buoyant density of <1.077 g/mL and can be separated from other cells, such as red blood cells (erythrocytes) and polymorphonuclear (PMN) leukocytes (granulocytes), which have a density of >1.077 g/ml. Centrifugation on an isoosmotic medium with a density of 1.077 g/mL allows the RBCs and PMN leukocytes to sediment through the medium while retaining the mononuclear cells and CTCs at the sample/medium interface. Density gradient separation systems are commonly used in the art for the separation of CTCs and include, but are not limited to, Ficoll-Hypaque (Amersham), Lymphoprep (Nycomed), and OncoQuick ((Hexyl Gentech/Geiner Bio-One) (see, e.g. International Pat. Pub. Nos. WO 99/40221 and WO 00/46585). Such methods can be used in combination with oncolytic virus infection for detection of CTCs in the enriched sample. In the OncoQuick method, a porous membrane and a discontinuous gradient medium are employed to deplete mononuclear cells from the CTC fraction.

In exemplary methods, CTCs are enriched in a sample by applying the sample to a density gradient and centrifuging the sample to obtain a CTC enriched cell fraction. The enriched sample is then infected with the oncolytic reporter virus and expression of the reporter gene is detected, thereby detecting the CTCs in the enriched sample.

In some examples, the sample applied to the density gradient is a sample from a subject that has been administered an oncolytic reporter virus. In exemplary methods, CTCs are enriched in a sample from a subject administered an oncolytic reporter virus by applying the sample to the density gradient, centrifuging the sample to obtain a CTC enriched cell fraction, and then detecting the expression of the reporter gene in the enriched fraction, thereby detecting the CTCs in the enriched sample.

For detection, typically the CTC enriched sample is extracted from gradient and layered onto slides using well known techniques (e.g., by the cytospin technique, or by culturing on poly-L-lysine-coated chamber slides). Following extraction, the cell can be washed in an appropriate buffer (e.g. PBS). In some examples, the cells are washed in an appropriate buffer (e.g. PBS) prior to virus infection.

In particular examples, the density gradient is an isoosmotic medium, such as Ficoll-Paque, with a density in the range of about 1.055 to 1.077 g/ml, such as for example, 1.055 to 1.065 g/ml. Generally, the cell separation medium does not to react with the body fluid or the cells present therein. Exemplary cell separation media include, but are not limited to, Ficoll (high mass polysaccharide that dissolves in aqueous solutions) or Percoll (medium containing colloidal silica particles coated with polyvinylpyrrolidone) or a Percoll- or Ficoll-like medium. Exemplary Ficoll-based density gradients include, but are not limited to, Ficoll-Isopaque, Ficoll-Paque Plus, Ficoll-Paque Premium and Ficoll-Hypaque.

In some examples, a porous barrier is layered on above the density gradient to prevent mixing of whole blood with the density gradient prior to centrifugation and to provide increased depletion of mononuclear cells from CTCs. The porous barrier can be made of any suitable material. Suitable examples include, but are not limited to, plastics, metal, ceramic or a mixture or special alloy of these materials. In a particular example, the porous barrier contains a hydrophobic material or is coated with a hydrophobic material. In some examples, the porous barrier has a thickness of at or about 0.5 to 10 mm, for example, 1 to 5 mm. In some examples, the porous barrier has a pore size of about 5-100 μm, such as, for example, 6-50 μm, such as, for example, about 8-30 μm, such as, for example, about 10-30 μm, such as, for example, about 20-30 μm.

In some examples, the sample is diluted with saline or other suitable buffer prior to application to the gradient. For example the sample can be diluted in a suitable buffer at a ratio of 1:1, 1:2, 1:3, 1:4, 1:5, 1:6, 1:7, 1:8, 1:9, 1:10 or greater.

In some examples, the centrifugation is performed at about 500 to 2,000×g, for example at about 1,000×g, for about 10 to 30 minutes, for example, about 20 to 30 minutes. The temperature during the centrifugation is typically about 4° C. to minimize catalytic activity of proteases, DNAses and RNAses.

g. Selective Cell Lysis (RBC lysis of blood cells)

In some examples, a tumor cell enrichment method involves removal of red blood cells from a blood cell sample. For example, red blood cells are sensitive to lysis in a hypotonic medium (i.e. low solute concentration), and thus can be selectively lysed in a sample containing a mix population of cells while leaving the remaining non-RBCs intact. The RBCs take up water by osmosis and burst open leaving an empty membrane sack, or ghost, behind.

In exemplary methods, hypotonic solution is added to a blood sample and the sample is incubated until the sample is clear or substantially clear, indicating that the red blood cells in the sample are lysed. The sample typically is then centrifuged to pellet the remaining enriched cells. The enriched cells are then resuspended in an appropriate buffer and infected with the oncolytic reporter virus for detection of CTCs according to the methods provided.

In some examples, the red blood cells in a blood sample from a subject is lysed and the enriched sample is layered onto one or more slides, for example, by cytospin. In particular examples, the red blood cells in a blood sample from a subject is lysed, and the enriched sample is infected with the oncolytic reporter virus prior to layering on the slides by cytospin. Following incubation with the virus, the infected sample is layered onto slide by cytospin, and the CTCs in the sample are then detected by detection of the reporter protein expressed by the oncolytic reporter virus.

h. Combinations of Tumor Cell Enrichment Methods

In some examples, two or more tumor cell enrichment methods are performed in combination with infection with an oncolytic reporter virus. In such examples, the sample can be infected prior to, during, or following performance of a first tumor cell enrichment method on the sample, or prior to, during, or following performance of a second or subsequent tumor cell enrichment method. In some examples, the sample is one obtained from a subject previously treated with an oncolytic reporter virus, and two or more tumor cell enrichment methods are performed on the sample prior to detection of the infected CTCs.

In particular examples, the red blood cells of a blood sample from a subject are lysed and then a second tumor cell enrichment method is applied to the sample. For example, the red blood cells of a blood sample from a subject can be lysed and then the enriched sample is further enriched by passing the sample through a microfilter or a microfluidic device. In another example, the red blood cells of a blood sample from a subject can be lysed and then the enriched sample is further enriched by performing immunomagnetic separation based on CTC specific cell markers on the sample (e.g. Ariol system; see, e.g. Deng et al. (2008) Breast Cancer Res. 10:R69).

3. Detection Methods

Any appropriate method known in the art can be employed to detect an expressed reporter protein, including, but not limited to, fluorescent, luminescent, spectrophotometric, chromogenic assays, or radioactive detection methods, which can be used to detect proteins, either directly, or indirectly, such as by enzymatic reaction or immunological detection. It is within the level of one of skill in the art to detect a reporter protein expressed by a cell infected with a reporter virus using an appropriate method based on the type of reporter protein employed.

In some examples, a fluorescent protein or a fluorescent product derived from a fluorogenic substrate is detected with a fluorometer, a fluorescence microscope (e.g., with an Olympus inverted fluorescence microscope (Olympus, Tokyo, Japan)), fluorescence confocal laser scanning microscope, a flow cytometer (e.g., a FACScan flow cytometer (BD Biosciences)) or a combination thereof. In some examples, a chromogenic or spectrophotometric substrate or signal is detected with a spectrophotometer. In some examples, a radioactive substrate or signal is detected by scintillation counter, scintigraphy, gamma camera, a β+ detector, a γ detector, or a combination thereof. In some examples, photon emission, such as that emitted by a luciferase, can be detected by light sensitive apparatus such as a luminometer or modified optical microscopes. In some examples, a signal can be detected with a Raman spectrometer.

In some examples, a substrate is detected when changes in fluorescent or optical properties, such as wavelength changes, intensity changes or changes in absorption, occur upon activation or cleavage by the reporter protein. In some examples, detection is effected by capturing with an antibody presented on a nanoparticle (see, e.g., Wang et al. (2011) Analyst. 136:4295-4300).

Detection of a signal produced by the reporter protein can be done by an automated system, such as software program or intelligence system that is part of, or compatible with, the equipment (e.g. computer platform) on which the assay is carried out. Alternatively, this comparison can be done by a physician or other trained or experienced professional or technician. In some examples, a signal can be detected and processed using an automated microscope, such as an automated fluorescence microscope (e.g. Ikoniscope imaging system, Ikonisys, Tokyo, Japan; see e.g. U.S. Patent Pub No. 2009/0123054) or an automated flow cytometer (e.g., a FACScan flow cytometer (BD Biosciences)). Data can be processed by means of computer software interfaced with the detecting means. The software can be configured to produce appropriate activating wavelengths or energies for the particular detectable protein used, such as a green fluorescent protein or a red fluorescent protein. Analysis can be based on input received from the detector such as whether signal is detected or not. Determination of whether the cell is a cancer cell, a CTC, can be based upon a pre-determined algorithm, such as for example, detection of multiple signals.

In exemplary methods, where the method involves a tumor cell enrichment method performed with a microfilter or a microfluidic device, detection of a reporter protein is performed directly on the microfilter or a microfluidic device. For example, CTCs infected with an oncolytic reporter virus can be detected directly on the microfilter or a microfluidic device without additional processing steps. In other examples, the CTCs infected with an oncolytic reporter virus can be recovered from the microfilter or microfluidic device and then detected for example in solution or transferred to solid support, such as a microscope slide.

4. Samples for Use in the Methods

Exemplary methods provided herein involve detecting a circulating tumor cell (CTC) in a sample from a subject. CTCs can be detected and characterized from any suitable sample type. The sample can be any sample that contains one or more CTCs for detection.

The sample can be from any tissue or fluid from an organism. Samples include, but are not limited, to whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In particular examples, the sample is from a fluid or tissue that is part of, or associated with, the lymphatic system or circulatory system. In some examples, the sample is a blood sample that is a venous, arterial, peripheral, tissue, cord blood sample. In particular examples the sample is anticoagulated whole blood.

In some examples a particular fluid sample can be selected for use in the methods based on the type of cancer exhibited by the subject and/or the location of the tumor in the subject. In non-limiting examples, a urine sample can be selected for detection of CTCs in a subject with a bladder cancer; bronchial lavage or pleural fluid sample can be selected for detection of CTCs in a subject with lung cancer or subject suspected of having lung metastases, cerebrospinal fluid sample can be selected for detection of CTCs in a subject with central nervous system metastases, and a pancreatic fluid sample for detection of CTCs in a subject with pancreatic cancer, an abdominal fluid or peritoneal fluid sample can be selected for detection of CTCs in a subject with an abdominal organ cancer.

Fluid samples include any liquid sample into which cells have been introduced. For example, fluid samples can include culture media and liquefied tissue samples, and cell suspensions. In some examples, the fluid sample is generated by dissociation of cells in a tissue sample in an appropriate fluid medium. The tissue sample can be a biopsy sample. The biopsy sample can be a tumor biopsy sample or a biopsy sample of a tissue suspected of containing one or more cancer cells. The fluid sample also can be generated from a bone marrow sample by dissociation of bone marrow cells in an appropriate fluid medium.

a. Sources

The sample for use in the methods provided can be from a subject that has cancer, is suspected of having cancer, or is at risk for developing a cancer. In some examples, the sample is from a subject that is in cancer remission or is at risk of cancer recurrence. The sample can be from a subject that has not received an anticancer therapy or can be from a subject that has been administered one or more anticancer therapies. In some examples, the sample is obtained from a subject prior to treatment with an anti cancer therapy. In some examples, the sample is obtained from a subject following treatment with an anti cancer therapy.

In some examples, the sample is from a subject that has cancer. In some examples, the sample is from a subject that has a tumor. In some examples, the tumor is a solid tumor. In some examples, the tumor is a metastatic tumor. In some examples, the sample is from a subject that has a pre-cancerous lesion (dysplasia), carcinoma, adenocarcinoma, or a sarcoma. In some examples, the subject has a tumor and is at risk of metastasis of the tumor. In some examples, the sample is from a subject having an advanced stage cancer. In some examples, the subject has a hemopoietic cancer.

In some examples, the subject has a cancer that is acute lymphoblastic leukemia, acute lymphoblastic leukemia, acute myeloid leukemia, acute promyelocytic leukemia, adenocarcinoma, adenoma, adrenal cancer, adrenocortical carcinoma, AIDS-related cancer, AIDS-related lymphoma, anal cancer, appendix cancer, astrocytoma, basal cell carcinoma, bile duct cancer, bladder cancer, bone cancer, osteosarcoma/malignant fibrous histiocytoma, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, Burkitt lymphoma, carcinoid tumor, carcinoma, central nervous system lymphoma, cervical cancer, chronic lymphocytic leukemia, chronic myelogenous leukemia, chronic myeloproliferative disorder, colon cancer, cutaneous T-cell lymphoma, desmoplastic small round cell tumor, endometrial cancer, ependymoma. epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, Hodgkin lymphoma, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, lymphomas, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenstrom's macroglobulinemia or Wilm's tumor. In particular examples, the cancer is a cancer of the bladder, brain, breast, bone marrow, cervix, colon/rectum, kidney, liver, lung/bronchus, ovary, pancreas, prostate, skin, stomach, thyroid, or uterus.

In some examples, the sample is obtained from a subject that is a mammal. Exemplary mammalian subjects include, but are not limited to primates, such as humans, apes and monkeys; rodents, such as mice, rats, rabbits, and ferrets; ruminants, such as goats, cows, deer, and sheep; horses, pigs, dogs, cats, and other animals. In some examples, the sample is obtained from a patient. In some examples, the patient is a human patient.

b. Methods of Obtaining Samples

The samples can be obtained from the subject by any suitable means of obtaining the sample using well-known and routine clinical methods. Procedures for obtaining fluid samples from a subject are well known. For example, procedures for drawing a processing whole blood and lymph are well-known and can be employed to obtain a sample for use in the methods provided. Typically, for collection of a blood sample, an anti-coagulation agent (e.g. EDTA, or citrate and heparin or CPD (citrate, phosphate, dextrose) or comparable substances) is added to the sample to prevent coagulation of the blood. In some examples, the blood sample is collected in a collection tube that contains an amount of EDTA to prevent coagulation of the blood sample.

In some examples, the sample is a tissue biopsy and is obtained, for example, by needle biopsy, CT-guided needle biopsy, aspiration biopsy, endoscopic biopsy, bronchoscopic biopsy, bronchial lavage, incisional biopsy, excisional biopsy, punch biopsy, shave biopsy, skin biopsy, bone marrow biopsy, and the Loop Electrosurgical Excision Procedure (LEEP). Typically, a non-necrotic, sterile biopsy or specimen is obtained that is greater than 100 mg, but which can be smaller, such as less than 100 mg, 50 mg or less, 10 mg or less or 5 mg or less; or larger, such as more than 100 mg, 200 mg or more, or 500 mg or more, 1 g or more, 2 g or more, 3 g or more, 4 g or more or 5 g or more. The sample size to be extracted for the assay can depend on a number of factors including, but not limited to, the number of assays to be performed, the health of the tissue sample, the type of cancer, and the condition of the patient. The tissue is placed in a sterile vessel, such as a sterile tube or culture plate, and can be optionally immersed in an appropriate media. Typically, the cells are dissociated into cell suspensions by mechanical means and/or enzymatic treatment as is well known in the art.

Samples can be obtained from the subject at regular intervals, such as, for example, one day, two days, three days, four days, five days, six days, one week, two weeks, weeks, four weeks, one month, two months, three months, four months, five months, six months, or one year, or daily, weekly, bimonthly, quarterly, biyearly or yearly. Collection of samples can be performed at a predetermined time or at regular intervals relative to treatment with one or more anticancer agents. For example, a sample can be collected at a predetermined time or at regular intervals prior to, during, or following treatment or between successive treatments. In particular examples, a sample is obtained from the subject prior to administration of an anticancer therapy and then again at regular intervals after treatment has been effected.

The volume of a fluid sample can be any volume that is suitable for the detection of a CTC in the methods provided. In some examples, the volume for the fluid sample is dependent on the particular tumor cell enrichment method used. For example, particular tumor cell enrichment methods can require a larger or smaller fluid sample volumes depending on factors such as, but not limited to, the capacity of the device or method used and level of throughput of the tumor cell enrichment method. In some examples a fluid sample is diluted in an appropriate medium prior to application of the tumor cell enrichment method. In some examples, a fluid sample is obtained from a subject and a portion or aliquot of the sample is used in the tumor cell enrichment method. The portion or aliquot can be diluted in an appropriate medium prior to application of the tumor cell enrichment method.

In some examples the volume of the fluid sample is about 0.01 mL to about 50 mL, such as, for example, about 0.1 mL to about 10 mL. In non-limiting examples, the volume of the sample can be at least about 0.01 ml, 0.05 ml, 0.1 ml, 0.2 ml, 0.3 ml, 0.4 ml, 0.5 ml, 1 ml, 2 ml, 3 ml, 4 ml, 5 ml, 6 ml, 7 ml, 8 ml, 9 ml, 10 ml, 15 ml, 20 ml, 25 ml, 30 ml, 35 ml, 40 ml, 45 ml, 50 mL or more.

c. Control Samples

In some examples of the methods provided herein, samples are analyzed for the detection and enumeration of CTCs and compared to a control or reference sample. Control samples to which the subject's samples are compared can be sample obtained from a cancer patient with the same cancer type and/or same stage of cancer where the control sample is known to contain a particular level of CTCs. In some examples, a control sample can be a sample from a subject without any detectable cancer. In some examples, a control sample can be a sample from normal tissue without any detectable cancer. In some examples, a control sample can be a sample from a subject prior to treatment with an anticancer therapy, where the control sample is compared to a sample from the subject following treatment with an anticancer therapy.

5. Viruses for Use in the Method

a. General Characteristics for Virus Selection

Any virus that preferentially infects tumor cells compared to non-tumor cells and is detectable can be can be used in the methods provided herein. Such viruses are typically known in the art as oncolytic viruses. Viruses for use in the methods can be modified to express a reporter gene for detection of infected tumor cells can be used in the methods provided herein. One of skill in the art can readily identify such viruses, and can adapt them for the methods described herein for detection and enumeration of CTCs. In particular examples, the oncolytic viruses used herein are vaccinia viruses, such as for example, LIVP viruses.

Viruses used in the methods described herein also can be further modified to improve the suitability of the virus for use as a reporter virus, such as the selection of an appropriate reporter gene and regulatory elements for expression of the reporter gene as described herein. In exemplary methods where the oncolytic reporter virus is administered to a subject, it is desirable to select an attenuated virus.

In some examples, where a sample is infected ex vivo, the virus employed in the methods has a relatively short time course of infection, such that expression of the reporter gene can be assayed within about 6-24 hours after infection. The use of such viruses in the method ensures that results can be obtained in the shortest possible time. Viruses that exhibit a longer time course of infection also can be used, and the time taken to complete the method can be lengthened.

Viruses can have a range of effects on their host cell, including inhibition of host RNA, DNA or protein synthesis and cell death. The presence of the virus often gives rise to morphological changes in the host cell. Any detectable changes in the host cell due to infection are known as cytopathic effects, and can include cell rounding, disorientation, swelling or shrinking, detachment from the growth surface and cell death. Cell death can be due to, for example, cell lysis following release of progeny viruses, or the induction of apoptosis. In some instances, however, cell death is not imminent following infection, such as in the case of a latent infection when the viral nucleic acid sequence is incorporated into the cell but the cell is not actively producing viral particles (e.g., Herpes simplex virus), or when there is continued, low-level release of virions in the absence of rapid and severe host cell damage (e.g., hepatitis B virus and HIV). The severity and the rate at which these effects are observed vary widely, and can influence the suitability of a virus for use as a reporter virus in the CTC detection methods. For the purposes herein, a virus that induces rapid cell death or apoptosis may not be suitable for use a reporter virus, as such changes will affect the accuracy of CTC detection method. Assays for determining the infection profile and effects on host cells are well-known in the art and can be employed for selecting an appropriate oncolytic reporter virus for use in the methods.

b. Expression of a Reporter Gene Product

The viruses used in the methods provided herein are modified to express one or more heterologous genes. Gene expression can include expression of a protein encoded by a gene and/or expression of an RNA molecule encoded by a gene. For use in the methods provided, the viruses are modified express one or more genes whose products are detectable or whose products can provide a detectable signal. These genes are often called “reporter genes”, and their products are called “reporter proteins” or “reporter gene products”. A reporter gene and its product are generally amenable to assays that are sensitive, quantitative, rapid, easy and reproducible. Many reporter genes have been described in the art, and their detection can be effected in a variety of ways. These heterologous genes can be introduced into the viruses and used to easily assess, for example, the activity of the promoter under which the reporter gene is controlled, the level of transcription and/or translation of the virally encoded genes, and in some instances, by inference, certain activities of the host cell in which the virus resides. In some examples, the reporter protein interacts with host cell proteins, resulting in a detectable change in the properties of the reporter protein. Expression of heterologous genes can be controlled by a constitutive promoter, or by an inducible promoter. Expression also can be influenced by one or more proteins or RNA molecules expressed by the virus. Host cell factors also can influence the expression of heterologous genes. Depending upon the factors that influence the expression of the reporter gene, the level of expression of the reporter gene can be used as an indicator for various processes within the virus, or within the host cell in which the virus grows. For example, if expression of the reporter gene relies on viral factors produced only after viral DNA replication occurs, then the level of the expression of the reporter gene can be used as a measure of the level of viral DNA replication.

### i. Exemplary Reporter Proteins

A variety of reporter genes that encode detectable proteins are known in the art, and can be expressed in the viruses in the methods provided herein. Detectable proteins include receptors or other proteins that can specifically bind a detectable compound, proteins that can emit a detectable signal such as a fluorescence signal, and enzymes that can catalyze a detectable reaction or catalyze formation of a detectable product. Thus, reporter proteins can be assayed by detecting endogenous characteristics, such as enzymatic activity or spectrophotometric characteristics, or indirectly with, for example, antibody-based assays.

### (1) Fluorescent Proteins

In some examples, the oncolytic reporter viruses can express a gene encoding a protein that is a fluorescent protein. Fluorescent proteins emit fluorescence by absorbing and re-radiating the energy of light. Fluorescence can yield relatively high levels of light, compared to, for example, chemiluminescence, and is readily detected by various means known in the art and described herein. Many fluorescent proteins are known in the art and have been widely used as reporter proteins. The first cloned of these, and the most well-known, is green fluorescent protein (GFP) from the Aequorea victoria (Prasher et al. (1987) Gene 111: 229-233), which is a 27 kDa protein that produces a green fluorescence emission with a peak wavelength at 507 nm following excitation at either 395 or 475 nm. GFP also has been cloned from Aequorea coerulescens (Gurskaya et al. (2003) Biochem J. 373:403-408). The wild-type GFP gene has been modified by, for example, point mutation, optimizing codon usage or introducing a Kozak translation initiation site, to generate multiple variants with improved and/or alternate properties. For example, a variant termed enhanced green fluorescent protein (EGFP) contains a single point mutation that shifts the excitation wavelength to 488 nm, which is in the cyan region, and optimized codon usage which yields greater expression in mammalian systems (Yang et al. (1996) Nucl Acids Res. 24 4592-4593). Other variants are spectral variants which display blue, cyan and yellowish-green fluorescent emissions, generally referred to as blue fluorescent protein (BFP), cyan fluorescent protein (CFP), and yellow fluorescent protein (YFP). Examples of these and other variants of GFP include, but are not limited to, those described in U.S. Pat. Nos. 5,625,048, 5,804,387, 6,027,881, 6,150,176, 6,265,548, and 6,608,189.

GFP-like proteins have been isolated from other organisms, particularly the reef corals in the class Anthazoa. While some of the GFP-like proteins emit a green fluorescence, such as the green fluorescent protein from the anthozoan coelenterates Renilla reniformis and Renilla kollikeri (sea pansies) (U.S. Pat. Pub. No. 2003/0013849), others fluoresce with an even wider range of colors than the GFP variants, including blue, green, yellow, orange, red and purple (see e.g., U.S. Pat. No. 7,166,444, Miyawaki et al. (2002) Cell Struct Func 27: 343-347, Labas et al. (2002) Proc. Natl. Acad. Sci. USA 99:4256-4261). Examples of the GFP-like fluorescent proteins include, but are not limited to, those set forth in Table 3.

TABLE 3
Examples of GFP-Iike proteins

| | | Excitation | Emission | |
|---|---|---|---|---|
| Protein ID | | maxima | maxima | |
| (alternate ID) | Species | (nm) | (nm) | Color |
| amajGFP | Anemonia majano | 458 | 486 | green |
| (amFP486) | | | | |
| dsfrGFP (DsFP483) | Discosoma striata | 456 | 484 | green |
| clavGFP (CFP484) | Clavularia sp. | 443 | 483 | green |
| cgigGFP | Condylactis gigantea | 399, 482 | 496 | green |
| hcriGFP | Heteractis crispa | 405, 481 | 500 | green |
| ptilGFP | Ptilosarcus sp. | 500 | 508 | green |
| rmueGFP | Renilla muelleri | 498 | 510 | green |
| zoanGFP (zFP560) | Zoanthus sp. | 496 | 506 | green |
| asulGFP (asFP499) | Anemonia sulcata | 403, 480 | 499 | green |
| dis3GFP | Discosoma sp.3 | 503 | 512 | green |
| dendGFP | Dendronephthya sp. | 494 | 508 | green |
| mcavGFP | Montastraea cavernosa | 506 | 516 | green |
| rfloGFP | Ricordea florida | 508 | 518 | green |
| scubGFP1 | Scolymia cubensis | 497 | 506 | green |
| scubGFP2 | Scolymia cubensis | 497 | 506 | green |
| zoanYFP | Zoanthus sp. | 494, 528 | 538 | yellow |
| DsRed (drFP583) | Discosoma sp. 1 | 558 | 583 | orange-red |
| dis2RFP (dsFP593) | Discosoma sp. 2 | 573 | 593 | orange-red |
| zoan2RFP | Zoanthus sp.2 | 552 | 576 | orange-red |
| cpFP611 | Entacmaea quadricolor | 559 | 611 | orange-red |
| mcavRFP | Montastraea cavernosa | 508, 572 | 520, 580 | orange-red |
| rfloRFP | Ricordea florida | 506, 566 | 517, 574 | orange-red |
| Kaede | Trachyphillia geoffroyi | 508, 572 | 518, 582 | orange-red |
| asulCP (asCP) | Anemonia sulcata | 568 | none | purple-blue |
| hcriCP (hcCP) | Heteracis crispa | 578 | none | purple-blue |
| cgigCP (cpCP) | Condylactis gigantea | 571 | none | purple-blue |
| cpasCP (cpCP) | Condylactis parsiflora | 571 | none | purple-blue |
| gtenCP (gtCP) | Goniopora tenuidens | 580 | none | purple-blue |

*Adapted from Miyawaki et al. (2002) Cell Struct Funct 27, 343-34.

Exemplary GFP variants and variants of GFP-like proteins from variety of species are known and can be employed for expression by an oncolytic virus provided herein. Such fluorescent proteins include monomeric, dimeric and tetrameric fluorescent proteins. Exemplary monomeric fluorescent proteins include, but are not limited to: violet fluorescent proteins, such as for example, Sirius; blue fluorescent proteins, such as for example, Azurite, EBFP, SBFP2, EBFP2, TagBFP; cyan fluorescent proteins, such as for example, mTurquoise, eCFP, Cerulean, SCFP, TagCFP, mTFP1; green fluorescent proteins, such as for example, GFP, mUkG1, aAG1, AcGFP1, TagGFP2, EGFP, mWasabi, EmGFP (Emerald); yellow fluorescent proteins, such as for example; TagYFP, EYFP, Topaz, SYFP2, YPet, Venus, Citrine; orange fluorescent proteins, such as for example, mKO, mKO2, mOrange, mOrange2, red fluorescent proteins, such as for example; TagRFP, TagRFPt, mStrawberry, mRuby, mCherry; far red fluorescent proteins, such as for example; mRasberry, mKate2, mPlum, and mNeptune; and fluorescent proteins having an increased stokes shift (i.e. >100 nm distance between excitation and emission spectra), such as for example, Sapphire, T-Sapphire, mAmetrine, and mKeima. Exemplary dimeric and tetrameric fluorescent proteins include, but are not limited to: AmCyan1, Midori-Ishi Cyan, copGFP (ppluGFP2), TurboGFP. ZsGreen, TurboYFP, ZsYellow1, TurboRFP, dTomato, DsRed2, DsRed-Express, DsRed-Express2, DsRed-Max, AsRed2, TurboFP602, RFP611, Katushka (TurboFP635), Katushka2, and AQ143. Excitation and emission spectra for exemplary fluorescent proteins are well-known in the art (see also e.g. Chudakov et al. (2010) Physiol Rev 90:1102-1163).

In particular examples, a GFP or GFP-like protein is selected for expression by an oncolytic virus for use in the methods provided herein. In other particular examples, a red or far-red fluorescent protein is selected for expression by an oncolytic virus for use in the methods provided herein. In further particular examples, the fluorescent protein Katushka (TurboFP635) protein is selected for expression by an oncolytic virus for use in the methods provided herein.

Selection of a particular fluorescent protein for use in the methods depends on variety of factors including, but not limited to, brightness, maturation rate, photostability, aggregation and pH stability of the fluorescent protein (see e.g. Chudakov et al. (2010) Physiol Rev 90:1102-1163). Typically, for the methods provided herein, a fluorescent protein for expression by an oncolytic reporter virus is selected to provide a detectable signal within a reasonable time following infection of the tumor cell. In exemplary methods provided herein, where detection of the fluorescent protein is performed on a microfilter or a microfluidic chip, a fluorescent protein for expression by an oncolytic reporter virus is typically selected to minimize background autofluorescence of the microfilter or microfluidic chip.

Other proteinaceous fluorophores include phycobiliproteins from certain cyanobacteria and eukaryotic algae. These proteins are among the most highly fluorescent known (Oi et al. (1982) J. Cell Biol. 93:981-986), and systems have been developed that are able to detect the fluorescence emitted from as little as one phycobiliprotein molecule (Peck et al. (1989) Proc. Natl. Acad. Sci. USA 86:4087-4091). Phycobiliproteins are classified on the basis of their color into two large groups, the phycoerythrins (red) and the phycocyanins (blue). Examples of fluorescent phycobiliproteins include, but are not limited to, R-Phycoerythrin (R-PE), B-Phycoerythrin (B-PE), Y-Phycoerythrin (Y-PE), C-Phycocyanin (P-PC), R-Phycocyanin (R-PC), Phycoerythrin 566 (PE 566), Phycoerythrocyanin (PEC) and Allophycocyanin (APC). The genes encoding the phycobiliproteins have been cloned from a multitude of species and have been used to express the fluorescent proteins in a heterologous host (Tooley et al. (2001) Proc. Natl. Acad. Sci. USA 98:10560-10565). The genes required for the expression of these or any other fluorophores can be cloned into the viruses used in the methods provided herein to generate a virus with a fluorescent reporter protein.

### (2) Bioluminescent Proteins

In some examples, the oncolytic reporter viruses can express a gene encoding a protein that is a bioluminescent protein. Chemiluminescence is a process in which photons are produced when molecules in an excited state transition to a lower energy level in an exothermic chemical reaction. The chemical reactions required to generate the excited states in this process generally proceed at a relatively low rate compared to, for example, fluorescence, and so yield a relatively low rate of photon emission. Because the photons are not required to create the excited states, they do not constitute an inherent background when measuring photon efflux, which permits precise measurement of very small changes in light. Bioluminescence is a form of chemiluminescence that has developed through evolution in a range of organisms, and is based on the interaction of the enzyme luciferase with a luminescent substrate luciferin. The luciferases can produce light of varying colors. For example, the luciferases from click beetles can produce light with emission peaks in the range of 547 to 593 nm, spanning four colors (Wood et al. (1989) Science 244:700-702).

Thus, luciferases for use in the methods provided are enzymes or photoproteins that catalyze a bioluminescent reaction (i.e., a reaction that produces bioluminescence). Some exemplary luciferases, such as firefly, Gaussia and Renilla luciferases, are enzymes which act catalytically and are unchanged during the bioluminescence generating reaction. Other exemplary luciferases, such as the aequorin photoprotein to which luciferin is non-covalently bound, are changed, such as by release of the luciferin, during bioluminescence-generating reaction. The luciferase can be a protein, or a mixture of proteins (e.g., bacterial luciferase). The protein or proteins can be native, or wild luciferases, or a variant or mutant thereof, such as a variant produced by mutagenesis that has one or more properties, such as thermal stability, that differ from the naturally-occurring protein. Luciferases and modified mutant or variant forms thereof are well known. For purposes herein, reference to luciferase refers to either the photoproteins or luciferases.

Exemplary genes encoding bioluminescent proteins include, but are not limited to, bacterial luciferase genes from Vibrio harveyi (Belas et al. (1982) Science 218:791-793), and Vibrio fischerii (Foran and Brown, (1988) Nucleic acids Res. 16:177), firefly luciferase (de Wet et al. (1987) Mol. Cell. Biol. 7:725-737), aequorin from Aequorea victoria (Prasher et al. (1987) Biochem. 26:1326-1332), Renilla luciferase from Renilla renformis (Lorenz et al. (1991) Proc. Natl. Acad. Sci. USA 88:4438-4442) and click beetle luciferase from Pyrophorus plagiophthalamus (Wood et al. (1989) Science 244:700-702). Other naturally occurring secreted luciferases include, for example, those from Vargula hilgendorfii, Cypridinia noctiluca, Oplophorus gracilirostris, Metridia longa and Gaussia princeps. Native and synthetic forms of the genes can be used in the methods provided herein. The luxA and luxB genes of bacterial luciferase can be fused to produce the fusion gene (Fab2), which can be expressed to produce a fully functional luciferase protein (Escher et al. (1989) Proc. Natl. Acad. Sci. USA 86:6528-6532). Transformation and expression of these and other genes encoding bioluminescent proteins in viruses can permit detection of viral infection, for example, using a low light and/or fluorescence imaging camera. In some examples, luciferases expressed by viruses can require exogenously added substrates such as decanal or coelenterazine for light emission. In other examples, viruses can express a complete lux operon, which can include proteins that can provide luciferase substrates such as decanal.

Bioluminescence substrates are the compounds that are oxidized in the presence of a luciferase and any necessary activators and which generates light. With respect to luciferases, these substrates are typically referred to as luciferins that undergo oxidation in a bioluminescence reaction. The bioluminescence substrates include any luciferin or analog thereof or any synthetic compound with which a luciferase interacts to generate light. Typical substrates include those that are oxidized in the presence of a luciferase or protein in a light-generating reaction. Bioluminescence substrates, thus, include those compounds that those of skill in the art recognize as luciferins. Luciferins, for example, include firefly luciferin, Cypridina (also known as Vargula) luciferin, coelenterazine, dinoflagellate luciferin, bacterial luciferin, as well as synthetic analogs of these substrates or other compounds that are oxidized in the presence of a luciferase in a reaction that produces bioluminescence.

### (3) Other Enzymes

In some examples, the oncolytic reporter viruses can express a gene encoding a protein that can catalyze a detectable reaction. Some commonly used reporter genes encode enzymes or other biochemical markers which, when active in the host cells, cause some visible change in the cells or their environment upon addition of the appropriate substrate. Two examples of this type of reporter are the E. coli genes lacZ (encoding β-galactosidase or “β-gal”) and gusA or iudA (encoding β-glucuronidase or “β-glu”). These bacterial sequences are useful as reporter genes because the cells in which they are expressed, prior to transfection, express extremely low levels (if any) of the enzyme encoded by the reporter gene. When host cells expressing the reporter gene (via heterologous expression from the virus) are incubated with an appropriate substrate, a detectable product is formed. The particular substrate used dictates the type of signal generated and the method of detection required. For example, β-galactosidase substrates include those that, when hydrolyzed by β-galactosidase, form products that can be detected, for example, by spectrophotometry (e.g., o-nitrophenyl-β-D-galactoside (ONPG) or 5-bromo-4-chloro-3-indolyl-β-D-galactopyranoside (X-gal)); fluorometry (e.g., a 4-methyl-umbelliferyl-β-galactopyranoside compound (MUG)); or via chemiluminescence (e.g., 1,2-dioxetane-galactopyranoside derivatives; Bronstein et al. (1996) Clin Chem. 42:1542-1546). Many substrates that facilitate the detection of enzymatic activity by various methods also exist for use with β-glucuronidase, including, but not limited to, 5-bromo-4-chloro-3-indolyl-β-D-glucuronic acid (X-Gluc), which produces a blue precipitate following hydrolysis; p-nitrophenyl β-D-glucuronide which also can be used in a spectrophotometrical format; 4-methylumbelliferyl-β-D-glucuronide (MUG), which can be used in a fluorometric assay; and sodium 3-(4-methoxyspiro{1,2-dioxetane-3,2′-(5′-chloro)-tricyclo[3.3.1.13,7]decan}-4-yl)phenyl-β-D-glucuronate (Glucurone; U.S. Pat. No. 6,586,196 and Bronstein et al. (1996) Clin Chem. 42:1542-1546), which can be used in a chemiluminescent assay.

Other exemplary reporter genes that can be expressed in the viruses used in the methods provided herein include secreted embryonic alkaline phosphatase (SEAP) and chloramphenicol acetyltransferase (CAT). SEAP is a truncated form of human placental alkaline phosphatase that is secreted into the cell culture supernatant following expression. The alkaline phosphatase activity can be readily assayed using any of the substrates known in the art, and can be visualized by chemiluminescence (e.g., using the substrate CSPD [disodium 3-(4-methoxyspiro[1,2-d]oxetane-3,2′(5′-chloro)-tricyclo[3,3,1,13,7)decan]-4-yl)phenyl phosphate]); fluorescence (e.g., using the substrate MUP [4-methylumbelliferyl phosphate]); or spectrometry (e.g., using the substrate p-nitrophenyl phosphate (PNPP)).

The bacterial gene encoding chloramphenicol acetyltransferase (CAT), which catalyzes the addition of acetyl groups to the antibiotic chloramphenicol also can be cloned into the viruses and used to express a reporter protein. CAT activity can be monitored in several ways. In one method, cells infected by the virus expressing the CAT reporter gene can be lysed and incubated in a reaction mix containing 14C- or 3H-labeled chloramphenicol and n-Butyryl Coenzyme A (n-Butyryl CoA). The expressed heterologous CAT transfers the n-butyryl moiety of the cofactor to chloramphenicol. The reaction products can be extracted, separated and the amount of radioactive n-butyryl chloramphenicol is assayed by liquid scintillation counting. The radioactive n-butyryl chloramphenicol resulting from CAT activity also can be analyzed using thin-layer chromatography.

Additional exemplary reporter genes include, but are not limited to enzymes, such as β-lactamase, alpha-amylase, peroxidase, T4 lysozyme, oxidoreductase and pyrophosphatase.

### (4) Proteins that Bind to Detectable Ligands

Exemplary detectable proteins also include proteins that can bind a contrasting agent, chromophore, or a compound or ligand that can be detected. In some examples, the ligand that binds to the detectable protein is covalently attached to a detectable moiety, such, for example a radiolabel, a chromogen, or a fluorescent moiety.

A variety of gene products that can specifically bind a detectable compound are known in the art, including, but not limited to receptors, metal binding proteins (e.g., siderophores, ferritins, transferrin receptors), ligand binding proteins, and antibodies. Any of a variety of detectable compounds can be used, and can be imaged by any of a variety of known imaging methods. Exemplary compounds include receptor ligands and antigens for antibodies. The ligand can be labeled according to the imaging method to be used. Exemplary imaging methods include, but are not limited to, X-rays, magnetic resonance methods, such as magnetic resonance imaging (MRI) and magnetic resonance spectroscopy (MRS), and tomographic methods, including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography and ultrasonic tomography.

Labels appropriate for X-ray imaging are known in the art, and include, for example, Bismuth (III), Gold (III), Lanthanum (III) or Lead (II); a radioactive ion, such as ⁶⁷Copper, ⁶⁷Gallium, ⁶⁸Gallium, ¹¹¹Indium, ¹¹³Indium, ¹²³Iodine, ¹²⁵Iodine, ¹³¹Iodine, ¹⁹⁷Mercury, ²⁰³Mercury, 186Rhenium, ¹⁸⁸Rhenium, ⁹⁷Rubidium, ¹⁰³Rubidium, ⁹⁹Technetium or ⁹⁰Yttrium; a nuclear magnetic spin-resonance isotope, such as Cobalt (II), Copper (II), Chromium (III), Dysprosium (III), Erbium (III), Gadolinium (III), Holmium (III), Iron (II), Iron (III), Manganese (II), Neodymium (III), Nickel (II), Samarium (III), Terbium (III), Vanadium (II) or Ytterbium (III); or rhodamine or fluorescein.

Labels appropriate for magnetic resonance imaging are known in the art, and include, for example, gadolinium chelates and iron oxides. Use of chelates in contrast agents is known in the art. Labels appropriate for tomographic imaging methods are known in the art, and include, for example, β-emitters such as ¹¹C, ¹³N, ¹⁵O or ⁶⁴Cu or γ-emitters such as ¹²³I. Other exemplary radionuclides that can, be used, for example, as tracers for PET include ⁵⁵Co, ⁶⁷Ga, ⁶⁸Ga, ⁶⁰Cu(II), ⁶⁷Cu(II), ⁵⁷Ni, ⁵² Fe and ¹⁸F (e.g., ¹⁸F-fluorodeoxyglucose (FDG)). Examples of useful radionuclide-labeled agents are a ⁶⁴Cu-labeled engineered antibody fragment (Wu et al. (2002) Proc. Natl. Acad. Sci. USA 97: 8495-8500), ⁶⁴Cu-labeled somatostatin (Lewis et al. (1999) J. Med. Chem. 42: 1341-1347), ⁶⁴Cu-pyruvaldehyde-bis(N4-methylthiosemicarbazone)(⁶⁴Cu-PTSM) (Adonai et al. (2002) Proc. Natl. Acad. Sci. USA 99: 3030-3035), ⁵²Fe-citrate (Leenders et al. (1994) J. Neural. Transm. Suppl. 43: 123-132), ⁵²Fe/^{52m}Mn-citrate (Calonder et al. (1999) J. Neurochem. 73: 2047-2055) and ⁵²Fe-labeled iron (III) hydroxide-sucrose complex (Beshara et al. (1999) Br. J. Haematol. 104: 288-295, 296-302).

### (5) Transporter Proteins (Transporters)

Membrane transport protein are involved in the movement of ions, small molecules, or macromolecules, such as other proteins, across a membrane. Transport proteins are integral membrane proteins that span the membrane across which they transport substances. Viruses for use in the methods provided herein can encode these proteins.

These proteins assist in the movement of substances by facilitated diffusion or active transport. Transporters can be located on the outer cell membrane, mitochondria or other intracellular organelles. When encoded by viruses as described herein, these transporters can function to transport and accumulate detectable and/or therapeutic substrates in cells, such as tumor cells, that are infected by the viruses. For example, transporters can provide signal amplification through transport-mediated concentrative intracellular accumulation of radiolabeled substrates for use in imaging, and can provide a means to deliver therapeutic substances to virally-targeted tumors. These transporters can be expressed on tumor cells, providing a target for capture of the tumor cells.

Transporters can be classified and identified using various systems and databases well known in the art. Such systems can be used to help identify transporters that can be expressed in the viruses using the methods described herein, and to identify the substrates for each transporter. For example, the Transporter Classification database (TCDB; www.tcdb.org/) is an IUBMB (International Union of Biochemistry and Molecular Biology)-approved classification system for membrane transport proteins, including ion channels (Saier et al., (2006) Nucl. Acids. Res. 34:D181-D186). This was designed to be analogous to the EC number system for classifying enzymes, but it also uses phylogenetic information. The TC system classifies approximately 3000 proteins into over 550 transporter families. Another system is the Solute Carrier (SLC) gene nomenclature system, which is the basis for the Human Genome Organization (HUGO) names of the genes that encode this group of transporters, and includes over 300 members organized into 47 families. Members within an individual SLC family have greater than 20% sequence homology to each other. The criteria for inclusion of a family into the SLC group is functional (i.e., an integral membrane protein which transports a solute) rather than evolutionary. The SLC group include transporters that are facilitative transporters (allow solutes to flow downhill with their electrochemical gradients) and secondary active transporters (allow solutes to flow uphill against their electrochemical gradient by coupling to transport of a second solute that flows downhill with its gradient such that the overall free energy change is still favorable). The SLC group does not include ATP-driven transporters, ion channels or aquaporins. Most members of the SLC group are located in the outer cell membrane, although some members are located in mitochondria (most notably SLC family 25) or other intracellular organelles. Table 4 provides the SLC families (e.g. SLC1), the subfamilies (e.g. SLC1A) and the member of the family (e.g. SLC1A1, corresponding to “Solute carrier family 1, member 1”).

TABLE 4
Solute Carrier (SLC) Transporter families

| Family | Members |
|---|---|
| SLC1: The high affinity | SLC1A1, SLC1A2, SLC1A3, SLC1A4, SLC1A5, SLC1A6, |
| glutamate and neutral | SLC1A7 |
| amino acid transporter | |
| family | |
| SLC2: The facilitative | SLC2A1, SLC2A2, SLC2A3, SLC2A4, SLC2A5, SLC2A6, |
| GLUT transporter family | SLC2A7, SLC2A8, SLC2A9, SLC2A10, SLC2A11, |
| | SLC2A12, SLC2A13, SLC2A14 |
| SLC3: The heavy | SLC3A1, SLC3A2 |
| subunits of the | |
| heteromeric amino acid | |
| transporters | |
| SLC4: The bicarbonate | SLC4A1, SLC4A2, SLC4A3, SLC4A4, SLC4A5, SLC4A6, |
| transporter family | SLC4A7, SLC4A8, SLC4A9, SLC4A10, SLC4A11 |
| SLC5: The sodium | SLC5A1, SLC5A2, SLC5A3, SLC5A4, SLC5A5, SLC5A6, |
| glucose cotransporter | SLC5A7, SLC5A8, SLC5A9, SLC5A10, SLC5A11, |
| family | SLC5A12 |
| SLC6: The sodium- and | SLC6A1, SLC6A2, SLC6A3, SLC6A4, SLC6A5, SLC6A6, |
| chloride-dependent | SLC6A7, SLC6A8, SLC6A9, SLC6A10, SLC6A11, |
| neurotransmitter | SLC6A12, SLC6A13, SLC6A14, SLC6A15, SLC6A16, |
| transporter family | SLC6A17, SLC6A18, SLC6A19, SLC6A20 |
| SLC7: The cationic | SLC7A1, SLC7A2, SLC7A3, SLC7A4, SLC7A5, SLC7A6, |
| amino acid | SLC7A7, SLC7A8, SLC7A9, SLC7A10, SLC7A11, |
| transporter/glycoprotein- | SLC7A13, SLC7A14 |
| associated family | |
| SLC8: The Na+/Ca2+ | SLC8A1, SLC8A2, SLC8A3 |
| exchanger family | |
| SLC9: The Na+/H+ | SLC9A1, SLC9A2, SLC9A3, SLC9A4, SLC9A5, SLC9A6, |
| exchanger family | SLC9A7, SLC9A8, SLC9A9, SLC9A10, SLC9A11 |
| SLC10: The sodium bile | SLC10A1, SLC10A2, SLC10A3, SLC10A4, SLC10A5, |
| salt cotransport family | SLC10A6, SLC10A7 |
| SLC11: The proton | SLC11A1, SLC11A2 |
| coupled metal ion | |
| transporter family | |
| SLC12: The | SLC12A1, SLC12A1, SLC12A2, SLC12A3, SLC12A4, |
| electroneutral cation-Cl | SLC12A5, SLC12A6, SLC12A7, SLC12A8, SLC12A9 |
| cotransporter family | |
| SLC13: The human | SLC13A1, SLC13A2, SLC13A3, SLC13A4, SLC13A5 |
| Na+-sulfate/carboxylate | |
| cotransporter family | |
| SLC14: The urea | SLC14A1, SLC14A2 |
| transporter family | |
| SLC15: The proton | SLC15A1, SLC15A2, SLC15A3, SLC15A4 |
| oligopeptide | |
| cotransporter family | |
| SLC16: The | SLC16A1, SLC16A2, SLC16A3, SLC16A4, SLC16A5, |
| monocarboxylate | SLC16A6, SLC16A7, SLC16A8, SLC16A9, SLC16A10, |
| transporter family | SLC16A11, SLC16A12, SLC16A13, SLC16A14 |
| SLC17: The vesicular | SLC17A1, SLC17A2, SLC17A3, SLC17A4, SLC17A5, |
| glutamate transporter | SLC17A6, SLC17A7, SLC17A8 |
| family | |
| SLC18: The vesicular | SLC18A1, SLC18A2, SLC18A3 |
| amine transporter family | |
| SLC19: The | SLC19A1, SLC19A2, SLC19A3 |
| folate/thiamine | |
| transporter family | |
| SLC20: The type III | SLC20A1, SLC20A2 |
| Na+-phosphate | |
| cotransporter family | |
| SLC21/SLCO: The | subfamily 1; SLCO1A2, SLCO1B1, SLCO1B3, SLCO1B4, |
| organic anion | SLCO1C1 |
| transporting family | subfamily 2; SLCO2A1, SLCO2B1 |
| | subfamily 3; SLCO3A1 |
| | subfamily 4; SLCO4A1, SLCO4C1 |
| | subfamily 5; SLCO5A1 |
| SLC22: The organic | SLC22A1, SLC22A2, SLC22A3, SLC22A4, SLC22A5, |
| cation/anion/zwitterion | SLC22A6, SLC22A7, SLC22A8, SLC22A9, SLC22A10, |
| transporter family | SLC22A11, SLC22A12, SLC22A13, SLC22A14, SLC22A15, |
| | SLC22A16, SLC22A17, SLC22A18, SLC22A19, SLC22A20 |
| SLC23: The Na+- | SLC23A1, SLC23A2, SLC23A3, SLC23A4 |
| dependent ascorbic acid | |
| transporter family | |
| SLC24: The Na+/(Ca2+- | SLC24A1, SLC24A2, SLC24A3, SLC24A4, SLC24A5, |
| K+) exchanger family | SLC24A6 |
| SLC25: The | SLC25A1, SLC25A2, SLC25A3, SLC25A4, SLC25A5, |
| mitochondrial carrier | SLC25A6, SLC25A7, SLC25A8, SLC25A9, SLC25A10, |
| family | SLC25A11, SLC25A12, SLC25A13, SLC25A14, SLC25A15, |
| | SLC25A16, SLC25A17, SLC25A18, SLC25A19, SLC25A20, |
| | SLC25A21, SLC25A22, SLC25A23, SLC25A24, SLC25A25, |
| | SLC25A26, SLC25A27, SLC25A28, SLC25A29, SLC25A30, |
| | SLC25A31, SLC25A32, SLC25A33, SLC25A34, SLC25A35, |
| | SLC25A36, SLC25A37, SLC25A38, SLC25A39, SLC25A40, |
| | SLC25A41, SLC25A42, SLC25A43, SLC25A44, SLC25A45, |
| | SLC25A46 |
| SLC26: The | SLC26A1, SLC26A2, SLC26A3, SLC26A4, SLC26A5, |
| multifunctional anion | SLC26A6, SLC26A7, SLC26A8, SLC26A9, SLC26A10, |
| exchanger family | SLC26A11 |
| SLC27: The fatty acid | SLC27A1, SLC27A2, SLC27A3, SLC27A4, SLC27A5, |
| transport protein family | SLC27A6 |
| SLC28: The Na+- | SLC28A1, SLC28A2, SLC28A3 |
| coupled nucleoside | |
| transport family | |
| SLC29: The facilitative | SLC29A1, SLC29A2, SLC29A3, SLC29A4 |
| nucleoside transporter | |
| family | |
| SLC30: The zinc efflux | SLC30A1, SLC30A2, SLC30A3, SLC30A4, SLC30A5, |
| family | SLC30A6, SLC30A7, SLC30A8, SLC30A9, SLC30A10 |
| SLC31: The copper | SLC31A1 |
| transporter family | |
| SLC32: The vesicular | SLC32A1 |
| inhibitory amino acid | |
| transporter family | |
| SLC33: The Acetyl-CoA | SLC33A1 |
| transporter family | |
| SLC34: The type II Na+- | SLC34A1, SLC34A2, SLC34A3 |
| phosphate cotransporter | |
| family | |
| SLC35: The nucleoside- | subfamily A; SLC35A1, SLC35A2, SLC35A3, SLC35A4, |
| sugar transporter family | SLC35A5 |
| | subfamily B; SLC35B1, SLC35B2, SLC35B3, SLC35B4 |
| | subfamily C; SLC35C1, SLC35C2 |
| | subfamily D; SLC35D1, SLC35D2, SLC35D3 |
| | subfamily E; SLC35E1, SLC35E2, SLC35E3, SLC35E4 |
| SLC36: The proton- | SLC36A1, SLC36A2, SLC36A3, SLC36A4 |
| coupled amino acid | |
| transporter family | |
| SLC37: The sugar- | SLC37A1, SLC37A2, SLC37A3, SLC37A4 |
| phosphate/phosphate | |
| exchanger family | |
| SLC38: The System A & | SLC38A1, SLC38A2, SLC38A3, SLC38A4, SLC38A5, |
| N, sodium-coupled | SLC38A6 |
| neutral amino acid | |
| transporter family | |
| SLC39: The metal ion | SLC39A1, SLC39A2, SLC39A3, SLC39A4, SLC39A5, |
| transporter family | SLC39A6, SLC39A7, SLC39A8, SLC39A9, SLC39A10, |
| | SLC39A11, SLC39A12, SLC39A13, SLC39A14 |
| SLC40: The basolateral | SLC40A1 |
| iron transporter family | |
| SLC41: The MgtE-like | SLC41A1, SLC41A2, SLC41A3 |
| magnesium transporter | |
| family | |
| SLC42: The Rh | RhAG, RhBG, RhCG |
| ammonium transporter | |
| family (pending) | |
| SLC43: Na+- | SLC43A1, SLC43A2, SLC43A3 |
| independent, system-L | |
| like amino acid | |
| transporter family | |
| SLC44: Choline-like | SLC44A1, SLC44A2, SLC44A3, SLC44A4, SLC44A5 |
| transporter family | |
| SLC45: Putative sugar | SLC45A1, SLC45A2, SLC54A3, SLC45A4 |
| transporter family | |
| SLC46: Heme transporter | SLC46A1, SLC46A2 |
| family | |
| SLC47: Multidrug and | SLC47A1, SLC47A2 |
| toxin extrusion | |

The viruses for use in the methods provided herein also can encode proteins, such as transporter proteins (e.g., the human norepinephrine transporter (hNET) and the human sodium iodide symporter (hNIS)), which can provide increase uptake diagnostic and therapeutic moieties across the cell membrane of infected cells for therapy, imaging or detection (see, e.g. U.S. Patent Pub. No. 2009-0117034). The sodium-iodide symporter (NIS) is an ion pump that transports iodide (I⁻) into thyroid epithelial cells across the basolateral plasma membrane, an important step in the process of iodide organification and the formation of triiodothyronine (T₃) and thyroxine (T₄). sNIS also is referred to as the “Sodium/iodide cotransporter,” “Na(+)/I(−) cotransporter,” “SLC5A5,” “TC 2.A.21.5.1” and “solute carrier family 5 member 5.” In addition, these proteins, when expressed in tumor cells, can provide a target for capture of the tumor cells, such as by an antibody that specifically binds to an epitope of the protein that is expressed on the surface of the tumor cells.

### (6) Proteins Detectable by Antibodies

Viruses also can be modified to express a heterologous reporter protein that can be detected with antibodies, typically by indirect or direct Enzyme Linked ImmunoSorbent Assay (ELISA). Any protein or epitope thereof against which an antibody can be can be raised can be employed for these purposes. For example, as a non-radioactive alternative, chloramphenicol acetyltransferase expression can be quantified in an ELISA via immunological detection of the CAT enzyme expressed in the virus (see e.g., Francois et al. (2005) Antimicrob. Agents Chemother. 49:3770-3775). In another example, the well-defined human Growth Hormone (hGH) reporter system can be utilized. When cloned into the viruses and expressed in the infected host cell, the hGH reporter protein can be secreted into the culture medium, which means that cell lysis is not necessary for quantifying the reporter protein. Detection of the secreted hGH can be carried out, for example, using ¹²⁵I-labeled antibodies against the growth hormone or with anti-hGH antibodies bound to the surface of a microtiter plate. For example, the hGH from the supernatant of the culture medium is added to the wells and binds to the antibody on the plate. The bound hGH can be detected in two steps via a digoxigenin-coupled anti-hGH antibody and a peroxidase-coupled anti-digoxigenin antibody. Bound peroxidase can then be quantified by incubation with a substrate.

### (7) Fusion Proteins

The viruses also can be modified to express reporter proteins that are fusion proteins, encoded by fusion genes. The fusion protein can contain all or part of an endogenous viral protein, or contain only heterologous amino acids sequences. The fusion protein can contain a polypeptide, protein or fragment thereof that is itself detectable, such as by spectrometry, fluorescence, chemiluminescence, or any other method known in the art, or catalyzes a detectable reaction or some visible change in the host cells or their environment upon addition of the appropriate substrate, or binds a detectable product. In one example, the fusion gene is a fusion of two individual genes that are required for a fully functional dateable product. For example, the luxA and luxB genes of bacterial luciferase can be fused to produce the fusion gene (Fab2), which can be expressed to produce a fully functional luciferase protein, as described above. In another example, the fusion protein can contain more than one detectable element. For example, a fluorescent protein, such as GFP, can be expressed as a fusion protein with a bioluminescent protein, such as luciferase, or another fluorescent protein that differs in the wavelength of light emitted, such as DsRed. In another non-limiting example, an enzyme, such as β-galactosidase, can be expressed as a fusion protein with a protein or polypeptide detectable by antibodies, such as hGH.

### (8) Proteins that Interact with Host Cell Proteins

The viruses also can be modified to express a reporter protein that directly interacts with one or more proteins that are expressed in the host cell. This interaction can result in a detectable change in the reporter protein such that the interaction can be measured. If the host cell proteins(s) are expressed during a particular biological process, then the reporter protein can be used to indicate the initiation of this process. In some examples, the reporter protein can be a substrate of a host cell protease. Once cleaved, one or more of the separate cleaved products can be differentially detected over the uncleaved protein. In one example, the virus can be modified to express a protein that contains a caspase target sequence, such as LEVD (SEQ ID NO:55) or DEVD (SEQ ID NO:56). For example, a reporter virus can be modified to express a fusion protein that contains a caspase target sequence that is flanked by two fluorescent molecules, such as CFP and YFP. Cleavage of the fusion protein results in fluorescent signals that can be differentiated from the uncleaved protein by fluorescence resonance energy transfer (FRET) analysis. FRET is a distance-dependent interaction between the electronic excited states of two dye molecules in which excitation is transferred from a donor molecule to an acceptor molecule without emission of a photon. When two suitable fluorescent molecules are separated by a sufficiently short distance, FRET will occur and observed emission at the wavelength corresponding to the donor will increase. When the molecules are separated further, FRET decreases (Zaccolo et al. (2004) Circ. Res. 94:866-873). The uncleaved fusion protein results in intense FRET, but when caspases are activated in the target cell during apoptosis, the fusion protein is cleaved and the molecules are separated, so FRET diminishes (He et al. (2004) Am. J. Pathol. 164:1901-1913). In other examples, a fusion protein is made of a luciferase and a fluorophore, linked by a cleavage sequence, and cleavage is detected by bioluminescence resonance energy transfer (BRET) analysis (Hu et al. (2005) J. Virol. Methods 128:93-103).

### ii. Operable Linkage to Promoter

The heterologous nucleic acid sequences encoding a reporter protein can be expressed in the viruses by being operably linked to a promoter. The heterologous nucleic acid can be operatively linked to a native promoter or a heterologous (with respect to the virus) promoter. Any promoter known to initiate transcription of an operably-linked open reading frame can be used. The choice of promoter can, however, affect the timing (in relation to viral infection and replication) and the level of the expression of the reporter gene. In some instances, certain requirements exist when operably linking heterologous nucleic acid to the promoter to ensure optimal expression. For example, when a reporter gene is operably linked to a promoter for expression in vaccinia viruses, the heterologous nucleic acid typically does not contain any intervening sequences, such as introns, as the virus does not splice its transcripts. Methods and parameters for operably linking heterologous nucleic acids sequences to promoters for successful expression are well known in the art (see, e.g., U.S. Pat. Nos. 4,769,330, 4,603,112, 4,722,848, 4,215,051, 5,110,587, 5,174,993, 5,922,576, 6,319,703, 5,719,054, 6,429,001, 6,589,531, 6,573,090, 6,800,288, 7,045,313; He et al. (1998) Proc. Natl. Acad. Sci. USA 95(5):2509-2514; Racaniello et al. (1981) Science 214:916-919; Hruby et al. (1990) Clin Micro Rev. 3:153-170).

### (1) Promoter Characteristics

The heterologous nucleic acid can be operatively linked to a native promoter or a heterologous (with respect to the virus) promoter. Any suitable promoters, including synthetic and naturally-occurring and modified promoters, can be used. The promoter region includes specific sequences that are involved in polymerase recognition, binding and transcription initiation. These sequences can be cis acting or can be responsive to trans acting factors. Promoters, depending upon the nature of the regulation, can be constitutive or regulated. Regulated promoters can be inducible or environmentally responsive (e.g., respond to cues such as pH, anaerobic conditions, osmoticum, temperature, light, or cell density). Inducible promoters can include, but are not limited to, a tetracycline-repressed regulated system, ecdysone-regulated system, and rapamycin-regulated system (Agha-Mohammadi and Lotze (2000) J. Clin. Invest. 105(9): 1177-1183). Many promoter sequences are known in the art. See, for example, U.S. Pat. Nos. 4,980,285; 5,631,150; 5,707,928; 5,759,828; 5,888,783; 5,919,670, and, Sambrook, et al. Molecular Cloning: A Laboratory Manual, 2nd Ed., Cold Spring Harbor Press (1989). Synthetic promoters also can be generated. Specific cis elements that can function to modulate a minimal promoter, such as one that contains only a TATA box and an initiator sequence, can be identified and used to generate a promoter that is optimized for the intended use (Edelman et al. (2000) Proc. Natl. Acad. Sci. USA 97:3038-3043). Synthetic promoters for the expression of proteins in vaccinia virus are known in the art, and can include various regulatory elements that dictate the expression profile of the protein (such as the stage in the viral life cycle at which the protein is expressed), and/or enhance expression (see e.g., Pfleiderer et al. (1995) J Gen Virol. 76:2957-2962, Hammond et al. (1997) J Virol Methods. 66:135-138, Chakrabarti et al. (1997) BioTechniques 23:1094-1097). Synthetic promoters also include chemically synthesized promoters, such as those described in U.S. Pat. Pub. No. 2004/0171573.

Promoters that are responsive to external factors, either directly or indirectly, can be selected for use. External factors can include, for example, drugs and inhibitors, such as chemotherapeutic drugs. In one example, the heterologous nucleic acid, such as that which encodes a reporter protein, is operably linked to a promoter that is sensitive to one or more chemotherapeutic drugs. That is, the expression of the heterologous protein from the promoter is inhibited by the chemotherapeutic agent. In another example, the heterologous nucleic acid, such as that which encodes a reporter protein, is operably linked to a promoter that is resistant to one or more chemotherapeutic drugs. That is, the expression of the heterologous protein from the promoter is unaffected by the chemotherapeutic agent. Such a promoter can be of any origin, including mammalian or viral, and be natural or synthetic.

Promoters also can be selected for use on the basis of the relative expression levels that they initiate. Strong promoters are those that support a relatively high level of expression, while weak promoters are those that support a relatively low level of expression. For example, the vaccinia virus synthetic early/late and late promoters are relatively strong promoters, whereas vaccinia synthetic early, P7.5k early/late, P7.5k early, and P28 late promoters are relatively weaker promoters (see e.g., Chakrabarti et al. (1997) BioTechniques 23(6):1094-1097).

### (a) Viral and Host Factors

Expression of heterologous proteins can be influenced by one or more proteins or molecules expressed by the virus, or one or more factors expressed by the host. For example, various viral transcription factors can bind other proteins or to the promoter sequence to initiate transcription, or various host factors can interact with one or more regions in the promoter sequence, or with one or more other factors, to initiate transcription. The expression or availability of these molecules and proteins can dictate, for example, level of expression, or the timing of expression, of the heterologous protein under the control of the promoter with which the factors interact.

In one example, the expression of a heterologous protein, such as a reporter protein, from a virus can be controlled temporally by using a promoter that requires interaction with one or more host or viral factors that are expressed, or are available, at a particular stage of the viral life cycle, to initiate transcription. Vaccinia virus coordinates its progression through its replicative cycle by expressing individual proteins at specific times. The temporal regulation of gene expression is controlled at the level of transcriptional initiation, and occurs through a cascade. The transcription factors required for intermediate genes are expressed as early proteins, factors required for late genes are intermediate gene products and the late genes products are packaged into the virions and act as transcription factors for early genes. For example, the vaccinia virus early transcription factor (ETF), which is a dimer made from the products of two late genes, interacts with two regions of the early promoters and recruits the RNA polymerase to the site of transcription. Initiation of transcription results in the synthesis of the early genes within minutes of viral entry into the cell, and is independent of de novo protein synthesis because ETF and the RNA polymerase are already present in the virion. In some instances, genes are expressed continuously, which can be achieved by a tandem arrangement of early and intermediate or late promoters operably linked to the open reading frame (Broyles et al. (1986) Proc. Natl. Acad. Sci. USA 83:3141-3145, Ahn et al. (1990) Mol Cell Biol. 10:5433-5441).

Nearly all viruses, including, but not limited to, poxviruses (including vaccinia virus), adenoviruses, herpesviruses, flaviviruses and caliciviruses link the switch from early to late gene expression to genome replication. The intermediate genes are expressed immediately post-replication, followed closely thereafter by transcription of the late genes. In the absence of nucleic acid synthesis, transcriptional switch does not occur. Because of this regulated expression, inhibition of genome synthesis by, for example, the addition of inhibitors of nucleic acid synthesis such as cytosine arabinoside (Ara-C), results in the inhibition of intermediate and late gene transcription (Vos et al. (1988) EMBO J. 7:3487-3492, Kao et al. (1987) Virology 159:399-407). Therefore, operably linking a heterologous gene to a viral intermediate or late promoter links its expression in the virally-infected host to certain stages of the viral life cycle i.e., after DNA replication. In contrast, operably linking a heterologous gene to a viral early promoter results in its expression immediately following viral entry into the host cell. By selecting the appropriate promoter, a reporter protein can therefore be used to reflect transcriptional activity at various stages of the viral life cycle, which can be linked to multiple viral and/or host factors, and/or external factors, such as drugs and inhibitors.

### (b) Exemplary Promoters

Exemplary promoters include synthetic promoters, including synthetic viral and animal promoters. Native promoters or heterologous promoters include, but are not limited to, viral promoters, such as vaccinia virus and adenovirus promoters. Vaccinia viral promoters can be synthetic or natural promoters, and include vaccinia early, intermediate, early/late and late promoters. Exemplary vaccinia viral promoters for use in the methods can include, but are not limited to, P7.5k, P11k, PSL, PSEL, PSE, HSR, TK, P28, C11R, G8R, F17R, I3L, I8R, A1L, A2L, A3L, H1L, H3L, H5L, H6R, H8R, D1R, D4R, D5R, D9R, D11L, D12L, D13L, M1L, N2L, P4b or K1 promoters. Other viral promoters can include, but are not limited to, adenovirus late promoter, Cowpox ATI promoter, T7 promoter, adenovirus late promoter, adenovirus E1A promoter, SV40 promoter, cytomegalovirus (CMV) promoter, thymidine kinase (TK) promoter, or Hydroxymethyl-Glutaryl Coenzyme A (HMG) promoter.

In some examples, it can be desirable to choose promoters that initiate expression at particular time points in the viral life cycle. An exemplary vaccinia early promoter is a synthetic early promoter (PSE), which typically initiates gene expression from 0-3 hours post infection. Exemplary vaccinia late promoters include, but are not limited to, a vaccinia 11k promoter (P11k) and a synthetic late promoter (PSL), which typically initiate gene expression 2-3 hours post-infection. Exemplary promoters in vaccinia virus that are expressed throughout the life cycle include tandem arrangements of vaccinia early and intermediate or late promoters (see e.g., Wittek et al. (1980) Cell 21: 487-493; Broyles and Moss (1986) Proc. Natl. Acad. Sci. USA 83:3141-3145; Ahn et al. (1990) Mol. Cell. Biol. 10: 5433-54441; Broyles and Pennington (1990) J. Virol. 64:5376-5382). Exemplary vaccinia early/late promoters that express throughout the vaccinia life cycle include, but are not limited to, a 7.5K promoter (P7.5k) and a synthetic early/late promoter (PSEL).

In some examples, it can be desirable to choose a promoter of a particular relative strength. For example, in vaccinia, synthetic early/late PSEL and many late promoters (e.g., P11k and PSL) are relatively strong promoters, whereas vaccinia synthetic early, PSE, P7.5k early/late, P7.5k early, and P28 late promoters are relatively weak promoters (see e.g., Chakrabarti et al. (1997) BioTechniques 23(6):1094-1097).

### iii. Expression of Multiple Reporter Proteins

A virus used in the methods provided herein can be modified to express two or more gene products that emit a detectable signal, catalyze a detectable reaction, bind a detectable compound, form a detectable product, or any combination thereof. Any combination of such gene products can be expressed by the viruses for use in the methods provided herein. Detection of the gene products, or reporter proteins, can be effected by, for example, spectrometry, fluorescence, chemiluminescence, MRI, PET, histology or any other method known in the art. A virus expressing two or more detectable gene products can be imaged in vitro or in vivo using such methods. In certain examples, the virus can express the two or more reporter proteins as a fusion protein, such as described above. For example, a virus can be modified to express a fusion protein containing two fluorescent proteins that differ in the wavelength of light emitted, such as GFP and DsRed. In certain examples the two or more gene products are expressed as individual transcripts, from separate promoters. The promoters can be of the same type and sequence, or a different type and sequence. For example, two or more reporter genes can be transcribed separately from the same type of promoter, such as for example, the vaccinia P7.5k early/late promoter, at different locations in the virus genome. Alternately, the two or more reporter genes can be transcribed from different promoters. For example, a vaccinia virus can be modified to express the β-galactosidase gene (lacZ) under the control of the vaccinia P7.5 early/late promoter, and the gene for Katushka fluorescent protein under the control of the vaccinia PSE synthetic early promoter, PSEL synthetic early/late promoter, or PSL synthetic late promoter.

c. Further Modifications of the Viruses

The viruses used in the methods provided herein can be further modified. Such modifications can, for example, enhance the ease with which the methods are performed, reduce the time taken to perform the methods, provide conditions of increased safety or suitability for administration, compared to unmodified viruses. Such characteristics can include, but are not limited to, attenuated pathogenicity, reduced toxicity, increased or decreased replication competence, increased, decreased or otherwise altered tropism, increased or decreased sensitivity to drugs, such as nucleoside analogs and any combination thereof. The viruses used in the methods provided herein can be modified by any known method for modifying a virus. For example, the viruses can be modified to express one or more heterologous genes. The heterologous genes can be expressed under the control of endogenous viral promoters, or exogenous (i.e., heterologous to the virus) promoters, including synthetic promoters.

Oncolytic viruses have been genetically altered to attenuate their virulence, to improve their safety profile, enhance their tumor specificity, and they have also been equipped with additional genes, for example cytotoxins, cytokines, prodrug converting enzymes to improve the overall efficacy of the viruses (see, e.g., Kim et al., (2009) Nat Rev Cancer 9:64-71; Garcia-Aragoncillo et al., (2010) Curr Opin Mol Ther 12:403-411; see U.S. Pat. Nos. 7,588,767, 7,588,771, 7,662,398 and 7,754,221 and U.S. Pat. Publ. Nos. 2007/0202572, 2007/0212727, 2010/0062016, 2009/0098529, 2009/0053244, 2009/0155287, 2009/0117034, 2010/0233078, 2009/0162288, 2010/0196325, 2009/0136917 and 2011/0064650).

The modifications can be effected by any method known in the art, and can be introduced into the virus before, after, simultaneously, or in the absence of, the introduction one or more reporter genes. In certain examples, the virus is modified to attenuate pathogenicity. In some examples, it can be desirable to generate a more attenuated virus. A more attenuated virus can be more suitable for in vivo administration and in in vitro assays, providing a safer environment for laboratory personnel and reducing the laboratory biosafety requirements. Attenuation of the virus can be effected by modification of one or more viral genes, such as by a point mutation, a deletion mutation, an interruption by an insertion, a substitution or a mutation of the viral gene promoter or enhancer regions. In such instances, it is advantageous to first identify a target gene involved in pathogenicity, although random mutagenesis can result in attenuation of the virus. The target genes also are typically non-essential, such that the ability of the virus to propagate without the need of a packaging cell lines is preserved when the genes are not expressed, or expressed at decreased levels. In viruses such as vaccinia virus, mutations in non-essential genes, such as the thymidine kinase (TK) gene or hemagglutinin (HA) gene have been employed to attenuate the virus (e.g., Buller et al. (1985) Nature 317:813-815, Shida et al. (1988) J. Virol. 62(12):4474-4480, Taylor et al. (1991) J. Gen. Virol. 72 (Pt 1):125-30, U.S. Pat. Nos. 5,364,773, 6,265,189 and 7,045,313). The inactivation of these genes decreases the overall pathogenicity of the virus without eliminating the ability of the viruses to replicate in certain cell types.

Attenuation also can be effected without eliminating or reducing the expression of one or more particular genes involved in pathogenicity. For example, increasing the number of genes that the virus expresses can cause competition for viral transcription and/or translation factors, which can result in changes in expression of endogenous viral genes. Such changes can affect viral processes involved in viral replication, thus contributing to the attenuation of the virus. For example, viral processes, such as viral nucleic acid replication, transcription of other viral genes, viral mRNA production, viral protein synthesis, or virus particle assembly and maturation, can be affected. Insertion of gene expression cassettes that require binding of host factors for efficient transcription can be used to compete the transcription and/or translation factors away from the endogenous viral promoters and transcripts. For example, insertion of gene expression cassettes that contain vaccinia strong late promoters into vaccinia virus can be used to attenuate expression of endogenous vaccinia late genes.

Viruses provided herein also can contain a modification that alters its infectivity or resistance to neutralizing antibodies. In one non-limiting example deletion of the A35R gene in an vaccinia LIVP strain can decrease the infectivity of the virus. In some examples, the viruses provided herein can be modified to contain a deletion of the A35R gene. Exemplary methods for generating such viruses are described in PCT Publication No. WO2008/100292, which describes vaccinia LIVP viruses GLV-1j87, GLV-1j88 and GLV-1j89, which contain deletion of the A35R gene.

In another non-limiting example, replacement of viral coat proteins (e.g., A34R, which encodes a viral coat glycoprotein) with coat proteins from either more virulent or less virulent virus strains can increase or decrease the clearance of the virus from the subject. In one example, the A34R gene in an vaccinia LIVP strain can be replaced with the A34R gene from vaccinia IHD-J strain. Such replacement can increase the extracellular enveloped virus (EEV) form of vaccinia virus and can increase the resistance of the virus to neutralizing antibodies.

### i. Expression of a Therapeutic Gene Product

In some examples provided herein, oncolytic reporter viruses can be administered to a subject for diagnosis and therapy of tumors, metastases and CTCs. In some examples, the oncolytic viruses provide oncolytic therapy of a tumor cell without the expression of a therapeutic gene. In other examples, the oncolytic reporter viruses can express one or more genes whose products are useful for tumor therapy. For example, a virus can express proteins that cause cell death or whose products cause an anti-tumor immune response. Such genes can be considered therapeutic genes. A variety of therapeutic gene products, such as toxic or apoptotic proteins, or siRNA, are known in the art, and can be used with the viruses provided herein. The therapeutic genes can act by directly killing the host cell, for example, as a channel-forming or other lytic protein, or by triggering apoptosis, or by inhibiting essential cellular processes, or by triggering an immune response against the cell, or by interacting with a compound that has a similar effect, for example, by converting a less active compound to a cytotoxic compound.

Exemplary therapeutic gene products that can be expressed by the oncolytic reporter viruses include, but are not limited to, gene products (i.e., proteins and RNAs), including those useful for tumor therapy, such as, but not limited to, an anticancer agent, an anti-metastatic agent, or an antiangiogenic agent. For example, exemplary proteins useful for tumor therapy include, but are not limited to, tumor suppressors, cytostatic proteins and costimulatory molecules, such as a cytokine, a chemokine, or other immunomodulatory molecules, an anticancer antibody, such as a single-chain antibody, antisense RNA, siRNA, prodrug converting enzyme, a toxin, a mitosis inhibitor protein, an antitumor oligopeptide, an anticancer polypeptide antibiotic, an angiogenesis inhibitor, or tissue factor. For example, a large number of therapeutic proteins that can be expressed for tumor treatment in the viruses and methods provided herein are known in the art, including, but not limited to, a transporter, a cell-surface receptor, a cytokine, a chemokine, an apoptotic protein, a mitosis inhibitor protein, an antimitotic oligopeptide, an antiangiogenic factor (e.g., hk5), angiogenesis inhibitors (e.g., plasminogen kringle 5 domain, anti-vascular endothelial growth factor (VEGF) scAb, tTF-RGD, truncated human tissue factor-αᵥβ₃-integrin RGD peptide fusion protein), anticancer antibodies, such as a single-chain antibody (e.g., an antitumor antibody or an antiangiogenic antibody, such as an anti-VEGF antibody or an anti-epidermal growth factor receptor (EGFR) antibody), a toxin, a tumor antigen, a prodrug converting enzyme, a ribozyme, RNAi, and siRNA.

Additional therapeutic gene products that can be expressed by the oncolytic reporter viruses include, but are not limited to, cell matrix degradative genes, such as but not limited to, relaxin-1 and MMP9, and genes for tissue regeneration and reprogramming human somatic cells to pluripotency, such as but not limited to, nAG, Oct4, NANOS, Neogenin-1, Ngn3, Pdx1 and Mafa.

Costimulatory molecules for use in the methods provided herein include any molecules which are capable of enhancing immune responses to an antigen/pathogen in vivo and/or in vitro. Costimulatory molecules also encompass any molecules which promote the activation, proliferation, differentiation, maturation or maintenance of lymphocytes and/or other cells whose function is important or essential for immune responses.

An exemplary, non-limiting list of therapeutic proteins includes tumor growth suppressors such as IL-24, WT1, p53, pseudomonas A endotoxin, diphtheria toxin, Arf, Bax, HSV TK, E. coli purine nucleoside phosphorylase, angiostatin and endostatin, p16, Rb, BRCA1, cystic fibrosis transmembrane regulator (CFTR), Factor VIII, low density lipoprotein receptor, beta-galactosidase, alpha-galactosidase, beta-glucocerebrosidase, insulin, parathyroid hormone, alpha-1-antitrypsin, rsCD40L, Fas-ligand, TRAIL, TNF, antibodies, microcin E492, diphtheria toxin, Pseudomonas exotoxin, Escherichia coli Shiga toxin, Escherichia coli Verotoxin 1, and hyperforin. Exemplary cytokines include, but are not limited to, chemokines and classical cytokines, such as the interleukins, including, but not limited to, interleukin-1, interleukin-2, interleukin-6 and interleukin-12, tumor necrosis factors, such as tumor necrosis factor alpha (TNF-α), interferons such as interferon gamma (IFN-γ), granulocyte macrophage colony stimulating factor (GM-CSF), erythropoietin and exemplary chemokines including, but not limited to CXC chemokines such as IL-8 GROα, GROβ, GROγ, ENA-78, LDGF-PBP, GCP-2, PF4, Mig, IP-10, SDF-1α/β, BUNZO/STRC33, I-TAC, BLC/BCA-1; CC chemokines such as MIP-1α, MIP-1β, MDC, TECK, TARC, RANTES, HCC-1, HCC-4, DC-CK1, MIP-3α, MIP-3β, MCP-1, MCP-2, MCP-3, MCP-4, Eotaxin, Eotaxin-2/MPIF-2, I-309, MIP-5/HCC-2, MPIF-1, 6Ckine, CTACK, MEC; lymphotactin; and fractalkine. Exemplary other costimulatory molecules include immunoglobulin superfamily of cytokines, such as B7.1 and B7.2.

Exemplary therapeutic proteins that can be expressed by the oncolytic reporter viruses used in the methods provided herein include, but are not limited to, erythropoietin (e.g., SEQ ID NO:28), an anti-VEGF single chain antibody (e.g., SEQ ID NO:29), a plasminogen K5 domain (e.g., SEQ ID NO:30), a human tissue factor-αvβ3-integrin RGD fusion protein (e.g., SEQ ID NO:31), interleukin-24 (e.g., SEQ ID NO:32), or immune stimulators, such as IL-6-IL-6 receptor fusion protein (e.g., SEQ ID NO:33).

In some examples, the oncolytic reporter viruses used in the methods provided herein can express one or more therapeutic gene products that are proteins that convert a less active compound into a compound that causes tumor cell death. Exemplary methods of conversion of such a prodrug compound include enzymatic conversion and photolytic conversion. A large variety of protein/compound pairs are known in the art, and include, but are not limited to, Herpes simplex virus thymidine kinase/ganciclovir, Herpes simplex virus thymidine kinase/(E)-5-(2-bromovinyl)-2′-deoxyuridine (BVDU), varicella zoster thymidine kinase/ganciclovir, varicella zoster thymidine kinase/BVDU, varicella zoster thymidine kinase/(E)-5-(2-bromovinyl)-1-beta-D-arabinofuranosyluracil (BVaraU), cytosine deaminase/5-fluorouracil, cytosine deaminase/5-fluorocytosine, purine nucleoside phosphorylase/6-methylpurine deoxyriboside, beta lactamase/cephalosporin-doxorubicin, carboxypeptidase G2/4-[(2-chloroethyl)(2-mesyloxyethyl)amino]benzoyl-L-glutamic acid (CMDA), carboxypeptidase A/methotrexate-phenylamine, cytochrome P450/acetominophen, cytochrome P450-2B1/cyclophosphamide, cytochrome P450-4B1/2-aminoanthracene, 4-ipomeanol, horseradish peroxidase/indole-3-acetic acid, nitroreductase/CB1954, rabbit carboxylesterase/7-ethyl-10-[4-(1-piperidino)-1-piperidino]carbonyloxy-camptothecin (CPT-11), mushroom tyrosinase/bis-(2-chloroethyl)amino-4-hydroxyphenylaminomethanone 28, beta galactosidase/1-chloromethyl-5-hydroxy-1,2-dihydro-3H-benz[e]indole, beta glucuronidase/epirubicin glucuronide, thymidine phosphorylase/5′-deoxy-5-fluorouridine, deoxycytidine kinase/cytosine arabinoside, and linamerase/linamarin.

Other therapeutic gene products that can be expressed by the oncolytic reporter viruses used in the methods provided herein include siRNA and microRNA molecules. The siRNA and/or microRNA molecule can be directed against expression of a tumor-promoting gene, such as, but not limited to, an oncogene, growth factor, angiogenesis promoting gene, or a receptor. The siRNA and/or microRNA molecule also can be directed against expression of any gene essential for cell growth, cell replication or cell survival. The siRNA and/or microRNA molecule also can be directed against expression of any gene that stabilizes the cell membrane or otherwise limits the number of tumor cell antigens released from the tumor cell. Design of an siRNA or microRNA can be readily determined according to the selected target of the siRNA; methods of siRNA and microRNA design and down-regulation of genes are known in the art, as exemplified in U.S. Pat. Pub. Nos. 2003-0198627 and 2007-0044164, and Zeng et al., (2002) Molecular Cell 9:1327-1333.

Therapeutic gene products include viral attenuation factors, such as antiviral proteins. Antiviral proteins or peptides can be expressed by the viruses provided herein. Expression of antiviral proteins or peptides can control viral pathogenicity. Exemplary viral attenuation factors include, but are not limited to, virus-specific antibodies, mucins, thrombospondin, and soluble proteins such as cytokines, including, but not limited to TNFα, interferons (for example IFNα, IFNβ, or IFNγ) and interleukins (for example IL-1, IL-12 or IL-18).

Another exemplary therapeutic gene product that can be expressed by the oncolytic reporter viruses used in the methods provided herein is a protein ligand, such as antitumor oligopeptide. Antitumor oligopeptides are short protein peptides with high affinity and specificity to tumors. Such oligopeptides could be enriched and identified using tumor-associated phage libraries (Akita et al. (2006) Cancer Sci. 97(10):1075-1081). These oligopeptides have been shown to enhance chemotherapy (U.S. Pat. No. 4,912,199). The oligopeptides can be expressed by the viruses provided herein. Expression of the oligopeptides can elicit anticancer activities on their own or in combination with other chemotherapeutic agents. An exemplary group of antitumor oligopeptides is antimitotic peptides, including, but not limited to, tubulysin (Khalil et al. (2006) Chembiochem. 7(4):678-683), phomopsin, hemiasterlin, taltobulin (HTI-286, 3), and cryptophycin. Tubulysin is from myxobacteria and can induce depletion of cell microtubules and trigger the apoptotic process. The antimitotic peptides can be expressed by the viruses provide herein and elicit anticancer activities on their own or in combination with other therapeutic modalities.

Another exemplary therapeutic gene product that can be expressed by the oncolytic reporter viruses used in the methods provided herein is a protein that sequesters molecules or nutrients needed for tumor growth. For example, the virus can express one or more proteins that bind iron, transport iron, or store iron, or a combination thereof. Increased iron uptake and/or storage by expression of such proteins not only, increases contrast for visualization and detection of a tumor or tissue in which the virus accumulates, but also depletes iron from the tumor environment. Iron depletion from the tumor environment removes a vital nutrient from the tumors, thereby deregulating iron hemostasis in tumor cells and delaying tumor progression and/or killing the tumor.

Additionally, iron, or other labeled metals, can be administered to a tumor-bearing subject, either alone, or in a conjugated form. An iron conjugate can include, for example, iron conjugated to an imaging moiety or a therapeutic agent. In some cases, the imaging moiety and therapeutic agent are the same, e.g., a radionuclide. Internalization of iron in the tumor, wound, area of inflammation or infection allows the internalization of iron alone, a supplemental imaging moiety, or a therapeutic agent (which can deliver cytotoxicity specifically to tumor cells or deliver the therapeutic agent for treatment of the wound, area of inflammation or infection). These methods can be combined with any of the other methods provided herein.

In some examples, the oncolytic reporter viruses used in the methods provided herein can be modified to express one or more antigens to elicit antibody production against an expressed gene product and enhance the immune response against the infected tumor cell. The sustained release of antigen can result in an immune response by the viral-infected host, in which the host can develop antibodies against the antigen, and/or the host can mount an immune response against cells expressing the antigen, including an immune response against tumor cells. Thus, the sustained release of antigen can result in immunization against tumor cells. In some embodiments, the viral-mediated sustained antigen release-induced immune response against tumor cells can result in complete removal or killing of all tumor cells. The immunizing antigens can be endogenous to the virus, such as vaccinia antigens on a vaccinia virus used to immunize against smallpox, measles, mumps, or the immunizing antigens can be exogenous antigens expressed by the virus, such as influenza or HIV antigens expressed on a viral capsid surface. In the case of smallpox, for example, a tumor specific protein antigen can be carried by an attenuated vaccinia virus (encoded by the viral genome) for a smallpox vaccine. Thus, the viruses provided herein, including the modified vaccinia viruses can be used as vaccines.

As shown previously, solid tumors can be treated with viruses, such as vaccinia viruses, resulting in an enormous tumor-specific virus replication, which can lead to tumor protein antigen and viral protein production in the tumors (U.S. Patent Publication No. 2005/0031643). Vaccinia virus administration to mice resulted in lysis of the infected tumor cells and a resultant release of tumor-cell-specific antigens. Continuous leakage of these antigens into the body led to a very high level of antibody titer (in approximately 7-14 days) against tumor proteins, viral proteins, and the virus encoded engineered proteins in the mice. The newly synthesized anti-tumor antibodies and the enhanced macrophage, neutrophils count were continuously delivered via the vasculature to the tumor and thereby provided for the recruitment of an activated immune system against the tumor. The activated immune system then eliminated the foreign compounds of the tumor including the viral particles. This interconnected release of foreign antigens boosted antibody production and continuous response of the antibodies against the tumor proteins to function like an autoimmunizing vaccination system initiated by vaccinia viral infection and replication, followed by cell lysis, protein leakage and enhanced antibody production.

The administered virus can stimulate humoral and/or cellular immune response in the subject, such as the induction of cytotoxic T lymphocytes responses. For example, the virus can provide prophylactic and therapeutic effects against a tumor infected by the virus or other infectious diseases, by rejection of cells from tumors or lesions using viruses that express immunoreactive antigens (Earl et al., (1986) Science 234:728-831; Lathe et al., Nature London) 32: 878-880 (1987)), cellular tumor-associated antigens (Bernards et al., Proc. Natl. Acad. Sci. USA 84: 6854-6858 (1987); Estin et al., Proc. Natl. Acad. Sci. USA 85: 1052-1056 (1988); Kantor et al., J. Natl. Cancer Inst. 84: 1084-1091 (1992); Roth et al., Proc. Natl. Acad. Sci. USA 93: 4781-4786 (1996)) and/or cytokines (e.g., IL-2, IL-12), costimulatory molecules (B7-1, B7-2) (Rao et al., J. Immunol. 156:3357-3365 (1996); Chamberlain et al., Cancer Res. 56:2832-2836 (1996); Oertli et al., J. Gen. Virol. 77: 3121-3125 (1996); Qin and Chatterjee, Human Gene Ther. 7:1853-1860 (1996); McAneny et al., Ann. Surg. Oncol.3:495-500 (1996)), or other therapeutic proteins.

Exemplary heterologous genes for modification of viruses herein are known in the art (see e.g. U.S. Pub. Nos. 2003-0059400, 2003-0228261, 2009-0117034, 2009-0098529, 2009-0053244, 2009-0081639 and 2009-0136917; U.S. Pat. Nos. 7,588,767 and 7,763,420; and International Pub. No. WO 2009/139921). A non-limiting description of exemplary genes encoding heterologous proteins for modification of virus strains is set forth in the following table. The sequence of the gene and encoded proteins are known to one of skill in the art from the literature. Hence, provided herein are virus strains, including any of the clonal viruses provided herein, that contain nucleotides encoding any of the heterologous proteins listed in Table 5.

```
TABLE 5
Detectable gene products
  Optical Imaging
    Luciferase
      bacterial luciferase
      luciferase (from Vibrio harveyi or Vibrio fischerii)
        luxA
        luxB
        luxC
        luxD
        luxE
        luxAB
        luxCD
        luxABCDE
      firefly luciferase
      Renilla luciferase from Renilla renformis
      Gaussia luciferase
      luciferases found among marine arthropods
      luciferases that catalyze the oxidation of Cypridina (Vargula) luciferin
      luciferases that catalyze the oxidation of Coleoptera luciferin
luciferase photoproteins
      aequorin photoprotein to which luciferin is non-covalently bound
click beetle luciferase
      CBG99
      CBG99-mRFP1
Fusion Proteins
      Ruc-GFP
Fluorescent Proteins
GFP
      aequorin from Aequorea victoria
      GFP from Aequorea victoria
      GFP from Aequorea coerulescens
      GFP from the anthozoan coelenterates Renilla reniformis and Renilla kollikeri (sea
      pansies)
      Emerald (Initrogen, Carlsbad, CA)
      EGFP (Clontech, Palo Alto, CA)
      Azami-Green (MBL International, Woburn, MA)
      Kaede (MBL International, Woburn, MA)
      ZsGreen1 (Clontech, Palo Alto, CA)
      CopGFP (Evrogen/Axxora, LLC, San Diego, CA)
      Anthozoa reef coral
      Anemonia sea anemone
      Renilla sea pansy
      Galaxea coral
      Acropora brown coral
      Trachyphyllia stony coral
      Pectiniidae stony coral
      GFP-like proteins
RFP
      RFP from the corallimorph Discosoma (DsRed) (Matz et al. (1999) Nature
      Biotechnology 17: 969-973)
      Heteractis reef coral, Actinia or Entacmaea sea anemone
      RFPs from Discosoma variants
          mRFP1 (Wang et al. (2004) Proc. Natl. Acad. Sci. U.S.A. 101: 16745-9)
          mCherry (Wang et al. (2004) PNAS USA. 101(48): 16745-9)
          tdTomato (Wang et al. (2004) PNAS USA. 101(48): 16745-9)
          mStrawberry (Wang et al. (2004) PNAS USA. 101(48): 16745-9)
          mTangerine (Wang et al. (2004) PNAS USA. 101(48): 16745-9)
          DsRed2 (Clontech, Palo Alto, CA)
          DsRed-T1 (Bevis and Glick (2002) Nat. Biotechnol. 20: 83-87)
          Anthomedusa J-Red (Evrogen)
          Anemonia AsRed2 (Clontech, Palo Alto, CA)
far-red fluorescent protein
      TurboFP635
      mNeptune monomeric far-red fluorescent protein
      Actinia AQ143 (Shkrob et al. (2005) Biochem J. 392(Pt 3): 649-54)
      Entacmaea eqFP611 (Wiedenmann et al. (2002) PNAS USA. 99(18): 11646-5I)
      Discosoma variants
          mPlum (Wang et al.. (2004) PNAS USA. 101(48): 16745-9)
          mRasberry (Wang et al. (2004) PNAS USA. 101(48): 16745-9)
          Heteractis HcRed1 and t-HcRed (Clontech, Palo Alto, CA)
IFP (infrared fluorescent protein)
near-infrared fluorescent protein
YFP
      EYFP (Clontech, Palo Alto, CA)
      YPet (Nguyen and Daugherty (2005) Nat Biotechnol. 23(3): 355-60)
      Venus (Nagai et al. (2002) Nat. Biotechnol. 20(1): 87-90)
      ZsYellow (Clontech, Palo Alto, CA)
      mCitrine (Wang et al. (2004) PNAS USA. 101(48): 16745-9)
OFP
      cOFP (Strategene, La Jolla, CA)
      mKO (MBL International, Woburn, MA)
      mOrange (Wang et al.. (2004) PNAS USA. 101(48): 16745-9)
CFP
      Cerulean (Rizzo (2004) Nat Biotechnol. 22(4): 445-9)
      mCFP (Wang et al. (2004) PNAS USA. 101(48): 16745-9)
      AmCyan1 (Clontech, Palo Alto, CA)
      MiCy (MBL International, Woburn, MA)
      CyPet (Nguyen and Daugherty (2005) Nat Biotechnol. 23(3): 355-60)
BFP
      EBFP (Clontech, Palo Alto, CA);
      phycobiliproteins from certain cyanobacteria and eukaryotic algae, phycoerythrins
      (red) and the phycocyanins (blue)
R-Phycoerythrin (R-PE)
B-Phycoerythrin (B-PE)
Y-Phycoerythrin (Y-PE
C-Phycocyanin (P-PC)
R-Phycocyanin (R-PC)
Phycoerythrin 566 (PE 566)
Phycoerythrocyanin (PEC)
Allophycocyanin (APC)
frp Flavin Reductase
CBP Coelenterazine-binding protein 1
PET imaging
      Cyp11B1 transcript variant 1
      Cyp11B1 transcript variant 2
      Cyp11B2
      AlstR
      PEPR-1
      LAT-4 (SLC43A2)
      Cyp51 transcript variant 1
      Cyp51 transcript variant 2
Transporter proteins
Solute carrier transporter protein families (SLC)
      SLC1 solute carrier 1 transporter protein family
      SLC1A1, SLC1A2, SLC1A3, SLC1A4, SLC1A5, SLC1A6, SLC1A7
      SLC2 solute carrier 2 transporter protein family
      SLC2A1, SLC2A2, SLC2A3, SLC2A4, SLC2A5, SLC2A6, SLC2A7, SLC2A8,
      SLC2A9, SLC2A10, SLC2A11, SLC2A12, SLC2A13, SLC2A14)
      SLC3 solute carrier 3 transporter protein family
      SLC3A1, SLC3A2
      SLC 4 solute carrier 4 transporter protein family
      SLC4A1, SLC4A2, SLC4A3, SLC4A4, SLC4A5, SLC4A6, SLC4A7, SLC4A8,
      SLC4A9, SLC4A10, SLC4A11
      SLC5 solute carrier 5 transporter protein family
      SLC5A1 sodium/glucose cotransporter 1
      SLC5A2 sodium/glucose cotransporter 2
      SLC5A3 sodium/myo-inositol cotransporter
      SLC5A4 low affinity sodium-glucose cotransporter
      SLC5A5 sodium/iodide cotransporter
      SLC5A6 sodium-dependent multivitamin transporter
      SLC5A7 high affinity choline transporter 1
      SLC5A8 sodium-coupled monocarboxylate transporter 1
      SLC5A9 sodium/glucose cotransporter 4
      SLC5A10 sodium/glucose cotransporter 5, isoform 1
      sodium/glucose cotransporter 5, isoform 2
      sodium/glucose cotransporter 5, isoform 3
      sodium/glucose cotransporter 5, isoform 4
      SLC5A11 sodium/myo-inositol cotransporter 2, isoform 1
      sodium/myo-inositol cotransporter 2, isoform 2
      sodium/myo-inositol cotransporter 2, isoform 3
      sodium/myo-inositol cotransporter 2, isoform 4
      SLC5A12 sodium-coupled monocarboxylate transporter 2, isoform 1
      sodium-coupled monocarboxylate transporter 2, isoform 2
          Sodium Iodide Symporter (NIS)
          hNIS (NM_000453)
          hNIS (BC105049)
          hNIS (BC105047)
          hNIS (non-functional hNIS variant containing an additional 11 aa)
      SLC6 solute carrier 6 transporter protein family
      SLC6A1 sodium- and chloride-dependent GABA transporter 1
      SLC6A2 norepinephrine transporter (sodium-dependent noradrenaline transporter)
      SLC6A3 sodium-dependent dopamine transporter
      SLC6A4 sodium-dependent serotonin transporter
      SLC6A5 sodium- and chloride-dependent glycine transporter 1
      SLC6A6 sodium-and chloride-dependent taurine transporter
      SLC6A7 sodium-dependent proline transporter
      SLC6A8 sodium- and chloride-dependent creatine transporter
      SLC6A9 sodium- and chloride-dependent glycine transporter 1, isoform 1
      sodium- and chloride-dependent glycine transporter 1, isoform 2
      sodium- and chloride-dependent glycine transporter 1, isoform 3
      SLC6A10 sodium- and chloride-dependent creatine transporter 2
      SLC6A11 sodium- and chloride-dependent GABA transporter 3
      SLC6A12 sodium- and chloride-dependent betaine transporter
      SLC6A13 sodium- and chloride-dependent GABA transporter 2
      SLC6A14 Sodium- and chloride-dependent neutral and basic amino acid transporter
      B(0+)
      SLC6A15 Orphan sodium- and chloride-dependent neurotransmitter transporter
      NTT73
      SLC6A16 Orphan sodium- and chloride-dependent neurotransmitter transporter
      NTT5
      SLC6A17 Orphan sodium- and chloride-dependent neurotransmitter transporter
      NTT4
      Sodium SLC6A18 Sodium- and chloride-dependent transporter XTRP2
      SLC6A19 Sodium-dependent neutral amino acid transporter B(0)
      SLC6A20 Sodium- and chloride-dependent transporter XTRP3
      Norepinephrine Transporter (NET)
          Human Net (hNET) transcript variant 1 (NM_001172504)
          Human Net (hNET) transcript variant 2 (NM_001172501)
          Human Net (hNET) transcript variant 3 (NM_001043)
          Human Net (hNET) transcript variant 4 (NM_001172502)
          Non-Human Net
      SLC7 solute carrier 7 transporter protein family
      SLC7A1, SLC7A2, SLC7A3, SLC7A4, SLC7A5, SLC7A6, SLC7A7, SLC7A8,
      SLC7A9, SLC7A10, SLC7A11, SLC7A13, SLC7A14
      SLC8 solute carrier 8 transporter protein family
      SLC8A1, SLC8A2, SLC8A3
      SLC9 solute carrier 9 transporter protein family
      SLC9A1, SLC9A2, SLC9A3, SLC9A4, SLC9A5, SLC9A6, SLC9A7, SLC9A8,
      SLC9A9, SLC9A10, SLC9A11
      SLC10 solute carrier 10 transporter protein family
      SLC10A1, SLC10A2, SLC10A3, SLC10A4, SLC10A5, SLC10A6, SLC10A7
      SLC11 solute carrier 11 transporter protein family
      SLC11A1
      SCL11A2 or hDMT
          SLC11A2 transcript variant 4
          SLC11A2 transcript variant 1
          SLC11A2 transcript variant 2
          SLC11A2 transcript variant 3
          SLC11A2 transcript variant 5
          SLC11A2 transcript variant 6
          SLC11A2 transcript variant 7
      SLC12 solute carrier 12 transporter protein family
      SLC12A1, SLC12A1, SLC12A2, SLC12A3, SLC12A4, SLC12A5, SLC12A6,
      SLC12A7, SLC12A8, SLC12A9
      SLC13 solute carrier 13 transporter protein family
      SLC13A1, SLC13A2, SLC13A3, SLC13A4, SLC13A5
      SLC14 solute carrier 14 transporter protein family
      SLC14A1, SLC14A2
      SLC15 solute carrier 15 transporter protein family
      SLC15A1, SLC15A2, SLC15A3, SLC15A4
      SLC16 solute carrier 16 transporter protein family
      SLC16A1, SLC16A2, SLC16A3, SLC16A4, SLC16A5, SLC16A6, SLC16A7,
      SLC16A8, SLC16A9, SLC16A10, SLC16A11, SLC16A12, SLC16A13,
      SLC16A14
      SLC17 solute carrier 17 transporter protein family
      SLC17A1, SLC17A2, SLC17A3, SLC17A4, SLC17A5, SLC17A6, SLC17A7,
      SLC17A8
      SLC18 solute carrier 18 transporter protein family
      SLC18A1, SLC18A2, SLC18A3
      SLC19 solute carrier 19 transporter protein family
      SLC19A1, SLC19A2, SLC19A3
      SLC20 solute carrier 20 transporter protein family
      SLC20A1, SLC20A2
      SLC21 solute carrier 21 transporter protein family
            subfamily 1; SLCO1A2, SLCO1B1, SLCO1B3, SLCO1B4,
            SLCO1C1
            subfamily 2; SLCO2A1, SLCO2B1
            subfamily 3; SLCO3A1
            subfamily 4; SLCO4A1, SLCO4C1
            subfamily 5; SLCO5A1
      SLC22 solute carrier 22 transporter protein family
      SLC22A1, SLC22A2, SLC22A3, SLC22A4, SLC22A5, SLC22A6, SLC22A7,
      SLC22A8, SLC22A9, SLC22A10, SLC22A11, SLC22A12, SLC22A13,
      SLC22A14, SLC22A15, SLC22A16, SLC22A17, SLC22A18, SLC22A19,
      SLC22A20
      SLC23 solute carrier 23 transporter protein family
      SLC23A1, SLC23A2, SLC23A3, SLC23A4
      SLC24 solute carrier 24 transporter protein family
      SLC24A1, SLC24A2, SLC24A3, SLC24A4, SLC24A5, SLC24A6
      SLC25 solute carrier 25 transporter protein family
      SLC25A1, SLC25A2, SLC25A3, SLC25A4, SLC25A5, SLC25A6, SLC25A7,
      SLC25A8, SLC25A9, SLC25A10, SLC25A11, SLC25A12, SLC25A13,
      SLC25A14, SLC25A15, SLC25A16, SLC25A17, SLC25A18, SLC25A19,
      SLC25A20, SLC25A21, SLC25A22, SLC25A23, SLC25A24, SLC25A25,
      SLC25A26, SLC25A27, SLC25A28, SLC25A29, SLC25A30, SLC25A31,
      SLC25A32, SLC25A33, SLC25A34, SLC25A35, SLC25A36, SLC25A37,
      SLC25A38, SLC25A39, SLC25A40, SLC25A41, SLC25A42, SLC25A43,
      SLC25A44, SLC25A45, SLC25A46
      SLC26 solute carrier 26 transporter protein family
      SLC26A1, SLC26A2, SLC26A3, SLC26A4, SLC26A5, SLC26A6, SLC26A7,
      SLC26A8, SLC26A9, SLC26A10, SLC26A11
      SLC27 solute carrier 27 transporter protein family
      SLC27A1, SLC27A2, SLC27A3, SLC27A4, SLC27A5, SLC27A6
      SLC28 solute carrier 28 transporter protein family
      SLC28A1, SLC28A2, SLC28A3
      SLC29 solute carrier 29 transporter protein family
      SLC29A1, SLC29A2, SLC29A3, SLC29A4
      SLC30 solute carrier 30 transporter protein family
      SLC30A1, SLC30A2, SLC30A3, SLC30A4, SLC30A5, SLC30A6, SLC30A7,
      SLC30A8, SLC30A9, SLC30A10
      SLC31 solute carrier 31 transporter protein family
      SLC31A1
      SLC32 solute carrier 32 transporter protein family
      SLC32A1
      SLC33 solute carrier 33 transporter protein family
      SLC33A1
      SLC34 solute carrier 34 transporter protein family
      SLC34A1, SLC34A2, SLC34A3
      SLC35 solute carrier 35 transporter protein family
          subfamily A; SLC35A1, SLC35A2, SLC35A3, SLC35A4, SLC35A5
          subfamily B; SLC35B1, SLC35B2, SLC35B3, SLC35B4
          subfamily C; SLC35C1, SLC35C2
          subfamily D; SLC35D1, SLC35D2, SLC35D3
          subfamily E; SLC35E1, SLC35E2, SLC35E3, SLC35E4
      SLC36 solute carrier 36 transporter protein family
      SLC36A1, SLC36A2, SLC36A3, SLC36A4
      SLC37 solute carrier 37 transporter protein family
      SLC37A1, SLC37A2, SLC37A3, SLC37A4
      SLC38 solute carrier 38 transporter protein family
      SLC38A1, SLC38A2, SLC38A3, SLC38A4, SLC38A5, SLC38A6
      SLC39 solute carrier 39 transporter protein family
      SLC39A1, SLC39A2, SLC39A3, SLC39A4, SLC39A5, SLC39A6, SLC39A7,
      SLC39A8, SLC39A9, SLC39A10, SLC39A11, SLC39A12, SLC39A13,
      SLC39A14
      SLC40 solute carrier 40 transporter protein family
      SLC40A1
      SLC41 solute carrier 41 transporter protein family
      SLC41A1, SLC41A2, SLC41A3
      SLC42 solute carrier 42 transporter protein family
      RHAG, RhBG, RhCG
      SLC43 solute carrier 43 transporter protein family
      SLC43A1
      SLC43A2
      SLC43A3
      SLC44 solute carrier 44 transporter protein family
      SLC44A1, SLC44A2, SLC44A3, SLC44A4, SLC44A5
      SLC45 solute carrier 45 transporter protein family
      SLC45A1, SLC45A2, SLC54A3, SLC45A4
      SLC46 solute carrier 46 transporter protein family
      SLC46A1, SLC46A2
      SLC47 solute carrier 47 transporter protein family
      SLC47A1, SLC47A2
MRI Imaging
      Human transferrin receptor
      Human transferrin receptor
      Mouse transferrin receptor
      Human ferritin light chain (FTL)
      Human ferritin heavy chain
      FTL 498-199InsTC, a mutated form of the ferritin light chain
      Bacterial ferritin
          E. coli
          E. coli strain K12
          S. aureus strain MRSA252
          S. aureus strain NCTC 8325
          H. pylori B8
      bacterioferritin
      codon optimized bacterioferritin
      MagA
Enzymes that modify a substrate to produce a detectable product or signal, or are
detectable by antibodies
      alpha-amylase
      alkaline phosphatase
      secreted alkaline phosphatase
      peroxidase
      T4 lysozyme
      oxidoreductase
      pyrophosphatase
Therapeutic genes
therapeutic gene product
antigens
          tumor specific antigens
          tumor-associated antigens
          tissue-specific antigens
          bacterial antigens
          viral antigens
          yeast antigens
          fungal antigens
          protozoan antigens
          parasite antigens
          mitogens
an antibody or fragment thereof
          virus-specific antibodies
antisense RNA
siRNA
      siRNA directed against expression of a tumor-promoting gene
          an oncogene
          growth factor
          angiogenesis promoting gene
          a receptor
      siRNA molecule directed against expression of any gene essential for cell growth,
      cell replication or cell survival.
      siRNA molecule directed against expression of any gene that stabilizes the cell
      membrane or otherwise limits the number of tumor cell antigens released from the
      tumor cell.
protein ligands
an antitumor oligopeptide
an antimitotic peptide
          tubulysin,
          phomopsin
          hemiasterlin
          taltobulin (HT1-286, 3)
          cryptophycin
      a mitosis inhibitor protein
      an antimitotic oligopeptide
      an anti-cancer polypeptide antibiotic
      anti-cancer antibiotics
tissue factors
      Tissue Factor (TF)
      αvβ3-integrin RGD fusion protein
Immune modulatory molecules
      GM-CSF
      MCP-1 or CCL2 (Monocyte Chemoattractant Protein-1) Human
      MCP-1 murine
      IP-10 or Chemokine ligand 10 (CXCL10)
      LIGHT
      P60 or SEQSTM1 (Sequestosome 1 transcript variant 1)
      P60 or SEQSTM1 (Sequestosome 1 transcript variant 3)
      P60 or SEQSTM1 (Sequestosome 1 transcript variant 2)
      OspF
      OspG
      STAT1alpha
      STAT1beta
Interleukins
      IL-18 (Interleukin-18)
      IL-11 (Interleukin-11)
      IL-6 (Interleukin-6)
      sIL-6R-IL-6
      interleukin-12
      interleukin-1
      interleukin-2
      IL-24 (Interleukin-24)
      IL-24 transcript variant 1
      IL-24 transcript variant 4
      IL-24 transcript variant 5
      IL-4
      IL-8
      IL-10
chemokines
      IP-10 (CXCL)
      Thrombopoetin
      members of the C-X-C and C-C chemokine families
      RANTES
      MIP1-alpha
      MIP1 -beta
      MIP-2
CXC chemokines
      GROα
      GROβ (MIP-2)
      GROγ
      ENA-78
      LDGF-PPBP
      GCP-2
      PF4
      Mig
      IP-10
      SDF-1α/β
      BUNZO/STRC33
      I-TAC
      BLC/BCA-1
      MDC
      TECK
      TARC
      HCC-1
      HCC-4
      DC-CK1
      MIP-3α
      MIP-3β
      MCP-2
      MCP-3 (Monocyte Chemoattractant Protein-3, CCL7)
      MCP-4
      MCP-5 (Monocyte Chemoattractant Protein-5; CCL12)
      Eotaxin (CCL11)
      Eotaxin-2/MPIF-2
      I-309
      MIP-5/HCC-2
      MPIF-1
      6Ckine
      CTACK
      MEC
      lymphotactin
      fractalkine
Immunoglobulin superfamily of cytokines
      B7.1
      B7.2.
Anti-angiogenic genes/angiogenesis inhibitors
      Human plasminogen k5 domain (hK5)
      PEDF (SERPINF1) (Human)
      PEDF (mouse)
      anti-VEGF single chain antibody (G6)
      anti-DLL4 s.c. antibody GLAF-3
      tTF-RGD (truncated human tissue factor protein fused to an RGD peptide)
viral attenuation factors
      Interferons
          IFN-γ
          IFN-α
          IFN-β
Antibody or scFv
      Therapeutic antibodies (i.e. anticancer antibodies)
          Rituximab (RITUXAN)
          ADEPT
          Trastuzumab (Herceptin)
          Tositumomab (Bexxar)
          Cetuximab (Erbitux ®)
          Ibritumomab (90Y-Ibritumomab tiuexetan; Zevalin ®)
          Alemtuzumab (Campathe ®-1H)
          Epratuzumab (Lymphocide ®)
          Gemtuzumab ozogamicin (Mylotarg ®)
          Bevacimab (Avastin ®) and Edrecolomab (Panorex ®)
          Infliximab
Metastasis suppressor genes
      NM23 or NME1 Isoform a
      NM23 or NME1 Isoform b
Anti-metastatic genes
      E-Cad
      Gelsolin
      LKB1 (STK11)
      RASSF1
      RASSF2
      RASSF3
      RASSF4
      RASSF5
      RASSF6
      RASSF7
      RASSF8
      Syk
      TIMP-1 (Tissue Inhibitor of Metalloproteinase Type-1)
      TIMP-2 (Tissue Inhibitor of Metalloproteinase Type-2)
      TIMP-3 (Tissue Inhibitor of Metalloproteinase Type-3)
      TIMP-4 (Tissue Inhibitor of Metalloproteinase Type-4)
      BRMS-1
      CRMP-1
      CRSP3
      CTGF
      DRG1
      KAI1
      KiSS1 (kisspeptin)
      kisspeptin fragments
          kisspeptin-10
          kisspeptin-13
          kisspeptin-14
          kisspeptin-54
      Mkk4
      Mkk6
      Mkk7
      RKIP
      RHOGDI2
      SSECKS
      TXNIP/VDUP1
Cell matrix-degradative genes
      Relaxin 1
      hMMP9
Hormones
      Human Erythropoietin (EPO)
MicroRNAs
      pre-miRNA 181a (sequence inserted into viral genome)
      miRNA 181a
      mmu-miR-181a MIMAT0000210 mature miRNA 181a
      pre-miRNA 126 (sequence inserted into the vial genome)
      miRNA 126
      hsa-miR-126 MI000471
      hsa-miR-126 MIMAT0000445
      pre-miRNA 335 (sequence inserted into the viral genome)
      miRNA 335
      hsa-miR-335 MI0000816
      hsa-miR-335 MIMAT0000765
Genes for tissue regeneration and reprogramming Human somatic cells to pluripotency
      nAG
      Oct4
      NANOG
      Ngn (Neogenin 1) transcript variant 1
      Ngn (Neogenin 1) transcript variant 2
      Ngn (Neogenin 1) transcript variant 3
      Ngn3
      Pdx1
      Mafa
Additional Genes
      Myc-CTR1
      FCU1
      mMnSOD
      HACE1
      nppa1
      GCP-2 (Granulocyte Chemotactic Protein-2, CXCL6)
      hADH
      Wildtype CDC6
      Mut CDC6
      GLAF-3 anti-DLL4 scFv
      GLAF-4 anti-FAP (Fibroblast Activation Protein) scFv (Brocks et al., (2001) Mol.
      Medicine 7(7): 461-469)
      GLAF-5 anti-FAP scFv
      BMP4
      wildtype F14.5L
Other Proteins
      WT1
      p53
      pseudomonas A endotoxin
      diphtheria toxin
      Arf or p16
      Bax
      Herpes simplex virus thymidine kinase
      E. coli purine nucleoside phosphorylase
      angiostatin
      endostatin
      Rb
      BRCA1
      cystic fibrosis transmembrane regulator (CFTR)
      Factor VIII
      low density lipoprotein receptor
      alpha-galactosidase
      beta-glucocerebrosidase
      insulin
      parathyroid hormone
      alpha-1-antitrypsin
      rsCD40L
      Fas-ligand
      TRAIL
      TNF
      microcin E492
      xanthineguanine phosphoribosyltransferase (XGPRT)
      E. coli guanine phosphoribosyltransferase (gpt)
      hyperforin
      endothelin-1 (ET-1)
      connective tissue growth factor (CTGF)
      vascular endothelial growth factor (VEGF)
      cyclooxygenase
      COX-2
      cyclooxygenase-2 inhibitor
      MPO (Myeloperoxidase)
      Apo A1 (Apolipoprotein A1)
      CRP (C Reactive Protein)
      Fibrinogen
      SAP (Serum Amyloid P)
      FGF-basic (Fibroblast Growth Factor-basic)
      PPAR-agonist
      PE37/TGF-alpha fusion protein
Replacement of the A34R gene with another A34R gene from a different strain in order
to increase the EEV form of the virus
      A34R from VACV IHD-J
      A34R with a mutation at codon 151 (Lys 151 to Asp)
      A34R with a mutation at codon 151 (Lys 151 to Glu)
Non-coding Sequence
      Non-proteins
      Non-coding nucleic acid
Ribozymes
      Group I introns
      Group II introns
      RNaseP
      hairpin ribozymes
      hammerhead ribozymes
Prodrug converting enzymes
      varicella zoster thymidine kinase
      cytosine deaminase
      purine nucleoside phosphorylase (e.g., from E. coli)
      beta lactamase
      carboxypeptidase G2
      carboxypeptidase A
      cytochrome P450
          cytochrome P450-2B1
          cytochrome P450-4B1
      horseradish peroxidase
      nitroreductase
      rabbit carboxylesterase
      mushroom tyrosinase
      beta galactosidase (lacZ) (i.e., from E. coli)
      beta glucuronidase (gusA)
      thymidine phosphorylase
      deoxycytidine kinase
      linamerase
Proteins detectable by antibodies
      chloramphenicol acetyl transferase
      hGH
Viral attenuation factors
      virus-specific antibodies
          mucins
          thrombospondin
      tumor necrosis factors (TNFs)
          TNFα
Superantigens
Toxins
      diphtheria toxin
      Pseudomonas exotoxin
      Escherichia coli Shiga toxin
      Shigella toxin
      Escherichia coli Verotoxin 1
      Toxic Shock Syndrome Toxin 1
      Exfoliating Toxins (EXft)
      Streptococcal Pyrogenic Exotoxin (SPE) A, B and C
      Clostridial Perfringens Enterotoxin (CPET)
      staphylococcal enterotoxins
          SEA, SEB, SEC1, SEC2, SED, SEE and SEH
      Mouse Mammary Tumor Virus proteins (MMTV)
      Streptococcal M proteins
      Listeria monocytogenes antigen p60
      mycoplasma arthritis superantigens
Proteins that can bind a contrasting agent, chromophore, or a compound or ligand that
can be detected
      siderophores
          enterobactin
          salmochelin
          yersiniabactin
          aerobactin
Growth Factors
      platelet-derived growth factor (PDG-F)
      keratinocyte growth factor (KGF)
      insulin-like growth factor-1 (IGF-1)
      insulin-like growth factor-binding proteins (IGFBPs)
      transforming growth factor (TGF-alpha)
      Growth factors for blood cells
          Granulocyte Colony Stimulating Factor (G-CSF)
      growth factors that can boost platelets
Other Groups
      BAC (Bacterial Artificial Chromosome) encoding several or all proteins of a
specific pathway, e.g. woundhealing-pathway
      MAC (Mammalian Artificial Chromosome) encoding several or all proteins of a
specific pathway, e. g. woundhealing-pathway
      tumor antigen
      RNAi
      ligand binding proteins
      proteins that can induce a signal detectable by MRI
      angiogenins
      photosensitizing agents
      anti-metabolites
      signaling modulators
      chemotherapeutic compounds
      lipases
      proteases
      pro-apoptotic factors
      anti-cancer vaccine
          antigen vaccines
          whole cell vaccines (i.e., dendritic cell vaccines)
          DNA vaccines
          anti-idiotype vaccines
      tumor suppressors
      cytotoxic protein
      cytostatic proteins
      costimulatory molecules
          cytokines and chemokines
      cancer growth inhibitors
      gene therapy
      BCG vaccine for bladder cancer
Proteins that interact with host cell proteins
```

### (ii) Anti-Metastatic Genes

The oncolytic reporter viruses used in the methods provided herein can encode one more anti-metastatic agents that inhibit one or more steps of the metastatic cascade. In some examples, the viruses provided herein encode one more anti-metastatic agents that inhibit invasion of local tissue. In other examples, the oncolytic reporter viruses used in the methods provided herein encode one more anti-metastatic agents that inhibit intravasation into the bloodstream or lymphatics. In other examples, the oncolytic reporter viruses used in the methods provided herein encode one more anti-metastatic agents that inhibit cell survival and transport through the bloodstream or lymphatics as emboli or potentially single cells. In other examples, the oncolytic reporter viruses used in the methods provided herein encode one more anti-metastatic agents that inhibit cell lodging in microvasculature at the secondary site. In other examples, the oncolytic reporter viruses used in the methods provided herein encode one more anti-metastatic agents that inhibit growth into microscopic lesions and subsequently into overt metastatic lesions. In other examples, the oncolytic reporter viruses used in the methods provided herein encode one more anti-metastatic agents that inhibit metastasis formation and growth within the primary tumor, where the inhibition of metastasis formation is not a consequence of inhibition of primary tumor growth. Anti-metastatic agents can inhibit specific steps in the metastatic cascade or multiple steps in the metastatic cascade.

An anti-metastatic agent expressed by a virus provided herein that inhibits metastasis of a tumor in one cell type can inhibit metastasis of other types of tumor cells. For example, an anti-metastatic agent expressed by a virus provided herein that inhibits metastasis of breast tumors also can inhibit metastasis of melanoma tumors (Welch et al. (2003) J. Natl. Cancer Inst. 95(12):839-841; Welch et al. (1999) J. Natl. Cancer Inst. 91:1351-1353; Kauffman et al. (2003) J. Urol. 169:1122-1133; Shevde et al., (2003) Cancer Lett. 198:1-20).

Anti-metastatic agents expressed by the viruses provided herein can directly or indirectly inhibit one or more steps of the metastatic cascade. Exemplary anti-metastatic agents that can be expressed by the oncolytic reporter viruses used in the methods provided herein include, but are not limited to, the following: BRMS-1 (Breast Cancer Metastasis Suppressor 1), CRMP-1 (Collapsin Response Mediator Protein-1), CRSP-3 (Cofactor Required for Sp1 transcriptional activation subunit 3), CTGF (Connective Tissue Growth Factor), DRG-1 (Developmentally-regulated GTP-binding protein 1), E-Cad (E-cadherin), gelsolin, KAI1, KiSS1 (Kisspeptin 1/Metastin), kispeptin-10, kispeptin-13, kispeptin-14, kispeptin-54, LKB1 (STK11 (serine/threonine kinase 11)), JNKK1/MKK4 (c-Jun-NH2-Kinase Kinase/Mitogen activated Kinase Kinase 4), MKK6 (mitogen activated kinase kinase 6), MKK7 (mitogen activated kinase kinase 7), Nm23 (NDP Kinase A), RASSF1-8 (Ras association (RalGDS/AF-6) domain family members), RKIP (Raf kinase inhibitor protein), RhoGDI2 (Rho GDP dissociation inhibitor 2), SSECKS (src-suppressed C-kinase substrate), Syk, TIMP-1 (Tissue inhibitor of metalloproteinase-1), TIMP-2 (Tissue inhibitor of metalloproteinase-2), TIMP-3 (Tissue inhibitor of metalloproteinase-3), TIMP-4 (Tissue inhibitor of metalloproteinase-4), TXNIP/VDUP1 (Thioredoxin-interacting protein). Such list of anti-metastatic agents is not meant to be limiting. Any gene product that can suppress metastasis formation via a mechanism that is independent of inhibition of growth within the primary tumor is encompassed by the designation of an anti-metastatic agent or metastasis suppressor and can be expressed by a virus as provided herein. One of skill in the art can identify anti-metastatic genes and can construct a virus expressing one or more anti-metastatic genes for therapy.

Exemplary anti-metastatic agents exist within many different types of cellular compartments and are not limited to any specific type of biomolecule. Anti-metastatic agents that are expressed by the viruses provided herein can localize within a variety of cellular compartments within the infected cell, on the surface of the infected cell and/or secreted by the infected cell. For example, anti-metastatic agents can be cell surface receptors, such as, for example KAI1, E-cadherin and CD44; intracellular signaling molecules, such as, for example, MKK4, SSeCKs, Nm23, RhoGDI2, DRG-1, and RKIP; secreted ligands, such as, for example TIMPs and KiSS1, nuclear transcription factors and cofactors, such as, for example BRMS1, TXNIP and CRSP3, and proteins localized to the mitochondria, such as, for example, caspase 8 (Welch et al. J. Natl. Cancer Inst. 95(12):839-841 (2003). Anti-metastatic agents also encompass intracellular signaling molecules including cytoskeletal associated proteins, such as, for example, RhoGDI2 and gelsolin, and cytosolic proteins, such as, for example, JNKK1/MKK4, nm23-H1 and RKIP (see, e.g., Dong et al. (1995) Science, 268:884-886; Yin and Stossel, (1979) Nature, 281:583-6; Shimizu et al. (1991) Biochem. Biophys. Res. Commun. 175:199-206; Boller et al., (1985) J Cell Biol. 100:327-332; Girgrah et al., (1991) Neuroreport 2:441-444; Nash et al., (2006) Front Biosci. 11:647-59; Yeung et al., (1999) Nature 401:173-177; Bosnar et al., (2004) Exp. Cell Res. 298:275-284; Rinker-Schaeffer et al., (2006) Clin. Cancer Res. 12:3882-3889).

d. Exemplary Oncolytic Reporter Viruses for Use in the Methods

Reporter viruses for use in the methods provided herein typically are replication competent viruses that selectively infect neoplastic cells (i.e. oncolytic viruses). Numerous oncolytic viruses have been identified or developed and are known to those of skill in the art. The methods herein can use any of these viruses for detection of tumor cells. In addition, the methods herein for assessing the effectiveness of such viruses for treating a subject's tumor can be employed for any such viruses. The methods herein detect infected circulating tumor cells. If detected soon after administration of a therapeutic oncolytic reporter virus, detection is indicative that the virus has infected tumors and is indicative that such virus will replicate in and lyse such tumors.

Oncolytic viruses include virus that preferentially infect and accumulate in tumor cells and viruses that are modified to do so. Viruses and viral vectors include, but are not limited to, poxviruses, herpesviruses, adenoviruses, adeno-associated viruses, lentiviruses, retroviruses, rhabdoviruses, papillomaviruses, vesicular stomatitis virus, measles virus, Newcastle disease virus, picornavirus, Sindbis virus, papillomavirus, parvovirus, reovirus, coxsackievirus, influenza virus, mumps virus, poliovirus, and semliki forest virus. Oncolytic viruses include, but are not limited to, vaccinia viruses, vesicular stomatitis viruses, herpes viruses, measles viruses and adenoviruses. Oncolytic viruses include cytoplasmic viruses that do not require entry of viral nucleic acid molecules in to the nucleus of the host cell during the viral life cycle. A variety of cytoplasmic viruses are known, including, but not limited to, poxviruses, African swine flu family viruses, and various RNA viruses such as picornaviruses, caliciviruses, togaviruses, coronaviruses and rhabdoviruses. Exemplary cytoplasmic viruses provided herein are viruses of the poxvirus family, including orthopoxviruses. Exemplary of poxviruses are vaccinia viruses.

Such viruses have been employed for the detection and therapy of tumors. One of skill in the art is familiar with and can readily identify such viruses, and can adapt them for the methods described herein. Viruses used in the methods described herein also can be further modified to render them detectable as a reporter virus.

Viruses for use in the methods provided herein typically are modified viruses, which are modified relative to the wild-type virus. Such modifications of the viruses provided can enhance one or more characteristics of the virus. Such characteristics can include, but are not limited to, attenuated pathogenicity, reduced toxicity, preferential accumulation in tumor, increased ability to activate an immune response against tumor cells, increased immunogenicity, increased or decreased replication competence, and ability to express additional exogenous proteins, and combinations thereof. For examples, the viruses can be modified to express one or more detectable gene products, including proteins that can be used for detecting, imaging and monitoring of CTCs. In other examples, the viruses can be modified to express one or more gene products for the therapy of a tumor.

Viruses for use in the methods provided herein can contain one or more heterologous nucleic acid molecules inserted into the genome of the virus. A heterologous nucleic acid molecule can contain an open reading frame operatively linked to a promoter for expression or can be a non-coding sequence that alters the attenuation of the virus. In some cases, the heterologous nucleic acid replaces all or a portion of a viral gene.

i. Poxviruses

In some examples, the virus used in the methods provided herein is selected from the poxvirus family. Poxviruses include Chordopoxyiridae such as orthopoxvirus, parapoxvirus, avipoxvirus, capripoxvirus, leporipoxvirus, suipoxvirus, molluscipoxvirus and yatapoxvirus, as well as Entomopoxyirinae such as entomopoxvirus A, entomopoxvirus B, and entomopoxvirus C. One skilled in the art can select a particular genera or individual chordopoxyiridae according to the known properties of the genera or individual virus, and according to the selected characteristics of the virus (e.g., pathogenicity, ability to elicit an immune response, preferential tumor localization, preferential tumor cell infection), the intended use of the virus, the tumor type and the host organism. Exemplary chordopoxyiridae genera are orthopoxvirus and avipoxvirus.

Avipoxviruses are known to infect a variety of different birds and have been administered to humans. Exemplary avipoxviruses include canarypox, fowlpox, juncopox, mynahpox, pigeonpox, psittacinepox, quailpox, peacockpox, penguinpox, sparrowpox, starlingpox, and turkeypox viruses.

Orthopoxviruses are known to infect a variety of different mammals including rodents, domesticated animals, primates and humans. Several orthopoxviruses have a broad host range, while others have narrower host range. Exemplary orthopoxviruses include buffalopox, camelpox, cowpox, ectromelia, monkeypox, raccoon pox, skunk pox, tatera pox, uasin gishu, vaccinia, variola, and volepox viruses. In some embodiments, the orthopoxvirus selected can be an orthopoxvirus known to infect humans, such as cowpox, monkeypox, vaccinia, or variola virus. Optionally, the orthopoxvirus known to infect humans can be selected from the group of orthopoxviruses with a broad host range, such as cowpox, monkeypox, or vaccinia virus.

### (1) Vaccinia Viruses

One exemplary orthopoxvirus for use in the methods of detection and therapy of CTCs provided herein is vaccinia virus. Vaccinia virus strains have been shown to specifically colonize solid tumors, while not infecting other organs (see, e.g., Zhang et al. (2007) Cancer Res 67:10038-10046; Yu et al., (2004) Nat Biotech 22:313-320; Heo et al., (2011) Mol Ther 19:1170-1179; Liu et al. (2008) Mol Ther 16:1637-1642; Park et al., (2008) Lancet Oncol, 9:533-542). Vaccinia is a cytoplasmic virus, thus, it does not insert its genome into the host genome during its life cycle. The linear dsDNA viral genome of vaccinia virus is approximately 200 kb in size, encoding a total of approximately 200 potential genes. A variety of vaccinia virus strains are available for uses in the methods provided, including Western Reserve (WR) (SEQ ID NO: 34), Copenhagen (SEQ ID NO: 35), Tashkent, Tian Tan, Lister, Wyeth, 1HD-J, and IHD-W, Brighton, Ankara, MVA, Dairen I, LIPV, LC16M8, LC16MO, LIVP, WR 65-16, Connaught, New York City Board of Health.

Exemplary vaccinia viruses are Lister or LIVP vaccinia viruses. In one embodiment, the Lister strain can be an attenuated Lister strain, such as the LIVP (Lister virus from the Institute of Viral Preparations, Moscow, Russia) strain, which was produced by further attenuation of the Lister strain. The LIVP strain was used for vaccination throughout the world, particularly in India and Russia, and is widely available. In another embodiment, the viruses and methods provided herein can be based on modifications to the Lister strain of vaccinia virus.

Lister (also referred to as Elstree) vaccinia virus is available from any of a variety of sources. For example, the Elstree vaccinia virus is available at the ATCC under Accession Number VR-1549. The Lister vaccinia strain has high transduction efficiency in tumor cells with high levels of gene expression. LIVP and its production are described, for example, in U.S. Pat. Nos. 7,588,767, 7,588,771, 7,662,398 and 7,754,221 and U.S. Patent Publication Nos. 2007/0202572, 2007/0212727, 2010/0062016, 2009/0098529, 2009/0053244, 2009/0155287, 2009/0117034, 2010/0233078, 2009/0162288, 2010/0196325, 2009/0136917 and 2011/0064650.

Vaccinia virus possesses a variety of features for use in cancer gene therapy and vaccination including broad host and cell type range, a large carrying capacity for foreign genes (up to 25 kb of exogenous DNA fragments (approximately 12% of the vaccinia genome size) can be inserted into the vaccinia genome), high sequence homology among different strains for designing and generating modified viruses in other strains, and techniques for production of modified vaccinia strains by genetic engineering are well established (Moss (1993) Curr. Opin. Genet. Dev. 3: 86-90; Broder and Earl (1999) Mol. Biotechnol. 13: 223-245; Timiryasova et al. (2001) Biotechniques 31: 534-540). A variety of vaccinia virus strains are available, including Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health. Exemplary of vaccinia viruses for use in the methods provided herein include, but are not limited to, Lister strain or LIVP strain of vaccinia viruses.

The exemplary modifications of the Lister strain described herein (see Example 1) also can be adapted to other vaccinia viruses (e.g., Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health). The modifications of the Lister strain described herein also can be adapted to other viruses, including, but not limited to, viruses of the poxvirus family, adenoviruses, herpes viruses and retroviruses.

LIVP strains that can be used in the methods provided herein include LIVP clonal strains derived from LIVP that have a genome that is or is derived from or is related to a the parental sequence set forth in SEQ ID NO: 2 (see U.S. Patent Pub. No. 2012-0308484, which is incorporated herein by reference). These include LIVP clonal strains that have been shown to exhibit greater anti-tumorigenicity and/or reduced toxicity compared to the recombinant or modified virus strain designated GLV-1h68 (having a genome set forth in SEQ ID NO:1; and U.S. Patent Pub. No. 2012-0308484). In particular, the clonal strains are present in a virus preparation propagated from LIVP. Exemplary LIVP clonal strains include but are not limited to LIVP 1.1.1 (SEQ ID NO: 36), LIVP 2.1.1 (SEQ ID NO: 37), LIVP 4.1.1 (SEQ ID NO: 38), LIVP 5.1.1 (SEQ ID NO: 39), LIVP 6.1.1 (SEQ ID NO: 40), LIVP 7.1.1 (SEQ ID NO: 41), and LIVP 8.1.1 (SEQ ID NO: 42).

For purposes herein, the methods are exemplified with GLV-1h68 and GLV-1h254, but it is understood that the methods can be employed with any oncolytic virus that can be detected and that accumulates in CTC cells.

The LIVP and clonal strains for use in the methods provided herein have a sequence of nucleotides that have at least 70%, such as at least 75%, 80%, 85% or 90% sequence identity to SEQ ID NO: 2. For example, the clonal strains have a sequence of nucleotides that has at least 91%, 92%, 93%, 94%, 95%, 95%, 96%, 97%, 98%, 99%, 99.1%, 99.2%, 99.3%, 99.4%, 99.5%, 99.6%, 99.7%, 99.8%, 99.9%, or 100% identical SEQ ID NO: 2. Such LIVP clonal viruses include viruses that differ in one or more open reading frames (ORF) compared to the parental LIVP strain that has a sequence of amino acids set forth in SEQ ID NO: 2. The LIVP clonal virus strains provided herein can contain a nucleotide deletion or mutation in any one or more nucleotides in any ORF compared to SEQ ID NO: 2, or can contain an addition or insertion of viral DNA compared to SEQ ID NO: 2.

In some examples, the LIVP strain for use in the methods is a clonal strain of LIVP or a modified form thereof containing a sequence of nucleotides that has at least 97% sequence identity to a sequence of nucleotides 2,256-180,095 of SEQ ID NO:36, nucleotides 11,243-182,721 of SEQ ID NO:37, nucleotides 6,264-181,390 of SEQ ID NO:38, nucleotides 7,044-181,820 of SEQ ID NO:39, nucleotides 6,674-181,409 of SEQ ID NO:40, nucleotides 6,716-181,367 of SEQ ID NO:41 or nucleotides 6,899-181,870 of SEQ ID NO:42.

### (a) Modified Vaccinia Viruses

Exemplary vaccinia viruses for use in the methods provided herein include vaccinia viruses with insertions, mutations or deletions. Exemplary insertions, mutations or deletions include those that result in an attenuated vaccinia virus relative to the wild type strain. For example, vaccinia virus insertions, mutations or deletions can decrease pathogenicity of the vaccinia virus, for example, by reducing the toxicity, reducing the infectivity, reducing the ability to replicate, or reducing the number of non-tumor organs or tissues to which the vaccinia virus can accumulate. Other exemplary insertions, mutations or deletions include, but are not limited to, those that increase antigenicity of the virus, those that permit detection, monitoring, or imaging, those that alter attenuation of the virus, and those that alter infectivity. For example, the ability of vaccinia viruses provided herein to infect and replicate within tumors can be enhanced by mutations that increase the extracellular enveloped form of the virus (EEV) that is released from the host cell, as described elsewhere herein. Modifications can be made, for example, in genes that are involved in nucleotide metabolism, host interactions and virus formation or at other nonessential gene loci. Any of a variety of insertions, mutations or deletions of the vaccinia virus known in the art can be used herein, including insertions, mutations or deletions of: the thymidine kinase (TK) gene, the hemagglutinin (HA) gene, and F14.5L gene, among others (e.g., A35R, E2L/E3L, K1L/K2L, superoxide dismutase locus, 7.5K, C7-K1L, J2R, B13R+B14R, A56R, A26L or 14L gene loci). The vaccinia viruses for use in the methods provided herein also can contain two or more insertions, mutations or deletions. Thus, included are vaccinia viruses containing two or more insertions, mutations or deletions of the loci provided herein or other loci known in the art. The viruses can be based on modifications to the Lister strain and/or LIVP strain of vaccinia virus. Any known vaccinia virus, or modifications thereof that correspond to those provided herein or known to those of skill in the art to reduce toxicity of a vaccinia virus. Generally, however, the mutation will be a multiple mutant and the virus will be further selected to reduce toxicity.

The modified viruses for use in the methods provided herein can encode heterologous gene products. The heterologous nucleic acid is typically operably linked to a promoter for expression of the heterologous gene in the infected cells. Suitable promoter include viral promoters, such as a vaccinia virus natural and synthetic promoters. Exemplary vaccinia viral promoters include, but are not limited to, P11k, P7.5k early/late, P7.5k early, P28 late, synthetic early P_{SE}, synthetic early/late P_{SEL}, and synthetic late P_{SL} promoters.

### (b) Exemplary Modified Vaccinia Viruses

Exemplary vaccinia viruses include those derived from vaccinia virus strain GLV-1h68 (also designated RVGL21 and for clinical trial as GL-ONC1; see SEQ ID NO:1), which has been described in U.S. Pat. Pub. No. 2005-0031643, now U.S. Pat. No. 7,588,767; see, also U.S. Provisional Application Ser. No. 61/517,297 (U.S. Patent Pub. No. 2012-0308484), which provides sequences of clonal strains of LIVP and derivatives thereof, including GLV-1h68).

GLV-1h68 contains DNA insertions into gene in an LIVP strain of vaccinia virus (SEQ ID NO: 2). The LIVP vaccinia virus strain was originally prepared by adapting the Lister strain (ATCC Catalog No. VR-1549) to calf skin (Institute of Viral Preparations, Moscow, Russia, Al'tshtein et al., (1983) Dokl. Akad. Nauk USSR 285:696-699)). It is available from the Institute of Viral Preparations. GLV-1h68 contains expression cassettes encoding detectable marker proteins in the F14.5L (also designated in LIVP as F3), thymidine kinase (TK) and hemagglutinin (HA) gene loci. An expression cassette containing a Ruc-GFP cDNA molecule (a fusion of DNA encoding Renilla luciferase and DNA encoding GFP) under the control of a vaccinia synthetic early/late promoter P_{SEL} ((P_{SEL})Ruc-GFP) is inserted into the F14.5L gene locus; an expression cassette containing a DNA molecule encoding beta-galactosidase under the control of the vaccinia early/late promoter P_{7.5k} ((P_{7.5k})LacZ) and DNA encoding a rat transferrin receptor positioned in the reverse orientation for transcription relative to the vaccinia synthetic early/late promoter P_{SEL} ((P_{SEL})rTrfR) is inserted into the TK gene locus (the resulting virus does not express transferrin receptor protein since the DNA molecule encoding the protein is positioned in the reverse orientation for transcription relative to the promoter in the cassette); and an expression cassette containing a DNA molecule encoding β-glucuronidase under the control of the vaccinia late promoter P₁₁ₖ ((P₁₁ₖ)gusA) is inserted into the HA gene locus. The GLV-1h68 virus exhibits a strong preference for accumulation in tumor tissues compared to non-tumorous tissues following systemic administration of the virus to tumor bearing subjects. This preference is significantly higher than the tumor selective accumulation of other vaccinia viral strains, such as WR (see, e.g. U.S. Pat. Pub. No. 2005-0031643 and Zhang et al. (2007) Cancer Res. 67(20):10038-10046).

Modified viruses for use in the methods provided herein include the strain designed GLV-1h68 (SEQ ID NO: 1) and all strains, derivatives, and modified forms thereof that contain different or additional insertions, deletions, and also variants thereof (see, e.g., U.S. Pat. Nos. 7,588,767, 7,588,771, 7,662,398 and 7,754,221 and U.S. Patent Publication Nos. 2007/0202572, 2007/0212727, 2010/0062016, 2009/0098529, 2009/0053244, 2009/0155287, 2009/0117034, 2010/0233078, 2009/0162288, 2010/0196325, 2009/0136917 and 2011/0064650). Exemplary viruses are generated by replacement of one or more expression cassettes of the GLV-1h68 strain with heterologous DNA encoding gene products for therapy and/or imaging.

Non-limiting examples of viruses that are derived from attenuated LIVP viruses, such as GLV-1h68, and that are reporter viruses that can be employed for CTC detection, include, but are not limited to, LIVP viruses described in U.S. Pat. Nos. 7,588,767, 7,588,771, 7,662,398 and 7,754,221 and U.S. Patent Publication Nos. 2007/0202572, 2007/0212727, 2010/0062016, 2009/0098529, 2009/0053244, 2009/0155287, 2009/0117034, 2010/0233078, 2009/0162288, 2010/0196325 and 2009/0136917, which are incorporated herein by reference in their entirety. For example, the vaccinia virus can be selected from among GLV-1h22, GLV-1h68, GLV-1i69, GLV-1h70, GLV-1h71, GLV-1h72, GLV-1h73, GLV-1h74, GLV-1h81, GLV-1h82, GLV-1h83, GLV-1h84, GLV-1h85, or GLV-1h86, which are described in U.S. Patent Publication No. 2009/0098529 and GLV-1h104, GLV-1h105, GLV-1h106, GLV-1h107, GLV-1h108 and GLV-1h109, which are described in U.S. Patent Publication No. 2009/0053244; GLV-1h99, GLV-1h100, GLV-1h101, GLV-1h139, GLV-1h146, GLV-1h151, GLV-1h152 and GLV-1h153, which are described in U.S. Patent Publication No. 2009/0117034.

Exemplary reporter viruses provided herein that encode the far-red fluorescent protein TurboFP635 (scientific name “Katushka”) from the sea anemone Entacmaea quadricolor include GLV-1h188 (SEQ ID NO:3), GLV-1h189 (SEQ ID NO:4), GLV-1h190 (SEQ ID NO:5), GLV-1h253 (SEQ ID NO:6) and GLV-1h254 (SEQ ID NO:7).

Exemplary of viruses which have one or more expression cassettes removed from GLV-1h68 and replaced with a heterologous non-coding DNA molecule include GLV-1h70, GLV-1h71, GLV-1h72, GLV-1h73, GLV-1h74, GLV-1h85, and GLV-1h86. GLV-1h70 contains (P_{SEL})Ruc-GFP inserted into the F14.5L gene locus, (P_{SEL})rTrfR and (P_{7.5k})LacZ inserted into the TK gene locus, and a non-coding DNA molecule inserted into the HA gene locus in place of (P₁₁ₖ)gusA. GLV-1h71 contains a non-coding DNA molecule inserted into the F14.5L gene locus in place of (P_{SEL})Ruc-GFP, (P_{SEL})rTrfR and (P_{7.5k})LacZ inserted into the TK gene locus, and (P₁₁ₖ)gusA inserted into the HA gene locus. GLV-1h72 contains (P_{SEL})Ruc-GFP inserted into the F14.5L gene locus, a non-coding DNA molecule inserted into the TK gene locus in place of (P_{SEL})rTrfR and (P_{7.5k})LacZ, and P₁₁ₖgusA inserted into the HA gene locus. GLV-1h73 contains a non-coding DNA molecule inserted into the F14.5L gene locus in place of (P_{SEL})Ruc-GFP, (P_{SEL})rTrfR and (P_{7.5k})LacZ inserted into the TK gene locus, and a non-coding DNA molecule inserted into the HA gene locus in place of (P₁₁ₖ)gusA. GLV-1h74 contains a non-coding DNA molecule inserted into the F14.5L gene locus in place of (P_{SEL})Ruc-GFP, a non-coding DNA molecule inserted into the TK gene locus in place of (P_{SEL)}rTrfR and (P_{7.5k})LacZ, and a non-coding DNA molecule inserted into the HA gene locus in place of (P₁₁ₖ)gusA. GLV-1h85 contains a non-coding DNA molecule inserted into the F14.5L gene locus in place of (P_{SEL})Ruc-GFP, a non-coding DNA molecule inserted into the TK gene locus in place of (P_{SEL})rTrfR and (P_{7.5k})LacZ, and (P₁₁ₖ)gusA inserted into the HA gene locus. GLV-1h86 contains (P_{SEL})Ruc-GFP inserted into the F14.5L gene locus, a non-coding DNA molecule inserted into the TK gene locus in place of (P_{SEL})rTrfR and (P_{7.5k})LacZ, and a non-coding DNA molecule inserted into the HA gene locus in place of (P₁₁ₖ)gusA.

Other exemplary viruses include, but are not limited to, LIVP viruses that encode additional imaging agents such as ferritin and/or a transferrin receptor (e.g., GLV-1h82 and GLV-1h83 which encode E. coli ferritin at the HA locus; GLV-1h82 addition encodes the human transferrin receptor at the TK locus) or a click beetle luciferase-red fluorescent protein fusion protein (e.g., GLV-1h84, which encodes CBG99 and mRFP1 at the TK locus). During translation, the two proteins are cleaved into two individual proteins at picornavirus 2A element (Osborn et al., (2005) Mol. Ther. 12: 569-574). CBG99 produces a more stable luminescent signal than does Renilla luciferase with a half-life of greater than 30 minutes, which makes in vitro and in vivo assays more convenient. mRFP1 provides improvements in in vivo imaging relative to GFP since mRFP1 can penetrate tissue deeper than GFP.

Other exemplary viruses include, but are not limited to, LIVP viruses that express one or more therapeutic gene products, such as angiogenesis inhibitors (e.g., GLV-1h81, which contains DNA encoding the plasminogen K5 domain (SEQ ID NO: 30) under the control of the vaccinia synthetic early-late promoter in place of the gusA expression cassette at the HA locus in GLV-1h68; GLV-1h104, GLV-1h105 and GLV-1h106, which contain DNA encoding a truncated human tissue factor fused to the αᵥβ₃-integrin RGD binding motif (tTF-RGD) (SEQ ID NO:31) under the control of a vaccinia synthetic early promoter, vaccinia synthetic early/late promoter or vaccinia synthetic late promoter, respectively, in place of the LacZ/rTFr expression cassette at the TK locus of GLV-1h68; GLV-1h107, GLV-1h108 and GLV-1h109, which contain DNA encoding an anti-VEGF single chain antibody G6 (SEQ ID NO: 29) under the control of a vaccinia synthetic early promoter, vaccinia synthetic early/late promoter or vaccinia synthetic late promoter, respectively, in place of the LacZ/rTFr expression cassette at the TK locus of GLV-1 h68) and proteins for tumor growth suppression (e.g., GLV-1h90, GLV-1h91 and GLV-1h92, which express a fusion protein containing an IL-6 fused to an IL-6 receptor (sIL-6R/IL-6) (SEQ ID NO: 33) under the control of a vaccinia synthetic early promoter, vaccinia synthetic early/late promoter or vaccinia synthetic late promoter, respectively, in place of the gusA expression cassette at the HA locus in GLV-1h68; and GLV-1h96, GLV-1h97 and GLV-1h98, which express IL-24 (melanoma differentiation gene, mda-7; SEQ ID NO: 32) under the control of a vaccinia synthetic early promoter, vaccinia synthetic early/late promoter or vaccinia synthetic late promoter, respectively, in place of the Ruc-GFP fusion gene expression cassette at the F14.5L locus of GLV-1h68). Additional therapeutic gene products that can be engineered in the viruses provided herein also are described elsewhere herein.

Exemplary transporter proteins that can be encoded by the viruses for in vivo imaging and therapy provided herein include, for example, the human norepinephrine transporter (hNET; SEQ ID NO: 43) and the human sodium iodide symporter (hNIS; SEQ ID NO: 44). Exemplary viruses that can be employed in the methods and use provided herein that encode the human norepinephrine transporter (hNET) include, but are not limited to, GLV-1h99, GLV-1h100, GLV-1h101, GLV-1h139, GLV-1h146, and GLV-1h150. GLV-1h99 encodes hNET under the control of a vaccinia synthetic early promoter in place of the Ruc-GFP fusion gene expression cassette at the F14.5L locus of GLV-1h68. GLV-1h100, GLV-1h101 encode hNET under the control of a vaccinia synthetic early promoter or vaccinia synthetic late promoter, respectively, in place of the LacZ/rTFr expression cassette at the TK locus of GLV-1h68. GLV-1h139 encodes hNET under the control of a vaccinia synthetic early promoter in place of the gusA expression cassette at the HA locus in GLV-1h68. GLV-1h146 and GLV-1 h 150, encode hNET under the control of a vaccinia synthetic early promoter or vaccinia synthetic late promoter, respectively, in place of the LacZ/rTFr expression cassette at the TK locus of GLV-1h100 and GLV-101, respectively. Thus, GLV-1h146 and GLV-1h150 encode hNET and IL-24. Exemplary viruses that can be employed in the methods and use provided herein that encode the human sodium iodide transporter (hNIS) include, but are not limited to, GLV-1h151, GLV-1h152 and GLV-1h153. GLV-1h151, GLV-1h152 and GLV-1h153 encode hNIS under the control of a vaccinia synthetic early promoter, vaccinia synthetic early/late promoter or vaccinia synthetic late promoter, respectively, in place of the gusA expression cassette at the HA locus in GLV-1h68.

ii. Other Oncolytic Viruses

Oncolytic viruses for use in the methods provided here are well known to one of skill in the art and include, for example, vesicular stomatitis virus, see, e.g., U.S. Pat. Nos. 7,731,974, 7,153,510, 6,653,103 and U.S. Pat. Pub. Nos. 2010/0178684, 2010/0172877, 2010/0113567, 2007/0098743, 20050260601, 20050220818 and EP Pat. Nos. 1385466, 1606411 and 1520175; herpes simplex virus, see, e.g., U.S. Pat. Nos. 7,897,146, 7731,952, 7,550,296, 7,537,924, 6,723,316, 6,428,968 and U.S. Pat. Pub. Nos. 2011/0177032, 2011/0158948, 2010/0092515, 2009/0274728, 2009/0285860, 2009/0215147, 2009/0010889, 2007/0110720, 2006/0039894 and 20040009604; retroviruses, see, e.g., U.S. Pat. Nos. 6,689,871, 6,635,472, 6,639,139, 5,851,529, 5,716,826, 5,716,613 and U.S. Pat. Pub. No. 20110212530; and adeno-associated viruses, see, e.g., U.S. Pat. Nos. 8,007,780, 7,968,340, 7,943,374, 7,906,111, 7,927,585, 7,811,814, 7,662,627, 7,241,447, 7,238,526, 7,172,893, 7,033,826, 7,001,765, 6,897,045, and 6,632,670.

Also included are other therapeutic vaccinia viruses, such as the virus designated JX-594, which is a vaccinia virus that expresses GM-CSF described, for example, in U.S. Pat. No. 6,093,700, and the Wyeth strain vaccinia virus designated JX-594, which is a TK-deleted vaccinia virus that expresses GM-CSF (see, International PCT application No WO 2004/014314, U.S. Pat. No. 5,364,773; Mastrangelo et al. (1998) Cancer Gene Therapy 6:409-422; Kim et al. (2006) Molecular Therapeutics 14:361-370).

In addition, adenoviruses, such as the ONYX viruses and others, have been modified, such as be deletion of EA1 genes, so that they selectively replicate in cancerous cells, and, thus, are oncolytic. Adenoviruses also have been engineered to have modified tropism for tumor therapy and also as gene therapy vectors.

e. Production and Preparation of Virus

The viruses for use in the methods provided herein can be formed by standard methodologies well known in the art for producing and/or modifying viruses. Briefly, the methods can include introducing into viruses one or more genetic modifications, followed by screening the viruses for properties reflective of the modification or for other desired properties.

### Methods for Generating Recombinant Virus

Standard techniques in molecular biology can be used to generate the modified viruses for use in the methods provided herein. Methods for the generation of recombinant viruses using recombinant DNA methods are well known in the art (e.g., see U.S. Pat. Nos. 4,769,330, 4,603,112, 4,722,848, 4,215,051, 5,110,587, 5,174,993, 5,922,576, 6,319,703, 5,719,054, 6,429,001, 6,589,531, 6,573,090, 6,800,288, 7,045,313, He et al. (1998) Proc. Natl. Acad. Sci. USA 95(5): 2509-2514, Racaniello et al. (1981) Science 214:916-919, Hruby et al. (1990) Clin Micro Rev. 3:153-170). Such methods include, but are not limited to, various nucleic acid manipulation techniques, nucleic acid transfer protocols, nucleic acid amplification protocols, and other molecular biology techniques known in the art. For example, point mutations can be introduced into a gene of interest through the use of oligonucleotide mediated site-directed mutagenesis. Alternatively, homologous recombination can be used to introduce a mutation or exogenous sequence into a target sequence of interest. In an alternative mutagenesis protocol, point mutations in a particular gene also can be selected for using a positive selection pressure. See, e.g., Current Techniques in Molecular Biology, (Ed. Ausubel, et al.). Nucleic acid amplification protocols include but are not limited to the polymerase chain reaction (PCR). Use of nucleic acid tools such as plasmids, vectors, promoters and other regulating sequences, are well known in the art for a large variety of viruses and cellular organisms. Nucleic acid transfer protocols include calcium chloride transformation/transfection, electroporation, liposome mediated nucleic acid transfer, N-[1-(2,3-Dioloyloxy)propyl]-N,N,N-trimethylammonium methylsulfate meditated transformation, and others. Further a large variety of nucleic acid tools are available from many different sources including ATCC, and various commercial sources. One skilled in the art will be readily able to select the appropriate tools and methods for genetic modifications of any particular virus according to the knowledge in the art and design choice.

Any of a variety of modifications can be readily accomplished using standard molecular biological methods known in the art. The modifications will typically be one or more truncations, deletions, mutations or insertions of the viral genome. In one example, the modification can be specifically directed to a particular sequence. The modifications can be directed to any of a variety of regions of the viral genome, including, but not limited to, a regulatory sequence, to a gene-encoding sequence, or to a sequence without a known role. Any of a variety of regions of viral genomes that are available for modification are readily known in the art for many viruses, including the viruses specifically listed herein. As a non-limiting example, the loci of a variety of vaccinia genes provided herein and elsewhere exemplify the number of different regions that can be targeted for modification in the viruses provided herein. In some examples, the modification can be fully or partially random, whereupon selection of any particular modified virus can be determined according to the desired properties of the modified the virus. These methods include, for example, in vitro recombination techniques, synthetic methods and in vivo recombination methods as described, for example, in Sambrook et al. Molecular Cloning: A Laboratory Manual, 2nd edition, Cold Spring Harbor Laboratory Press, cold Spring Harbor N.Y. (1989), and in the Examples disclosed herein.

The viruses for use in the diagnostic and therapeutic methods provided herein encode a reporter protein, such as, for example, a fluorescent protein, a luminescent protein, a receptor or an enzyme. In some examples, the virus can be modified to express an additional exogenous gene. Exemplary exogenous gene products include proteins and RNA molecules. The modified viruses can express an additional detectable gene product, a therapeutic gene product, a gene product for manufacturing or harvesting, or an antigenic gene product for antibody harvesting. The characteristics of such gene products are described herein and elsewhere. In some examples of modifying an organism to express an exogenous gene, the modification also can contain one or more regulatory sequences to regulate expression of the exogenous gene. As is known in the art, regulatory sequences can permit constitutive expression of the exogenous gene or can permit inducible expression of the exogenous gene. Further, the regulatory sequence can permit control of the level of expression of the exogenous gene. In some examples, inducible expression can be under the control of cellular or other factors present in a tumor cell or present in a virus-infected tumor cell. In other examples, inducible expression can be under the control of an administrable substance, including IPTG, RU486 or other known induction compounds. Any of a variety of regulatory sequences are available to one skilled in the art and can be selected according to known factors and design preferences. In some examples, such as gene product manufacture and harvesting, the regulatory sequence can result in constitutive, high levels of gene expression. In some examples, such as anti-(gene product) antibody harvesting, the regulatory sequence can result in constitutive, lower levels of gene expression. In tumor therapy examples, a therapeutic protein can be under the control of an internally inducible promoter or an externally inducible promoter.

In other examples, organ or tissue-specific expression can be controlled by regulatory sequences. In order to achieve expression only in the target organ, for example, a tumor, the foreign nucleotide sequence can be linked to a tissue specific promoter and used for gene therapy. Such promoters are well known to those skilled in the art (see e.g., Zimmermann et al. (1994) Neuron 12:11-24; Vidal et al. (1990) EMBO J. 9:833-840; Mayford et al. (1995) Cell 81: 891-904; and Pinkert et al. (1987) Genes & Dev. 1:268-276).

In some examples, the viruses can be modified to express two or more proteins, where any combination of the two or more proteins can be one or more detectable gene products, therapeutic gene products, gene products for manufacturing or harvesting or antigenic gene products for antibody harvesting. In one example, a virus can be modified to express a detectable protein and a therapeutic protein. In another example, a virus can be modified to express two or more gene products for detection or two or more therapeutic gene products. For example, one or more proteins involved in biosynthesis of a luciferase substrate can be expressed along with luciferase. When two or more exogenous genes are introduced, the genes can be regulated under the same or different regulatory sequences, and the genes can be inserted in the same or different regions of the viral genome, in a single or a plurality of genetic manipulation steps. In some examples, one gene, such as a gene encoding a detectable gene product, can be under the control of a constitutive promoter, while a second gene, such as a gene encoding a therapeutic gene product, can be under the control of an inducible promoter. Methods for inserting two or more genes into a virus are known in the art and can be readily performed for a wide variety of viruses using a wide variety of exogenous genes, regulatory sequences, and/or other nucleic acid sequences.

Methods of producing recombinant viruses are known in the art (Falkner F G & Moss B (1990) J Virol 64(6):3108-3111). Provided herein for exemplary purposes are methods of producing a recombinant vaccinia virus. A recombinant vaccinia virus with an insertion in the F14.5L gene (NotI site of LIVP) can be prepared by the following steps: (a) generating (i) a vaccinia shuttle plasmid containing the modified F14.5L gene inserted at restriction site X and (ii) a dephosphorylated wt VV (VGL) DNA digested at restriction site X; (b) transfecting host cells infected with PUV-inactivated helper VV (VGL) with a mixture of the constructs of (i) and (ii) of step a; and (c) isolating the recombinant vaccinia viruses from the transfectants. One skilled in the art knows how to perform such methods, for example by following the instructions given in U.S. Pat. Nos. 7,588,7667 and 7,588,771; see also Timiryasova et al. (2001) Biotechniques 31:534-540. In one example, restriction site X is a unique restriction site.

A variety of suitable host cells also are known to the person skilled in the art and include many mammalian, avian and insect cells and tissues which are susceptible for vaccinia virus infection, including chicken embryo, rabbit, hamster and monkey kidney cells, for example, HeLa cells, RK13, CV-1, Vero, BSC40 and BSC-1 monkey kidney cells.

6. Antibodies for Capture of Virally-Infected Tumor Cells

Any antibody that binds to a virally encoded protein can be used in the methods provided herein. The antibodies provided herein can bind to any protein described herein that is virally encoded. The antibody can bind to a virally encoded membrane protein, such as a receptor protein or transporter protein. One of skill in the art can readily identify such antibodies and can adapt them for the methods described herein for detection and enumeration of CTCs. In particular examples, the antibodies used herein are antibodies that bind to a virally encoded NIS protein.

The antibodies and antigen-binding fragments thereof, provided herein that can bind a virally encoded protein described herein or known to one of skill in the art, such as a virally encoded membrane protein, can have an amino acid sequence that resembles a mammalian antibody light or heavy chain. For example, a polypeptide can have additional amino acid residues C-terminal to the CDR and framework sequences. The additional residues can form a sequence resembling that of the constant region of the light or heavy chain of a human or other mammalian antibody. Mammalian antibody constant regions are known in the art. Examples of mammalian constant region sequences are described in Kabat et al., Sequences of proteins of immunological interest edn 5th: National Institutes of Health Publication No. 91-3242 (1991).

Thus, the C-terminal segment of an antibody or antibody fragment provided herein can have one or more than one immunoglobulin domains as is typically present in the light and heavy chain constant regions of human or other mammalian antibodies. The constant region of a mammalian antibody light chain typically has one immunoglobulin domain, while the constant region of a mammalian antibody heavy chain typically has three or four immunoglobulin domains. The antibody also can have one or more than one cysteine residues that allow for formation of intra-chain disulfide bond between amino acid residues within the antibody or fragment thereof or for formation of inter-chain disulfide bonds between two antibodies or fragments thereof. Further, the antibody or antibody fragment can have a region that resembles the hinge region of a mammalian antibody heavy chain. The hinge region, when present in an antibody provided herein, is located between the first and second immunoglobulin domains and can have from 10 to over 60 amino acid residues. A portion of the hinge region can adopt a random and flexible conformation allowing for molecular motion.

In instances where the constant region is included in the antibodies that bind a virally encoded protein, the constant region can have an amino acid sequence of a contant region of any of the immunoglobulin classes. The constant region can be from the light chain or heavy chain, including any known in the art. Exemplary Fab′ human consensus constant region sequences include, for example, those provided within the genebank of the National Center for Biotechnology Information. Typically, the antibody contains a constant region from an IgG immunoglobulin, such IgG1, IgG2, IgG3 or IgG4.

Included among such antibodies are full length antibodies, or antigen-binding fragments thereof, including, for example, scFv, Fab, Fab′, F(ab′)₂, Fv, dsFv, diabody, Fd, or Fd′ fragments. The antibodies or antigen-binding fragments thereof can selectively bind to a virally encoded protein. In some examples, the antibodies or antigen-binding fragments thereof bind to NIS. For example, the antibodies can bind to hNIS. In some examples, the antibodies or antigen-binding fragments thereof selectively bind to NIS (or hNIS) expressed on the surface of a CTC. Also included are antibodies that bind to the same epitope as any of the antibodies described herein.

a. General Structure of Antibodies

Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable region (V_{H}) followed by a number of constant regions. Each light chain has a variable region at one end (V_{L}) and a constant region at its other end. The constant region of the light chain is aligned with the first constant region of the heavy chain, and the light chain variable region is aligned with the variable region of the heavy chain. The variable region of either chain has a triplet of hypervariable or complementarity determining regions (CDR's) spaced within a framework sequence as explained below. The framework and constant regions of the antibody have highly conserved amino acid sequences such that a species consensus sequence may typically be available for the framework and constant regions. Particular amino acid residues are believed to form an interface between the light and heavy chain variable regions (Chothia et al., (1985) J. Mol. Biol. 186:651-63; Novotny and Haber, (1985) Proc. Nail. Acad. Sci. USA 82:4592-4596). Antibodies are produced naturally by B cells in membrane-bound and secreted forms. Antibodies specifically recognize and bind antigen epitopes through cognate interactions. Antibody binding to cognate antigens can initiate multiple effector functions, which cause neutralization and clearance of toxins, pathogens and other infectious agents.

Diversity in antibody specificity arises naturally due to recombination events during B cell development. Through these events, various combinations of multiple antibody V, D and J gene segments, which encode variable regions of antibody molecules, are joined with constant region genes to generate a natural antibody repertoire with large numbers of diverse antibodies. A human antibody repertoire contains more than 10¹⁰ different antigen specificities and thus theoretically can specifically recognize any foreign antigen. Antibodies include such naturally produced antibodies, as well as synthetically, i.e. recombinantly, produced antibodies, such as antibody fragments, including the anti-NIS antibodies or antigen-binding fragments provided herein.

In folded antibody polypeptides, binding specificity is conferred by antigen-binding site domains, which contain portions of heavy and/or light chain variable region domains. Other domains on the antibody molecule serve effector functions by participating in events such as signal transduction and interaction with other cells, polypeptides and biomolecules. These effector functions cause neutralization and/or clearance of the infecting agent recognized by the antibody. Domains of antibody polypeptides can be varied according to the methods herein to alter specific properties.

i. Structural and Functional Domains of Antibodies

Full-length antibodies contain multiple chains, domains and regions. A full length conventional antibody contains two heavy chains and two light chains, each of which contains a plurality of immunoglobulin (Ig) domains. An Ig domain is characterized by a structure called the Ig fold, which contains two beta-pleated sheets, each containing anti-parallel beta strands connected by loops. The two beta sheets in the Ig fold are sandwiched together by hydrophobic interactions and a conserved intra-chain disulfide bond. The Ig domains in the antibody chains are variable (V) and constant (C) region domains. Each heavy chain is linked to a light chain by a disulfide bond, and the two heavy chains are linked to each other by disulfide bonds. Linkage of the heavy chains is mediated by a flexible region of the heavy chain, known as the hinge region.

Each full-length conventional antibody light chain contains one variable region domain (V_{L}) and one constant region domain (C_{L}). Each full-length conventional heavy chain contains one variable region domain (V_{H}) and three or four constant region domains (C_{H}) and, in some cases, hinge region. Owing to recombination events discussed above, nucleic acid sequences encoding the variable region domains differ among antibodies and confer antigen-specificity to a particular antibody. The constant regions, on the other hand, are encoded by sequences that are more conserved among antibodies. These domains confer functional properties to antibodies, for example, the ability to interact with cells of the immune system and serum proteins in order to cause clearance of infectious agents. Different classes of antibodies, for example IgM, IgD, IgG, IgE and IgA, have different constant regions, allowing them to serve distinct effector functions.

Each variable region domain contains three portions called complementarity determining regions (CDRs) or hypervariable (HV) regions, which are encoded by highly variable nucleic acid sequences. The CDRs are located within the loops connecting the beta sheets of the variable region Ig domain. Together, the three heavy chain CDRs (CDR1, CDR2 and CDR3) and three light chain CDRs (CDR1, CDR2 and CDR3) make up a conventional antigen-binding site (antibody combining site) of the antibody, which physically interacts with cognate antigen and provides the specificity of the antibody. A whole antibody contains two identical antibody combining sites, each made up of CDRs from one heavy and one light chain. Because they are contained within the loops connecting the beta strands, the three CDRs are non-contiguous along the linear amino acid sequence of the variable region. Upon folding of the antibody polypeptide, the CDR loops are in close proximity, making up the antigen combining site. The beta sheets of the variable region domains form the framework regions (FRs), which contain more conserved sequences that are important for other properties of the antibody, for example, stability.

ii. Antibody Fragments

Antibodies provided herein include antibody fragments, which are derivatives of full-length antibodies that contain less than the full sequence of the full-length antibodies but retain at least a portion of the specific binding abilities of the full-length antibody. The antibody fragments also can include antigen-binding portions of an antibody that can be inserted into an antibody framework (e.g., chimeric antibodies) in order to retain the binding affinity of the parent antibody. Examples of antibody fragments include, but are not limited to, Fab, Fab′, F(ab′)₂, single-chain Fvs (scFv), Fv, dsFv, diabody, Fd and Fd′ fragments, and other fragments, including modified fragments (see, for example, Methods in Molecular Biology, Vol 207: Recombinant Antibodies for Cancer Therapy Methods and Protocols (2003); Chapter 1; p 3-25, Kipriyanov). Antibody fragments can include multiple chains linked together, such as by disulfide bridges and can be produced recombinantly. Antibody fragments also can contain synthetic linkers, such as peptide linkers, to link two or more domains. Methods for generating antigen-binding fragments are well-known in the art and can be used to modify any antibody provided herein. Fragments of antibody molecules can be generated, such as for example, by enzymatic cleavage. For example, upon protease cleavage by papain, a dimer of the heavy chain constant regions, the Fc domain, is cleaved from the two Fab regions (i.e. the portions containing the variable regions).

Single chain antibodies can be recombinantly engineered by joining a heavy chain variable region (V_{H}) and light chain variable region (V_{L}) of a specific antibody. The particular nucleic acid sequences for the variable regions can be cloned by standard molecular biology methods, such as, for example, by polymerase chain reaction (PCR) and other recombination nucleic acid technologies. Methods for producing sFvs are described, for example, by Whitlow and Filpula (1991) Methods, 2: 97-105; Bird et al. (1988) Science 242:423-426; Pack et al. (1993) Bio/Technology 11:1271-77; and U.S. Pat. Nos. 4,946,778, 5,840,300, 5,667,988, 5,658,727 and 5,258,498). Single chain antibodies also can be identified by screening single chain antibody libraries for binding to a target antigen. Methods for the construction and screening of such libraries are well-known in the art.

The antibodies or antigen-binding fragment thereof provided include polyclonal antibodies, monoclonal antibodies, multispecific antibodies, bispecific antibodies, human antibodies, humanized antibodies, camelised antibodies, chimeric antibodies, single-chain Fvs (scFv), single chain antibodies, single domain antibodies, Fab fragments, F(ab′) fragments, disulfide-linked Fvs (sdFv), and anti-idiotypic (anti-Id) antibodies, intrabodies, or antigen-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The antibodies or antigen-binding fragments thereof provided herein can contain any constant region known in the art, such as any human constant region known in the art, including, but not limited to, human light chain kappa (κ), human light chain lambda (λ), the constant region of IgG1, the constant region of IgG2, the constant region of IgG3 or the constant region of IgG4.

Also included in the antibodies and antigen-binding fragments provided herein are those that bind to an epitope in the extracellular region of hNIS. For example, also included are antibodies and antigen-binding fragments that bind to an epitope located within amino acids 208-241 of hNIS (RGVMLVGGPRQVLTLAQNHSRINLMDFNPDPRSR (SEQ ID NO: 50)). Also included are antibodies and antigen-binding fragments that bind to an epitope located within amino acids 466-525 of hNIS (YPPSEQTMRVLPSSAARCVALSVNASGLLDPALLPANDSSRAPSSGMDASRPALADS FYA (SEQ ID NO: 51)). In some examples, the antibodies and antigen binding fragments provided herein bind to an epitope located within amino acids 225-238 of hNIS ((NHSRINLMDFNPDP (SEQ ID NO: 52)), amino acids 468-481 of hNIS (PSEQTMRVLPSSAA (SEQ ID NO: 54)); or amino acids 502-515 of hNIS (NDSSRAPSSGMDAS, SEQ ID NO: 53)).

b. Additional Modifications of Antibodies

The antibodies and fragments thereof provided herein can be modified by the attachment of a heterologous peptide to facilitate purification. Generally such peptides are expressed as a fusion protein containing the antibody fused to the peptide at the C- or N-terminus of the antibody or antigen-binding fragment thereof. Exemplary peptides commonly used for purification include, but are not limited to, hexa-histidine peptides, hemagglutinin (HA) peptides, and flag tag peptides (see e.g., Wilson et al. (1984) Cell 37:767; Witzgall et al. (1994) Anal Biochem 223(2):291-298). The fusion does not necessarily need to be direct, but can occur through a linker peptide. In some examples, the linker peptide contains a protease cleavage site which allows for removal of the purification peptide following purification by cleavage with a protease that specifically recognizes the protease cleavage site.

The antibodies or antigen-binding fragments thereof can also be attached to solid supports, which are useful for immunomagnetic capture of CTCs. Exemplary solid supports include, but are not limited to, glass, cellulose, polyacrylamide, nylon, polystyrene, polyvinyl chloride, polypropylene or magnetic beads.

i. PEGylation

The antibodies or antigen-binding fragments thereof provided herein can be conjugated to polymer molecules such as high molecular weight polyethylene glycol (PEG) to increase half-life and/or improve their pharmacokinetic profiles. Conjugation can be carried out by techniques known to those skilled in the art. Conjugation of therapeutic antibodies with PEG has been shown to enhance pharmacodynamics while not interfering with function (see, e.g., Deckert et al., Int. J. Cancer 87:382-390, 2000; Knight et al., Platelets 15:409-418, 2004; Leong et al., Cytokine 16:106-119, 2001; and Yang et al., Protein Eng. 16:761-770, 2003). PEG can be attached to the antibodies or antigen-binding fragments with or without a multifunctional linker either through site-specific conjugation of the PEG to the N- or C-terminus of the antibodies or antigen-binding fragments or via epsilon-amino groups present on lysine residues. Linear or branched polymer derivatization that results in minimal loss of biological activity can be used. The degree of conjugation can be monitored by SDS-PAGE and mass spectrometry to ensure proper conjugation of PEG molecules to the antibodies.

Unreacted PEG can be separated from antibody-PEG conjugates by, e.g., size exclusion or ion-exchange chromatography. PEG-derivatized antibodies or antigen-binding fragments thereof can be tested for binding activity to antigens as well as for in vivo efficacy using methods known to those skilled in the art, for example, by immunoassays described herein.

c. Methods for Producing Antibodies

The antibodies or antigen-binding fragments thereof provided herein can be generated by any suitable method known in the art for the preparation of antibodies, including chemical synthesis and recombinant expression techniques. Various combinations of host cells and vectors can be used to receive, maintain, reproduce and amplify nucleic acids (e.g. nucleic acids encoding antibodies such as antibodies or antigen-binding fragments thereof provided that bind to virally encoded genes), and to express polypeptides encoded by the nucleic acids. In general, the choice of host cell and vector depends on whether amplification, polypeptide expression, and/or display on a genetic package, such as a phage, is desired. Methods for transforming host cells are well known. Any known transformation method (e.g., transformation, transfection, infection, electroporation and sonoporation) can be used to transform the host cell with nucleic acids. Procedures for the production of antibodies, such as monoclonal antibodies and antibody fragments, such as, but not limited to, Fab fragments and single chain antibodies are well known in the art.

Antibodies may be produced using techniques well known to those of skill in the art and disclosed in, for example, U.S. Pat. Nos. 4,011,308; 4,722, 890; 4,016,043; 3,876,504; 3,770,380; and 4,372,745. See also Antibodies-A Laboratory Manual, Harlow and Lane, eds., Cold Spring Harbor Laboratory, N.Y. (1988). For example, polyclonal antibodies are generated by immunizing a suitable animal, such as a mouse, rat, rabbit, sheep, or goat, with an antigen of interest. In order to enhance immunogenicity, the antigen can be linked to a carrier prior to immunization. Such carriers are well known to those of ordinary skill in the art. Immunization is generally performed by mixing or emulsifying the antigen in saline, preferably in an adjuvant such as Freund's complete adjuvant, and injecting the mixture or emulsion parenterally (generally subcutaneously or intramuscularly). The animal is generally boosted 2-6 weeks later with one or more injections of the antigen in saline, preferably using Freund's incomplete adjuvant. Antibodies may also be generated by in vitro immunization, using methods known in the art. Polyclonal antiserum is then obtained from the immunized animal.

Monoclonal antibodies can be prepared using a wide variety of techniques known in the art including, but not limited to, the use of hybridoma, recombinant expression, phage display technologies or a combination thereof. For example, monoclonal antibodies can be produced using hybridoma techniques including those known in the art and taught for example in Harlow et al. Antibodies: A Laboratory Manual (Cold Spring Harbor Laboratory Press, 2nd ed. 1988); Hammerling, Monoclonal Antibodies and T-Cell Hybridomas 5630681 (Elsevier N.Y. 1981).

The antibodies or fragments thereof provided herein, can be produced by any method known to those of skill in the art including in vivo and in vitro methods. Desired polypeptides can be expressed in any organism suitable to produce the required amounts and forms of the proteins, such as for example, needed for analysis, administration and treatment. Expression hosts include prokaryotic and eukaryotic organisms such as E. coli, yeast, plants, insect cells, mammalian cells, including human cell lines and transgenic animals (e.g., rabbits, mice, rats, and livestock, such as, but not limited to, goats, sheep, and cattle), including production in serum, milk and eggs. Expression hosts can differ in their protein production levels as well as the types of post-translational modifications that are present on the expressed proteins. The choice of expression host can be made based on these and other factors, such as regulatory and safety considerations, production costs and the need and methods for purification.

i. Nucleic Acids

Provided herein are isolated nucleic acid molecules encoding a polypeptide described above, that, alone or in combination with another polypeptide, can bind a virally encoded gene. These nucleic acids can be inserted into an expression cassette or expression vector such that they are operably linked to expression control sequences.

Nucleic acid molecules encoding the antibodies or antigen-binding fragments thereof provided herein can be prepared using well-known recombinant techniques for manipulation of nucleic acid molecules (see, e.g., techniques described in Sambrook et al. (1990) Molecular Cloning, A Laboratory Manual, 2d Ed., Cold Spring Harbor Laboratory, Cold Spring Harbor, N.Y. and Ausubel et al., eds. (1998) Current Protocols in Molecular Biology, John Wiley & Sons, NY). In some examples, methods, such as, but not limited to, recombinant DNA techniques, site directed mutagenesis, and polymerase chain reaction (PCR) can be used to generate modified antibodies or antigen-binding fragments thereof having a different amino acid sequence, for example, to create amino acid substitutions, deletions, and/or insertions.

Polypeptides and antibodies also can be produced by recombinant expression. First, nucleic acids encoding these polypeptides and antibodies can be constructed by switching the regions of these molecules that encode the CDR and/or framework sequences. In particular, the nucleic acid encoding a first polypeptide can be modified by insertion or replacement of nucleic acid regions encoding, for example, a CDR region, a framework region or a constant region, from another nucleic acid encoding a second polypeptide using known recombinant techniques.

Nucleic acids encoding selected CDR and framework sequences can be joined by splicing using overlapping extension PCR, and the resulting nucleic acid inserted into an expression vector for expression in a bacterial or mammalian host cell as described below. See, for example, Horton et al., Biotechniques 8:528-535 (1990). Nucleic acid sequences encoding constant regions of the light and heavy chains of human and other mammalian antibodies are known in the art and can be obtained from the public databases such as Genbank. Examples of nucleic acid sequences encoding constant regions are also described in Kabat et al., Sequences of Proteins of Immunological Interest, 5^{th} Edition, National Institutes of Health Publication No. 91-3242 (1991). See the Cold Spring Harbor Laboratory Manuals cited below for the details involved in DNA sequence engineering. Nucleic acid sequences encoding individual CDR and framework sequences also can be synthesized using known techniques such as, for example, solid phase synthesis. Polypeptides also can be produced through synthetic methods well-known in the art (Merrifield, Science, 85:2149 (1963)).

ii. Purification

Methods for purification of polypeptides, including the antibodies or antigen-binding fragments thereof provided, from host cells will depend on the chosen host cells and expression systems. For secreted molecules, proteins generally are purified from the culture media after removing the cells. For intracellular expression, cells can be lysed and the proteins purified from the extract. In one example, polypeptides are isolated from the host cells by centrifugation and cell lysis (e.g. by repeated freeze-thaw in a dry ice/ethanol bath), followed by centrifugation and retention of the supernatant containing the polypeptides. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary the proteins can be extracted and further purified using standard methods in the art.

The antibodies or antigen-binding fragments thereof provided, can be purified, for example, from lysed cell extracts, using standard protein purification techniques known in the art including but not limited to, SDS-PAGE, size fraction and size exclusion chromatography, ammonium sulfate precipitation and ionic exchange chromatography, such as anion exchange. Affinity purification techniques also can be utilized to improve the efficiency and purity of the preparations. For example, antibodies, receptors and other molecules that bind proteases can be used in affinity purification. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis and staining and spectrophotometric techniques.

The isolated polypeptides then can be analyzed, for example, by separation on a gel (e.g. SDS-Page gel), size fractionation (e.g. separation on a Sephacryl™ S-200 HiPrep™ 16×60 size exclusion column (Amersham from GE Healthcare Life Sciences, Piscataway, N.J.). Isolated polypeptides also can be analyzed in binding assays, typically binding assays using a binding partner bound to a solid support, for example, to a plate (e.g. ELISA-based binding assays) or a bead, to determine their ability to bind desired binding partners. The binding assays described in the sections below, which are used to assess binding of precipitated phage displaying the polypeptides, also can be used to assess polypeptides isolated directly from host cell lysates. For example, binding assays can be carried out to determine whether antibody polypeptides bind to one or more antigens, for example, by coating the antigen on a solid support, such as a well of an assay plate and incubating the isolated polypeptides on the solid support, followed by washing and detection with secondary reagents, e.g. enzyme-labeled antibodies and substrates.

7. Applications of the Method

The methods provided herein for the detection and enumeration of CTCs can be employed for a variety of applications including, but not limited to, cancer detection, cancer diagnosis, identification of subjects for oncolytic therapy or other anticancer therapies, staging of cancers, prognosis, monitoring cancer progression, stabilization and regression, monitoring an anti-cancer therapy, such as an oncolytic virus therapy, and monitoring subjects for cancer recurrence following surgery or remission. Further applications include, but are not limited to, detection of residual tumor cells in the bone marrow of patients undergoing high-dose radiotherapy. The detection methods also can be employed in the development and evaluation of new cancer therapies, such as oncolytic virus, vaccine or gene therapies. In some examples, a threshold level of CTCs is used to establish where a sample is considered positive for the particular condition above the threshold value.

In some examples, the methods provided herein for detection and enumeration of CTCs can be used for monitoring efficacy of treatment with an oncolytic virus. For example, an oncolytic reporter virus can be administered to the subject having a tumor, where detection of one or more infected CTCs in a body fluid sample from the subject using the methods provided herein is indicative that treatment with the virus is or will be efficacious. In some examples, the virus can be administered at or about a dosage of 1×10² pfu, 1×10³ pfu, 1×10⁴ pfu, 1×10⁵ pfu, 1×10⁶ pfu, 1×10⁷ pfu or 1×10⁸ pfu. Typically the virus is administered at a dosage that is lower than the dosage that is typically administered for treatment.

In some examples, the methods provided herein for detection and enumeration of CTCs can be used for determining a cancer prognosis. For example, an increase in the level of CTCs detected relative to a control sample is indicative of a poor prognosis. In other examples, a decrease in the level of CTCs detected relative to a control sample is indicative of a favorable prognosis. In some examples, a prognosis is determined by comparing the level of CTCs detected to a control or reference sample or database of values corresponding to a known prognosis. A prognosis can be determined based on whether the level of CTCs detected is at or above a threshold level. In some examples, the level of CTCs in a particular subject is monitored over time by performing a CTC detection method provided herein at consecutive predetermined time points. In such examples, an increase in the level of CTCs detected between two successive time points is indicative of a poor prognosis and a decrease in the level of CTCs detected between two successive time points is indicative of a favorable prognosis.

In some examples, the methods provided herein for detection and enumeration of CTCs can be used for determining whether a subject has a metastasizing tumor. In some examples, detection of one or more CTCs in a subject using the methods provided herein is indicative that the subject has a metastasizing tumor.

In some examples, the methods provided herein for detection and enumeration of CTCs can be used for evaluating the risk in a subject for the development of a metastatic tumor. In some examples, detection of one or more CTCs in a subject using the methods provided herein is indicative that the subject is at risk for developing a metastatic tumor. In some examples, the subject has a tumor, such as a metastatic tumor, is at risk of having a tumor, or is in remission following cancer treatment.

In some examples, the methods provided herein for detection and enumeration of CTCs can be used for staging of cancer or assessing the severity of disease. For example, detection and enumeration of CTCs using the methods provided can be compared to a control or reference or database of values that correlates a particular level of CTCs with a particular stage of cancer. If the level of CTCs detected in the sample is at or above a particular threshold level, it indicates that the cancer is at or has advanced past the particular stage associated with the threshold level of CTCs. If the level of CTCs detected in the sample is lower than a particular threshold level, it indicates that the cancer has not advanced past the particular stage associated with the threshold level of CTCs.

In some examples, the methods provided herein for detection and enumeration of CTCs can be used for monitoring the progression of cancer. The level of CTCs in a particular subject can be monitored over time by performing a CTC detection method provided herein at consecutive predetermined time points. In some examples, an increase in the level of CTCs detected between two successive time points is indicative of cancer progression. In some examples, a decrease in the level of CTCs detected between two successive time points is indicative that the cancer is not advancing or is in regression/remission. In some examples, no difference in the level of CTCs detected between two successive time points is indicative of arrest or stability in the progression of the cancer.

In exemplary methods, where the level of CTCs are compared at two successive time points, the level of CTCs are detected at a first time point and the level of CTC are detected at a second time point 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or later following the first time point. In some examples, the level of CTCs is detected at multiple time points, such as, for example, 2, 3, 4, 5, 6, 7, 8, 9, 10, 15, 20 or more time points.

In exemplary methods, where the level of CTCs in a sample from a subject are compared at two successive time points, the level of CTCs are detected in a first sample collected at a first time point and the level of CTC are detected in a second sample 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or later following the collection of the first sample. In some examples, the level of CTCs is detected multiple sample collected at multiple time points, such as, for example, 2, 3, 4, 5, 6, 7, 8, 9, 10, 15, 20 or more time points.

Generally, in examples where the level of CTCs in two samples are compared to determine an increase or decrease in the level of CTCs, the samples are of the same type and collected in the same manner (i.e. using the same or similar procedures). For example, the level of CTCs in a first blood sample is typically compared to the level of CTCs in a second blood sample.

In some examples, the methods provided herein for detection and enumeration of CTCs can be used for monitoring an anti-cancer therapy or determining the efficacy of an anti-cancer therapy. In some examples, an increase in the level of CTCs detected relative to a control sample is indicative that the anti-cancer therapy is not effective for treatment of the cancer. In some examples, a decrease in the level of CTCs detected relative to a control sample is indicative that the anti-cancer therapy is effective for treatment of the cancer.

The methods for detecting the level of CTCs can be performed before, during or after the patient has undergone one or more rounds of anti-cancer therapy, such as therapy with a chemotherapeutic agent or oncolytic viral therapy. The results obtained with the methods can provide a measure of the therapeutic efficacy of an anti-cancer agent or combinations of anti-cancer agents against particular tumors or different types of tumors. The results can therefore be used to aid in the design of an appropriate therapy protocol, or to monitor the predicted effectiveness of a current protocol. Serial monitoring of CTCs can direct treatment selection during therapy, allow the clinician to make informed decisions about continued or alternative therapies and reduce the cost of drug treatments by eliminating ineffective therapies early in treatment. For example, detection of CTCs or a particular level of CTCs in a body sample can indicate that the treatment should be increased, decreased, accelerated or discontinued. Such changes include, for example, changes in treatment regimen, including, but not limited to a increase or decrease in the frequency of administration, an increase or decrease in the amount of the anticancer agent administered, or the addition or subtraction of anticancer therapies from the regimen. In some examples, where the anticancer agent is an oncolytic virus, a change in the treatment regimen can include an increase or decrease in the frequency of administration, an increase or decrease in the amount of the oncolytic virus administered, or the addition or subtraction of one or more additional anticancer therapies from the regimen, such as the addition of an additional oncolytic virus or a chemotherapeutic agent.

In some examples, the methods are employed for the monitoring of a single anti-cancer therapy. In some examples, the methods are employed for the monitoring of a combination of two or more anti-cancer therapies. Exemplary anti-cancer therapies for monitoring are provided elsewhere herein and include, but are not limited to, radiation, chemotherapy, gene therapy, and treatment with therapeutic viruses.

In some examples, the methods provided herein for detection and enumeration of CTCs can thus be used for stratification of subjects for anti-cancer therapy. For example, a subject can be selected for anti-cancer therapy if one or more CTCs are detected in the sample. In some examples, a subject can be selected for treatment with an anti-metastatic agent. As described herein, the oncolytic viruses, including vaccinia viruses, that are administered to a subject with a metastatic cancer preferentially infect metastasizing cells of the tumor and colonize newly formed metastases, and also clear circulating tumor cells from the subject. Accordingly, a subject can be selected for anti-cancer therapy with an oncolytic virus, for example, an LIVP vaccinia virus, if one or more CTCs are detected in the sample.

Thus, the diagnostic methods can be used in combination with a method for treatment of a cancer where the method involves detection of CTC in a sample from a subject and, if CTCs are detected, administering to the subject an effective amount of an anti-cancer therapy, such as an oncolytic virus, for example, an LIVP vaccinia virus, for the treatment of the metastasis. In some examples, the subject is administered an oncolytic reporter virus and the infected CTCs are detected in a sample from the subject using the methods provided herein. In other examples, a sample is obtained from a subject and infected with the oncolytic reporter virus for the detection of CTCs using the methods provided herein.

Surgical removal of cancer is most successful when the cancer is detected early and is confined to the primary tumor site. If metastasis has already occurred prior to surgery, then a subject is at a higher risk of relapse and subsequent tumor growth. Treatment of subjects either prior to or following surgery to excise the primary tumor can aid in the clearance of metastatic cells that have detached from the tumor. Clearance of the metastases can lower the risk of additional tumor growth. Accordingly, provided herein are methods of treatment of a cancer where the method involves detection of CTC in a sample from a subject and, if CTCs are detected, administering to the subject an effective amount of an oncolytic virus for the treatment of the metastasis where once the metastasis is treated, the primary tumor is removed. In some examples, the subject is administered an oncolytic reporter virus and the infected CTCs are detected in a sample from the subject using the methods provided herein. In other examples, a sample is obtained from a subject and infected with the oncolytic reporter virus for the detection of CTCs using the methods provided herein. After removal of the primary tumor, the patient can undergo regular checks for recurrence and be immediately treated if there is a positive finding.

As one skilled in the art will recognize, the time period for effective treatment with an anti-cancer agent will vary. For example, the time period for infection of a virus will vary depending on the virus, the organ(s) or tissue(s), the immunocompetence of the host and dosage of the virus. Such times can be empirically determined if necessary.

The methods provided herein for detecting and enumerating CTCs can be used to monitor the treatment of cancers and tumors, such as, but not limited to, acute lymphoblastic leukemia, acute lymphoblastic leukemia, acute myeloid leukemia, acute promyelocytic leukemia, adenocarcinoma, adenoma, adrenal cancer, adrenocortical carcinoma, AIDS-related cancer, AIDS-related lymphoma, anal cancer, appendix cancer, astrocytoma, basal cell carcinoma, bile duct cancer, bladder cancer, bone cancer, osteosarcoma/malignant fibrous histiocytoma, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, Burkitt lymphoma, carcinoid tumor, carcinoma, central nervous system lymphoma, cervical cancer, chronic lymphocytic leukemia, chronic myelogenous leukemia, chronic myeloproliferative disorder, colon cancer, cutaneous T-cell lymphoma, desmoplastic small round cell tumor, endometrial cancer, ependymoma. epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, Hodgkin lymphoma, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, lymphomas, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenstrom's macroglobulinemia and Wilm's tumor.

The methods provided herein can be used in combination with one or more additional methods for detecting or monitoring a cancer or tumor or monitoring an anti-cancer therapy. For example, a tumor or metastasis can be detected by physical examination of subject, laboratory tests, such as blood or urine tests, imaging and genetic testing, such as testing for gene mutations that are known to cause cancer. A tumor or metastasis can be detected using in vivo imaging techniques, such as digital X-ray radiography, mammography, CT (computerized tomography) scanning, MRI (magnetic resonance imaging), ultrasonography and PET (positron emission tomography) scanning. Alternatively, a tumor can be detected using tumor markers in blood, serum or urine, that is, by monitoring substances produced by tumor cells or by other cells in the body in response to cancer. For example, prostate specific antigen (PSA) levels are used to detect prostate cancer in men. Additionally, tumors can be detected and monitored by biopsy.

Any of a variety of monitoring steps can be used to monitor an anti-cancer therapy, including, but not limited to, monitoring tumor size, monitoring anti-(tumor antigen) antibody titer, monitoring anti-virus antibody titer, monitoring the presence and/or size of metastases, monitoring the subject's lymph nodes, monitoring the subject's weight or other health indicators including blood or urine markers, monitoring expression of a detectable gene product, and monitoring titer of the oncolytic reporter virus, in a tumor, tissue or organ of a subject.

8. Additional Analysis of Identified CTCs and Validation of Results

Additional analysis can be performed on the CTCs that have been detected using the methods provided herein. For example, assays to confirm tumor cell identity, analyze gene expression, or identify subpopulations of CTCs with differences in gene expression or other physical and/or biological properties can be performed. Exemplary methods include, but are not limited to, morphological analysis, immunohistochemistry with one or more tumor cell markers, or gene expression analysis (e.g., genetic profiling). Such methods are known in the art and can be performed during or following detection of the CTCs using the methods provided. Further analysis of detected CTCs also can include determining the origin of the tumor, such as for example, by immunostaining or gene expression analysis.

Any appropriate method known in the art can be employed to detect expressed gene products, including, but not limited to, quantitative PCR, quantitative RT-PCR, Northern analysis, ELISA, Western blotting and other immunodetection techniques. In particular example, antibodies conjugated to a detectable moiety can be employed for detection. For example, antibodies can be conjugated to fluorescent proteins or molecules, such as for example, but not limited to, Rhodamine, Fluorescein, Cy3, Alexa Fluor 405, Alexa Fluor 488, Alexa Fluor 546, Alexa Fluor 555, Alexa Fluor 633, Alexa Fluor 647, Allophycocyanin (APC), APC-Cy7, fluorescein isothiocyanate (FITC), Pacific Blue, R-phycoerythrin (R-PE), PE-Cy5, PE-Cy7, Texas Red, PE-Texas Red, peridinin chlorophyll protein (PerCP), PerCP-Cy5.5, or can be conjugated to enzymes, such as for example, but not limited to, horseradish peroxidase (HRP) or alkaline phosphatase (AP). Cytological stains that detect, for example, the nucleus (e.g. nucleic acid stains Hoechst 33342 (H33342) and 4′,6-diamidino-2-phenyl indole dihydrochloride (DAPI)) or other cell organelles also can be employed.

In some examples, the detected CTCs are further analyzed for cancer stem cell (CSC) properties. In some examples, immunohistochemistry or RT-PCR is performed to analyze the presence of CSC markers such as, but not limited to, CD24, CD34, CD44, CD133, and CD166. In particular examples, an AdnaTest is performed to analyze ALDH1 activity. Exemplary procedures for performing an AdnaTest are provided herein.

In some examples, immunohistochemistry or RT-PCR is performed to analyze the presence of epithelial cell markers, such as cytokeratins, for example cytokeratins 1-20, for example cytokeratins 1, 4-8, 10-11 and 13-20 or a combination thereof. In particular examples, cytokeratins 8, 18, 19 and/or 20 are detected. Examples of further epithelial markers or tumor cell markers include, but are not limited to, CD44, epidermal growth factor receptor (EGFR), human epidermal growth factor receptor 2 (HER2), prostate specific antigen (PSA, Israeli et al., (1994) Cancer Res 54, 6306-6310), prostate specific membrane antigen (PSMA), the human melanoma antigen (MAGE)-encoding gene family (De Plaen et al. (1994) Immunogenetics 40:360-369), Hasegawa et al. (1998) Pathol Lab Med 122, 551-554), breast-specific antigens such as MAS-385, SB-6 (Ross et al. (1993) Blood 82:2605-2610), Mucin-1 (MUC-1) (Brugger et al. (1999) J Clin Oncol 17:1535-1544) and GA733-2 (Zhong et al. (1999) Tumor Diagn. Ther. 20:39-44), adhesion molecules such as TFS-2, EpCAM (Racila et al. (1998) Proc Natl Acad Sci USA 95:4589-4594), E-Cadherin, or CEACAM1 (Thies et al. (2002) J Clin Oncol 20: 2530-2536), receptor molecules, such as leukocyte associated receptor (LAR), cMET/hepatocyte growth factor receptor (HGFR), androgen receptor, and estrogen-progesterone receptors (Bitran et al. (1992) Dis Mon 38: 213-260), carcinoembryonic antigen (CEA) (Liefers et al. (1998) N Engl J Med 339:223-228), PRL-3 protein, a tyrosine phosphatase (Saha et al. (2001) Science 294, 1343-1346) or maspin, a protein from the serpin family (Sabbatini et al. (2000) J Clin Oncol 18, 1914-1920); CA15.3 CA125, mesothelin, S100, and glial fibrillary acidic protein (GFAP), CD34, ErbB-2/I-IER2, ERcc1, CXCR4, ribonucleotide reductase subunit M1 (RRM1), insulin-like growth factor-I (IGF1), echinoderm microtubule-associated protein-like 4 (EML-4), RecQ-mediated genome instability protein 1 (RMI1), and DNA excision repair protein ERCC-1. In some examples, the presence of a specific genetic modification are analyzed, such as for example, a gene mutation, insertion or deletion.

### D. THERAPEUTIC METHODS

The diagnostic methods provided herein for detection and enumeration of CTCs can be used in combination with other diagnostic methods and with therapeutic methods for the treatment of cancer and metastases. As shown herein in the examples provided, administration of oncolytic reporter viruses results in inhibition of metastasis and metastatic tumor formation and regression of primary and metastatic tumors. The oncolytic viruses also treat CTCs that have been shed from the tumor. Inhibition of metastasis results in decreased shedding of tumor cells. Treatment with an oncolytic virus thus results in decreased tumor cells found in body fluids of the subject which can be monitored using the methods of detection provided herein.

In some examples, subjects are selected for treatment with an anti-cancer agent based on the detection of one more CTCs in a sample from the subject. Upon detection of one or more tumor cells in a body fluid sample, the subject can be prescribed a particular regimen or course of therapy. In some examples, the subject is administered one or more anticancer agents. In some examples, the anticancer agent is an oncolytic virus. The reporter viruses used in the methods provided are oncolytic viruses and can be used for therapy. In some examples, a different oncolytic virus is administered for therapy. As described herein, oncolytic viruses can also be modified to express therapeutic proteins, such as anti-cancer proteins or additional diagnostic proteins.

Additional exemplary anticancer agents that can be administered for cancer therapy in the methods provided include, but are not limited to, chemotherapeutic compounds (e.g., toxins, alkylating agents, nitrosoureas, anticancer antibiotics, antimetabolites, antimitotics, topoisomerase inhibitors), cytokines, growth factors, hormones, photosensitizing agents, radionuclides, signaling modulators, anticancer antibodies, anticancer oligopeptides, anticancer oligonucleotides (e.g., antisense RNA and siRNA), angiogenesis inhibitors, radiation therapy, or a combination thereof. Exemplary chemotherapeutic compounds include, but are not limited to, Ara-C, cisplatin, carboplatin, paclitaxel, doxorubicin, gemcitabine, camptothecin, irinotecan, cyclophosphamide, 6-mercaptopurine, vincristine, 5-fluorouracil, and methotrexate. As used herein, reference to an anticancer or chemotherapeutic agent includes combinations or a plurality of anticancer or chemotherapeutic agents unless otherwise indicated. Anticancer agents include anti-metastatic agents. In some examples, the anti-cancer agent is an oncolytic virus, such as an LIVP vaccinia virus.

In some examples, a oncolytic reporter virus is administered to a subject for the detection of CTCs in a sample from the subject or in vivo using the methods provided herein. In some examples, where the subject has cancer, tumor, metastasis, or one or more CTCs, the administered oncolytic reporter virus can simultaneously provide therapy of the cancer, tumor, metastasis, or one or more CTCs. For example, the oncolytic reporter virus can provide oncolytic therapy of the cancer, tumor, metastasis, or CTCs. The oncolytic virus also can express one or more therapeutic genes for therapy of the cancer, tumor, metastasis, or CTCs. Exemplary therapeutic genes for expression are provided elsewhere herein and include, but are not limited to, tumor suppressors, cytostatic proteins and costimulatory molecules, such as a cytokine, a chemokine, or other immunomodulatory molecules, an anticancer antibody, such as a single-chain antibody, antisense RNA, siRNA, prodrug converting enzyme, a toxin, a mitosis inhibitor protein, an antitumor oligopeptide, an anticancer polypeptide antibiotic, an angiogenesis inhibitor, or tissue factor.

Metastatic tumor cells such as circulating tumor cells (CTCs), and tumor cells in the cerebrospinal fluid (CSF) and the ascites, are surrogate markers in evaluating cancer prognosis and for monitoring therapeutic response. In addition, these metastatic tumor cells are targets for treatment. As exemplified herein, live metastatic tumor cells were detected by the method herein and shown by the methods provided herein to be eliminated by oncolytic vaccinia virus (VACV) treatment. Live CTCs in the blood drawn from mice bearing human prostate and lung cancer xenografts as well as in the blood drawn from patients with metastatic breast, colorectal, lung cancers, and melanoma were detected and enumerated using a tumor cell-specific recombinant reporter VACV that over-expresses the bright far-red fluorescent protein TurboFP635, in an epithelial biomarker-independent manner. Similarly, live tumor cells in the CSF obtained from a patient with late-stage metastatic breast cancer were specifically detected by the methods herein. The methods herein also demonstrate that early treatment with a single intravenous injection of the oncolytic VACV prevented CTC formation, and late treatment resulted in elimination of CTCs in mice bearing human prostate cancer xenografts. A single intra-peritoneal delivery of VACV resulted in a dramatic decline in the number of tumor cells in the ascitic fluid from a patient with peritoneal carcinomatosis from gastric cancer 7 days after treatment. Thus, the methods herein provide a reliable tool for quantitative detection of live tumor cells in liquid biopsies and also are concomitantly effective as a treatment for reducing or eliminating live tumor cells in body fluids of cancer patients with metastatic disease.

### E. COMBINATIONS, KITS, AND ARTICLES OF MANUFACTURE

The oncolytic reporter viruses and reagents, materials and devices for detecting a reporter gene, performing a tumor cell enrichment method, or further analyzing detected CTCs and combinations thereof, can be provided as combinations of the agents, which optionally can be packaged as kits. In non-limiting examples, an oncolytic reporter virus can be provided in combination with a microfilter or a microfluidic device. In non-limiting examples, an oncolytic reporter virus can be provided in combination with a substrate or ligand that binds to the expressed reporter protein. In other non-limiting examples, an oncolytic reporter virus can be provided in combination with reagents for the lysis of red blood cells in a blood sample or antibodies for the removal of non-CTCs from the sample. In other non-limiting examples, an oncolytic reporter virus can be provided in combination with reagents for additional analysis of detected CTCs, such as for example, reagent to measure one or more additional tumor cell markers. For example, kit can include reagents to fix, permeabilize, stain, or lyse tumor cells, reagents for amplification of nucleic acid, antibodies for immunohistochemical analysis and/or primers for RT-PCR or qPCR.

Kits can optionally include one or more components such as instructions for use, additional reagents such as diluents, culture media, substrates, antibodies and ligands, and material components, such as sample collection devices, microfilters, microfluidic chips, microscope slides, tubes, microtiter plates (e.g., multi-well plate) and containers for practice of the methods. Those of skill in the art will recognize many other possible containers and plates that can be used for contacting the various materials.

Exemplary kits can include the viruses provided herein, and can optionally include instructions for use, and additional reagents used in detection of virus infection, such as expression of a reporter gene by the reporter virus. Such reagents can include one or more substrates for detection of a reporter enzyme. Examples of such reagents are described herein. In some examples, the kit includes a device, such as a fluorometer, luminometer, or spectrophotometer for assay detection.

In some examples, the viruses can be supplied in a lyophilized form, and the kit can optionally include one or more solutions for reconstitution of the virus. In a further example, the lyophilized viruses can be supplied in the kit in appropriate amounts in the wells of one or more microtiter plates or sample tubes.

In some examples, a kit can contain instructions. Instructions typically include a tangible expression describing the virus and, optionally, other components included in the kit, and methods for assay, including methods for preparing the virus, methods for preparing the samples, methods for detection of the reporter protein expressed by the viruses, and methods for performing the tumor cell enrichment method.

The articles of manufacture provided herein contain the reporter viruses and packaging materials. Packaging materials for use in packaging products are known to those of skill in the art. See, e.g., U.S. Pat. Nos. 5,323,907, 5,052,558 and 5,033,252. Examples of packaging materials include, but are not limited to, blister packs, bottles, tubes, bags, vials, containers, and any packaging material suitable for a selected formulation and intended use. Articles of manufacture include a label with instructions for use of the packaged material.

One of skill in the art will appreciate the various components that can be included in a kit, consistent with the methods and systems disclosed herein.

### F. EXAMPLES

The following examples are included for illustrative purposes only and are not intended to limit the scope of the invention. It is to be understood that the methods and compositions provided herein are exemplified with the LIVP virus GLV-1h68 but that any oncolytic virus, particularly any vaccinia virus, but also any virus that accumulates in and replicates in tumor cells, can be employed in the methods and compositions herein. The methods detect CTC cells, include cancer stem cells, in body fluids by virtue of accumulation and replication of a detectable oncolytic virus (oncolytic reporter virus) in such cells.

### Example 1

### Analysis of the Metastatic Spread of the Human Prostate Carcinoma Cell Line PC-3

In this example, the metastatic spread of the human prostate carcinoma cell line, PC-3, is shown. A mouse xenograft model of human prostate cancer was developed in which PC-3 cells were injected subcutaneously into the right rear flank of immunocompromised mice. The mice then were assessed for subsequent metastasis at multiple time points post-injection.

### A. Analysis of Lymph Node Size Following PC-3 Tumor Cell Implantation

Tumors were established by subcutaneous implantation of 2×10⁶ PC-3 human prostate cancer cells (ATCC# CRL-1435), suspended in phosphate buffered saline (PBS), or PBS only, into the right flank of homozygous nude mice (Hsd:Athymic Nude-FoxnInu; Harlan, Indianapolis, Ind.; n=4 per treatment group, 24 mice total). Mice were sacrificed at 7, 14, 21, 28, 35, and 42 days post-tumor cell implantation, and the lumbar and renal lymph nodes in the abdominal cavity were examined following ventral incision and removal of internal organs. The number and volume (mm³) of enlarged lymph nodes per mouse were assessed. Volume was measured by digital caliper. The average volume of lumbar and renal lymph node per mouse and the average volume for all lymph nodes was calculated. A lymph node with a diameter greater than 2 mm was considered to be enlarged. The number of enlarged lymph nodes per mouse increased from week to week from ˜2 enlarged lymph nodes per mouse at 7 days post implantation to ˜4 enlarged lymph nodes per mouse at 28 days post implantation to ˜5 enlarged lymph nodes per mouse at 42 days post implantation. The total volume of the enlarged lymph nodes also increased with time from approximately 1 mm³ at 7 days post implantation to greater than 20 mm³ at 28 days post implantation to greater than 30 mm³ at 42 days post implantation.

The lymph nodes were then classified as either renal or lumbar lymph nodes based on their location and assessed individually. The volumes of two renal lymph nodes (RN1 and RN2) and two lumbar lymph nodes (LN1 and LN2) were individually measured at 7, 14, 21, and 28 days post-inoculation to determine if the volume of the enlarged lymph node correlated with its location. At 21 days post implantation, LN1, located on the right-hand side of the mouse, closest to the tumor cell implantation site, demonstrated a significantly greater increase in volume than any of the other three lymph nodes (LN1 was greater than 20 (21 mm3) mm³ compared to the size of the other lymph nodes, which were 7 mm³ or less. At 28 days post tumor cell implantation LN1 was still larger (˜40 mm³) than LN2 (˜33 mm³), RN1 (˜21 mm³), and RN2 (˜12 mm³). Thus, the increase in volume depended on the localization of the lymph node in relation to the tumor. The lymph nodes closer to the primary tumor exhibited more rapid growth than the lymph nodes farther from the tumor.

### B. Presence of Exogenous PC-3 Tumor Cells in Enlarged Lymph Nodes

Lymph nodes obtained from mice bearing human PC-3 xenograft tumors (from part A above) were analyzed for the presence of PC-3 tumor cells. At 21, 28, 35, and 42 days post-implantation, the lymph nodes measured in part A above were homogenized, and messenger RNA was isolated and analyzed by reverse transcriptase polymerase chain reaction (RT-PCR) using primers for human β-actin to test for the presence of human-derived PC-3 cells, and primers for mouse β-actin as a positive control for murine tissue.

Human β-actin Forward Primer:
(SEQ ID NO: 22)
5′-CCTCTCCCAAGTCCACACAG-3′
Human β-actin Reverse Primer:
(SEQ ID NO: 23)
5′-CTGCCTCCACCCACTC-3′
Murine β-actin Forward Primer:
(SEQ ID NO: 24)
5′-CGTCCATGCCCTGAGTC-3′
Murine β-actin Reverse Primer:
(SEQ ID NO: 25)
5-GCTGCCTCAACACCTCAAC-3′

The presence of human β-actin at each time point is set forth in Table 4 below. At 42 days post-implantation, PC-3 cells, as determined by the presence of human β-actin, were detected in 90% of all enlarged lymph nodes, indicating that the PC-3 cells of the implanted tumor metastasized into the lymph nodes.

TABLE 4
Lymph Nodes Positive for β-Actin

| Days Post- | fraction human β-actin- | % human β-actin- |
|---|---|---|
| Implantation | positive lymph nodes | positive lymph nodes |
| 21 | 3/14 | 21% |
| 28 | 11/16 | 69% |
| 35 | 14/17 | 83% |
| 42 | 18/20 | 90% |

### Example 2

### Visualization of PC-3 Cell Metastasis

In this example, a PC-3 cell line that expresses red fluorescent protein was established to facilitate discrimination of PC-3 cells from murine cells and allow tracking of metastatic cells. The cell line was used to visualize the metastatic spread of PC-3 cells from xenograft tumors in mice.

### A. Generation of PC-3 Cells Constitutively Expressing Red Fluorescent Protein (RFP)

cDNA encoding monomeric red fluorescent protein (mRFP) (SEQ ID NO:19 (protein) SEQ ID NO:18 (cDNA)) was stably inserted into the PC-3 cell genome by lentiviral transduction using the ViraPower™ Lentiviral Expression System Kit (Invitrogen GmbH, Germany) in accordance with the manufacturer's instructions. The RFP gene for cloning into the lentiviral vector was obtained by PCR from the mRFP-encoding plasmid pCR-TK-Sel-mRFP (SEQ ID NO:16) using the following primers, which contain attB recombination sites for gateway cloning.

Forward-attB1-mRFP:
(SEQ ID NO: 26)
5′-GGGGACAAGTTTGTACAAAAAAGCAGGCTGCCACCATGGCCTCCTCC
GAGG-3′
Reverse-attB2-mRFP:
(SEQ ID NO: 27)
5′-GGGGACCACTTTGTACAAGAAAGCTGGGTCAGAATTCGCCCTTTCAT
TAGG-3′

The PCR product was cloned into a Gateway entry vector (Invitrogen). Site-specific recombination was then carried out between the Gateway vector and the pLENTI6/V5-DEST retroviral vector (Invitrogen Cat. No. V496-10) according to the manufacturer's instructions to produce the pLENTI6/V5-DEST-mRFP expression plasmid, which contains the mRFP gene under the control of the human CMV immediate early promoter for constitutive expression. Replication-incompetent mRFP-coding Lentiviruses were produced in 293FT cells via a co-transfection of the Vira Power™ Packaging Mix and the pLENTI6/V5-DEST-mRFP expression plasmid using Lipofectamine™2000. After transduction of PC-3 cells with mRFP-coding. Lentiviruses, stable RFP-expressing PC-3 clones were selected using 10 μg/mL blasticidin. Approximately 3 months were required for the selection of a stable cell line. Expression of RFP in the PC-3 cell line was observed in 100% of the cells as 90 days post-transduction as confirmed by observation using a fluorescent microscope equipped with the appropriate filter.

### B. Visualization of PC-3-RFP Cells in Mice

2×10⁶ PC-3-RFP cells were injected into female nude mice (n=6) as described in Example 1A. Imaging in anesthetized whole living mice to detect the red fluorescent signal in the tumor was performed every week. Imaging was performed by the Maestro EX Imaging System (Cri, Woburn, USA). RFP fluorescence was readily visible in the right flank where the tumor had developed.

At 55 and 65 days post-injection, the mice were sacrificed, the internal organs were removed, and the remaining renal and lumbar lymph nodes were examined in situ by RFP fluorescence. Imaging of lymph node metastases in the abdominal cavity was performed with the MZ 16 FA Stereo-Fluorescence microscope (Leica, Wetzlar, Germany). At 55 days post-injection, the lumbar lymph nodes, particularly the lymph node proximal to the site of PC-3-RFP tumor cell injection exhibited strong RFP fluorescence. The renal lymph nodes also exhibited RFP fluorescence, but to a lesser extent. Detection of RFP in the enlarged lymph nodes was evidentiary of tumor metastasis. At 65 days post-injection, RFP fluorescence was further increased and was additionally detectable in vessel-like structures, connected to, and between the lumbar and renal lymph node metastases, indicating a pathway for migration of metastatic tumor cells from the lumber lymph node to the renal lymph node.

### Example 3

### Analysis of PC-3-RFP Cell Migration

In this example, the method of cell migration from the lumber lymph node to the renal lymph node was investigated. To determine if PC-3-RFP use blood vessels or lymphatic vessels for migration, histological studies were conducted. Tissues containing the RFP-positive vessel-like structure between the lumbar and renal lymph node metastases in Example 2B were surgically removed, fixed for 16 hours in 4% paraformaldehyde/PBS, pH 7.4. After fixation, samples were washed and embedded into 5% w/v low melt agarose (AppliChem, Darmstadt, Germany) in PBS. Preparation of 100 μm sections was performed using the Leica VT1000 Vibratome (Leica, Heerbrugg, Switzerland). Sections were permeabilized in PBS containing 0.3% Triton X-100 for 1 hour.

The sections were then immunostained overnight using a hamster monoclonal anti-CD31 antibody (Chemicon International, Temecula, USA; Cat. No. MAB1398Z) as a marker for endothelial cells of blood vessels, or a rabbit polyclonal anti-LYVE-1 (lymphatic vessel endothelial hyaluronan receptor) antibody (Abeam, Cambridge, UK; ab14917) as a marker for lymphatic endothelial cells. All primary and secondary antibodies were diluted in PBS/0.3% Triton-X-100 for the incubation steps. After washing the sections with PBS, the sections were incubated with secondary antibody, donkey DyLight488-conjugated secondary antibody (Jackson ImmunoResearch, Pennsylvania), for 4 hours. Following incubation, sections were washed again with PBS. After labeling, tissue sections were mounted in Mowiol 4-88 (Sigma-Aldrich, Taufkirchen, Germany). The sections were visualized by fluorescence microscopic analysis using the appropriate filters. The endothelial markers were visible in the green channel and PC-3-RFP cells were visible in the red channel. The red and green channel images were overlaid to determine the pathway of PC-3-RFP cell migration.

CD31-stained sections indicated the location of blood vessel endothelial cells relative to the PC-3-RFP cells. In one CD31-stained section, the endothelial ring, corresponding to the cross-section of the abdominal aorta was observed adjacent to a cross-section of RFP-positive tissue, but did not surround the RFP-positive tissue indicating that the PC-3-RFP cells do not migrate via blood vessels. In contrast, LYVE-1-stained sections, revealed a lymphatic endothelial ring surrounding the PC-3-RFP cells, demonstrating that PC-3-RFP cells use lymphatic vessels for migration.

### Example 4

### Metastases in Non-Lymphatic Tissue

To determine the extent of metastases of PC-3-RFP cells, other tissues were examined for RFP fluorescence. PC-3-RFP cells were injected into nude mice (n=6) as described in Example 2B. 76 days post cell injection, the lungs were harvested from the animals and placed into a PBS-filled well of a 12-well plate and examined for RFP fluorescence and under bright field microscopy using a MZ 16 FA Stereo-Fluorescence microscope (Leica, Wetzlar, Germany). At 76 days post-implantation, RFP fluorescence was detectable throughout the lung tissue, indicating the presence of hematogenous metastases.

### Example 5

### Colonization of PC-3 Tumors and Metastases by Vaccinia Virus GLV-1h68

In this example, preferential colonization of the Lister strain vaccinia virus GLV-1h68 (SEQ ID NO: 1; U.S. Pat. Pub. No.: US2005/0031643) in lymph node metastases was examined. The GLV-1h68 virus contains an expression cassette containing a Ruc-GFP cDNA (a fusion of DNA encoding Renilla luciferase and DNA encoding GFP) under the control of a vaccinia synthetic early/late promoter P_{SEL} in the F14.5L gene of the virus genome. Infected cells can be detected by GFP fluorescence microscopy.

PC-3-RFP xenograft tumors were developed in 6-7 week-old female nude mice by implanting 2×10⁶ PC-3-RFP cells subcutaneously on the right hind leg as described in Example 2B. At 50 days post PC-3-RFP tumor cell implantation, 3 groups of 6 mice per groups were injected with a single intravenous dose of 1×10⁷ pfu of GLV-1h68 in 100 μL phosphate-buffered saline (PBS) or 100 μL PBS only via the tail vein. Analysis of enlarged lymph nodes and tumors was performed at 3, 7, and 14 days post virus infection (dpi). Animals were sacrificed and prepared as described in Example 1, and the tumors, lymph nodes, and lymphatic vessels were visualized by fluorescence microscopy, using filters to visualize RFP and GFP fluorescence. Images were taken in the green (GLV-1h68) and red (PC-3-RFP) channels and were overlaid to permit co-localization analysis.

At 3 dpi, PC-3-RFP metastases were detected in the renal and lumbar lymph nodes and lymphatic vessels in addition to the solid primary tumor at the site of tumor cell inoculation, consistent with the observations in Example 2B. At the same time point, GLV-1h68 was also detected in the lymph nodes and lymphatic vessels and, to a lesser extent, in the tumor. GLV-1h68 colonization of the lymph node metastases and PC-3 cells in lymphatic vessels was further confirmed at 7 days post virus infection. At each time point, a higher intensity of GFP fluorescence was detected in lymph node metastases compared to the PC-3 tumor.

To confirm that viral colonization occurred preferentially in the metastases compared to the tumors, standard plaque assays were performed to determine viral titer in the tumor and lymph node tissue. Tumor and renal and lumbar lymph nodes were harvested, weighed, homogenized, and microcentrifuged to pellet debris at 3, 7, and 14 dpi (6 mice per time point) as previously described. The virus titer in each of the tissues was quantified by standard plaque assay on CV-1 cells. Virus titers were expressed as plaque forming units (pfu) and corresponded to the amount of infectious virus per gram tissue. The results are set forth in Table 5. At all three time points after virus injection, a higher GLV-1h68 titer was measured in the lymph node metastases compared to the PC-3 tumor. At 3 and 7 days post infection, a higher titer of GLV-1h68 was detected in renal lymph node metastases compared to lumbar metastases, indicating a correlation between a higher viral titer and metastases that arose at later time points.

TABLE 5
Viral Titer in Metastases and Tumors.

| | Viral Titer ± SEM | | |
|---|---|---|---|
| Tissue | 3 dpi | 7 dpi | 14 dpi |
| Tumor | 2.87 × 10⁶ | 3.23 × 10⁷ | 3.25 × 10⁷ |
| LN1^{a} | 4.13 × 10⁷ | 2.42 × 10⁸ | 4.75 × 10⁸ |
| LN2^{b} | 8.74 × 10⁷ | 4.91 × 10⁸ | 1.05 × 10⁹ |
| RN1^{c} | 2.37 × 10⁸ | 1.57 × 10⁹ | 7.92 × 10⁸ |
| RN2^{d} | 3.73 × 10⁷ | 1.27 × 10⁹ | 4.50 × 10⁸ |

^{a}LN1: lumbar lymph node proximal to the injection site
^{b}LN2: lumbar lymph node distal to the injection site
^{c}RN1: renal lymph node proximal to the injection site
^{d}RN2: renal lymph node distal to the injection site

### Example 6

### Analysis of GLV-1h68 Amplification in the Lymph System of Nude Mice

Because preferential viral amplification was observed in the lymph node metastases in Example 5, GLV-1h68 amplification in the lymph nodes of nude mice without tumors was analyzed and compared to metastasized lymph nodes to examine whether preferential accumulation was due to metastasis or the lymphatic tissue itself. To this end, GLV-1h68 accumulation was first measured in various lymph nodes, independent of metastases in non-tumor bearing mice. Next, the GLV-1h68 accumulation was compared in lymph nodes containing metastases with those that were unmetastasized. Finally, it was shown that lymphatic tissue inside the metastases was infected by GLV-1h68.

### A. GLV-1h68 Amplification in the Lymph System of Non-Metastasized Mice

The amplification of GLV-1h68 in non-tumor bearing nude mice was tested to determine if there was lymphatic tissue preference for GLV-1h68 amplification. 1×10′ pfu GLV-h168 were administered to 6-7 week-old female mice (n=3 per treatment group) via intravenous injection into the tail vein. At 7, 14, 21, and 42 dpi, the lumbar (LN), renal (RN), sciatic (SN), axillary (AN), and brachial (BN) lymph nodes proximal (1) and distal (2) to the injection site were harvested, homogenized and the viral titer was determined by standard plaque assay as described in Example 5.

Maximal virus titer of only 140 pfu was observed at 14 dpi in the axillary lymph node proximal to the injection site (AN 1). The lumbar and renal lymph nodes also produced similarly low viral titers of 22 and 2 pfu, respectively at 14 dpi. No statistically significant differences were detected between any of the lymph nodes at any of the time points considered.

### B. Comparison of GLV-1h68 Amplification in Non-Metastasized and Metastasized Lymph Nodes

To determine if amplification of GLV-1h68 occurs preferentially in metastases, the colonization of unmetastasized lymph nodes was compared with those containing metastases. 6-7 week old female mice (n=6 per treatment group) were injected with 2×10⁶ PC-3-RFP cells in the right hind leg as described in Example 2B. 1×10⁷ pfu GLV-1h68 in 100 μl PBS or PBS alone were administered via tail vein injection into PC-3-RFP implanted mice at 50 days post tumor cell implantation. The lymph and renal lymph nodes were harvested at 7 and 14 dpi and analyzed for virus titer by plaque assay as described in Example 5 and compared to the colonization data obtained in Part A.

At 7 and 14 dpi, there were about 10-20 million pfu GLV-1h68 detected in the metastasized lumbar and renal lymph nodes and 0-22 pfu detected in the unmetastasized lymph nodes. The differences in viral titer between metastasized and unmetastasized lymph nodes was statistically significant at 7 dpi (p<0.01) and 14 dpi (p<0.001). The metastasized renal lymph nodes also exhibited significantly higher levels (˜40 million pfu) of GLV-1h68 than control lymph nodes at 14 dpi (p<0.05). Overall, there was significantly higher infection of lymph node metastases than unmetastasized lymph nodes.

### C. Lymphatic Tissue and PC-3-RFP Metastases

To determine whether lymphatic tissue inside the PC-3 metastases contributed to the preferential amplification of GLV-1h68 in these tissues, the location of lymphatic cells was determined relative to the metastases. Nude mice were injected with 2×10⁶PC-3-RFP cells as described in Example 2B. At 21 and 88 days post tumor cell implantation, corresponding to early and late stages, respectively, of PC-3 metastatic cell invasion, the lumbar lymph nodes were excised, cross-sectioned, and fixed for immunofluorescence as described in Example 3. Lymphatic tissue was visualized by immunostaining using an antibody directed against the lymphatic endothelial cell marker (LYVE-1), rabbit polyclonal anti-LYVE-1 antibody (Abcam, Cambridge, UK; ab14917) and a donkey DyLight488-conjugated secondary antibody (Jackson ImmunoResearch, Pennsylvania).

For detection of antigen presenting cells (APCs) within the lumen of the lymph nodes, expression of major histocompatibility complex II (MHC-II) was examined. For

MHC-II staining, sections representing early and late metastasis were prepared from excised lymph nodes at 57 days post tumor cell implantation. Early and late metastasis samples were selected based on the relative size of the tumors (e.g., a small PC-3-RFP tumor was selected to represent early metastasis). The sections were prepared as described in Example 3 and stained for MHC-II using a monoclonal rat anti-MHC Class II (I-A/I-E) antibody (eBioscience, San Diego, Calif.; Cat. No. 14-5321) and a donkey DyLight488-conjugated secondary antibody (Jackson ImmunoResearch, Pennsylvania).

Sections were mounted and analyzed by fluorescence microscopy as described in Example 3. Images from the red channel, illustrating the locations of RFP-positive PC-3 cells, and the green channel, depicting the locations of lymphatic endothelial cells or APCs, were overlaid to determine the relative locations of the different cell types.

At early and late stages of PC-3 cell invasion, no co-localization or intermingling staining was observed between PC-3 cells and native lymph node constituent cells. As PC-3 cells invaded the lymph node tissue, the developing tumor displaced the lymphatic tissue and MHC-II positive cells. Thus, lymphatic tissue was not detected within the metastases. The lymphatic tissue itself is likely not a cause of preferential GLV-1h68 amplification within the lymph node metastases; however contributing factors to virus amplification produced by adjacent lymphatic tissue was not conclusively ruled out. Staining for LYVE-1 and MHC-II in early and late metastatic lymph nodes was repeated in two subsequent experiments, and similar results were obtained.

### Example 7

### Necrotic Tissue in PC-3 Tumors and Metastases

In this example, necrotic tissue in PC-3 tumors and metastases was measured to show that the presence of necrotic tissue contributed to preferential amplification of GLV-1h68 in lymph node metastases. Because viral replication is not possible in necrotic tissue, the tissue was examined to show whether a high amount of necrotic tissue was present in the tumors compared to metastases. 6-7 week-old nude mice were injected with 2×10⁶ PC-3 cells as described in Example 1. PC-3-derived tumors were permitted to grow and metastasize for 57 days, and then the tumors at the sites of injection and the lumbar and renal lymph nodes were removed, fixed and sectioned into 100 μm sections using a Vibratome, as described in Example 3. The sections were then stained with Hoechst dye to stain the DNA and enable visualization of the nuclei. Loss of nuclei is evidentiary of necrotic tissue. The fluorescence signals in whole section images (10× magnification) were analyzed. Two sections were measured per sample. The area of a section that was not stained by Hoechst, due to nuclei degradation, was defined as necrotic and quantified using Image) analysis software.

The percent necrotic area was determined for the tumor and lymph nodes. The necrotic area in PC-3 tumors was about 25%, whereas the necrotic areas in the lumbar and renal lymph nodes was about 10% and 15%, respectively. The difference in necrotic area was statistically significant between the tumor and the lumbar lymph node (p<0.005) and between the tumor and the renal lymph node (p<0.01), as determined by two-tailed Student's t test was used for statistical analysis. P values of ≦0.05 were considered statistically significant. Thus, there was less necrotic tissue in lymph node metastases than in PC-3 tumor, indicating that the lower GLV-1h68 accumulation in the primary tumor results, at least in part from the necrosis of the tumor.

### Example 8

### Analysis of Blood Vessels in PC-3 Tumors and Metastases

The blood vessels in PC-3 tumors and metastases were analyzed show that preferential amplification of GLV-1h68 in lymph node metastases was related to increased blood vessel density and/or increased permeability. The platelet endothelial cell adhesion molecule (PECAM-1/CD31), which is present on endothelial cells, platelets, macrophages and Kupffer cells, granulocytes, T/NK cells, lymphocytes, megakaryocytes, osteoclasts, neutrophils, was used as a marker of lymph node blood vessels. It is expressed in numerous physiological and pathological processes characterized by an increase of vascular permeability.

100 μm Vibratome sections of tumors and lumbar and renal lymph nodes from 5 mice 57 days post PC-3 tumor cell implantation (from Example 7) were prepared as described in Example 3 and immunostained using antibodies directed against CD31 (Hamster monoclonal anti-CD31 antibody, Chemicon International, Temecula, USA; MAB1398Z). Blood vessel density and CD31 fluorescence intensity were determined.

Blood vessel density was measured at 100× magnification. Eight images per tumor, LN and RN, were analyzed per anti-CD31 staining. Images were taken with individual exposure times to capture all detectable blood vessels and cross-sected with 8 horizontal lines at identical positions using Photoshop 7.0. All blood vessels that crossed these lines were counted to yield the vessel density.

Measurement of the CD31 intensity was performed on digital images of the 100 μm stained sections of PC-3 tumors and metastases. For each staining, 8 images per sample were captured with identical settings. RGB-images were converted into 8-bit gray scale with an intensity range from 0-255. The fluorescence intensity of CD31 staining represents the average brightness of all staining related pixels and was measured using Image) software. Images of CD31 staining were taken at 100× magnification.

The mice exhibited indistinguishable blood vessel density in tumor and lumbar lymph node sections, and slightly increased blood vessel density (number of blood vessels per unit area) in the renal lymph nodes, compared to the lumbar lymph nodes. In contrast, there was a statistically significant increase in CD31 mean fluorescence intensity between the tumor and lumbar lymph node metastasis populations (p<0.05) and between the tumor and renal lymph node metastasis populations (p<0.005).

To confirm that the increased fluorescence intensity in the lymph node metastases compared to the tumor was due to an increase in CD31 protein levels, homogenates of tumors and lumbar and renal lymph node metastases were analyzed by quantitative Western blot, using fluorescent secondary antibodies, and a NightOWL Imaging System (Berthold) to measure relative light emission. Significantly increased CD31 protein expression was detected in lumbar (p<0.05) and renal (p<0.05) lymph node metastases than in the tumor. These results indicated that there was increased blood vessel permeability in the lymph node metastases, which would facilitate GLV-1h68 access to these tissues. Thus, increased vascular permeability results in preferential amplification of GLV-1h68 in lymph node metastases.

### Example 9

### Effects of GLV-1h68 Therapy on Metastasis Growth

In this example, the effect of GLV-1h68 on the size of lymph node metastases was analyzed. 2×10⁶ PC-3 cells were injected into 6-7-wk-old female and male nude mice using methods described in Example 1 (11 mice in PBS treated group and 11 mice in the GLV-1h68 treated group were used; male: n=5, female: n=6). At 30 days after cell implantation, 5×10⁶ pfu GLV-1h68 in 100 μL PBS or 100 μL PBS alone were administered by tail vein injection. The animals were sacrificed at 21 days post viral infection (dpi) and the number and volume of enlarged lymph nodes was determined, as described in Example 1A. A reduction in the number and volume of enlarged lymph nodes was observed in GLV-1h68-injected animals. In the female mice the number of enlarged lymph nodes decreased from about 5 to 2 enlarged lymph nodes per mouse and the average volume of the enlarged lymph nodes decreased from about 61 mm³ to 20 mm³. In the male mice the number of enlarged lymph nodes decreased from about 4.5 to 3 and the average volume of the enlarged lymph nodes decreased from about 48 mm³ to 12 mm³.

All enlarged lymph nodes were harvested and analyzed for the presence of exogenous PC-3 tumor cells by the detection of mRNA corresponding to the human β-Actin gene by RT-PCR (see Example 1B for experimental details; 11 mice per group were analyzed). There was a significant (p<0.005) reduction of lymph node metastases that were positive for human β-Actin 21 days post GLV-1h68 injection, compared to PBS-injected controls. Specifically, PC-3 cells were detected in 91% (45/49) of PBS-injected mice and 21% (6/28) of mice administered GLV-1h68.

The lungs of 12 PC-3 tumor-bearing mice (6 from the PBS control group and 6 from GLV-1h68 treatment group) were analyzed for the presence of PC-3 cells. Lung tissue was extracted from the mice 21 days following GLV-1h68 or PBS injection and analyzed for human β-Actin, as a marker for PC-3 cells, by RT-PCR as described above for the enlarged lymph nodes. RNA isolation was performed using a standard TRIzol RNA isolation protocol. Human β-Actin was detected in 83% (5/6) mice administered PBS alone, compared to 0% (0/6) of GLV-1h68-injected mice. GLV-1h68 treatment thus resulted in the reduction of hematogenous metastases in lungs in addition to the reduction in lymph node metastases described above.

### Example 10

### Effects of GLV-1h68 Therapy on Blood and Lymphatic Vessel Density

The influence of GLV-1h68 administration on blood and lymph vessel density was examined in PC-3 tumors and metastases. 2×10⁶ PC-3 cells were injected into 6-7 week-old female nude mice using methods described in Example 1 (n=10). At 50 days after cell implantation, 1×10⁷ pfu GLV-1h68 in 100 μL PBS or 100 μL PBS alone were administered by tail vein injection. At 7 dpi, or 57 days post PC-3-implantation, tumors and lumbar and renal lymph nodes were excised from the GLV-1h68 infected (n=5) and PBS control mice (n=5). 100 μm Vibratome sections of the tumors and lumbar and renal lymph nodes were prepared as described in Example 3. The sections of tumors and metastases were stained for CD31 expression, for analysis of blood vessels, with a hamster monoclonal anti-CD31 antibody (Chemicon International, Temecula, Calif.; Cat. No. MAB1398Z) or LYVE-1 expression, for analysis of lymphatic vessels, with a rabbit polyclonal anti-LYVE-1 antibody (Abcam, Cambridge, UK; Cat. No. ab14917) as described in Example 8. Vessel density was calculated as described in Example 8 for four images at 100× magnification from each of 2 sections.

Control mice, administered PBS, exhibited indistinguishable blood vessel density in tumor and lumbar lymph node sections, and slightly increased blood vessel density in the renal lymph nodes, compared to the lumbar lymph nodes (p<0.05) GLV-1h68-administered mice contained similar levels of blood vessel density between the tumor, lumbar lymph node, and renal lymph node tissues. Mice to whom GLV-1h68 was administered exhibited about a 50% reduction in blood vessel density, compared to PBS controls, in each of the three tissues (p<0.001). An identical pattern was observed for LYVE-1 stained sections, except that the lymphatic vessel density was reduced by ⅔ in each of the three tissue types examined (p<0.001).

In summary, by 7 dpi, GLV-1h68 administration significantly reduced the density of blood and lymphatic vessels in tumors and lymph node metastases. As described in Example 9, GLV-1h68 administration also resulted in a significant reduction of the number of lymphatic and hematogenous metastases and the size of metastatic tumors. The reduction of blood and lymph vessel density observed in this study can contribute to the GLV-1h68 metastasis inhibition indirectly by reducing delivery of nutrient and oxygen supplies and/or directly by eliminating pathways for hematogenous and lymphatic metastasis.

### Example 11

### Capture of Circulating Tumor Cells (CTCs) Using A Microfiltration Biochip

In this example, circulating tumor cells (CTCs) were isolated from mouse blood using a microfiltration biochip that captures CTCs based on size and cell deformability.

A. Efficiency of Capturing CTCs from Spiked Mouse Blood 100 μL of blood were drawn from a nu/nu mouse and spiked with 10 μL of DMEM-10 (DME containing 10% fetal bovine serum (FBS)) containing 100-300 PC-3-RFP cells (see Example 2). 80 μL of the spiked blood sample were run through a mounted biochip, CTChip® chip (Clearbridge Biomedics Pte Ltd., Singapore; see, Tan S. J. et al. (2009) Biomedical Microdevices 11(4): 883-892 and Tan et al. (2010) Biosens and Bioelect 26:1701-1705; see, also International PCT application No. WO 2011/109762), at −2000 Pa for 1.5 hours, as a part of a CTC0 Capture System Prototype (Clearbridge Biomedics Pte Ltd., Singapore). The CTChip® chip contains a pre-filter containing filter gaps of about 20 μm in size that receives fluid from a sample inlet. For capture of CTCs, the chip contains three sections of arrays of cell traps. The cell traps are crescent shaped structures with two filter gaps of 5 μm. The cell traps are arranged in staggered rows with alternating left and right tilted orientations. Each cell trap in each row is spaced 50 μm apart, and is offset 25 μm horizontally from a cell trap in the successive row. The chip also contains a waste outlet for untrapped cells and a retrieval outlet to retrieve trapped cells by reversing the pressure differential between the inlet and waste outlets.

RFP and bright field images of cells detained on the biochip were captured and streamed using a camera module coupled to NI USB-6211 data acquisition unit (National Instruments, Austin, Tex.). The captured RFP-positive cells were counted and divided by the number of cells injected to determine the isolation efficiency. The average PC-3-RFP capture efficiency for this system was 82.1%±7.4%, N=3.

B. Capturing CTCs from Mice Bearing PC-3-RFP Tumors

Mice were subcutaneously injected with 5×10⁶ PC-3-RFP cells in the right hind leg. At 44 days after tumor cell implantation, blood was drawn from the mouse via cardiac puncture, and 100 μL of the extracted blood were run through the biochip −2000 Pa for 1.5 hours as described in part A above. Visualization of cells captured by the biochip confirmed that the biochip was capable of isolating CTCs from the blood of mice bearing PC-3-RFP tumors.

At 65 days post tumor cell implantation, blood was drawn via cardiac puncture from the dying mouse, and 70 μL of the blood were run through the CTC0 Capture System Prototype at −2000 Pa for 1.5 hours as described above. Cells were imaged on the chip using an Olympus 1×71 inverted fluorescence microscope (Olympus, Tokyo, Japan), using bright field illumination and red fluorescence detection. Images were captured with an attached MicroFire® True Color Firewire microscope digital charge-coupled device camera (Optronics, Goleta, Calif., USA). A substantial increase in the number of captured CTCs was detected on the biochip, demonstrating that the number of CTCs captured on the biochip is indicative of the severity of the metastasis.

C. Capturing CTCs from Cancer Patient Samples and Spiked Samples in Combination With CTC Marker Immunostaining

1. Capture and Immunostaining of Prostate Cancer CTCs

Peripheral blood samples were obtained from a cancer patient with prostate cancer. 1 mL of the peripheral blood sample was run through the CTC0 Capture System Prototype at −2000 Pa for 15 hours as described above. The microchip captured cells were then immunostained directly on the chip for cytokeratin to confirm the epithelial identity of the cells and the leukocyte marker CD45 as a negative control.

For immunostaining directly on the chip, the flow pressure was adjusted to −200 Pa. The cells were fixed with 4% paraformaldehyde (PFA) for 30 minutes, washed with 1×DPBS for 30 minutes, permeabilized in 20% methanol for 30 minutes, and then washed with 1×DPBS for 30 minutes. The fixed cells were then blocked with 10% goat serum for 30 minutes and stained with PE conjugated anti-CD45 antibody (eBioscience, Cat. 12-0459) and FITC conjugated anti-cytokeratin antibody cocktail (anti-CK8-FITC (eBioscience, Cat. 11-9938), anti-CK18-FITC (Sigma, Cat. F4772), and anti-CK19-FITC (eBioscience, Cat. 11-9898)) for 1 hour. The cells were washed with 1×DPBS for 30 minutes and then stained with 5 μg/mL Hoechst 33342 dye for 30 minutes.

Cells were imaged on the chip using an Olympus 1×71 inverted fluorescence microscope (Olympus, Tokyo, Japan), using bright field illumination and green and red fluorescence detection. Images were captured with an attached MicroFire® True Color Firewire microscope digital charge-coupled device camera (Optronics, Goleta, Calif., USA). The cells captured by the chip were positive for cytokeratin staining, but not CD45 staining, indicating that the captured cells are CTCs and not blood cells. CTC identity also was confirmed by morphological analysis of the phase contrast images of the captured cells and Hoechst staining of the cell nuclei.

2. Capture and Immunostaining of Lung Cancer CTCs

Peripheral blood samples were obtained from a cancer patient with lung cancer. 1 mL of the peripheral blood sample was run through the CTC0 Capture System Prototype at −2000 Pa for 15 hours as described above. The microchip captured cells were then immunostained for cytokeratin to confirm the epithelial identity of the cells and the leukocyte marker CD45 as a negative control.

For immunostaining directly on the chip, the flow pressure was adjusted to −200 Pa. The cells were fixed with 4% paraformaldehyde (PFA) for 30 minutes, washed with 1×DPBS for 30 minutes, permeabilized in 20% methanol for 30 minutes, and then washed with lx DPBS for 30 minutes. The fixed cells were then blocked with 10% goat serum for 30 minutes and stained with PE conjugated anti-CD45 antibody (eBioscience, Cat. 12-0459) and FITC conjugated anti-cytokeratin antibody cocktail (anti-CK8-FITC (eBioscience, Cat. 11-9938), anti-CK18-FITC (Sigma, Cat. F4772), and anti-CK19-FITC (eBioscience, Cat. 11-9898)) for 1 hour. The cells were washed with 1×DPBS for 30 minutes and then stained with 5 μg/mL Hoechst 33342 dye for 30 minutes.

Cells were imaged on the chip using an Olympus 1×71 inverted fluorescence microscope (Olympus, Tokyo, Japan), using bright field illumination and green and red fluorescence detection. Images were captured with an attached MicroFire® True Color Firewire microscope digital charge-coupled device camera (Optronics, Goleta, Calif., USA). The cells captured by the chip were positive for cytokeratin staining, but not CD45 staining, indicating that the captured cells are CTCs and not blood cells. CTC identity also was confirmed by morphological analysis of the phase contrast images of the captured cells and Hoechst staining of the cell nuclei.

3. Capture and Immunostaining of Breast Cancer CTCs

Peripheral blood samples were obtained from a cancer patient with lung cancer. 0.5 mL of the peripheral blood sample was run through the CTC0 Capture System Prototype at −2000 Pa for 15 hours as described above. The microchip captured cells were then immunostained for cytokeratin to confirm the epithelial identity of the cells. For immunostaining directly on the chip, the flow pressure was adjusted to −200 Pa. The cells were fixed with 4% paraformaldehyde (PFA) for 30 minutes, washed with 1×DPBS for 30 minutes, permeabilized in 20% methanol for 30 minutes, and then washed with 1×DPBS for 30 minutes. The fixed cells were then blocked with 10% goat serum for 30 minutes and stained with PE-conjugated anti-CD45 antibody (eBioscience, Cat. 12-0459) and FITC-conjugated anti-cytokeratin antibody cocktail (anti-CK8-FITC (eBioscience, Cat. 11-9938), anti-CK18-FITC (Sigma, Cat. F4772), and anti-CK19-FITC (eBioscience, Cat. 11-9898)) for 1 hour. The cells were washed with 1×DPBS for 30 minutes and then stained with 5 μg/mL Hoechst 33342 dye for 30 minutes.

Cells were imaged on the chip using an Olympus 1×71 inverted fluorescence microscope (Olympus, Tokyo, Japan), using bright field illumination and green and red fluorescence detection. Images were captured with an attached MicroFire® True Color Firewire microscope digital charge-coupled device camera (Optronics, Goleta, Calif., USA). The cells captured by the chip were positive for cytokeratin staining, indicating that the captured cells are CTCs and not blood cells. CTC identity also was confirmed by morphological analysis of the phase contrast images of the captured cells and Hoechst staining of the cell nuclei.

4. Capture and Immunostaining of CTCs from GBM Samples Spiked with PC-3 Tumor Cells

In order to determine whether CTCs could be detected in a blood sample from a cancer patient with glioblastoma multiform (GBM), blood samples from a GBM patient were spiked with PC-3-RFP cells and examined. 0.5 mL of a peripheral blood sample from a cancer patient with GBM was spiked with 10 μL of DMEM-10 (DME containing 10% fetal bovine serum (FBS)) containing 1,000 PC-3-RFP cells.

The samples were run through the CTC0 Capture System Prototype at −2000 Pa for 15 hours as described above. The cells were washed with 1×DPBS for 15 minutes and imaged on the chip using an Olympus 1×71 inverted fluorescence microscope (Olympus, Tokyo, Japan), using bright field illumination and red fluorescence detection. Images were captured with an attached MicroFire® True Color Firewire microscope digital charge-coupled device camera (Optronics, Goleta, Calif., USA). RFP-positive cells were detected on the chip indicating that CTCs can be isolated from the spiked GBM sample.

5. Capture and Immunostaining of CTCs from Healthy Mouse Whole Blood Spiked with PC-3 Tumor Cells

Peripheral blood samples were obtained from a healthy nu/nu mouse. 0.1 mL of the peripheral blood sample was spiked with 10 μL of DMEM-10 (DME containing 10% fetal bovine serum (FBS)) containing 1,000 PC-3-RFP cells. The samples were run through the CTC0 Capture System Prototype at −2000 Pa for 1 hour as described above. The microchip captured cells were then immunostained for cytokeratin to confirm the epithelial identity of the cells.

For immunostaining directly on the chip, the flow pressure was adjusted to −200 Pa. The cells were fixed with 4% paraformaldehyde (PFA) for 30 minutes, washed with 1×DPBS for 30 minutes, permeabilized in 20% methanol for 30 minutes, and then washed with 1×DPBS for 30 minutes. The fixed cells were then blocked with 10% goat serum for 30 minutes and stained with FITC-conjugated anti-cytokeratin antibody cocktail (anti-CK8-FITC (eBioscience, Cat. 11-9938), anti-CK18-FITC (Sigma, Cat. F4772), and anti-CK19-FITC (eBioscience, Cat. 11-9898)) for 1 hour. The cells were washed and then stained with 5 μg/mL Hoechst dye for 30 minutes.

Cells were imaged on the chip using an Olympus 1×71 inverted fluorescence microscope (Olympus, Tokyo, Japan), using bright field illumination and green and red fluorescence detection. Images were captured with an attached MicroFire® True Color Firewire microscope digital charge-coupled device camera (Optronics, Goleta, Calif., USA). The captured cells were positive for cytokeratin staining and RFP, indicating that the captured cells are CTCs and not blood cells. CTC identity also was confirmed by morphological analysis of the phase contrast images of the captured cells and Hoechst staining of the cell nuclei.

### Example 12

### Monitoring GLV-1h68 Therapy by Circulating Tumor Cell (CTC) Capture and Analysis

In this example, PC-3-RFP xenograft tumors were developed in 6-wk-old male nude mice by implanting 5×10⁶ PC-3-RFP cells subcutaneously on the right hind leg. At 48 days after tumor cell implantation, groups of 6 mice each were injected with a single intravenous (tail vein) dose of 5×10⁶ pfu GLV-1h68 in 100 μL PBS or 100 μL PBS only (n=3 for each treatment group). Blood was collected weekly from each mouse via cardiac puncture, and 80 μL of the blood was run through the biochip at −2000 Pa as described in Example 11 to capture and analyze CTCs. The progress of tumor development and the net body weight of the mice also were measured over time post-treatment to compare CTC observations with other symptoms of tumor/disease progression.

First, blood samples were analyzed to determine if GLV-1h68 treatment resulted in changes in the amount of captured CTCs. The % change in captured CTCs from weeks 0-4 post treatment from 80 μL blood are set forth in Table 6. By the second week post treatment (62 days post cell injection), CTCs completely disappeared in two out of three GLV-1h68-treated mice, whereas the PBS treated group maintained an average of nearly 78% more CTCs when compared to the number of CTCs detected before treatment.

Captured CTCs also were analyzed for GLV-1h68 infection at one week after GLV-1h68 treatment by overlaying images taken in the RFP and GFP fluorescence channels using an Olympus 1×71 inverted fluorescence microscope (Olympus, Tokyo, Japan) equipped with a MicroFire® True Color Firewire microscope and a digital charge-coupled device camera (Optronics, Goleta, Calif., USA). Detection of GFP signal indicated infection of the CTCs with GLV-1h68, which encodes the Ruc-GFP fusion protein (see Example 5 and U.S. Pat. Pub. No. US2005/0031643). As expected, no GFP fluorescence was detected in the PBS control group. Co-localization of RFP and GFP signals revealed that 78% of CTCs in mice bearing PC-3-RFP tumors are infected with GLV-1h68 within one week post treatment.

Next, the relative changes in PC-3-RFP tumor volumes were determined, using caliper measurement at the site of tumor cell implantation on a weekly basis (0-5 weeks), for animals treated with GLV-1h68 and compared to those of PBS-treated control animals. Results reporting the average relative change in tumor volume (compared to 48 days post tumor cell injection) are provided in Table 6. PBS-treated animals displayed a steadily increasing average change in tumor volume throughout the course of the study, reaching a final volume increase of 300% at 28 dpi (76 days after tumor implantation). The average change in tumor volume GLV-1h68-treated animals increased to an average of 100% at 7 dpi (55 days post tumor implantation) and remained at that level 14 dpi and then decreased at 21 and 28 dpi until the end of the study.

Because tumor-implanted animals often undergo dramatic weight loss over time, the relative net body weight change was measured for each animal on a weekly basis from 0 to 4 weeks post viral treatment. The three treatment groups measured were: GLV-1h68-treated, PC-3-RFP injected animals; PBS-control, PC-3-RFP animals, and PBS control animals without tumor burden. Results are presented in Table 6. Animals receiving only PBS and no PC-3 cells exhibited no relative change in net body weight percentage. PBS-treated PC-3 tumor-bearing animals exhibited a progressive loss in net body weight over the course of the study. By the end of the study, these animals exhibited an average weight loss of 15% net body weight. The average relative body weight for GLV-1h68-treated tumor-burdened animals decreased by about 11% in the first week post-treatment, but then recovered to about a 4% loss in body weight by the second week post viral treatment. The 4% body loss was maintained through the remainder of the study.

The mice used in this study also were qualitatively assessed for general appearance.

GLV-1h68-treated mice had a generally overall healthier appearance than PBS-treated mice.

TABLE 6
Percent change CTCs ± SEM

| Time post | Relative Change in Number | | Relative Change in | | Relative Change in Net | |
|---|---|---|---|---|---|---|
| treatment | of CTCs (%) | | Tumor Volume (%) | | Body Weight (%) | |
| (days) | PBS Control | GLV-1h68 | PBS Control | GLV-1h68 | PBS Control | GLV-1h68 |
| 0 | 0 | 0 | 0 | 0 | 0 | 0 |
| 7 | 77.9 ± 94.3 | 170.9 ± 121.4 | 65.9 ± 67.7 | 80.5 ± 12.4 | −4.1 ± 8.6 | −11.2 ± 13.3 |
| 14 | 163.9 ± 159.5 | −6.7 ± 92.5 | 98.5 ± 128.9 | 99.7 ± 11.3 | −11.4 ± 8.9 | −3.7 ± 11.3 |
| 21 | 77.5 ± 145.0 | 0.0 ± 173.2 | 208.7 ± 205.1 | 99.7 ± 72.4 | −15.2 ± 4.6 | −3.5 ± 16.3 |

### Example 13

### Effect of EDTA on GLV-1h68 Infectivity and Replication

Because patient blood collection tubes necessarily contain anti-coagulants, such as the chelating agent ethylenediaminetetraacetic acid (EDTA), it was necessary to determine if this reagent has any adverse effects on GLV-1h68 activity. Therefore, the infectivity and replication of GLV-1h68 were tested in the presence of varying concentrations of EDTA. EDTA blood collection tubes used in the study contain 4.8 mM EDTA when filled.

To show the effect of EDTA on virus infectivity, 1×10⁷ pfu GLV-1h68 were added to DMEM-2 containing different concentrations of EDTA-Na₂ (0, 0.2, 1, 4.8, and 48 mM) in triplicate and incubated at 37° C. for 1 hour. The virus was then titrated in CV-1 cells by standard plaque assay. EDTA had no effect on GLV-1h68 infectivity up to 4.8 mM. The infectivity of the virus incubated in 48 mM EDTA was reduced to one half of that achieved in the presence of lesser EDTA concentrations.

The effect of EDTA on GLV-1h68 replication in tumor cells also was tested. 8×10⁴ PC-3-RFP cells were suspended in 0.5 mL DMEM-2 containing 0, 0.2, 1, 4.8, or 48 mM EDTA-Na₂. GLV-1h68 was added at a multiplicity of infection (MOI) of 0.01 or 10 in triplicate and incubated at 37° C. The infected cells were harvested at 24, 48 and 72 hours post infection. The viral titer was then measured using CV-1 cells by standard plaque assay.

At MOI of 0.01, the starting titer of GLV-1h68 was about 1×10⁴ pfu/10⁶ cells. In the presence of 4.8 mM EDTA, the titer remained constant at about 1×10⁴ pfu/10⁶ cells over the course of the study. For lower EDTA concentrations (e.g. 0.2 mM and 1 mM) and the no EDTA control sample, the virus exhibited steadily increasing viral titers over time. In the presence of 48 mM EDTA, no virus was recovered at all time points tested.

At MOI of 10, the starting titer of GLV-1h68 was about 1×10⁷ pfu/10⁶ cells. In the presence of 4.8 mM EDTA, the viral titer again remained constant at about 1×10⁷ pfu/10⁶ cells up to 72 hr. In the presence of 0, 0.2 mM or 1 mM EDTA, the viral titer increased to about 1×10⁸ pfu/10⁶ cells at 24 hr, and remained at that titer at 48 and 72 hours post infection. Incubation in the presence of 48 mM EDTA resulted in decreasing viral titer over time to 1×10⁶ pfu/10⁶ at 24 hours post infection and further decreasing to about 5×10⁵ pfu/10⁶ at 48 and 72 hours post infection.

These results indicate that at concentrations of EDTA present in standard blood collection tubes, vaccinia virus infectivity and replication were not negatively affected.

### Example 14

### Identification of Circulating Tumor Cells (CTCs) using GLV-1h68

In this example, experiments were performed to analyze the use of GLV-1h68 for detection of CTCs in a sample. The ability of GLV-1h68 to specifically infect circulating tumor cells and the capture of GLV-1h68-infected CTCs was demonstrated.

### A. Specificity of Tumor Cell Infection

To show that GLV-168 specifically infects tumor cells, but not other cells contained within blood, blood was drawn from a normal mouse into an EDTA blood collection tube. 100 μL of blood was transferred to a new 1.5 mL microcentrifuge tube. The blood was then spiked with 10 μL of a PC-3-RFP suspension (2.475×10⁵ cells/mL). 1 mL DMEM-2 was then added to the blood/PC-3-RFP cell mixture, and the sample was subjected to centrifugation at 1,000×g for 5 min to pellet the cells. The supernatant was removed and the cells were resuspended in 100 μl DMEM-2. 10 μL it of GLV-1h68 (1.84×10⁹ pfu/mL) was added to the cell suspension and the tube was incubated at 37° C. in a CO₂ incubator 15 hours. The infected cells were transferred into a well of a 24-well plate and visualized using an Olympus 1×71 inverted fluorescence microscope (Olympus, Tokyo, Japan). Images were taken using a MicroFire® True Color Firewire microscope digital charge-coupled device camera (Optronics, Goleta, Calif., USA) using bright field illumination to reveal the location of PC-3-RFP and normal cells, RFP fluorescence to identify the tumor cells, and GFP fluorescence to identify GLV-1h68-infected cells. Overlaying the images demonstrated that GLV-1h68 specifically infected the tumor cells and did not infect normal cells within the mouse blood.

### B. Biochip Capture of Infected Tumor Cells Following In Vitro GLV-1h68 Infection

GLV-1h68-infected mouse blood was run through the Clearbridge microfiltration biochip at −2000 Pa (see Example 11) to show that the biochip captures tumor cells in blood infected with GLV-1h68 in vitro. 40 μL of the GLV-1h68-infected cell suspension from part A above were run through the ClearBridge biochip described in Example 11. Pictures of the biochip containing captured cells were taken using an Olympus inverted fluorescence microscope, equipped with a digital camera as described in part A above, using bright field illumination and RFP and GFP fluorescence. Images of biochip-captured cells showed overlap of the RFP and GFP signals, demonstrating capture of GLV-1h68-infected tumor cells.

C. GLV-1h68 Infection of Tumor-Like Cells from a Gastric Cancer Patient

To determine whether GLV-1h68 could infect tumor-like cells in a sample from a tumor-bearing patient, 1 mL of cerebral spinal fluid (CSF) from a patient with advanced gastric cancer, which had metastasized to the brain, was dispensed in a well of a 24-well tissue culture plate, and infected with 10 μL of GLV-1h68 (1.84×10⁹ pfu/ml) 15 hours. Pictures of infected tumor-like cells were taken as described in part A above, using bright field illumination and GFP fluorescence. Images revealed that that the tumor-like cells were infected with GLV-1h68.

### D. In Situ GLV-1h68 Infection of CTCs Captured on a Microfiltration Biochip

Peripheral blood samples were obtained from a healthy nu/nu mouse. 0.1 mL of the peripheral blood sample was spiked with 10 μL of DMEM-10 (DME containing 10% fetal bovine serum (FBS)) containing 1,000 PC-3-RFP cells. The samples were run through the CTC0 Capture System Prototype at −2000 Pa for 1 hour as described above. The biochip was then washed with DMEM containing 2% FBS for 15 min. The captured cells were infected with 1 mL GLV-1h68 at the concentration of 1×10⁶ pfu/mL and incubated in 37° C. for 36 hours. The cells were then imaged on the chip using an Olympus 1×71 inverted fluorescence microscope (Olympus, Tokyo, Japan), using bright field illumination and green and red fluorescence detection. Images captured with an attached MicroFire® True Color Firewire microscope digital charge-coupled device camera (Optronics, Goleta, Calif., USA) revealed that RFP positive cells also were GFP positive indicating infection of the PC-3-RFP cells by GLV-1h68. GFP expression following virus infection on the chip was slightly delayed compared to virus infection prior to running on the chip due to fluidic stress on the cells.

### Example 15

### In Situ GLV-1h68-Infection of Parylene Microfilter Biochip-Captured Cells

In this example, the USC biochip and CTC detection platform, as described in Xu et al. (2010) Cancer Res 70(16):6420-6426 and U.S. Pat. Pub. No. 2011/0053152, was used to capture tumor and tumor-like cells. The USC biochip system captures tumor and tumor-like cells by size segregation on a parylene-C slot microfilter, using a constant low pressure delivery system. The USC chip employed was 6 mm×6 mm in total size with a membrane thickness of 10 μm and an optimized slot size of 6 μm×40 μm. The ability of GLV-1h68 to infect USC biochip-captured cells in situ was shown using this system.

### A. In Situ GLV-1h68 Infection of Captured GI-101A Cells

Cells of the metastatic breast tumor cell line, GI-101A (Dr. A. Aller, Rumbaugh-Goodwin Institute for Cancer Research, Inc.) were cultured in RPMI 1640 supplemented with 5 ng/mL of β-estradiol and progesterone (Sigma, St. Louis, Calif.), 10 mmol/L HEPES, 1 mmol/L sodium pyruvate, 20% fetal bovine serum (FBS; Mediatech, Inc., Manassas, Va.), and 1% antibiotic-antimycotic solution (Mediatech, Inc., Manassas, Va.) at 37° C. under 5% CO₂. The GI-101A cells (100 cells) were suspended in 2 mL Dulbecco's Phosphate Buffered Saline (DPBS), and run through the USC biochip over 10 minutes. The biochip with captured cells was then immersed into 0.5 mL DMEM2 containing 1×10⁶ pfu/mL GLV-1h68 and incubated 15 hours at 37° C. in a CO₂ incubator. Cells were examined for GLV-1h68 infection by imaging the chip using an Olympus 1×71 inverted fluorescence microscope (Olympus, Tokyo, Japan), using bright field illumination and green fluorescence detection. Images captured with an attached MicroFire® True Color Firewire microscope digital charge-coupled device camera (Optronics, Goleta, Calif., USA) revealed GFP-positive GI-101A cells, indicating in situ GLV-1h68 infection of captured cells.

B. In Situ Infection of Captured Tumor-Like Cells from a Gastric Cancer Patient

To show that tumor-like cells from a patient with advanced gastric cancer can be infected with GLV-1h68 in situ, following capture by the USC biochip, 2 mL of cerebral spinal fluid from a patient with metastatic gastric cancer were run through a USC biochip. The chip with captured cells was then incubated in 0.5 mL DMEM containing 1×10⁶ pfu GLV-1h68/ml, in a well of a 24-well plate. The chip was incubated and imaged as described in part A above. Analysis of the captured cells by bright field microscopy revealed that the USC biochip captured a mixture of small and large cells. GLV-1h68-infected cells, detectable by GFP fluorescence, indicated that the larger, tumor-like cells were successfully infected in situ.

The GLV-1h68-infected cells were further analyzed by staining nuclei with 4′,6-diamidino-2-phenylindole (DAPI) and by immunofluorescence using antibodies directed against carcinoembryonic antigen (CEA), which is glycoprotein involved in cell adhesion that is upregulated in cancer and is employed as a marker for identification of tumor cells. The infected cells were fixed with 4% paraformaldehyde for 10 min at room temperature, washed with DPBS, and incubated with PE-conjugated anti-CEA antibody (1:100 dilution, BD Biosciences)/DAPI (5 μg/mL) for one hour at room temperature. Imaging was performed as described above using an Olympus 1×71 inverted fluorescence microscope (Olympus, Tokyo, Japan) equipped with a MicroFire® True Color Firewire microscope digital charge-coupled device camera (Optronics, Goleta, Calif., USA). Comparing bright field, GFP, DAPI, and CEA immunofluorescent images showed that the GLV-1h68 (GFP-positive) cells also were CEA-positive, indicating that the infected cells were tumor cells.

### Example 16

### In Situ GLV-1h68-Infection of CellSieve™ Microfilter Captured Cells

### A. CellSieve™ MicroFilter Capture and Immunostaining of PC-3 Tumor Cells

1,000 PC-3-RFP cells in 10 μL were added to 1 mL 1×DPBS and processed by CellSieve™ Microfilters (Creatv MicroTech, Inc. Potomac, Md.). The CellSieve™ microfilter is a polymer filter that has a thickness of 10 μm and contains rows of pores, 7-8 μm in diameter, with a pore periodicity of 20 μm. Adjacent rows of pores are offset by 10 μm.

The 1 mL sample was placed into a syringe attached to a filter holder containing the microfilter. The sample was drawn through the filter by negative pressure according to the manufacturer's instructions. The microfilter was remove from the filter holder and place in microwell plate for staining. The captured cells were fixed with fixation buffer (Creatv MicroTech, Inc.) for 20 minutes, permeabilized in permeabilization buffer (Creatv MicroTech, Inc.) for 20 minutes and washed with 1×DPBS. The cells were then stained with FITC-conjugated anti-cytokeratin antibody cocktail (anti-CK8-FITC (eBioscience, Cat. 11-9938), anti-CK18-FITC (Sigma, Cat. F4772), and anti-CK19-FITC (eBioscience, Cat. 11-9898)) and 5 μg/mL Hoechst solution for 1 hour and then washed with 1×DPBS. The filter was transferred onto a microscope slide and imaged on the filter using an Olympus 1×71 inverted fluorescence microscope (Olympus, Tokyo, Japan), using bright field illumination and green and red fluorescence detection. Images were captured with an attached MicroFire® True Color Firewire microscope digital charge-coupled device camera (Optronics, Goleta, Calif., USA). Imaging showed that CTCs can be captured by the chip at ˜60% capture efficiency and the cells that are captured also are cytokeratin positive.

### B. In Situ GLV-1h68 Infection of Captured PC-3 Cells

1,000 PC-3-RFP cells in 10 μl were added to 1 mL 1×DPBS and processed by CellSieve™ Microfilters (Creatv MicroTech, Inc. Potomac, Md.) as described in Part A. After the cells were captured, the filter was placed in a well of a 24-well with 0.2 mL DMEM-2% FBS containing 1×10⁶ pfu GLV-1h68. The plate was incubated at 37° C. in a CO₂ incubator (5% CO₂) for 15 hours. Then the filter was transferred onto a microscope slide and imaged on the filter using an Olympus 1×71 inverted fluorescence microscope (Olympus, Tokyo, Japan), using bright field illumination and green and red fluorescence detection. Images were captured with an attached MicroFire® True Color Firewire microscope digital charge-coupled device camera (Optronics, Goleta, Calif., USA). The RFP positive cells captured by the microfilter also were positive for GFP expression indicating that the GLV-1h68 virus can infect CTCs in situ on the microfilter.

C. In Situ GLV-1h68 Infection of Captured CTCs from Mice Bearing a PC-3 Xenograft Tumor

Mice were subcutaneously injected with 5×10⁶PC-3-RFP cells in the right hind leg. At 44 days after tumor cell implantation, blood was drawn from the mouse via cardiac puncture, and 100 μL of the extracted blood was processed by a CellSieve™ Microfilter as described in Part A. After the cells were captured, the filter was placed in a well of a 24-well with 0.2 mL DMEM-2% FBS containing 1×10⁶ pfu GLV-1h68. The plate was incubated at 37° C. in a CO₂ incubator (5% CO₂) for 15 hours. Then the microfilter was transferred onto a slide and imaged on the filter using an Olympus 1×71 inverted fluorescence microscope (Olympus, Tokyo, Japan), using bright field illumination and green and red fluorescence detection. Images were captured with an attached MicroFire® True Color Firewire microscope digital charge-coupled device camera (Optronics, Goleta, Calif., USA). The RFP positive cells captured by the microfilter also were positive for GFP expression indicating that the GLV-1h68 virus can infect CTCs in situ on the microfilter.

### Example 17

Generation of TurboFP635 Vaccinia Virus Strains

In this Example vaccinia virus strains expressing the far-red fluorescent protein TurboFP635 (scientific name “Katushka”) from the sea anemone Entacmaea quadricolor (Shcherbo et al. (2007) Nat Methods 4(9):741-746) were generated. TurboFP635 has an excitation/emission maxima at 588/635 nm and is 7 to 10-fold brighter compared to other far-red fluorescent proteins such as HcRed (Gurskaya et al. (2001) FEBS Lett. 507(1):16-20) or mPlum (Wang et al. (2004) Proc Natl Acad Sci USA. 101 (48):16745-16749). TurboFP635 also exhibits a fast maturation rate which makes it useful for expression by vaccinia virus for rapid detection of infected CTCs. In addition, the excitation/emission profile for TurboFP635 minimizes autofluorescence for imaging CTCs directly on microfilters and biochips (e.g. microfluidic devices).

### A. Construction of Modified Vaccinia Viruses

Modified vaccinia viruses containing DNA encoding TurboFP635 (SEQ ID NO:21 (protein); SEQ ID NO:20 (DNA)) were generated by removing and inserting nucleic acid at the hemagglutinin (HA) gene locus in a vaccinia virus genome. The heterologous DNA inserted into the virus genome included expression cassettes containing protein-encoding DNA operably linked to a vaccinia virus promoter.

The starting strains used for the construction of the modified vaccinia viruses were vaccinia virus (VV) strain GLV-1h68 (also named RVGL21, SEQ ID NO:1) and GLV-1h71 (see U.S. Patent Publication No. US2009/0098529). GLV-1h68, contains DNA insertions in the F14.5L, thymidine kinase (TK) and hemagglutinin (HA) genes and is described in U.S. Patent Publication No. 2005/0031643. GLV-1h71 is a derivative strain of GLV-1h68, that contains DNA insertions in the thymidine kinase (TK) and hemagglutinin (HA) genes and a deletion of the insertion at the F14.5L locus.

GLV-1h68 was prepared from the vaccinia virus strain designated LIVP, which is a vaccinia virus strain, originally derived by adapting the vaccinia Lister strain (ATCC Catalog No. VR-1549) to calf skin (Research Institute of Viral Preparations, Moscow, Russia, Al'tshtein et al. (1983) Dokl. Akad. Nauk USSR 285:696-699). The LIVP strain, whose genome sequence is set forth in SEQ ID NO:2 and from which GLV-1h68 was generated, contains a mutation in the coding sequence of the TK gene, in which a substitution of a guanine nucleotide with a thymidine nucleotide (nucleotide position 80207 of SEQ ID NO:2) introduces a premature STOP codon within the coding sequence.

As described in U.S. Patent Publication No. 2005/0031643 (see, particularly, Example 1 of the application), GLV-1h68 was generated by inserting expression cassettes encoding detectable marker proteins into the F14.5L (also referred to as F3; see U.S. Patent Publication No. 2005/0031643), thymidine kinase (TK; J2R), and hemagglutinin (HA; A56R) gene loci of the vaccinia virus LIVP strain. Specifically, an expression cassette containing a Ruc-GFP cDNA (a fusion of DNA encoding Renilla luciferase and DNA encoding GFP) under the control of a vaccinia synthetic early/late promoter P_{SEL} was inserted into the F14.5L gene; an expression cassette containing DNA encoding beta-galactosidase under the control of the vaccinia early/late promoter P_{7.5k} (denoted (P_{7.5k})LacZ) and DNA encoding a rat transferrin receptor positioned in the reverse orientation for transcription relative to the vaccinia synthetic early/late promoter P_{SEL} (denoted (P_{SEL})rTrfR) was inserted into the TK gene (the resulting virus does not express transferrin receptor protein since the DNA encoding the protein is positioned in the reverse orientation for transcription relative to the promoter in the cassette); and an expression cassette containing DNA encoding β-glucuronidase under the control of the vaccinia late promoter P₁₁ₖ (denoted (P₁₁ₖ)gusA) was inserted into the HA gene.

Insertion of the expression cassettes into the LIVP genome to generate the GLV-1h68 strain resulted in disruption of the coding sequences for each of the F14.5L, TK and HA genes. Accordingly, all three genes in the resulting strains are nonfunctional in that they do not encode the corresponding full-length proteins. As described in U.S. Patent Publication No. 2005/0031643, disruption of these genes not only attenuates the virus, but also enhances its tumor-specific accumulation. Previous data have shown that systemic delivery of the GLV-1h68 virus in a mouse model of breast cancer resulted in the complete eradication of large subcutaneous GI-101A human breast carcinoma xenograft tumors in nude mice (see U.S. Patent Publication No. 2005/0031643).

As described in U.S. Patent Publication No. US2009/0098529 (see, particularly, Example 1 of the application), GLV-1h71 was generated by insertion of short non-coding nucleic acid in place of the Ruc-GFP expression cassette at the F14.5L locus of GLV-1h68.

1. Modified Viral Strains

Modified recombinant vaccinia viruses containing heterologous DNA inserted into one or more loci of the vaccinia virus genome were generated via homologous recombination between DNA sequences in the vaccinia virus genome and a transfer vector, using methods described herein and known to those of skill in the art (see, e.g., Falkner and Moss (1990) J. Virol. 64:3108-2111; Chakrabarti et al. (1985) Mol. Cell. Biol. 5:3403-3409; and U.S. Pat. No. 4,722,848). In these methods, the existing target gene in the starting vaccinia virus genome is replaced by an interrupted copy of the gene contained in the transfer vector through two crossover events: a first crossover event of homologous recombination between the vaccinia virus genome and the transfer vector and a second crossover event of homologous recombination between direct repeats within the target locus. The interrupted version of the target gene that is in the transfer vector contains the insertion DNA flanked on each side by DNA corresponding to the left portion of the target gene and right portion of the target gene, respectively. The transfer vector also contains a dominant selection marker, e.g., the E. coli guanine phosphoribosyltransferase (gpt) gene, under the control of a vaccinia virus early promoter (e.g., P_{7.5kE}). Including such a marker in the vector enables a transient dominant selection process to identify recombinant virus grown under selective pressure that has incorporated the transfer vector within its genome. Because the marker gene is not stably integrated into the genome, it is deleted from the genome in a second crossover event that occurs when selection is removed. Thus, the final recombinant virus contains the interrupted version of the target gene as a disruption of the target loci, but does not retain the selectable marker from the transfer vector.

Homologous recombination between a transfer vector and a starting vaccinia virus genome occurred upon introduction of the transfer vector into cells that have been infected with a starting vaccinia virus, GLV-1h68 or GLV-1h71. A series of transfer vectors was constructed as described below and the following modified vaccinia strains were constructed: GLV-1h188 (SEQ ID NO:3), GLV-1h189 (SEQ ID NO:4), GLV-1h190 (SEQ ID NO:5), GLV-1h253 (SEQ ID NO:6), and GLV-1h254 (SEQ ID NO:7). The oncolytic reporter virus GLV-1h86, which is described herein and in U.S. Patent Publication 2009/0098529, is a reporter virus that expresses the Ruc-GFP fusion protein and also exhibits a high replication rate and was also employed for in vitro infection. The construction of these strains is summarized in the following Table, which lists the modified vaccinia virus strains, including the previously described GLV-1h68, their respective genotypes, and the transfer vectors used to engineer the viruses:

TABLE 7
Generation of engineered vaccinia viruses

| Name of | Parental | VV Transfer | |
|---|---|---|---|
| Virus | Virus | Vector | Genotype |
| GLV-1h68 | — | — | F14.5L: (P_{SEL})Ruc-GFP |
| | | | TK: (P_{SEL})rTrfR-(P_{7.5k})LacZ |
| | | | HA: (P₁₁ₖ)gusA |
| GLV-1h72 | GLV-1h68 | pCR-TKLR-gpt2 | F14.5L: (P_{SEL})Ruc-GFP |
| | | | TK: SacI-BamHI |
| | | | HA: (P₁₁ₖ)gusA |
| GLV-1h86 | GLV-1h72 | pNCVVhaT | F14.5L: (P_{SEL})Ruc-GFP |
| | | | TK: Sac I-BamHI |
| | | | HA: Hind III-BamHI |
| GLV-1h188 | GLV-1h68 | HA-SE-FUKW2 | F14.5L: (P_{SEL})Ruc-GFP |
| | | | TK: (P_{SEL})rTrfR-(P_{7.5k})LacZ |
| | | | HA: (P_{SE})FUKW |
| GLV-1h189 | GLV-1h68 | HA-SEL-FUKW4 | F14.5L: (P_{SEL})Ruc-GFP |
| | | | TK: (P_{SEL})rTrfR-(P_{7.5k})LacZ |
| | | | HA: (P_{SEL}) FUKW |
| GLV-1h190 | GLV-1h68 | HA-SL-FUKW2 | F14.5L: (P_{SEL})Ruc-GFP |
| | | | TK: (P_{SEL})rTrfR-(P_{7.5k})LacZ |
| | | | HA: (P_{SL}) FUKW |
| GLV-1h71 | | | F14.5L: ko |
| | | | TK: (P_{SEL})rTrfR-(P_{7.5k})LacZ |
| | | | HA: (P₁₁ₖ)gusA |
| GLV-1h253 | GLV-1h71 | HA-SE-FUKW2 | F14.5L: ko |
| | | | TK: (P_{SEL})rTrfR-(P_{7.5k})LacZ |
| | | | HA: (P_{SE}) FUKW |
| GLV-1h254 | GLV-1h71 | HA-SL-FUKW-2 | F14.5L: ko |
| | | | TK: (P_{SEL})rTrfR-(P_{7.5k})LacZ |
| | | | HA: (P_{SL}) FUKW |

GLV-1h188 (SEQ ID NO:3) was generated by insertion of an expression cassette encoding TurboFP635 (far-red fluorescent protein “Katushka”'; FUKW) under the control of the vaccinia P_{SE} promoter into the HA locus of starting strain GLV-1h68 thereby deleting the gusA expression cassette at the HA locus of starting GLV-1h68. Thus, in strain GLV-1h188, the vaccinia HA gene is interrupted within the coding sequence by a DNA fragment containing DNA encoding TurboFP635 operably linked to the vaccinia synthetic early promoter.

GLV-1h189 (SEQ ID NO:4) was generated by insertion of an expression cassette encoding TurboFP635 under the control of the vaccinia P_{SEL} promoter into the HA locus of starting strain GLV-1h68 thereby deleting the gusA expression cassette at the HA locus of starting GLV-1h68. Thus, in strain GLV-1h189, the vaccinia HA gene is interrupted within the coding sequence by a DNA fragment containing DNA encoding TurboFP635 operably linked to the vaccinia synthetic early late promoter.

GLV-1h190 (SEQ ID NO:5) was generated by insertion of an expression cassette encoding TurboFP635 under the control of the vaccinia P_{SL} promoter into the HA locus of starting strain GLV-1h68 thereby deleting the gusA expression cassette at the HA locus of starting GLV-1h68. Thus, in strain GLV-1 h190, the vaccinia HA gene is interrupted within the coding sequence by a DNA fragment containing DNA encoding TurboFP635 operably linked to the vaccinia synthetic late promoter.

GLV-1h253 (SEQ ID NO:6) was generated by insertion of an expression cassette encoding TurboFP635 under the control of the vaccinia P_{SE} promoter into the HA locus of starting strain GLV-1h71 thereby deleting the gusA expression cassette at the HA locus of starting GLV-1h71. Thus, in strain GLV-1h253, the vaccinia HA gene is interrupted within the coding sequence by a DNA fragment containing DNA encoding TurboFP635 operably linked to the vaccinia synthetic early promoter.

GLV-1h254 (SEQ ID NO:7) was generated by insertion of an expression cassette encoding TurboFP635 under the control of the vaccinia P_{SL} promoter into the HA locus of starting strain GLV-1h71 thereby deleting the gusA expression cassette at the HA locus of starting GLV-1h71. Thus, in strain GLV-1h254, the vaccinia HA gene is interrupted within the coding sequence by a DNA fragment containing DNA encoding TurboFP635 operably linked to the vaccinia synthetic late promoter.

2. VV Transfer Vectors Employed for the Production of Modified Vaccinia Viruses

a. HA-SE-FUKW2: For Insertion of an Expression Cassette Encoding FUKW Under the Control of the Vaccinia P_{SE} Promoter into the Vaccinia HA locus

The HA-SE-FUKW2 transfer vector (SEQ ID NO:13) was employed to create vaccinia virus strain GLV-1h188 (SEQ ID NO:3), having the following genotype: F14.5L: (P_{SEL})Ruc-GFP, TK: (P_{SEL})rTrfR-(P_{7.5k})LacZ, HA: (P_{SE})FUKW, and vaccinia virus strain GLV-1h253 (SEQ ID NO: 6), having the following genotype: F14.5L: ko, TK: (P_{SEL})rTrfR-(P_{7.5k})LacZ, HA: (P_{SE})FUKW. HA-SE-FUKW2 contains the TurboFP635 (far-red fluorescent protein “Katushka”; FUKW) gene under the control of the vaccinia P_{SE} promoter, flanked by sequences of the HA gene.

To generate vector HA-SE-FUKW2, cDNA encoding TurboFP635 was PCR-amplified using plasmid FUKW (Dr. Marco J. Herold, University of Wurzburg) as the template with the following primers:

FUKW-5:
(SEQ ID NO: 8; SalI site underlined)
5′-GTCGACCACCATGGTGGGTGAGGATAGCGTGC-3′
FUKW-3:
(SEQ ID NO: 9; PacI site underlined)
5′-TTAATTAATCAGCTGTGCCCCAGTTTGC-3′.

The PCR product was gel-purified, and cloned into the pCR-Blunt II-TOPO vector (SEQ ID NO:17) using Zero Blunt TOPO PCR Cloning Kit (Invitrogen). The resulting construct pCRII-FUKW was sequence confirmed. The TurboFP635 cDNA was then released from pCRII-FUKW with SalI and PacI digestion, and subcloned into same cut vector HA-SE-hNET1 (SEQ ID NO: 10) to generate HA-SE-FUKW2 (SEQ ID NO:13). The resulting HA-SE-FUKW2 construct was confirmed by sequencing.

b. HA-SEL-FUKW4: For Insertion of an Expression Cassette Encoding FUKW Under the Control of the Vaccinia P_{SEL} Promoter into the Vaccinia HA Locus

The HA-SEL-FUKW4 transfer vector (SEQ ID NO:15) was employed to create vaccinia virus strain GLV-1 h189 (SEQ ID NO:4), having the following genotype: F14.5L: (P_{SEL})Ruc-GFP, TK: (P_{SEL})rTrfR-(P_{7.5k})LacZ, HA: (P_{SEL})FUKW. HA-SE-FUKW2 contains the TurboFP635 (FUKW) gene under the control of the vaccinia P_{SEL} promoter, flanked by sequences of the HA gene.

To generate vector HA-SEL-FUKW4, the TurboFP635 cDNA was released from pCRII-FUKW with SalI and PacI digestion, and subcloned into same cut vector HA-SEL-hNET2 (SEQ ID NO:11) to generate HA-SEL-FUKW4 (SEQ ID NO:15). The resulting HA-SE-FUKW4 construct was confirmed by sequencing.

c. HA-SL-FUKW2: For Insertion of an Expression Cassette Encoding FUKW Under the Control of the Vaccinia P_{SL} Promoter into the Vaccinia HA Locus

The HA-SL-FUKW2 transfer vector (SEQ ID NO:13) was employed to create vaccinia virus strain GLV-1h190 (SEQ ID NO:5), having the following genotype: F14.5L: (P_{SEL})Ruc-GFP, TK: (P_{SEL})rTrfR-(P_{7.5k})LacZ, HA: (P_{SL})FUKW, and vaccinia virus strain GLV-1h254 (SEQ ID NO:7), having the following genotype: F14.5L: ko, TK: (P_{SEL})rTrfR-(P_{7.5k})LacZ HA: (P_{SL})FUKW. HA-SL-FUKW2 contains the TurboFP635 (FUKW) gene under the control of the vaccinia Pₘ, promoter, flanked by sequences of the HA gene.

To generate vector HA-SL-FUKW2, the TurboFP635 cDNA was released from pCRII-FUKW with SalI and PacI digestion, and subcloned into same cut vector HA-SL-hNET1 (SEQ ID NO:12) to generate HA-SL-FUKW2 (SEQ ID NO:14). The resulting HA-SL-FUKW2 construct was confirmed by sequencing.

3. Preparation of Recombinant Vaccinia Viruses

African green monkey kidney fibroblast CV-1 cells (American Type Culture Collection (Manassas, Va.); CCL-70) were employed for viral generation and production. The cells were grown in Dulbecco's modified Eagle's medium (DMEM) supplemented with 1% antibiotic-antimycotic solution (Mediatech, Inc., Herndon, Va.) and 10% fetal bovine serum (FBS; Mediatech, Inc., Herndon, Va.) at 37° C. under 5% CO₂. For virus generation of recombinant viruses, the CV-1 cells were infected with the parental virus GLV-1h68 or GLV-1h71 (see Table 7) at MOI of 0.1 for 1 hr. The infected cells were then transfected using Fugene (Roche, Indianapolis, Ind.) with the designated transfer vector (see Table 7 and description of viral transfer vectors above). At two days post infection, infected/transfected cells were harvested and the recombinant viruses were selected and plaque purified using standard methods as described previously (Falkner and Moss (1990) J. Virol. 64:3108-3111).

The genotype of the vaccinia viruses was verified by PCR and restriction enzyme digestion. The lack of expression of the gusA gene for GLV-1h188, GLV-1h189, GLV-1h190, GLV-1h253, and GLV-1h254 was confirmed by standard glucuronidase assay. In vitro infection assays were performed to compare fluorescent protein expression between vaccinia viruses that encode TurboFP635 and the parental GLV-1h68 vaccinia virus that encodes GFP. TurboFP635 and GFP were detectable in cells infected with the respective viruses. In addition, the TurboFP635 signal was stronger and detectable earlier than that of GFP following infection.

### B. Vaccinia Virus Purification

Ten T225 flasks of confluent CV-1 cells (seeded at 2×10⁷ cells per flask the day before infection) were infected with each virus at MOI of 0.1. The infected cells were harvested two days post infection and lysed using a glass Dounce homogenizer. The cell lysate was clarified by centrifugation at 1,800 g for 5 min, and then layered on a cushion of 36% sucrose, and centrifuged at 13,000 rpm in a HB-6 rotor, Sorvall RC-5B Refrigerated Superspeed Centrifuge for 2 hours. The virus pellet was resuspended in 1 mL of 1 mM Tris, pH 9.0, loaded on a sterile 24% to 40% continuous sucrose gradient, and centrifuged at 26,000 g for 50 min. The virus band was collected and diluted using 2 volumes of 1 mM Tris, pH 9.0, and then centrifuged at 13,000 rpm in a HB-6 rotor for 60 min. The final virus pellet was resuspended in 1 mL of 1 mM Tris, pH 9.0 and the titer was determined in CV-1 cells (ATCC No. CCL-70).

### Example 18

### Vaccinia Virus Infection of CTCs in Red Blood Cell (RBC) Cleared Samples

A. In Situ GLV-1h68 Infection of Captured PC-3 Tumor Cells from Spiked Blood Samples Following RBC Lysis

Peripheral blood samples were obtained from a healthy nu/nu mouse by cardiac puncture and collection in anti-coagulant EDTA tubes. 0.1 mL of the peripheral blood sample was transferred to 12×75 culture (VWR, Cat. 60818-565) and spiked with 10 μL of DMEM-10 (DME containing 10% fetal bovine serum (FBS)) containing 1,000 PC-3-RFP cells. Triplicate spiked samples were lysed for red blood cells by adding 1 mL 1×RBC lysis buffer (eBioscience, Cat. 00-4333) to each tube. The samples were incubated at room temperature for 5-10 minutes with occasional shaking. The lysis reaction was stopped by diluting the lysis buffer with 3 mL 1×DPBS (Mediatech, Cat. 10-090-CV) when the blood became clear. The cells were spun down at 300×g at 4° C. and the buffer was carefully removed. The cells were resuspended in 0.5 mL DMEM-2% FBS containing 1×10⁶ pfu GLV-1h68 and incubated at 37° C. in a CO₂ incubator (5% CO₂) 15 hours.

One sample was transferred to a well of a 24-well plate for imaging at 150× magnification on the plates using an Olympus 1×71 inverted fluorescence microscope (Olympus, Tokyo, Japan), using bright field illumination and green and red fluorescence detection. Images were captured with an attached MicroFire® True Color Firewire microscope digital charge-coupled device camera (Optronics, Goleta, Calif., USA).

The second sample was processed by a CellSieve™ Microfilter (Creatv MicroTech, Inc. Potomac, Md.) as described in Example 16. After cell capture, the microfilter was transferred onto a slide and imaged on the filters using an Olympus 1×71 inverted fluorescence microscope (Olympus, Tokyo, Japan), using bright field illumination and green and red fluorescence detection. Images were captured with an attached MicroFire® True Color Firewire microscope digital charge-coupled device camera (Optronics, Goleta, Calif., USA).

The third sample was run through the CTC0 Capture System Prototype (Clearbridge Biomedics Pte Ltd., Singapore) for 1 hour at −2000 Pa as described in Example 11, and the biochip was imaged on the chips using an Olympus 1×71 inverted fluorescence microscope (Olympus, Tokyo, Japan), using bright field illumination and green and red fluorescence detection. Images captured with an attached MicroFire® True Color Firewire microscope digital charge-coupled device camera (Optronics, Goleta, Calif., USA). RFP positive cells in the RBC cleared sample also were GFP positive indicating that GLV-1h68 efficiently infects CTCs in a sample that have been enriched via lysis of RBCs. In addition, RFP positive cells that were captured by either the CellSieve™ Microfilter or CTC Microfiltration Biochip following RBC lysis were GFP positive indicating that the CellSieve™ and CTC Biochip can capture CTCs infected with GLV-1h68 following RBC lysis.

B. GLV-1h190 Infection of CTCs in an RBC-cleared Peripheral Blood Sample from a Colorectal Cancer Patient

Peripheral blood samples were obtained from a human colorectal cancer patient. The blood sample was collected in anti-coagulated tubes and put in slow speed shaker at room temperature before processing. Aliquots of the blood sample were transferred to 50 mL Falcon tubes (1 mL/tube) and 10 mL 1×RBC lysis buffer (eBioscience, Cat. 00-4333) was added to each tube to lyse the RBCs. The sample was incubated for 10-15 minutes at room temperature with occasional shaking. The reaction was stopped by diluting the lysis buffer with 30 mL 1×DPBS (Mediatech, Cat. 10-090-CV) when the blood became clear. The remaining intact cells were spun down with 300×g at 4° C. and the buffer was carefully removed. The cells were resuspended in 1.5 mL DMEM-2% FBS containing 3.15×10⁶ pfu GLV-1h190, which encodes the far-red fluorescent protein TurboFP635 and the Ruc-GFP fusion protein (see Example 17), and incubated at 37° C. in a CO₂ incubator (5% CO₂) overnight.

The cells were then transferred to a well of a 6-well plate for imaging on the slides using an Olympus 1×71 inverted fluorescence microscope (Olympus, Tokyo, Japan), using bright field illumination and green and red fluorescence detection. Images were captured with an attached MicroFire® True Color Firewire microscope digital charge-coupled device camera (Optronics, Goleta, Calif., USA). CTCs expressing GFP and TurboFP635 were readily detectable by green and red fluorescence, respectively, indicating that GLV-1h190 can infect CTCs and permit their detection by green and red fluorescence.

C. GLV-1h254 Infection of CTCs in an RBC-Cleared Peripheral Blood Sample from a Metastatic Breast Cancer Patient

Peripheral blood samples were obtained from a human metastatic breast cancer patient. The RBCs in 1 mL of the blood sample were lysed as described in Part B. The remainder intact cells were pelleted with 300×g at 4° C., and the buffer was carefully removed. The cells were resuspended in 0.5 mL DMEM-2% FBS containing 1×10⁷ pfu GLV-1h254, which encodes the far-red fluorescent protein TurboFP635 (see Example 17), and incubated at 37° C. in a CO₂ incubator (5% CO₂) overnight.

The cells were then spun with 300×g at 4° C. and resuspended in 4% PFA to fix the samples for 30 minutes. The cells were spun down again and washed with 1×DPBS. The samples were incubated with FITC-conjugated EpCAM antibody diluted in 10% goat serum for 30 minutes. The samples were washed with 1×DPBS and stained with Hoechst dye. The stained samples were then washed with 1×DPBS, spun down, resuspended in 1×DPBS, and filtered through a CellSieve™ Microfilter (Creatv MicroTech, Inc. Potomac, Md.) as described in Example 16. After cell capture, the microfilter was transferred onto a slide and imaged under a microscope as described above. Phase contrast, blue, green and red fluorescent images were recorded. CTCs captured by the microfilter were positive for EpCAM expression as detected by green fluorescence and TurboFP635 expression as detected by far-red fluorescence. This indicated that the infected cells captured by the microfilter are CTCs.

### Example 19

### Detection of CTCs in Patient Sample by RBC Lysis and Cytospin

A. Detection of CTCs in a Peripheral Blood Sample from a Lung Cancer Patient

Peripheral blood samples were obtained from a human lung cancer patient. 0.5 mL aliquots of the blood sample were lysed for RBCs as described in Example 18. The remainder intact cells were pelleted with 300×g at 4° C., and the buffer was carefully removed. The RBC-cleared samples were tested for background staining, cytokeratin staining alone, and vaccinia virus infection with cytokeratin staining as indicated below.

To test for background autofluorescence, a first sample of RBC-cleared cells was resuspended in 0.5 mL DMEM-2% FBS and incubated at 37° C. in a CO₂ incubator (5% CO₂) overnight. The cells were then loaded onto assembled cytology funnels (VWR, Cat. 89184-098) and grid slides (Scientific Device, customer designed) and spun down with 1500×rpm for 5 min. The cytology funnels and grid slides were disassembled. The samples were mounted on the grid slides with mounting medium (Vector Laboratories, H-1000) and coverslips were used to protect the samples. Enumerating and imaging the CTCs was performed on the slides using an Olympus 1×71 inverted fluorescence microscope (Olympus, Tokyo, Japan), using bright field illumination and green and red fluorescence detection. Images were captured with an attached MicroFire® True Color Firewire microscope digital charge-coupled device camera (Optronics, Goleta, Calif., USA). No blue, red or green fluorescence was detected in non-infected and unstained cells.

To identify and characterize CTCs by cytokeratin immunostaining in the cancer patient blood sample, a second sample of RBC-cleared cells was resuspended in 0.5 mL DMEM-2% FBS and immunostained with a FITC-conjugated cytokeratin (CK) antibodies and Hoechst 33342 dye. The cells were spun with 300×g at 4° C. and resuspended in 4% PFA to fix the samples for 30 minutes. The cells were spun down again and washed with 1×DPBS. The samples were incubated with FITC-conjugated cytokeratin antibody cocktail (anti-CK8-FITC (eBioscience, Cat. 11-9938), anti-CK18-FITC (Sigma, Cat. F4772), anti-CK19-FITC (eBioscience, Cat. 11-9898)) diluted in 10% goat serum for 30 minutes. The samples were washed with 1×DPBS and stained with 200 μL Hoechst solution (5 μg/ml) The stained samples were then washed with 1×DPBS, spun down, resuspended in 1×DPBS. The cells were then processed by cytospin onto grid slides and imaged as described above. Hoechst staining was detected by UV fluorescence, and CK staining by green fluorescence. CK positive cells among approximately 1 million cells were detected in the cytospun sample indicating that CTCs were present in the sample.

To show that oncolytic viruses, such as vaccinia virus, infect CTCs in the cancer patient blood sample, a third sample of RBC-cleared cells was resuspended in 0.5 mL DMEM-2% FBS containing 1×10⁷ pfu GLV-1h254, which encodes the far-red fluorescent protein TurboFP635 (see Example 17), and incubated at 37° C. in a CO₂ incubator (5% CO₂) overnight. Following infection, the cells were immunostained with a cytokeratin (CK) antibody and Hoechst dye as described above. The cells were then spun down onto slides by cytospin as described above and imaged by phase contrast and fluorescence microscopy. 223 CK positive cells were detected in the cytospun sample, and all 223 CK positive cells also were positive for TurboFP635 signal, indicating that the tumor cells were infected by GLV-1h254. Thus, GLV-1h254 specifically infects CTCs and permits their detection.

B. Detection of CTCs in a Peripheral Blood Sample from a Colorectal Cancer Patient

Peripheral blood samples were obtained from a human patient with colorectal cancer. 1.5 mL aliquots of the blood sample were lysed for RBCs and the remainder intact cells were pelleted as described in Example 18. The RBC-cleared samples were tested for background staining, cytokeratin staining alone, and vaccinia virus infection with cytokeratin staining as indicated below.

To test for background autofluorescence, a first sample of RBC-cleared cells was resuspended in 0.5 mL DMEM-2% FBS and incubated at 37° C. in a CO₂ incubator (5% CO₂) overnight. The cells were then spun down onto slides by cytospin as described in Part A. The cells were imaged by phase contrast microscopy and checked for background fluorescence under a microscope as described above. No blue, red or green background fluorescence was detected in the cytospun cells.

To identify and characterize CTCs by cytokeratin immunostaining in the cancer patient blood sample, a second sample of RBC-cleared cells was resuspended in 0.5 mL DMEM-2% FBS and immunostained with FITC-conjugated anti-cytokeratin (CK) antibodies and Hoechst dye as described in Part A. The cells were then spun down onto slides by cytospin as described above and imaged by phase contrast and fluorescence microscopy. Hoechst staining was detected by UV fluorescence, and CK staining by green fluorescence. CK positive cells were detected in the cytospun sample indicating that CTCs were present in the sample.

To show that vaccinia virus infects CTCs in the cancer patient blood sample, a third sample of RBC-cleared cells was resuspended in 0.5 mL DMEM-2% FBS containing 1×10⁷ pfu GLV-1h254, which encodes the far-red fluorescent protein TurboFP635 (see Example 17), and incubated at 37° C. in a CO₂ incubator (5% CO₂) overnight. Following infection, the cells were immunostained with FITC-conjugated anti-cytokeratin (CK) antibodies and Hoechst dye as described in Part A. The cells were then spun down onto slides by cytospin as described above and imaged by phase contrast and fluorescence microscopy. 6230 CK positive cells were detected in the cytospun sample, and all 6230 CK positive cells also were positive for TurboFP635 signal, indicating that the tumor cells were infected by GLV-1h254. Thus, GLV-1h254 specifically infects CTCs and permits their detection.

C. Detection of CTCs in a Peripheral Blood Sample from a Breast Cancer Patient

Peripheral blood samples were obtained from a human breast cancer patient. 2.0 mL aliquots of the blood sample were lysed for RBCs and the remainder intact cells were pelleted as described in Example 18. The RBC-cleared samples were tested for background staining, cytokeratin staining alone, and vaccinia virus infection with cytokeratin staining as indicated below.

To test for background autofluorescence, a first sample of RBC-cleared cells was resuspended in 0.5 mL DMEM-2% FBS and incubated at 37° C. in a CO₂ incubator (5% CO₂) overnight. The cells were then spun down onto slides by cytospin as described in Part A. The cells were detected by phase contrast microscopy and checked for background fluorescence under a microscope. No blue, red or green background fluorescence was detected in the cytospun cells.

To identify and characterize CTCs by cytokeratin immunostaining in the cancer patient blood sample, a second sample of RBC-cleared cells was resuspended in 0.5 mL DMEM-2% FBS and immunostained with FITC-conjugated anti-cytokeratin (CK) antibodies and Hoechst dye as described in Part A. The cells were then spun down onto slides by cytospin as described above and imaged by phase contrast and fluorescence microscopy. Hoechst staining was detected by UV fluorescence, and CK staining by green fluorescence. 39 CK positive cells were detected in the cytospun sample indicating that CTCs were present in the sample.

To show that vaccinia virus infects CTCs in the cancer patient blood sample, a third sample of RBC-cleared cells was resuspended in 0.5 mL DMEM-2% FBS containing 1×10′ pfu GLV-1h254, which encodes the far-red fluorescent protein TurboFP635 (see Example 17), and incubated at 37° C. in a CO₂ incubator (5% CO₂) overnight. Following infection, the cells were immunostained with FITC-conjugated anti-cytokeratin (CK) antibodies and Hoechst dye as described in Part A. The cells were then spun down onto slides by cytospin as described above and imaged by phase contrast and fluorescence microscopy. 137 CK positive cells were detected in the cytospun sample, and 84 of the CK positive cells also were positive for TurboFP635 signal, indicating infection by GLV-1h254. Thus, GLV-1h254 specifically infects CTCs and permits their detection.

D. Detection of CTCs in a Peripheral Blood Sample from a Prostate Cancer Patient

Peripheral blood samples were obtained from a human prostate cancer patient. 1.65 mL aliquots of the blood sample were lysed for RBCs and the remainder intact cells were pelleted as described in Example 18. The RBC-cleared samples were tested for background staining, cytokeratin staining alone, and vaccinia virus infection with cytokeratin staining as indicated below.

To test for background autofluorescence, a first sample of RBC-cleared cells was resuspended in 0.5 mL DMEM-2% FBS and incubated at 37° C. in a CO₂ incubator (5% CO₂) overnight. The cells were then spun down onto slides by cytospin as described in Part A. The cells were detected by phase contrast microscopy and checked for background fluorescence under a microscope. No blue, red or green background fluorescence was detected in the cytospun cells.

To identify and characterize CTCs by cytokeratin immunostaining in the cancer patient blood sample, a second sample of RBC-cleared cells was resuspended in 0.5 mL DMEM-2% FBS and immunostained with FITC-conjugated anti-cytokeratin (CK) antibodies and Hoechst dye as described in Part A. The cells were then spun down onto slides by cytospin as described above and imaged by phase contrast and fluorescence microscopy. Hoechst staining was detected by UV fluorescence, and CK staining by green fluorescence. CK positive cells were detected in the cytospun sample indicating that CTCs were present in the sample.

To show that vaccinia virus infects CTCs in the cancer patient blood sample, a third sample of RBC-cleared cells was resuspended in 0.5 mL DMEM-2% FBS containing 1×10⁷ pfu GLV-1h254, which encodes the far-red fluorescent protein TurboFP635 (see Example 17), and incubated at 37° C. in a CO₂ incubator (5% CO₂) overnight. Following infection, the cells were immunostained with FITC-conjugated anti-cytokeratin (CK) antibodies and Hoechst dye as described in Part A. The cells were then spun down onto slides by cytospin as described above and imaged by phase contrast and fluorescence microscopy. 844 CK positive cells were detected in the cytospun sample, and all 844 CK positive cells also were positive for TurboFP635 signal, indicating that the tumor cells were infected by GLV-1h254. Thus, GLV-1h254 specifically infects CTCs and permits their detection.

E. Detection of CTCs in a Peripheral Blood Sample from a Metastatic Breast Cancer Patient

Peripheral blood samples were obtained from a human prostate cancer patient. 1 mL aliquots of the blood sample were lysed for RBCs and the remainder intact cells were pelleted as described in Example 18. The RBC-cleared samples were tested for vaccinia virus infection with cytokeratin and EpCAM staining as indicated below.

To show that vaccinia virus infects CTCs in the cancer patient blood sample, the sample of RBC-cleared cells was resuspended in 0.5 mL DMEM-2% FBS containing 1×10⁷ pfu GLV-1h254, which encodes the far-red fluorescent protein TurboFP635 (see Example 17), and incubated at 37° C. in a CO₂ incubator (5% CO₂) overnight. Following infection, the cells were immunostained with PE (phycoerythrin)-conjugated anti-pan cytokeratin (CK) antibody (Abcam, Cat. ab52460), a FITC-conjugated EpCAM antibody and Hoechst dye as described in Part A.

The cells were then spun down onto slides by cytospin as described above and imaged by phase contrast and fluorescence microscopy. CK positive cells were detected in the cytospun sample, and CK positive cells also were positive for EpCAM staining and the TurboFP635 signal, indicating that the tumor cells were CTCs that were infected by GLV-1h254. Thus, GLV-1h254 specifically infects CTCs and permits their detection.

### Example 20

### Examination of General Characteristics of Breast Cancer Cell Lines

### A. Clinical and Pathological Features of Tumors Used to Derive Various Breast Cancer Cell Lines

Several features of the breast cancer cell lines used in subsequent experiments are set forth in Table 8 below. These features include source, clinical and pathological features of tumor from which the cancer cell lines were derived according to published data (Neve et al. (2006) Cancer Cell 10(6): 515-527). Included in the Table is molecular profiling information, such as the similarity of gene expression (Gene cluster) of the given tumor cell to the luminal (Lu) or basal B (BaB) epithelium, and the expression of estrogen receptor (ER), progesterone receptor (PR), and human epithelial growth factor receptor 2 (HER2), and tumor protein 53 (TP53; also called P53). Square brackets indicate that levels are inferred from mRNA levels alone where protein data is not available. In some cases, mRNA was present, but the protein was undetectable. This is designated by an asterisk (*) in the table below. The mutational status of TP53 (WT, wild-type protein; M, mutant protein) also is listed (obtained from the Wellcome Trust Sanger Institute listing of tumor cell lines; sanger.ac.uk/perl/genetics/CGP/core_line_viewer?action=cell_lines). Also listed are the source of the tumor: XG, xenograft; PE, pleural effusion; PBr, primary breast, the tumor type: AC, adenocarcinoma; IDC, invasive ductal carcinoma; InfDC, inflammatory ductal carcinoma, and the tumorigenicity of the cell lines.

TABLE 8
Features of Breast Cancer Cell Lines

| | Gene | | | | | | Tumor | |
|---|---|---|---|---|---|---|---|---|
| Cell Line | Cluster | ER | PR | HER2 | TP53 | Source | Type | Tumorigenicity |
| GI-101A | Lu | +/−* | − | + | +^{M} | XG | IDC | Y |
| MCF7 | Lu | + | [−] | low | +/−^{WT} | PE | IDC | Y** |
| MDA-MB-231 | BaB | − | [−] | − | ++^{M} | PE | AC | Y |
| Hs 578T | BaB | − | [−] | | +^{M} | PBr | IDC | N |
| SUM149T | BaB | [−] | [−] | | [+] | PBr | InfDC | Y |

Abbreviations: AC, adenocarcinoma; BaB Basal B; IDC, invasive ductal carcinoma; InfDC, inflammatory ductal carcinoma; ER, estrogen receptor; M, mutant protein; PBr, primary breast; PE, pleural effusion; PR, progesterone receptor; PR, progesterone receptor; TP53, tumor protein 53; WT, wild-type protein; XG, xenograft
[ ] levels inferred from mRNA alone
*positive for mRNA and negative for protein
**with estrogen supplement

### B. Side Population Cells in Human Breast Cancer Cell Lines

In this example, a selection of the human breast cancer cell lines, MCF-7 and GI-101A, were tested for the presence of cancer stem cells using a Hoechst 33342 staining and flow cytometry protocol developed by Goodell et al. (1996) J Exp Med 183:1797-1806. Cancer stem cells have the ability to efflux Hoechst 33342, which is lipophilic DNA binding dye. ATP-binding cassette (ABC) transporters like P-glycoprotein/ABCB1/MDR1 or ABCG2/BCRP, which are preferentially expressed in cancer stem cells, causes the Hoechst dye to efflux mediating a side population phenotype. When excited at a wavelength of 352 nm in a flow cytometer equipped with a UV laser, the Hoechst dye emits in two wavelengths, Hoechst blue (450/20 nm) and Hoechst red (670/40 nm). The cancer stem cells stand out as a distinct and small side population of cells, as compared to the rest of the cells, having low Hoechst emission characteristics, which indicates low levels of the dye within these cells. Distinct regions of the cell population profile also mark different phases of the cell cycle (G0/G1, S, G2/M).

Cells from mouse bone marrow and the A549 lung cancer cell line served as positive controls for comparing to the breast cancer cell lines. Bone marrow was obtained from femurs of one week old C57BI/6 mice. Cells were detached with Accutase and cultured in DMEM+(supplemented with 2% FBS and 10 mM Hepes).

Cells were stained with Hoechst 33342 by adding 1 mg/mL Hoechst 33342 to a final concentration of 5 μg/mL and incubated at 37° C. for 90 minutes. After 90 minutes, the cells were collected by centrifugation and resuspended in cold HBSS+ and maintained at 4° C. to inhibit efflux of the Hoechst dye. Subsets of each of the cell samples, bone marrow cells and MCF-7 and GI-101A cell lines, were treated with the calcium channel blocker Verapamil, at a concentration of 50 μM or 100 μM, during Hoechst staining to prevent the nuclei from pumping out the Hoechst dye. For the A549 lung cancer cell line, a subset of cells was treated with 25 μM, 50 μM, 100 μM, or 200 μM Verapamil. Subsets of A549 cells also were treated with 25 μM, 50 μM, 100 μM, or 200 μM of the selective ATP-binding cassette sub-family G member 2 (ABCG 2) inhibitor Fumitremorgin C (FTC) or 12.5 μM, 25 μM, 50 μM, or 200 μM reserpine, which also block the efflux of the Hoechst dye.

The stained cells were analyzed using a Beckman Coulter Cell Lab Quanta SC flow cytometer equipped with a UV excitation laser and filters enabling the detection of Hoechst blue (450 nm; Hoe450) and Hoechst red (670 nm; Hoe670) emission. Samples were excited at 365 nm and blue fluorescence was collected with 465 bandpass (BP) filter and red fluorescence with a 670 nm edge filter long pass (EFLP) A 550 nm dichroic long pass (DLP) filter was used to separate the emission wavelengths. Hoe450 vs Hoe670 were plotted as the cells were run through the flow cytometer. Side populations were defined as the population of cells that was blocked by the use of Verapamil.

The one week mouse bone marrow sample yielded a side population that accounted for 4.52±0.2% of the registered events. The side population was confirmed by its reduction to 3.74±0.3% and 1.76±0.1% in the presence of 50 and 100 μM Verapamil, respectively. The A549 lung cancer cell line yielded a similar ratio of side population cells (4.49±0.3%) as the mouse bone marrow, and was reduced to 3.95±0.05%, 3.76±0.01%, 2.56±0.2%, or 1.79±0.04% when treated with 25 μM, 50 μM, 100 μM, or 200 μM Verapamil, respectively, 1.51±0.1%, 0.56±0.08%, 0.51±0.01%, or 0.48±0.06% when treated with 12.5 μM, 25 μM, 50 μM, or 200 μM reserpine, respectively, and 1.31±0.2%, 0.92±0.2%, 0.62±0.1%, or 0.46±0.08% when treated with 25 μM, 50 μM, 100 μM, or 200 μM FTC, respectively. In comparison, the side populations for the MCF-7 and GI-101 cells included 0.77±0.1% and 2.21±0.3% of the total cells, respectively. The gated populations were confirmed to be side population cells by their reduction following Verapamil treatment. For MCF-7 cells, the side population cells reduced to 0.50±0.05% and 0.17±0.08% of the population following 50 and 100 μM Verapamil treatment. Treatment of GI-101A cells with 50 and 100 mM Verapamil reduced the side populations to 0.21±0.05% and 0.17±0.02%, respectively. These results indicate that these human breast cancer cell lines contain very few side population cells, indicating low numbers of cancer stem-like cells.

### Example 21

### Aldehyde Dehydrogenase 1 (ALDH1) Activity in Human Breast Cancer Cell Lines

Aldehyde dehydrogenase (ALDH1) is a useful marker for isolating primitive stem cell populations including normal human mammary stem and progenitor cells as well as transformed tumor-initiating stem cells. In this example, GI-101A, MCF7, MDA-MB-231, Hs 578T, and SUM139PT breast cancer cell lines were tested for activity of the ALDH1 marker using a commercial assay to detect ALDH1 expression and thereby identify tumor stem cells (the ALDEFLUOR® assay (available from Stemcell™ Technologies, Vancouver BC, CA; see Ginestier et al. (2007) Cell Stem Cell 1(5):555-567)). Cells of each type were harvested, resuspended to a concentration of 1×10⁶ cells/mL in ALDEFLUOR® assay buffer containing ALDH substrate (1 μM per 1×10⁶ cells), with or without 50 mM of the specific ALDH1 inhibitor diethylaminobenzaldehyde (DEAB), according to the manufacturer's instructions (ALDEFLUO® kit, Stem Cell Technologies). The ALDH substrate fluoresces upon cleavage by ALDH.

Labeled cell suspensions were then analyzed by flow cytometry analysis using Beckman Coulter Cell Lab Quanta SC flow cytometer, using the green fluorescent channel, as detailed by the ALDEFLUOR® assay protocol (Stem Cell Technologies). ALDEFLUOR® assay fluorescence was excited at 488 nm and fluorescence emission was detected using a standard FITC 530/30 band pass filter. The sorting gates were established using propidium iodide stained cells for viability and the ALDEFLUOR® assay-stained cells treated with DEAB as negative controls.

ALDH1 activity (green fluorescence) vs. event count histograms were generated for cells with (negative control) and without (experimental) DEAB. Histograms from cells exhibiting ALDH1 activity demonstrated fluorescence shifting when comparing the profiles of the cells not treated with DEAB with those of cells that were treated with DEAB. The percent ALDH-positive (ALDH+) cells were calculated for each cell type: 6.43% of GI-101A cells were ALDH+; 0.28% of MCF7 cells were ALDH+; 1.74% of MDA-MB-231 cells were ALDH+; 1.45% Hs 578T cells were ALDH+; and 24.11% of SUM149PT cells were ALDH+.

To supplement the flow cytometric analysis, the presence of ALDH in GI-101A was assessed visually by fluorescence microscopy. ALDEFLUOR® assay-labeled cells, with or without DEAB, were counterstained with the nuclear dye 4′,6-diamidino-2-phenylindole (DAPI). Phase contrast and fluorescent images of the cells were taken using a fluorescence microscope, equipped with the appropriate filters and a digital camera, using a 100× objective. GI-101A cells exhibited ALDH1 activity in a fraction of the cells as indicated by green fluorescence that was reduced in the presence of ALDH inhibitor (DEAB). Thus, the microscopy results confirm the flow cytometry data.

### Example 22

### Isolation of ALDEFLUOR® Assay-Positive GI-101A Population

GI-101A cells were selected for further analysis and characterization of the cancer stem cell-like populations. GI-101A cells were sorted by flow cytometry, using a BD FACS Aria III flow cytometer (BD Biosciences), to isolate the subpopulation of ALDEFLUOR-positive (ALDH+) cells detected in Example 21 above. Dot plots were used to set up the parameters to sort the cells. First, intact cells of similar granularity were gated based on their forward scatter (FSC) vs. side scatter (SSC) profiles to select for viability. Next, single cells were gated based on the dot plot of FSC vs. pulse width. Third, to further exclude doublets, cells were gated based on their SSC vs. pulse width profiles. Gated cells that were then positive for green fluorescence were sorted to isolate the ALDH+(about 6% of the parent population) and ALDH−(about 80% of the patent population) cells based on ALDH activity. The sorted populations were then re-analyzed for green fluorescence to assess the purity and recovery of the ALDH+ and ALDH− populations. Due to the instability of the ALDEFLUOR dye in the cells, the fluorescence intensity of the dye decreases dramatically over time, Thus, the sorted ALDH+ cells contained a final recorded percentage of 60-70% ALDH+ cells.

### Example 23

### Tumorigenic Potential of GI-101A ALDEFLUOR-Positive Cells

GI-101A cells, fractionated into populations of ALDH+ or ALDH− cells as described in Example 22 above, or left unsorted, were examined for relative tumorigenic potential using a mammosphere/tumorsphere formation assay and a nude mouse mammary fat pad xenograft assay.

### A. Mammosphere Formation

GI-101A cells, from unsorted, ALDH1+ sorted, or ALDH1− sorted populations (see Example 22) were plated in ultra low-attachment 96-well plates in serum-free medium supplemented with growth factors (10 ng/mL EGF and 20 ng/mL bFGF added every 4 days) at a cell density of 1, 10, or 100 viable cells per well. The cells were incubated at 37° C., 5% CO₂ for 12 days to determine the capability of the different GI-101A cell populations to form mammospheres, a property of cancer stem cells (Fillmore and Kuperwasser (2008) Breast Cancer Res. 10(2):R25. Epub 2008 March 26; Charafe-Jauffret et al. (2009) Cancer Res. 69(4):1302-13). Mammosphere formation was assessed under a light microscope and imaged at 100× magnification. The number of mammospheres per 100 cells plated were counted for each group of cells. Mammosphere formation was not observed at the 1 and 10 cell densities. Statistical analysis showed the GI-101A ALDEFLUOR® assay-positive cells had significantly higher (P<0.05) mammosphere formation efficiency at the 100 cell/well density than ALDEFLUOR® assay-negative cells, indicating that the ALDH+ cells have higher tumorigenic potential.

### B. In Vivo Tumor Formation

Unsorted, ALDH1+ sorted, or ALDH1− sorted GI-101A cells (see Example 22; 5×10², 5×10³, or 5×10⁴ cells) were resuspended in 10 μL serum-free medium, were added to Matrigel and injected into the mammary fat pads of six week old athymic nu/nu female mice. Tumor occurrence and size were monitored weekly, and tumor volume was calculated using external caliper measurement and the modified ellipsoid formula:

Tumor Volume=(length×width×width)/2

The incidence of tumors per injection site, at 5, 6, 8, and 10 weeks post injection, is set forth in Table 9 below.

TABLE 9
Tumor incidence

| Population | 5 × 10² cells | 5 × 10³ cells | 5 × 10⁴ cells |
|---|---|---|---|
| | Tumors/Injection-Week 5 | | |
| Unsorted | 0/5 | 0/5 | 0/5 |
| ALDH1+ | 0/5 | 0/5 | 3/3 |
| ALDH1− | 0/5 | 0/5 | 0/3 |
| | Tumors/Injection-Week 6 | | |
| Unsorted | 0/5 | 0/5 | 2/5 |
| ALDH1+ | 0/5 | 2/5 | 3/3 |
| ALDH1− | 0/5 | 1/5 | 3/3 |
| | Tumors/Injection-Week 8 | | |
| Unsorted | 0/5 | 1/5 | 5/5 |
| ALDH1+ | 0/5 | 4/5 | 3/3 |
| ALDH1− | 0/5 | 1/5 | 3/3 |
| | Tumors/Injection-Week 10 | | |
| Unsorted | 0/5 | 3/5 | 5/5 |
| ALDH1+ | 0/5 | 5/5 | 3/3 |
| ALDH1− | 0/5 | 5/5 | 3/3 |

In addition to tumor incidence, the volumes of the tumors were measured over time to show the correlation between ALDH1 positively and tumor growth. Injection sites receiving 5×10² cells, regardless of cell phenotype, did not develop tumors. The fat pads injected with 5×10³ and 5×10⁴ALDH1+ cells generated tumors starting after 5 weeks inoculation and displayed the highest frequency of tumor formation in weeks 6, 8, and 10. The tumor sizes generated from the ALDH1+ cells also were dramatically higher compared to the ALDH1-populations. The size and latency of tumor formation correlated with the number of cells injected. 5×10³ and 5×10⁴ ALDH 1+ cells generated tumors more efficiently than ALDH 1− cells. At the end of the study, the median tumor volume for the GI-101A ALDH1+ tumors was approximately 2300 mm³, while the median tumor volume for the unsorted GI-101A tumors was just over 500 mm³ and the ALDH1− tumors was 200 mm³. These results are consistent with the in vitro mammosphere formation experiment.

In summary, ALH1+ GI-101A cells generated greater and more rapid tumor incidence than either the unsorted or ALDH1− GI-101A cells. Further, the tumor volumes resulting from the injection of ALDH1+ cells were greater than those from ALDH1− cells. Together, these results indicate that ALDH1+ cells have tumorigenic potential in vivo.

### Example 24

### Parental Cell Line Reconstitution from Sorted Cells

Cancer stem cells have the ability to self-renew and to differentiate into heterogeneous cell types. In this example, GI-101A cells sorted into populations containing increased (ALDH1+) and reduced (ALDH1−) ALDH1 activity (described in Example 22) or left unsorted, were passaged three times to determine if these cells could reconstitute the parental cell line over time. For each passage, each cell culture was expanded in vitro for 12 days. The ALDH1+ activity was measured by flow cytometry as described in Example 21 for each phenotype and compared to unsorted cells which were grown in parallel at each passage. The fraction of ALDH+ cells over time is set forth in Table 10 below for each condition.

TABLE 10
Percent ALDH+ cells

| Passage Number | Unsorted | ALDH+ | ALDH− |
|---|---|---|---|
| P0 | n/a | 64.2% | n/a |
| P1 | 2.52% | 12.6% | 2.12% |
| P2 | 6.41% | 6.36% | 7.08% |
| P3 | 7.14% | 7.08% | 7.74% |

At the time of sorting, P0, the ALDH+ population contained 64.2% ALDH1+ cells. The percentage of ALDH1+ cells progressively declined over passaging. By P3, the ALDH+ sorted sample contained only 7.08% ALDH+ cells, similar to the unsorted sample which contained 7.14% ALDH+ cells. The ALDH− sorted cell line increased expression of ALDH+ cells from 2.12% ALDH+ cells at P1 to 7.74% ALDH+ cells at P3.

### Example 25

### ALDH+ Cell Chemoresistance and Resistance to Ionizing Radiation

Cancer initiating cells in primary human leukemia and glioblastoma are resistant to chemotherapy. In this example, unsorted, ALDH1+ sorted, or ALDH1− sorted GI-101A cells (see Example 22) were examined for resistance to chemotherapeutic agents and ionizing radiation.

### A. Resistance to Chemotherapeutic Agents

The effects of 5-fluorouracil (5-FU), carboplatin, cisplatin, mitomycin C and salinomycin on cell viability of unsorted, ALDH+ sorted and ALDH− sorted GI-101A cells was examined. 1×10⁴ cells were plated in 96-well plates in 200 μL media per well and incubated for overnight at 37° C. in a 5% CO₂ incubator. The medium was replaced with 200 μL fresh medium containing varying doses of each chemotherapeutic agent (see Table 11 for concentrations tested for each agent) or medium alone. The cells were then incubated for 4 days at 37° C. in a 5% CO₂ incubator.

After incubation with the chemotherapeutic agent, the medium was aspirated and replaced with medium contain 20 to 50 μL of MTT solution for a total volume of 200 The cultures were incubated 4-6 hours at 37° C. in a 5% CO₂ incubator. The MTT solution was then removed and 200 μl stop solution was added to each well and gently mixed to dissolve the formazan crystals. The plate was then read on a microtiter plate reader at 550 to 570 nm absorbance. Absorbance in wells containing the chemotherapeutic agent were compared to untreated control cells.

Results were measured as the percentage of surviving cells compared to the control untreated cells. All samples were analyzed in triplicate. Table 11 represents cell viability at 4 days post treatment with the chemotherapeutic agent. Increasing doses of the chemotherapeutic agents generally decreased the percentage of viable cells in the sample. For all treatments tested, the ALDH+ sorted cells were more resistant to cell killing than the ALDH− sorted cells. For the 5-fluorouracil (5-FU), carboplatin, mitomycin C and salinomycin treatment, the ALDH+ sorted cells also were more resistant than the unsorted cells.

TABLE 11
Cell Viability (%) Following 4-Day Treatment with
Chemotherapeutic Agents

| | 5-FU | | | | |
|---|---|---|---|---|---|
| Population | 0.1 μM | 1 μM | 10 μM | 100 μM | 1 mM |
| Unsorted | 126 | 67 | 111 | 55 | 5 |
| ALDH1+ | 95 | 90 | 100 | 75 | 5 |
| ALDH1− | 60 | 37 | 62 | 35 | 5 |

| | Carboplatin | | | |
|---|---|---|---|---|
| Population | 0.1 μM | 1 μM | 10 μM | 100 μM |
| Unsorted | 92 | 90 | 93 | 7 |
| ALDH1+ | 113 | 121 | 99 | 3 |
| ALDH1− | 64 | 100 | 64 | 2 |

| | Cisplatin | | | | |
|---|---|---|---|---|---|
| Population | 0.1 μM | 1 μM | 10 μM | 100 μM | 1 mM |
| Unsorted | 78 | 95 | 106 | 66 | 0 |
| ALDH1+ | 73 | 65 | 65 | 44 | 0 |
| ALDH1− | 58 | 57 | 51 | 14 | 0 |
| | Mitomycin C | | | | |
| Population | 0.01 μM | 0.1 μM | 1 μM | 10 μM | 100 μM |
| Unsorted | 92 | 91 | 20 | 0 | 0 |
| ALDH1+ | 110 | 111 | 31 | 0 | 0 |
| ALDH1− | 78 | 69 | 17 | 0 | 0 |

| | Salinomycin | | | |
|---|---|---|---|---|
| Population | 0.01 μM | 0.1 μM | 1 μM | 10 μM |
| Unsorted | 36 | 41 | 66 | 0 |
| ALDH1+ | 54 | 55 | 66 | 0 |
| ALDH1− | 23 | 18 | 42 | 0 |

### B. Resistance to Ionizing Radiation

The effect of radiation on cell viability of unsorted, ALDH+ sorted and ALDH-sorted GI-101A cells was examined using an ionizing radiation clonogenic assay. Cells were plated in 35 mm culture dishes, one dish for each ionizing radiation dose. The cells were irradiated at a dosage of 0.5, 1, 2, or 4 Gy as a single fraction using a RS2000 X-ray biological irradiator (Rad Source Technologies Inc.) or received no radiation. The cells were harvested following treatment and counted using a Coulter counter and re-plated at varying densities from 100-10,000 cells per test dish in duplicate. The cells were then incubated at 37° C. in a 5% CO₂ incubator until the control dished formed sufficiently large clones. The medium was then removed and the cells were gently washed with DPBS. 2-3 mL of a 6% glutaraldehyde and 0.5% crystal violet mixture was added to the cell and incubated for 30 minutes. The staining mixture was then removed and the cells were washed with tap water and dried in normal air at room temperature. The colonies were counted, and plating efficiency (PE) and surviving fraction (SF) was calculated according to the following formulae:

Plating Efficiency(PE)=[(No. Colonies Formed)/(No. of cells seeded)]×100%

Surviving Fraction(SF)=[(No. Colonies Formed After Treatment)/(No. of cells seeded)×PE]×100%

To generate the radiation survival curve, the surviving fraction at each radiation dose was normalized to that of the non-irradiated control and curves were fitted using a linear quadratic model (surviving fraction=e^{(-α dose-β dose 2)}, in which α is the number of logs of cells killed per Gy from the linear portion of the survival curve and β is the number of logs of cells killed per [Gy]² from the quadratic component).

At all four radiation dosages tested, the ALDH+ cell population exhibited higher resistance to cell killing by radiation compared to the ALDH− and unsorted cell populations. At the 4 Gy radiation dosage, the resistance of the ALDH+ cells was significantly higher (p<0.05) than the ALDH− and unsorted cells.

### Example 26

ALDH+ Cell Invasiveness

ALDEFLUOR positive breast cancer cells have been reported to have cell invasion ability in vitro, which correlate with their ability to metastasize (Charafe-Jauffret et al (2009) Cancer Res. 69(4):1302-13; Crocker et al (2009) J Cell Mol Med 13(8B):2236-52. In this example, unsorted, ALDH1+ sorted, or ALDH1− sorted GI-101A cells (see Example 22) were examined for cell invasive ability using a CULTREX 96-well Basement Membrane Extract (BME) cell invasion assay kit (Trevigen). Cells were cultured to about 80% confluence. 50,000 cells were required for each assay well. The membrane of the top invasion chamber was coated with 50 μL of 0.1× to 1×BME solution (three chambers were left uncoated for controls) and incubated for 4 hours or overnight at 37° C. in a 5% CO₂ incubator. The cells were harvested, and centrifuged at 250×g for 10 minutes. The supernatant was removed and the cells were washed with 1× wash buffer. The cells were resuspended at a concentration of 1×10⁶ cells/mL of serum free medium. The BME solution was aspirated from the top chambers and 50 μL of cells were added to the top chambers. 150 μL of medium per well was added to the bottom chambers, with or without chemoattractants (10% FBS). The chambers were incubation at 37° C. in a 5% CO₂ incubator for 24 hours. Following incubation media from the top well was aspirated and the top chamber was washed with 100 μL wash buffer. Then the bottom chamber was aspirated and washed twice with 200 μL wash buffer. The top chambers were transferred to the assay chamber plate. 12 μL of Calcein-AM stock solution was added to 10 mL of Cell Dissociation Solution and 150 μL of the mixture was added to the bottom chamber of the assay chamber plate. The cell invasion device was assembled and incubated at 37° C. in a 5% CO₂ incubator for 1 hour. Then the top chamber was removed and the plate was read at 485 nm excitation, 520 emission.

A standard curve was generated by plotting the corrected relative fluorescence units (RFU) on the y-axis against the cell number on the x-axis and inserting the trend line (best fit) equation and R-squared value. The trend line equation was used to determine the number of cells present in each sample well. A standard curve was generated for each cell type. The number of cells was compared for each condition to evaluate relative invasion and the number of invaded cells was divided by the number of starting cells (e.g. 50,000) to determine the percent invasion.

Table 12 presents data for the percentage of cell invasion for each cell population in the presence or absence of the FBS chemoattractant. As shown in the table, the ALDH1+ cells were more invasive than the ALDH− and the unsorted cell populations.

TABLE 12
Percentage Cell Invasion by unsorted, ALDH+ sorted
and ALDH− sorted cells

| Cell Population | +10% FBS | −10% FBS |
|---|---|---|
| Unsorted | 1.8% | 0.8% |
| ALDH1+ | 2.1% | 0.75% |
| ALDH1− | 1.3% | 0.1% |

### Example 27

### CD44 and CD49f Expression in ALDH+ Cell Populations

In breast tumor, a CD44+/CD24^{-/low}/ESA⁺/Lineage⁻ subpopulation was originally identified as the tumorigenic fraction based on the enhanced ability of these cells to form tumors in non-obese diabetic/severe combined immunodeficiency (NOD/SCID) mice when injected at a very low number (Al-Hajj et al. (2003) Proc. Natl. Acad. Sci. USA 100(7):3983-3988). Human breast cancer cell lines contain CD44+/CD24^{-/low}/ESA⁺ cells that have stem cell properties including anchorage-independent growth at clonal densities (self-renewal) and the ability to reconstruct the parental cell fractions, along with in vivo tumorigenicity (Ponti et al. (2005) Cancer Res 65(13):5506-5511; Fillmore et al. (2008) Breast Cancer Res 10(2):R25). CD44+/CD24^{-/low} phenotype also is correlated with the enhanced expression of pro-invasive genes and the ability to form distant metastasis (Abraham et al. (2005) Clin Cancer Res. 11(3):1154-1159; Balic et al. (2006) Clin Cancer Res. 12(19):5615-5621; Sheridan et al. (2006) Cancer Res 8(5):R59). In addition, tumorigenicity of prospective breast CSCs has been linked to the expression of α6 integrin (CD49f) (Cariati et al. (2008) Int J Cancer 122(2):298-304 and β1 integrin (Crowe (2004) BMC Cancer 4:18).

In this example, ALDH1+ sorted, or ALDH1− sorted GI-101A cells (see Example 22) were examined for expression of CD44, CD24 and CD49f by flow cytometry analysis. First an ALDEFLUOR assay was performed on GI-101 cells in the presence or absence of DEAB inhibitor as described in Example 21. The ALDEFLUOR stained cells were then subjected to staining with CD44, CD24 and CD49f antibodies. The ALDEFLUOR stained cells were centrifuged for 10 minutes at 300×g at 4° C. and resuspended to a concentration of 2×10⁷ cells per mL in cold staining buffer. 50 μL it of cells were added to 12×75 round bottoms tubes on ice for each stain. 10 μL diluted antibody mixture was added and incubated for 30-45 minutes in an ice bath to minimize release of the Hoechst dye. One set of ALDEFLUOR stained cells was stained with allophycocyanin (APC)-conjugated mouse anti-human CD44 (BD Biosciences) and R-phycoerythrin (PE)-conjugated mouse anti-human CD24 (BD Biosciences). Another set of ALDEFLUOR stained cells was stained with allophycocyanin (APC)-conjugated mouse anti-human CD44 (BD Biosciences; APC has an excitation/emission maxima of 650 nm/660 nm) and R-phycoerythrin (PE)-conjugated mouse anti-human CD49f (BD Biosciences; PE has an excitation/emission maxima of 496 nm/578 nm). The cells were washed twice with 2 mL staining buffer at 4° C. The cells were resuspended in 400 μL it staining buffer and kept on ice until analyzed by flow cytometry.

ALDEFLUOR-positive (ALDH1+) and ALDEFLUOR-negative (ALDH1−) cells were sorted as described in Example 22 (FITC excitation/emission maxima 494 nm/520 nm). The percentage ALDEFLUOR-positive and ALDEFLUOR-negative cells for each set of staining is shown in Tables 13a and 13c. Flow cytometry was performed to analyze the expression of CD44/CD24 expression and CD44/CD49f expression in ALDEFLUOR-positive versus ALDEFLUOR-negative cells. The percentage of CD44⁺ in ALDEFLUOR-positive cells reached to 97.89% (Table 13b; 0.79%+97.1%) or 94.99% (Table 13d; 0.79%+94.2%). The percentage of CD44⁺ in ALDEFLUOR-negative cells dropped to 85.23% (Table 13b; 2.53%+82.7%) or 81.83% (Table 13d; 4.83%+77.0%). Similarly, the percentage of CD49f⁺ in ALDEFLUOR-positive cells reached 99.09% (Table 13d; 94.2%+4.89%) and the percentage of CD49r in ALDEFLUOR-negative cells dropped to 90.8% (Table 13d; 77.0%+13.8%). There was no significant difference of the CD24⁺ expression between ALDEFLUOR-positive cells (98.92%, Table 13b; 97.1%+1.82%) and ALDEFLUOR-negative cells (96.3%, Table 13b; 82.7%+13.6%). In combination, the percentage of CD44⁺/CD24⁻ in ALDEFLUOR-positive cells (0.79%, Table 13b) was unexpectedly lower than that in ALDEFLUOR-negative cells (2.53%, Table 13b). And the percentage of CD44⁺/CD49f⁺ in ALDEFLUOR-positive cells (94.2%, Table 13d) was higher than that in ALDEFLUOR-negative cells (77.0%, Table 13d).

TABLE 13a

| | ALDH+ | ALDH− |
|---|---|---|
| +DEAB | 0.09% | 99.9% |
| −DEAB | 7.59% | 92.4% |

TABLE 13b

| | CD44⁺/CD24⁻ | CD44⁺/CD24⁺ | CD44⁻/CD24⁺ | CD44⁻/CD24⁻ |
|---|---|---|---|---|
| ALDH+ | 0.79% | 97.1% | 1.82% | 0.25% |
| ALDH− | 2.53% | 82.7% | 13.6% | 1.24% |

TABLE 13c

| | ALDH+ | ALDH− |
|---|---|---|
| +DEAB | 0.09% | 99.9% |
| −DEAB | 8.37% | 91.6% |

TABLE 13d

| | CD44⁺/CD49f | CD44⁺/CD49f⁺ | CD44⁻/CD49f⁺ | CD44⁻/CD49f |
|---|---|---|---|---|
| ALDH+ | 0.79% | 94.2% | 4.89% | 0.12% |
| ALDH− | 4.83% | 77.0% | 13.8% | 4.35% |

### Example 28

### Replication of Vaccinia Virus in ALDH+Cell Populations

In this example, the ability of the vaccinia virus GLV-1h68 to replicated in unsorted, ALDH1+ sorted, or ALDH1− sorted GI-101A cells (Example 22) was shown.

Unsorted, ALDH1+ sorted, or ALDH1− sorted GI-101A cells were plated in 6-well plates and incubated overnight at 37° C. in a 5% CO₂ incubator. GLV-1h68 virus (SEQ ID NO: 1; U.S. Pat. Pub. No. US2005/0031643) was added at a multiplicity of infection (MOI) of 0.01 or 10 in triplicate and incubated at 37° C. in a 5% CO₂ incubator for 1 hour with gentle agitation every 20 minutes. After incubation, the virus solution was aspirated and fresh medium was added. The infected cells were harvested at 1, 18, 24, 48 and 72 hours post infection and viral titer was measured using CV-1 cells by standard plaque assay.

GLV-1h68 exhibited a higher replication rate in the ALDH+ cells compared to the unsorted and ALDH− cell populations at the lower MOI of 0.01 and higher MOI of 10. Viral titer at 72 hours post infection of ALDH+ cells at a MOI of 0.01 was approximately 3 times greater than that of the unsorted and ALDH− cells and approximately 2 times greater at a MOI 10. Thus, GLV-1h68 replicated more efficiently in the ALDH+ GI-101A cells

### Example 29

### Effect of Vaccinia Virus on Growth ALDH+ Xenograft Tumors

In this example, the effects of GLV-1h68 on tumor growth in a mouse xenograft tumor model generated from implantation of unsorted, ALDH1+ sorted, or ALDH1− sorted GI-101A cells are shown.

Xenograft tumors were developed in 6-week-old female nude mice by implanting 50,000 or 5,000 cells (unsorted, ALDH1+ sorted, or ALDH1− sorted GI-101A cells (see Example 22)) mammary fat pad as described in Example 23. For comparison, each mouse was implanted with two different fraction of cells, one in each of the left and right mammary fat pad. For example, one mouse received 50,000 ALDH1+ cells in the right fat pad and 50,000 ALDH1− cells in the left fat pad. In another example, one mouse received 5,000 ALDH1+ cells in the right fat pad and 50,000 ALDH1+ cells in the left fat pad.

At 12 weeks post tumor cell implantation, the mice were injected with a single dose of 5×10⁶ pfu of GLV-1h68 in 100 μL phosphate-buffered saline (PBS) or 100 μL PBS only, delivered retro-orbitally. Analysis of tumor size by caliper measurement was performed weekly at 0, 7, 14, 28, 35, 42, and 49 days post virus infection (dpi). Tumor-bearing mice treated with GLV-1h68 also were visualized by fluorescence whole body imaging.

Tumor growth was significantly inhibited after the virus treatment. In the 5,000 cells/injection group, tumors derived from ALDH1+ cells showed dramatic response upon virus treatment compared to tumors derived from ALDH1− or unsorted cells. In mice bearing ALDH+ (right side) and ALDH− (left side) GI-101A tumors, the GFP signal was stronger in the ALDH+ tumor compared to the ALDH− tumor, indicating that the GLV-1h68 virus primarily infected the ALDH+ tumor. Thus, increased vaccinia virus replication correlated with greater tumor regression in the ALDH+ tumor. In mice bearing tumors generated from 50,000 unsorted GI-101A cells (left flank) and 5,000 unsorted GI-101A cells (right flank), the GFP signal was stronger in the tumor generated from larger number of unsorted GI-101A cells.

### Example 30

### Effect of Epithelial-Mesenchymal Transition (EMT) on Expression Cell Adhesion and Cell Surface Markers in Human Mammary Epithelial Cells

Epithelial-mesenchymal transition (EMT) is a process by which cells lose cell adhesion mediated by repression of the cell adhesion molecule E-cadherin and exhibit an increase in cell mobility. EMT is characteristic of cells undergoing proliferation and is involved in the initiation of metastasis. In this example, EMT was induced in mammary epithelia cells or GI-101A cells by transforming growth factor β (TGF-β) and/or other growth factors. Cell morphology and expression of cell adhesion and surface markers over time was observed using immunostaining and fluorescence microscopy and cell sorting. For this experiment, expression of E-cadherin, vimentin, fibronectin were detected by fluorescence microscopy to confirm EMT transition. CD44 and CD24 expression were analyzed by fluorescence activated cell sorting (FACS).

### A. EMT Induction in Human Mammary Epithelial Cells (HMLE)

Human mammary epithelial cells (HMLE; Dr. Robert A. Weinberg) were plated at a density of about 50% in DMEM/F-12 medium and incubated overnight at 37° C. in a CO₂ incubator. The cells were then cultured in inducing medium (DMEM/F-12 (1:1) medium supplemented with insulin, hydrocortisone, 5% FBS, and 2.5 ng/mL TGF-β1) to induce EMT or non-inducing medium (i.e. without TGF-β1). The inducing medium or non-inducing medium was refreshed every 3 days for 12 days. Following EMT treatment, one set of cells were immunostained for E-cadherin, vimentin and fibronectin expression, three widely used EMT markers. Another set of cells was harvested, stained for CD44 and CD24 expression and analyzed by flow cytometry as described in Example 27.

For immunostaining of E-cadherin, vimentin and fibronectin, cells were fixed and permeabilized on the culture plates and incubated with FITC-conjugated antibodies against E-cadherin, vimentin and fibronectin using a standard cell staining protocol. Following removal of unbound antibody, the cells were counterstained with the nuclear dye 4′,6-diamidino-2-phenylindole (DAPI). After staining, phase contrast and fluorescent images of the cells were taken using a fluorescence microscope, equipped with the appropriate filters and a digital camera, using a 100× objective.

After 12 days, there was significant change of HMLE cells' morphology in the absence or presence of TGF-β1 as observed by phase contrast microscopy. Epithelial and mesenchymal cells have historically been identified on the basis of their unique visual appearance and the morphology of the multicellular structures they create (Shook et al. (2003) Mech. Dev. 120(11):1351-83). The TGF-β1-induced HMLE cells exhibited typical mesenchymal characteristics which are in spindle and irregular shape with migratory protrusions compared to non-induced controls which displayed regularly spaced cell-cell junctions.

Prior to treatment, moderate E-cadherin and low levels of vimentin and fibronectin were detected. At 12 days post-EMT induction, E-cadherin expression was down-regulated in TGF-β1-induced HMLE cells compared to non-induced cells, vimentin expression was up-regulated in induced cells compared to non-induced cells, and fibronectin expression was up-regulated in induced cells compared to non-induced cells.

The flow cytometry analysis showed that prior to TGF-β treatment, most of the cell population in the HMLE sample exhibited the CD44⁻/CD24⁻ phenotype, and the amount of CD44⁺/CD24^{-/low} cells was only 1.1±0.4%. After 12 days EMT induction in the presence of TGF-β1, a large enrichment of CD44⁺/CD24^{-/low} cells was observed (51.9%±3%). The percentage of CD44⁺/CD24^{-/low} cells of the non-induced HMLE cells did not significantly change compared to initial cells: only 3.2±0.8% of the cells were CD44⁺/CD2^{-/low} in the non-induced culture after 12 days, and some enrichment of CD44⁻/CD24⁺ was observed.

### B. EMT Induction in GI-101A Cells

In this experiment, combinations of growth factors were assessed for their ability to induce EMT transition in GI-101A cells. GI-101 cells were plated as in Part A and exposed to various combinations of EGF, bFGF and TGF-β1 in the induction medium: TGF-β1 only, EGF+TGF-β1, EGF+bFGF, and EGF+bFGF+TGF-β1. For this experiment, 2.5 ng/mL TGF-β1, 1 ng/mL of EGF, and/or 10 ng/mL of bFGF was added to the induction medium. Following induction of EMT, the cells were immunostained for E-cadherin, vimentin and fibronectin expression on the plate or analyzed for CD44 and CD24 expression by flow cytometry as described in Part A.

After 12 days of EMT induction, the morphology of cells in the presence of growth factors changed significantly compared to the control. According to the morphology, the growth factor combination of EGF+bFGF+TGF-β1 induced EMT more efficiently than other combinations. Flow cytometry analysis of CD44/CD24 expression showed that GI-101A cell induced by the combination of EGF+bFGF+TGF-β1 exhibited the highest percentage of CD44⁺/CD24^{-/low} cells (10.32%) compared to uninduced cells (1.29%). The percentage of CD44⁺/CD24^{-/low} cells in samples that were treated with other combinations of growth factors also was higher than in non-induced cells (e.g., TGF-β1 only (3.13% CD44⁺/CD24^{-/low} cells), EGF+TGF-β1 (8.36% CD44⁺/CD24^{-/low} cells), and EGF+bFGF (8.75% CD44⁺/CD24^{-/low} cells).

Immunostaining of EMT markers also confirmed EMT transition in the induced cells. E-cadherin expression was down-regulated in EGF+bFGF+TGF-β1-induced GI-101A cells compared to non-induced cells; the vimentin expression was up-regulated in induced cells compared to non-induced cells; and the fibronectin expression was up-regulated in induced cells compared to non-induced cells.

### Example 31

### Chemoresistance of EMT Induced GI-101A Cells

Cancer stem cells (CSCs) are resistant to many current cancer treatments, including chemo- and radiation therapy (Dean et al. (2005) Nat Rev Cancer 5(4): 275-284; Bao et al. (2006) Nature 444(7120): 756-60; Woodward et al. (2007) Proc Natl Acad Sci USA 104(2):618-623; Eyler et al. (2008) J Clin Oncol 26(17): 2839-2845; Li et al. (2008) J Natl Cancer Inst 100(9): 672-679; Diehn et al. (2009) Nature 458(7239): 780-783). This indicates that many cancer therapies, while killing the bulk of tumor cells, may ultimately fail because they do not eliminate CSCs, which survive to regenerate new tumors. The induction of an epithelial-mesenchymal transition (EMT) in normal or neoplastic mammary epithelial cell populations has been shown to result in the enrichment of cells with stem-like properties (Mani et al. (2008) Cell 133(4): 704-715).

To determine whether EMT-induced breast cancer cells were enriched in cancer stem-like cells that display chemo-resistant ability, cells induced with various combinations of growth factors were treated with different doses breast cancer chemotherapeutic drugs: 5-FU: 10⁻⁶ mol/L, 10⁻⁵ mol/L, 104 mol/L, 10⁻³ mol/L; Carboplatin: 10⁻⁷ mol/L, 10⁻⁶ mol/L, 10⁻⁵ mol/L, 10⁻⁴ mol/L; Etoposide: 10⁻⁶ mol/L, 10⁻⁵ mol/L, 10⁻⁴ mol/L, 10⁻³ mol/L; Mitomycin-C: 10⁻⁷ mol/L, 10⁻⁶ mol/L, 10⁻⁵ mol/L, 10⁻⁴ mol/L; and Salinomycin: 10⁻⁷ mol/L, 10⁻⁶ mol/L, 10⁻⁵ mol/L, 10⁻⁴ mol/L. Resistance to cytotoxic agents was measured by incubation with or without the cytotoxic agent followed by assessment of cell viability with an MTT assay as described in Example 25.

Briefly, 1×10⁴ GI-101A cells (induced with EGF, bFGF, and/or TGF-β1 or non-induced as described in Example 30) were plated in 96-well plates in 200 μL, media (inducing or non-inducing media) per well and incubated for overnight at 37° C. in a 5% CO₂ incubator. The medium was replaced with 200 μL fresh medium (inducing or non-inducing media) containing varying doses of each chemotherapeutic agent or medium alone. The cells were then incubated for 4 days at 37° C. in a 5% CO₂ incubator. After incubation with the chemotherapeutic agent, the medium was aspirated and replaced with medium contain 20 to 50 μL of MTT solution for a total volume of 200 μL. The cultures were incubated 4-6 hours at 37° C. in a 5% CO₂ incubator. The MTT solution was then removed and 200 tit stop solution was added to each well and gently mixed to dissolve the formazan crystals. The plate was then read on a microtiter plate reader at 550 to 570 nm absorbance. Absorbance in wells containing the chemotherapeutic agent were compared to untreated control cells. Results were measured as the percentage of surviving cells compared to the control untreated cells. All samples were analyzed in triplicate.

As expected for chemo-resistance of cancer stem-like cells which are enriched in EMT induced GI-101A cells, the growth factor combinations of EGF+bFGF+TGF-β1- and EGF+bFGF-induced GI-101A cells showed significant survival ability compared to other growth factor combination-induced or non-induced cells. EMT cells did not exhibit chemo-resistant ability upon the treatment of Salinomycin, an inhibitor of cancer stem cells (Gupta et al. (2009) Cell 138(4): 645-659).

### Example 32

### EMT Induction of GI-101A Cell Invasion and Migration

As described in Example 30, EMT-induced GI-101A cells exhibited changes in morphology indicative of increased cell motility and migratory capacity. The invasive and migrating ability of EMT-induced cells was examined using a CULTREX 96-well Basement Membrane Extract (BME) cell invasion assay kit (Trevigen). For the assay, GI-101A cells were induced with various combinations of growth factors as indicated in Table 14 for 6 days as described in Example 30, then plated in invasion chambers. The invasion assay was conducted as described in Example 26.

Cells which were undergoing EMT migrated through BME more efficiently than the control cells. As a chemoattractant, 10% Fetal Bovine Serum (FBS) showed less effect on invasion and migration of EMT cells.

TABLE 14
Percentage Cell Invasion by EMT-induced Cells

| Cell Population | +10% FBS | −10% FBS |
|---|---|---|
| Mock | 1.1% | 0.8% |
| TGF-β1 | 3.6% | 3.3% |
| EGF + TGF-β1 | 4.5% | 4.1% |
| EGF + bFGF + TGF-β1 | 3.7% | 3.4% |
| EGF + bFGF | 4.3% | 3.8% |

### Example 33

### Vaccinia Virus Replication in EMT-Induced Cells

In this experiment the ability of vaccinia virus to replicate in EMT-induced cells was examined. GLV-1h68, which encodes GFP, and GLV-1h190, which encodes TurboFP635 (far-red fluorescence “Katushka”) were used to infect HMLE or GI-101A cells undergoing EMT.

HMLE or GI-101A cells were plated in 6-wells plates and incubated overnight at 37° C. in a 5% CO₂ incubator. The cells were then induced in inducing medium containing 2.5 ng/mL TGF-β1 for 12 days as described in Example 30. At 12 days post EMT induction, GLV-1h68 or GLV-1h190 virus was added to the cells at an MOI of 10. GLV-1h68 was used to infect the GI-101A cells and GLV-1h190 was used to infect the HMLE cells. Cell morphology and GFP and far-red fluorescence was monitored at 6, 8, 10 and 12 hours post infection by phase contrast and fluorescence microscopy with the appropriate filters. Images were taken at the same position of the plates every 2 hours at 100× magnification.

After 12 hours post infection of the HMLE cells with GLV-1h190, there were only few HMLE cells of the non-induced culture that expressed TurboFP635 protein as detected by red fluorescence. In contrast, in the TGF-β1-induced cell culture, all mesenchymal type cells expressed the TurboFP635 protein and only few epithelial type cells expressed the far-red fluorescent protein. As confirmed by phase contrast microscopy, there was a sharp contrast between the morphology of the mesenchymal versus epithelial type cells, and GLV-1h190 preferentially infected the EMT induced cells.

At 6 hours to 12 hours post infection of GI-101A with GLV-1h68, the mesenchymal type GI-101A cells expressed GFP earlier and more efficiently compared to epithelia type cells. The cell types were distinguished by phase contrast microscopy. The cytopathogenic effect (CPE) in mesenchymal type cells also was more significant than in epithelia type cells.

These results indicate that the vaccinia virus preferentially replicated in EMT induced cells from normal tissue and tumor cell lines.

### Example 34

### Isolation of a CD44⁺/CD24⁻ ESA⁺ Population from EMT-Induced GI-101A Cells

The intracellular marker profile CD44⁺/CD24⁻/ESA⁺ has been widely used in identification and isolation of human breast cancer stem cell in patient samples, including primary tumors, or cancer cell lines (ESA=epithelial specific antigen, also called EpCAM herein; Al-Hajj et al. (2003) Proc Natl Acad Sci USA 100(7): 3983-3988; Sheridan et al. (2006) Breast Cancer Res 8(5): R59; Fillmore (2008) Breast Cancer Res 10(2): R25; Meyer et al. (2009) Breast Cancer Res 11(6): R82; Ginestier et al. (2007) Cell Stem Cell 1(5): 555-567; Wright et al. (2008) Breast Cancer Res 10(1): R10; Mine et al. (2009) Cancer Immunol Immunother 58(8):1185-1194, Epub Dec. 2, 2008).

CD44⁺/CD24⁻ cells can be enriched by inducing EMT from normal mammary epithelia cells or cancer cell lines, as described in Example 30 and in the art (Mani et al. (2008) Cell 133(4): 704-715; Morel et al. (2008) PLoS One 3(8): e2888; Gupta et al. (2009) Nat Med 15(9): 1010-1012). In this example, CSC populations were isolated from EMT-induced GI-101A cells for further study. CD44⁺/CD24⁻/ESA⁺, CD44⁺/CD24^{mid}/ESA⁺ CD44⁺/CD24⁺/ESA⁺, and CD44⁻/CD24^{all}/ESA⁺ cell populations were isolated and examined.

GI-101A cells were induced to EMT by combination treatment with three growth factors, EGF (5 ng/mL), bFGF (10 ng/mL) and TGF-β1 (2.5 ng/mL) for 12 days as described in Example 30. Then the cells were stained with stained with allophycocyanin (APC)-conjugated mouse anti-human CD44 (BD Biosciences), R-phycoerythrin (PE)-conjugated mouse anti-human CD24 (BD Biosciences), and FITC-conjugated mouse anti-human EpCAM-1/ESA and sorted by using BDFACS Aria III cells sorter. The cells were first electronically gated to exclude dead cells, aggregates and doublets. Cells were first selected for viability (P1) and then for single cells (P2 and P3) based on forward and side scatter plots. Then the CD44/CD24 and ESA antigens were analyzed and selected based on CD44, CD24 and ESA signal intensity. Table 15 shows the relative percentages of the subpopulations for CD44 and CD24 expression. ESA expression in EMT induced GI-101A cells was similar to non-induced cells (98.9% compared to 97.2%). To better study the different fractions of EMT induced cells, the cells were gated into the following four groups for induced and non-induced cells: CD44⁺/CD24⁻/ESA⁺, CD44⁺/CD24^{mid}/ESA⁺, CD44⁺/CD24⁺/ESA⁺, and CD44⁻/CD24^{all}/ESA⁺.

TABLE 15
Percentage Cell Populations for CD44 and CD24
expression in Induced versus Non-induced Cells

| Cell Population | Induced | Non-Induced |
|---|---|---|
| CD44⁺/CD24⁻ | 12.6% | 9.45% |
| CD44⁺/CD24^{mid} | 46.7% | 50.9% |
| CD44⁺/CD24⁺ | 8.54% | 9.45% |
| CD44⁻/CD24^{all} | 12.1% | 12.0% |

### Example 35

### Tumorigenic Potential of EMT-Induced Cell Populations In Vivo

In this Example, the different cells populations isolated in Example 34 were examined for tumorigenic potential in a mouse xenograft model. 10,000 purified CD44⁺/CD24⁻/ESA⁺, CD44⁺/CD24^{mid}/ESA⁺, CD44⁺/CD24⁺/ESA⁺, and CD44⁻/CD24^{all}/ESA⁺ cells were injected with Matrigel into the mammary fat-pad of six-week-old athymic nu/nu nude mice to assess the in vivo tumorigenicity of the cell fractions. Tumor occurrence and tumor size were monitored after injection.

TABLE 16
Tumor Frequency in Mice Injected with ESA+ cell populations

| | Tumors/Injection (10,000 Cells per Injection) | | | | | | | | | |
|---|---|---|---|---|---|---|---|---|---|---|
| | Wk | Wk | Wk | Wk | Wk | Wk | Wk | Wk | Wk | Wk |
| Population | 7 | 8 | 9 | 10 | 11 | 12 | 13 | 15 | 18 | 22 |
| CD44⁺/CD24⁻/ESA⁺ | 0/5 | 1/5 | 2/5 | 2/5 | 2/5 | 3/5 | 3/5 | 3/5 | 2/4 | 2/4 |
| CD44⁺/CD24⁺/ESA⁺, | 1/5 | 1/5 | 1/5 | 1/5 | 2/5 | 3/5 | 4/5 | 4/5 | 3/4 | 3/4 |
| CD44⁺/CD24^{mid}/ESA⁺, | 0/5 | 0/5 | 0/5 | 3/5 | 4/5 | 4/5 | 4/5 | 5/5 | 5/5 | 4/4 |
| CD44⁻/CD24^{all}/ESA⁺ | 0/5 | 0/5 | 0/5 | 0/5 | 0/5 | 0/5 | 0/5 | 0/5 | 1/5 | 1/5 |

The CD44⁺/CD24⁺/ESA⁺ cells initiated tumors earlier than any of the other three fractions, however, the CD44⁺/CD24^{mid}/ESA⁺ cells had higher tumor occurrence than other three fractions. Regarding to the tumor growth potential, the CD44⁺/CD24⁺/ESA⁺ cells showed the tumor growth advantage after 17 weeks xenograft compared to the other three fractions. Between weeks 17 and week 22, the CD44⁺/CD24⁺/ESA⁺ tumors rapidly increased in size from approximately 160 mm³ to 750 mm³, while the CD44⁺/CD24^{mid}/ESA⁺ tumors increased in size from approximately 110 mm³ to 290 mm³, and the CD44⁺/CD24⁻/ESA⁺ tumors increased in size from approximately 100 mm³ to 200 mm³. The CD44⁻/CD24^{all}/ESA⁺ cell exhibited the lowest tumor incidence and growth in this experiment.

### Example 36

### Viral replication in CD44⁺/CD24⁺/ESA⁺ and CD44⁺/CD24⁻/ESA⁺ cells

To test the replication efficiency of vaccinia virus strain GLV-1h68 in sorted EMT-induced GI-101A CD44⁺/CD24⁺/ESA⁺ and CD44⁺/CD24⁻/ESA⁺ cells (Example 34), replication assays were performed as described above (Example 28). The cells were infected with GLV-1h68 at an MOI of 0.01 or 10, followed by determination of viral titers by standard plaque assay at the time points 1, 12, 24, 48 and 72 hours post infection. Average data including standard deviation was calculated for parental GI-101A and CD44⁺/CD24⁻/ESA⁺ cells in comparison to CD44⁺/CD24⁺/ESA⁺ cells. 72 hours post infection, the virus titer of CD44⁺/CD24⁺/ESA⁺ cells was about three times higher than CD44⁺/CD24⁻/ESA⁺ cells upon infection at MOI 0.01 and about eight times higher upon infection at MOI 10.

### Example 37

### Vaccinia Virus GLV-1h68 Treatment of GI-101A CD44⁺/CD24⁺/ESA⁺ Cell-Derived Xenografts

As shown in Example 35, GI-101A CD44⁺/CD24⁺/ESA⁺ cells had more tumorigenic potential in nude mice and vaccinia virus GLV-1h68 replicated more efficiently in the CD44⁺/CD24⁺/ESA⁺ cells compared to the CD44⁺/CD24⁻/ESA⁺ cells. To test the efficacy of oncolytic vaccinia virus to target and kill breast CSCs in vivo, palpable tumors were established in the mammary fat pads of athymic nu/nu nude mice using sorted GI-101A CD44⁺/CD24⁺/ESA⁺, CD44⁺/CD24⁻/ESA⁺, CD44⁺/CD24⁻/ESA⁺, and CD44⁻/CD24^{all}/ESA⁺ cells (Example 34), and unsorted cells with the dose of 10,000 cells/injection. For comparable results, each mouse was implanted two different cell fractions in left and right mammary fat pads (e.g. 10,000 CD44⁺/CD24⁺/ESA⁺ cells in right fat pad and 10,000 CD44⁺/CD24⁻/ESA⁺ cells in left fad pad and 10,000 CD44⁻/CD24^{all}/ESA⁺ cells in right fat pad and 10,000 CD44⁺/CD24^{mid}/ESA⁺ cells in left fad pad). After 22 weeks tumor implantation, each mouse was injected with 5×10⁶ pfu GLV-1h68 virus via the retro-orbital path. Then the tumor size and tumor GFP expression was monitored weekly. The CD44⁺/CD24⁺/ESA⁺ cell-derived tumor showed dramatic response upon virus treatment and tumor growth was significantly inhibited after the virus treatment. The tumor fluorescence images also indicated that infected tumors derived from CD44⁺/CD24⁺/ESA⁺ cells showed a more efficient vaccine virus replication, which correlation with tumor regression. Vaccinia virus treatment did not show any inhibitory effects on tumor growth of CD44⁻/CD24^{all}/ESA⁺ cells and no GFP expression was detected in vivo.

These results indicate that GLV-1h68 is selective and effective for infecting and inhibiting tumor derived from cell populations with high tumorigenic potential.

### Example 38

### Detection of CTC Cells in Treated Human Subjects

Following intravenous injection of the vaccinia virus (3×10⁹ pfu) into a human subject who had colorectal cancer and liver metastases, circulating tumor cells were shown to be infected and were detected 8 days after administration. The patient is part of a clinical trial for which the treatment protocol is as follows (Table 17).

TABLE 17
Treatment protocol

| | Number of treatment days, | |
|---|---|---|
| | starting day 1, day 29, day 57, | |
| Dose per day | day 85, day 113 and day 141 *** | |
| 3 × 10⁹ pfu | 1 day | Final volume of |
| 1.667 × 10⁹ | 3 days | preparation is be |
| pfu | | 50 mL, to be infused |
| | | within 30 minutes |

*** Patients who demonstrate a complete response after 12 weeks are not retreated. Patients with stable disease or a partial response after 12 weeks of treatment will continue to receive repeat dosing in 28-day cycles if they have Grade 2 or less drug-related toxicities.

Treated patients are those with high Circulating Tumour Cells (CTC) levels (>10) with solid tumors (e.g., prostate, colorectal or breast cancer) whose disease can be safely serially biopsied. CTCs are measured the same time as the baseline biopsy, and further circulating tumor cell counts and analyses occur on Cycle 1 Day 8 (±3 days), and prior to dosing on Day 1 of Cycles 2, 3, and 4 to evaluate anti-tumour efficacy and viral delivery.

### Example 39

### Detecting/Isolating Tumor Cells with Antibodies

### A. Anti-NIS Polyclonal Antibodies

The sequence of human NIS (hNIS) protein is set forth in SEQ ID NO:46. Cells infected with a virus, such as GLV-1h153, which encodes hNIS will express NIS on the surface of the cell such that the extracellular domain should be accessible and bind to antibodies that specifically bind to the extracellular domain.

To test this, anti-hNIS antibodies sc-134515, sc-48055, sc-48056 and sc-48052 (Santa Cruz Biotechnology, Inc.) were purchased. Antibody sc-134515 is a rabbit polyclonal antibody raised against a peptide corresponding to amino acids 11-52 of hNIS (TFGAWDYGVFALMLLVSTGIGLWVGLARGGQRSAEDFFTGGR (SEQ ID NO:47)). Antibody sc-48055 is an affinity purified goat polyclonal antibody raised against a peptide corresponding to amino acids 1-50 of hNIS (MEAVETGERPTFGAWDYGVFALMLLVSTGIGLWVGLARGGQRSAEDFFTG (SEQ ID NO:48). Antibody sc-48056 is an affinity purified goat polyclonal antibody raised against a peptide corresponding to amino acids 500-550 of hNIS PANDSSRAPSSGMDASRPALADSFYAISYLYYGALGTLTTVLCGALISCLT (SEQ ID NO: 49). Antibody sc-48052 is an affinity purified goat polyclonal antibody raised against a peptide corresponding to amino acids 550-600 of human NIS. Antibody sc-134515 is an affinity purified rabbit polyclonal antibody raised against amino acids 11-52 mapping near the N-terminus of NIS of human origin. The manufacturer of these antibodies, Santa Cruz Biotechnology, Inc., suggested to use the antibody sc-134515 as the most appropriate reagent for this application. This antibody, however, was not effective in capturing virus-infected (GLV-1h153) cells that express hNIS encoded by virus. It appears that the epitope recognized by this antibody is not presented on the surface of these cells.

Thus, the amino acid sequences of two extracellular regions of hNIS (RGVMLVGGPRQVLTLAQNHSRINLMDFNPDPRSR (SEQ ID NO:50) and YPPSEQTMRVLPSSAARCVALSVNASGLLDPALLPANDSSRAPSSGMDASRPALADS FYA (SEQ ID NO: 51)) were analyzed, and two 14-amino acid polypeptides were identified and were selected as immunizing antigens: 1) hNIS₂₂₅₋₂₃₈, corresponding to amino acids 225-238 of hNIS (NHSRINLMDFNPDP (SEQ ID NO:52)) and 2) hNIS₅₀₂₋₅₁₅, corresponding to amino acids 502-515 of hNIS (NDSSRAPSSGMDAS (SEQ ID NO: 53)).

Polypeptides hNIS₂₂₅₋₂₃₈ and hNIS₅₀₂₋₅₁₅ were conjugated to keyhole limpet hemocyanin. Rabbits were immunized with each peptide conjugate, using T-Max® Adjuvant (GenScript, Piscataway, N.J.). Polyclonal antibodies raised against peptide hNIS₅₀₂₋₅₁₅ (designated Ab502) and peptide hNIS₂₂₅₋₂₃₈ (designated Ab225) were purified by affinity purification. Binding of the purified polyclonal antibodies Ab502 and Ab225 to hNIS₅₀₂₋₅₁₅ and hNIS₂₂₅₋₂₃₈, respectively, was confirmed by ELISA.

### B. Fluorescence Microscopy

In vitro binding of polyclonal antibody preparation designated Ab502 to hNIS was measured by fluorescence microscopy. A549 cells infected with GLV-1h153 (hNIS virus) or GLV-1h68 (control virus) were incubated with Ab502 (1.5 μg/mL). Secondary antibody (Donkey anti-rabbit-PE, eBioscience Cat No. 12-4739-81) (0.4 μg/mL) was added, and the cells were observed under a fluorescence microscope. In cells infected with GLV-1h153 and incubated with Ab502, the cell membrane-associated hNIS was clearly visible by fluorescence. This fluorescence was comparable to or greater than the fluorescence observed in cells stained with an anti-CD44 antibody (Mouse Anti-Human CD44-PE, BD Pharmingen Cat No 555479) as a positive control. In control cell stains where Ab502 or the secondary antibody was omitted, fluorescence was not detectable.

### C. Flow Cytometry

Flow cytometry experiments confirmed that Ab502 binds specifically to GLV-1 h153-infected A549 cells. For detection by flow cytometry, both the primary and the secondary antibody were titrated, and the optimal concentration of Ab502 was determined to be 1 μg/mL, while the optimal secondary antibody concentration (Donkey anti-rabbit-PE, eBioscience Cat No. 12-4739-81) was determined to be 0.5 μg/mL. The flow cytometry experiments were performed using standard protocols, briefly: a) virus-infected or control cells were harvested, counted, washed and incubated with the primary anti-hNIS antibody for 30 minutes on ice; b) the labeled cells were washed and the secondary antibody was added for another 30-min incubation on ice; c) the cells were then washed and fixed with 2% paraformaldehyde; d) flow cytometry analysis was performed on a BD Biosciences FACSCanto II flow cytometer.

Accordingly, antibody that specifically binds to the epitope recognized by the new antibody Ab502, can be employed to detect and/or isolate cells, particularly tumor cells, infected with a virus, such as vaccinia virus, that encodes hNIS. For ease of detection or isolation of such cells, the antibody can be immobilized on a solid support, such as magnetic beads. Thus, provided is a method for isolating tumor cells from body fluids by administering a virus that encodes hNIS (or other cell surface protein), contacting the cells with antibody that specifically binds to the protein expressed on the surface of the cells, and detecting binding of the antibody and/or isolating bound cells.

### Example 40

### Optimization of VACV-Cytospin CTC Detection Assay

In this example, an optimal viral dose for infection of tumor cells in blood samples using TurboFP635-expressing vaccinia virus GLV-1h254 was determined. The optimal viral dose was then used to evaluate the capture efficiency, detection efficiency and specificity, as well as infection efficiency, of the VACV-cytospin based CTC assay.

### A. Determining Optimal Viral Dose

Approximately 90% infection efficiency was observed with a viral dose of 10⁷ pfu/mL and 10⁸ pfu/mL whole blood. Thus, the optimal viral dose for infection of tumor cells in blood samples using TurboFP635-expressing VACV, GLV-1h254, was 10′ pfu/mL of whole blood.

### B. Capture Efficiency, Detection Efficiency and Detection Specificity

Blood samples were obtained from healthy human donors or from healthy mice. To collect mouse blood samples, mice were anesthetized with 1% to 1.5% isoflurane and the blood was collected from the left ventricle of the heart into EDTA tubes using a 26-G needle (BD Bioscience, San Jose, Calif., USA).

1×10⁶ PC-3 cells (ATCC# CRL-1435) were labeled with the green fluorescence dye PKH-67 using the PKH-67 Green Fluorescent Cell Linker Kit for General Cell Membrane Labeling (Sigma-Aldrich, St. Louis, Mo., USA). To spike the accurate number of tumor cells into blood samples, labeled cells were diluted properly so that every 3 μL cell suspension contained about 30˜100 single cells. Three μL of the diluted cell suspension were then loaded onto a glass slide and cells were counted under an epifluorescence microscope (Olympus, Center Valley, Pa., USA), followed by washing cells into a blood sample twice each with 20 μL 1×DPBS (Mediatech, Manassas, Va., USA). Thirty to sixty PC-3 human prostate cancer cells labeled with PKH-67 were spiked into 1 mL of whole blood from healthy human donors or 100 μL whole blood from healthy mice in triplicate. Blood samples from six healthy donors and six healthy mice were tested. All procedures were performed by a single operator.

The spiked whole blood samples were subjected to red blood cell lysis as previously described below and infected with vaccinia virus. The GLV-1h254 virus stock was diluted in DMEM supplemented with 2% FBS to yield a concentration of 2×10⁷ plaque-forming units (pfu)/mL. Nucleated cells from 1 mL of the human whole blood following red blood cell lysis were resuspended in 0.5 mL of the diluted virus and incubated at 37° C. for 24 h. Nucleated cells from 100 μL of the mouse whole blood following red blood cell lysis were resuspended in 50 μL of the diluted virus and incubated at 37° C. for 24 h.

Hettich cytospin chambers (Tuttlingen, Germany) were assembled and the cell suspension was directly added into cytospin reservoirs using a funnel card that creates cytospins with a diameter of 8.7 mm. The cells were deposited onto clean glass grid slides (VWR, West Chester, Pa., USA) by centrifugation for 5 minutes at 1,500 rpm using a Hettich Universal 16 centrifuge (Hettich, Germany). Slides were dried for 10 minutes at room temperature. An Olympus IX71 inverted epifluorescence microscope with PictureFrame® software was used to image cells on grid slides. The grids of each slide were checked for CTCs under the microscope one by one. Each experiment was performed in triplicate and six individual human and mouse blood samples were used. Expression of TurboFP635 indicated infection with GLV-1h254 and expression of green fluorescent protein PKH67 indicated tumor cells.

The capture efficiency was defined as a percentage of spiked cells (PKH67+/DAPI+) captured on a slide over all PKH67 labeled cells spiked into the blood sample. The detection efficiency was defined as a percentage of infected tumor cells (PKH67+/TurboFP635+/DAPI+) captured on a slide over all PKH67 labeled cells spiked into the blood sample. The specificity was defined as a percentage of infected tumor cells (PKH67+/TurboFP635+/DAPI+) over all infected cells (TurboFP635+/DAPI+) captured on a slide. The infection efficiency was defined as a percentage of infected tumor cells (PKH67+/TurboFP635+/DAPI+) over all PKH67 labeled cells captured on a slide

The assay yielded similar results with the human and mouse blood samples (see Table 18 below). More than 70% of spiked tumor cells were captured on cytospin slides (70% capture efficiency), and more than 65% of spiked tumor cells were detected by virus infection (65% detection efficiency). More than 92% of the cells captured on the cytospin slides were infected by the virus (92% infection efficiency). All (100%) of the infected cells identified on the slides were spiked tumor cells (100% detection specificity).

TABLE 18
Capture efficiency, detection efficiency, infection efficiency
and infection specificity

| | Capture | Detection | Infection | Infection |
|---|---|---|---|---|
| Samples | efficiency | efficiency | efficiency | specificity |
| PC-3/human | 70.61 ± 3.51 | 65.67 ± 4.57 | 92.97 ± 3.72 | 100 |
| blood | | | | |
| PC-3/mouse | 72.69 ± 3.78 | 68.04 ± 4.79 | 93.62 ± 4.70 | 100 |
| blood | | | | |

### C. Specificity of Infection

To further demonstrate that GLV-1h254 specifically infects only spiked tumor cells, but not healthy blood cells, human and mouse whole blood samples with or without spiked PC-3 cells were infected in parallel with GLV-1h254 after red blood cell lysis. The infected samples were then stained with anti-human or anti-mouse CD45 monoclonal antibodies (FITC conjugated mouse anti-human monoclonal antibody CD45 (clone HI30) (BD Bioscience, San Jose, Calif., USA); FITC conjugated mouse anti-mouse monoclonal antibody CD45 (clone 104) (Abcam, Cambridge, Mass., USA)) to identify the human or mouse leukocytes in the samples. The infected and stained cells were subjected to cytospin deposition and imaging as described in section B above.

All cells showing high-level expression of TurboFP635 indicating infection with GLV-1h254 were CD45-negative (tumor cells), whereas all cells staining positive for CD45 (healthy cells) showed no TurboFP635 expression, thereby demonstrating that GLV-1h254 specifically infects the tumor cells.

### Example 41

### VACV-Cytospin Assay

The following example demonstrates the general procedure for the VACV-cytospin assay for detection of CTCs in blood samples using GLV-1h254 as a model virus.

### A. Red Blood Cell Lysis

Mononuclear cells and circulating tumor cells were enriched from whole blood samples by removing red blood cells with 1×RBC lysis buffer (eBioscience, San Diego, Calif., USA) according to the manufacturer's instruction. For human blood samples, 1 mL of the whole blood was transferred to a 50 mL Falcon tube (Corning, Lowell, Mass., USA) and gently mixed with 10 mL of 1×RBC lysis buffer, followed by incubation for approximately 5 to 10 minutes at room temperature. When the color of the blood changed to a transparent cherry red, the lysis reaction was immediately stopped by diluting the lysis buffer with 30 mL of 1×DPBS. For mouse blood samples, 0.1 mL of the whole blood was transferred to a 15 mL Falcon tube (Corning Life Science, Union City, Calif., USA) and gently mixed with 1 mL of 1×RBC lysis buffer, followed by incubation for approximately 5 to 10 minutes at room temperature. When the color of the blood changed to a transparent cherry red, the lysis reaction was immediately stopped by diluting the lysis buffer with 3 mL of 1×DPBS. The cells were then centrifuged using the Sorvall® Legend RT centrifuge (Sorvall, Germany) at 300×g for 5 minutes at room temperature. The cell pellet was carefully resuspended in an appropriate buffer (see below).

### B. Vaccinia Virus (VACV) Infection

The GLV-1h254 virus stock was diluted in DMEM supplemented with 2% FBS to yield a concentration of 2×10′ plaque-forming units (pfu)/mL. Nucleated cells from 1 mL of the whole blood following red blood cell lysis were resuspended in 0.5 mL of the diluted virus (e.g., half the volume of the sample) and incubated at 37° C. for 24 h.

### C. Immunofluorescence Staining and Cell Deposition

The following antibodies were used to identify and characterize CTCs: FITC conjugated mouse anti-human monoclonal antibody EpCAM (clone EBA-1) (BD Bioscience, San Jose, Calif., USA), FITC conjugated mouse anti-human monoclonal antibody CD45 (clone HI30) (BD Bioscience, San Jose, Calif., USA), FITC conjugated mouse anti-human monoclonal antibody pan-cytokeratin (CK, clone C-11) (Abcam, Cambridge, Mass., USA), FITC conjugated mouse anti-human monoclonal antibody CD44 (clone G44-26) (BD Bioscience, San Jose, Calif., USA), FITC conjugated mouse anti-human monoclonal antibody CD45 (clone 104) (Abcam, Cambridge, Mass., USA), FITC conjugated mouse anti-human monoclonal antibody carcinoembryonic antigen (CEA, CD66) (clone B1.1/CD66) (BD Bioscience, San Jose, Calif., USA), FITC conjugated mouse anti-human monoclonal antibody Progesterone Receptor (clone SP2) (Abcam, Cambridge, Mass., USA), FITC conjugated mouse anti-human monoclonal antibody HER-2/neu (clone Neu 24.7) (BD Bioscience, San Jose, Calif., USA), purified mouse anti-human monoclonal antibody MITF (clone D5) (Santa Cruz Biotechnology, Santa Cruz, Calif., USA), purified mouse anti-human monoclonal antibody Melan-A (clone A103) (Santa Cruz Biotechnology, Santa Cruz, Calif., USA), purified mouse anti-human monoclonal antibody ALDH (clone 44/ALDH) (BD Bioscience, San Jose, Calif., USA), purified mouse anti-human monoclonal antibody N-Cadherin (clone 32/N-Cadherin) (BD Bioscience, San Jose, Calif., USA) and purified mouse anti-human monoclonal antibody Vimentin (clone V9), FITC conjugated goat anti-mouse polyclonal secondary antibody IgG-H & L (Abcam, Cambridge, Mass., USA).

Immunofluorescence staining procedures were performed according to the manufacturer's instructions. In brief, nucleated cells from 1 mL of the whole blood following red blood cell lysis were resuspended in 0.5 mL of 4% paraformaldehyde and fixed for 5 minutes, followed by washing with 1×DPBS and incubation with antibodies at room temperature for 30 minutes. For staining of cells with non-fluorescence conjugated primary antibodies, the further incubation with secondary antibodies conjugated with a fluorescence dye was applied. An additional permeabilization step with 0.5 mL of cold methanol for 5 minutes before antibody incubation was required if an intracellular antigen (e.g. cytokeratin) was needed to be detected.

After staining, the cells were washed once with 1×DPBS and resuspended in 1 mL of 1×DPBS. Hettich cytospin chambers (Tuttlingen, Germany) were assembled and the stained cell suspension was directly added into cytospin reservoirs using a funnel card that creates cytospins with a diameter of 8.7 mm. The cells were deposited onto clean glass grid slides (VWR, West Chester, Pa., USA) by centrifugation for 5 minutes at 1,500 rpm using a Hettich Universal 16 centrifuge (Hettich, Germany). Slides were dried for 10 minutes at room temperature. The 4′,6-diamidino-2-phenylindole (DAPI) HardSet mounting medium (Vector Laboratories, Burlingame, Calif., USA) was used for cell nuclei staining.

D. Visualization and enumeration of CTCs

An Olympus IX71 inverted epifluorescence microscope with PictureFrame® software was used to image cells on grid slides. The grids of each slide were checked for CTCs under the microscope one by one. Captured images (at 640× of total magnification) were carefully examined and the objects that met preset criteria were counted. Color, brightness, and morphometric characteristics such as cell size, shape, and nuclear size were considered in identifying potential CTCs and excluding nonspecific cells. Infected CTCs showed very strong TurboFP635 expression together with staining positive for epithelial cell adhesion molecule (EpCAM), pan-cytokeratin (CK) and DAPI, but negative for CD45, and met the morphologic characteristics consistent with malignant cells, including large cellular size, high nuclear to cytoplasmic ratio, and visible nucleoli, were scored as CTCs. Cell counts were expressed as the number of cells per actual volume of the blood sample.

### Example 42

### Detection and Identification of Live Human CTCs in Blood Samples from Mice Bearing Human Tumor Xenografts

In this example, live human CTCs were detected and identified in blood samples from mice bearing human tumor xenografts, including a prostate cancer model using PC-3 tumor cells and a late-stage non-small cell lung cancer model using A549 cells.

### A. Mouse Tumor Xenograft Models

The human prostate cancer cell line PC-3 and the human lung carcinoma cell line A549 were purchased from the American Type Culture Collection (ATCC). A549 cells were cultured in RPMI 1640 (Mediatech, Manassas, Va., USA) supplemented with 10% FBS (Mediatech, Manassas, Va., USA). PC-3 cells were cultured in DMEM (Mediatech, Manassas, Va., USA) supplemented with 10% FBS (DMEM-10).

Mice were cared for in accordance with approved protocols by the Institutional Animal Care and Use Committee of Explora Biolabs (San Diego Science Center, San Diego, Calif., USA). Cardiac puncture was used for serial studies of CTC detection. Blood samples (˜100 μL) were collected into EDTA tubes using a 26-G needle (BD Bioscience, San Jose, Calif., USA) inserted into the chest over the point of maximal impulse from the heart and blood samples (˜1 mL) were collected by this same route when mice were euthanized at the end of experiments. This procedure was performed without assistance from a needle holder or other external guidance system.

Five- to six-week old nude mice (NCI:Hsd:Athymic Nude-Foxn1nu; Harlan, Indianapolis, Ind., USA) were implanted subcutaneously with 5×10⁶ PC-3 or A549 cells (in 100 μL PBS) on the right hind leg. Tumor growth and mouse weight were monitored weekly. 100 μL of whole blood samples were taken from these mice by cardiac puncture for CTC analysis were lysed, infected with 50 μL 2×10⁷ pfu/mL GLV-1h254 and analyzed using the VACV-cytospin assay described in Example 41.

### B. Human Prostate Cancer Xenograft

Infection of blood samples from PC-3 xenografts with GLV-1h254 ex vivo revealed microscopically that infected cells were much larger than surrounding CD45⁺ immune cells, displayed bright TurboFP635 fluorescent signal, contained nuclei, and were CD45. These infected cells were also CK⁺ or EpCAM⁺, indicating that the infected cells were of epithelial origin, as expected for PC-3-derived CTCs.

### C. Human Late-Stage Non-Small Cell Lung Cancer Xenograft

Infection of blood samples from A5649 xenografts with GLV-1h254 ex vivo revealed microscopically that CTCs were detected and identified as TurboFP635⁺/CD45⁻/DAPI⁺ cells.

The results indicate GLV-1 h254 is tumor-specific for human CTCs in mice bearing human cancer xenografts.

### Example 43

### Detection and Identification of Live CTCs in Blood Samples from Patients with Cancer

In this example, CTCs were detected and identified in blood samples from human cancer patients.

### A. Breast Cancer

Whole blood samples from seven patients with breast cancer were analyzed using the VACV-cytospin assay described in Example 41. The volume of each sample varied from 5 to 15 mL.

Live CTCs were detected in three patients. CTCs were not detected in the remaining four patients (4-7 mL samples). To confirm the absence of CTCs in these patients, the same blood samples were analyzed using immunostaining for CK and EpCAM. Again, CTCs were not detected.

Patient BC1 had stage III breast cancer with histological diagnosis of estrogen receptor (ER)− negative, progesterone receptor (PR)-negative and human epidermal growth factor receptor 2 (HER2/neu)−negative, indicating an aggressive disease, and had been undergoing chemotherapy and irradiation treatments before the blood sample was drawn. The VACV-cytospin assay detected a total of 66 live CTCs in a 5.5 mL whole blood sample. The CTCs identified by GLV-1h254 (TurboFP635⁺/CD45⁻/DAPI⁺) were also CK⁺ or EpCAM⁺.

Another patient, BCS, had stage I breast cancer with histological diagnosis of ER⁻, PR⁺ and HER2/neu⁺. Fifteen live CTCs were detected in a 5 mL blood sample from this patient. These live CTCs showed not only EpCAM expression, but also PR and HER2/neu expression consistent with the histological diagnosis. Similar to patient BC1, patient BC7 also had stage III breast cancer with histological diagnosis of ER⁻, PR⁻ and HER2/neu⁻. Patient BC7 had been undergoing chemotherapy, irradiation and trastuzumab treatments before the blood sample was drawn. Only 3 live CTCs were found in a 3 mL blood sample. The CTCs were identified as TurboFP635⁺/CK⁺/DAPI⁺ cells.

### B. Other Types of Cancer

Blood samples were analyzed from patients with metastatic colorectal cancer, lung cancer and melanoma using the VACV-cytospin assay described in Example 41.

Patient CC 1 with metastatic colorectal cancer had been undergoing chemotherapy and bevacizumab treatments before the whole blood sample was drawn. Forty-one live CTCs were detected in a 5 mL blood sample from this patient. These infected live CTCs were confirmed as TurboFP635⁺/EpCAM⁺ or CK⁺/CD45⁻/DAPI⁺ cells.

The lung cancer patient LC1 with brain metastases had not been undergoing any treatment. Fourteen live CTCs were identified in a 5 mL blood sample from this patient. These live CTCs also displayed EpCAM expression.

Twenty-four live CTCs were detected by GLV-1h254 in a 5 mL whole blood sample from the patient MM1 with malignant metastatic cutaneous melanoma and these CTCs were confirmed to express melanoma markers microphthalmia-associated transcription factor or Melan-A.

The results indicate GLV-1h254 is tumor-specific for human CTCs in patients with cancer.

### Example 44

### Side-by-Side Comparison of VACV-Cytospin Assay and CellSearch® System

In this example, blood samples from 10 patients with metastatic breast cancer and colon cancer were evaluated for CTCs in a side-by-side comparison using the VACV-cytospin assay and CTC detection using the CellSearch® system.

From each patient, one 7.5 mL blood sample was collected in a CellSave® tube and shipped to the Genoptix Laboratory (Carlsbad, Calif.) for CTC detection using the CellSearch® system and a second 7.5 mL blood sample from the same blood draw was collected in EDTA tubes and analyzed for CTCs using the VACV-cytospin assay described in Example 41. The live CTCs identified by VACV were confirmed with immunostaining as turboFP635⁺/CK⁺/CD45⁻/DAPI⁺, as well as having morphologic characteristics consistent with malignant cells, including large cellular size, a high nuclear to cytoplasmic ratio, and visible nucleoli. CellSearch® samples were analyzed as previously described (Miller et al. (2010) J Oncol 2010:617421; Allard et al. (2004) Clin Cancer Res 10:6897-6904). A CTC was defined according to the criteria of round to oval morphology, cell size more than 4 μm, DAPI positive nucleus, CK positive staining, and absence of CD45 expression. CTC number was reported per 7.5 ml of blood. The sensitivity, accuracy, linearity, and reproducibility of the CellSearch® system have been previously described (Allard et al. (2004) Clin Cancer Res 10:6897-6904; Reithdorf et al. (2007) Clin Cancer Res 13:920-928).

The results are set forth in Table 19 below, where the values indicate the number of CTCs per 7.5 mL of blood. The results indicate both assays detected CTCs in patients 1-4. In addition, the VACV-cytospin assay detected 2 CTCs in patient 7, but no CTCs were detected in this patient by the CellSearch® system. Neither assay detected any CTCs in the remaining 5 patients. The VACV-cytospin assay detected slightly more CTCs in patients 1 and 2, but a few less CTCs in patient 3 than the CellSearch® system. The CellSearch® system detected 103 CTCs in patient 4 while only 5 CTCs were identified using the VACV-cytospin assay, indicating most of the CTCs in patient 4 might not be alive since the CellSearch® assay detects live and dead CTCs whereas the VACV-cytospin assay only detects live CTCs.

TABLE 19
Comparison of VACV-cytospin assay and CellSearch ® system

| Patient ID | 01 | 02 | 03 | 04 | 05 | 06 | 07 | 08 | 09 | 10 |
|---|---|---|---|---|---|---|---|---|---|---|
| CTC # | 7 | 27 | 5 | 103 | 0 | 0 | 0 | 0 | 0 | 0 |
| (CellSearch ®) | | | | | | | | | | |
| Live CTC # | 10 | 35 | 2 | 5 | 0 | 0 | 2 | 0 | 0 | 0 |
| (VACV) | | | | | | | | | | |

### Example 45

### Characterization of CTCs Identified in Blood Samples from Mice with Human Tumor Xenografts and Patients with Cancer

Studies have indicated that circulating tumor cells (CTCs) are linked to cancer stem cells (CSCs) and the epithelial-mesenchymal transition (EMT) process (see, e.g., Bonnomet et al. (2010) J Mammary Gland Biol Neoplasia 15:261-273 and Pierga et al. (2008) Clin Cancer Res 14:7004-7010). To elucidate the relationship of CTCs with CSCs and EMT, CTCs were analyzed for the expression of CSC and EMT markers, including CD44, aldehyde dehydrogenase 1 (ALDH1), vimentin and N-cadherin, as described in Example 41, In addition, single-cell measurements were performed to compare the nuclear size of identified CTCs and adjacent nucleated blood cells. Cell images were analyzed by ImageJ software (NIH, Bethesda, Md., USA) and the nuclear diameter was measured using the plugins of ImageJ.

The CTCs identified with GLV-1h254 in mice bearing human PC-3 prostate cancer xenografts (Example 42) displayed high levels of expression of the CSC markers CD44 and aldehyde dehydrogenase 1 (ALDH1) as well as the EMT markers vimentin and N-cadherin. Furthermore, the CTCs identified with GLV-1h254 in the breast cancer patients BC 1 and BCS (Example 43) showed high-level expression of CD44 and ALDH1, respectively. The features of CSCs as well as phenotypic change characteristics of the EMT possessed by CTCs might allow them to disseminate effectively during the progress of cancer metastases, resulting in the formation of secondary tumors by extravasation and colonization in distant organs.

The diameters of the live CTC nuclei ranged from 1.3 to 5.5 times greater than that of neighboring white blood cells, demonstrating evidence of the heterogeneity of CTCs in size.

### Example 46

### Detection and Identification of Live Cancer Cells in CSF Samples from Patients with Cancer

Cerebrospinal fluid (CSF) samples from seven patients with glioblastoma multiforme, metastatic colorectal carcinoma, metastatic breast cancer and metastatic esophageal cancer were analyzed using the VACV-cytospin assay described in Example 41.

CSF samples were collected at the Moores Cancer Center, University of California, San Diego (La Jolla, Calif., USA). All enrolled patients gave their informed consent for study inclusion and were enrolled using institutional review board approved protocols. Three to five mL CSF from each patient was collected. CSF samples were maintained at room temperature for delivery and processed within a maximum of 24 hours after CSF draw. The collected CSF samples were concentrated by centrifugation using the Sorvall® Legend RT centrifuge (Sorvall, Germany) at 300×g for 5 minutes at room temperature. The cell pellet was carefully resuspended in an appropriate buffer. Cells were counted after staining with trypan blue (Mediatech, Manassas, Va., USA) to determine the number of viable cells. Vaccinia virus (VACV) GLV-1h254 infection and immunofluorescence staining were performed as described in Example 41 above. Subsequently all the cells from CSF of one patient were deposited in one grid slide by cytospin as described for CTCs above. Thereafter, cells on the slide were imaged and enumerated under an epifluorescence microscope.

The results showed that a total of 23 TurboFP635⁺ cells (vaccinia infected cells) with large nuclei were found in the 3 mL CSF sample from patient CSF7 having metastatic breast cancer. Among these, 16 cells showed high-level expression of CK and the rest of infected cells showed very low level or no expression of CK. No infected cells were found in the CSF samples from other six patients. To confirm the absence of cancer cells in these six CSF samples, infected samples were further analyzed using immunostaining for CK. No CK⁺ cells were detected in these six patients that were negative for TurboFP635 (not infected with vaccinia virus).

### Example 47

### Prevention and Therapy of CTCs in Mice Bearing Human Prostate Cancer Xenografts

Nude mice were implanted with the modified human prostate cancer cell line PC-3-RFP expressing red fluorescent protein (RFP) (see Example 2A above) to facilitate CTC detection using the ClearBridge biochip.

Five- to six-week old male nude mice (NCI:Hsd:Athymic Nude-Foxn1nu; Harlan, Indianapolis, Ind., USA) were implanted subcutaneously with 5×10⁶PC-3-RFP cells in 100 μL PBS on the right hind leg. Tumor growth and mouse weight were monitored weekly. Groups of 8 mice were treated with a single dose of 5×10⁶ pfu of GLV-1h68 (in 100 μL of PBS) either at 4 weeks (early treatment) or 7 (late treatment) weeks after tumor cell implantation. Mice treated with PBS at 4 weeks after tumor cell implantation were used as controls. Mice were monitored weekly for CTCs using Clearbridge BioMedics CTC0 Capture System Prototype (as described in Example 11A above). 100 μL of blood were drawn from mice through heart puncture and 80 μL were run through the biochip. CTCs captured on the biochip were visualized and counted under a fluorescent microscope.

No CTCs were detected in any of the mice through 4 weeks after tumor cell implantation (prior to any treatment). All mice in the PBS-treated control group were CTC positive at one or more time points starting at 5 weeks after tumor cell implantation. In contrast, only one animal in the GLV-1h68 early treatment group had any detectable CTC's after virus treatment (3, 1, and 2 CTCs at 2, 3, and 4 weeks after treatment, respectively). Thus, early treatment significantly reduced CTC formation in mice bearing human prostate cancer tumors.

Mice in the late treatment group had a significant number of CTCs before virus treatment, ranging from 12 to 103 CTCs per 80 μL of blood. A decrease in CTC numbers was observed one week after virus treatment. At 1 week after treatment, 52.9% of CTCs were GFP positive, and thus were infected by GLV-1h68. At 2 weeks after treatment, the number of CTCs remained at reduced levels, and almost all CTCs (99.6%) were GFP positive (infected by GLV-1h68).

While primary tumors kept growing in the PBS group, early and late treatments with GLV-1h68 resulted in tumor regression and significantly prolonged survival in comparison with PBS treatment. The average survival days were 126.3 and 97.3 days for the early and late treatment groups, respectively, versus 52.7 days for the PBS treatment group.

At death or at the end of the experiment, all mice were dissected and metastases were examined under a fluorescent stereo microscope. All mice in the PBS control group had detectable lumbar and renal lymph node metastases. In contrast, only 1 out of 8 mice in the early treatment group had a slightly enlarged lumbar lymph node, with the other mice in this group having no detectable lumbar or renal lymph node metastases. Although all mice in the late treatment group had detectable lumbar and renal lymph node metastases, these metastases were smaller in size compared to those in mice in the PBS group.

### Example 48

### Therapy of Metastatic Cancer Cells in the Ascites of a Patient with Peritoneal Carcinomatosis from Gastric Cancer

Tumor cell-containing ascites from a patient with peritoneal carcinomatosis (PC) from gastric cancer that was intraperitoneally treated with GL-ONC1 (clinical version of GLV-1h68) were analyzed.

Ascites were isolated three and seven days after the first intraperitoneal treatment with 10⁷ pfu of GLV-1h68. First, the concentration of cells found in the ascitic fluid of the patient was determined. Then, the cells were spun down by centrifugation, followed by fixation of the cell pellet in formalin (4%, Fischer, Germany) to a final concentration of 1×10⁶ cells/mL. After a repeated centrifugation of this cell suspension, the supernatant was discarded and the cell pellet was resuspended in a few drops of the remaining supernatant. This suspension was collected and mixed with hot agar (1% agarose), cooled down, placed into a histology cassette and fixed again in formalin (4%). The cassette was then processed like a routine surgical specimen and was embedded in paraffin. From the resulting cell blocks, sections of 4 μm thickness were cut, deparaffinised and rehydrated by passages through xylene and graded alcohol and finally stained with haematoxylin and eosin for morphologic evaluation. Then, subsequent sections were mounted on slides and collected for IHC staining.

Staining was performed using an automated immunohistochemistry staining system (VENTANA Benchmark; Ventana Medical Systems, Tucson, Ariz., USA), using reagents from VENTANA according to the manufacturer's protocol. Shortly, the slides were incubated with primary antibodies VACV-A27L (Genelux, Calif., USA); anti-EpCAM antibody Ber-EP4 (Dako, Germany) and visualized using iView DAB detection kit (Ventana) with horseradish peroxidase and DAB as chromogen. After DAB staining, slides were counterstained with haematoxylin, washed, dehydrated in a graded alcohol series and mounted with Cytoseal™ mounting medium (Fisher Scientific, Germany). The study was approved by the Paul-Ehrlich-Institut, Germany and the trial was registered on clinicaltrials.gov (number NCT01443260). Written, informed consent was obtained from the patient.

Using either anti-EpCAM or anti-vaccinia specific antibodies, about 5% of all cells were found to be EpCAM-positive three days after treatment, and only about 5-10% of these cancer cells were vaccinia virus positive at the same time point. In contrast, four days later (i.e., 7 days after treatment), less than 2% of all ascitic cells were still EpCAM-positive, and more than 90% of these cancer cells were vaccinia virus positive. These results indicate that GLV-1h68 effectively removes live tumor cells in the ascites of patients with peritoneal carcinomatosis (PC).

Since modifications will be apparent to those of skill in this art, it is intended that this invention be limited only by the scope of the appended claims.

What is claimed:

1. A method for testing for or monitoring efficacy of treatment with an oncolytic virus for treatment of solid tumors, other cancers and metastatic diseases, comprising testing a body fluid sample obtained from a subject to whom an oncolytic reporter virus has been administered to identify any tumor cells that circulate in the body fluid by detecting the oncolytic reporter virus in tumor cells the sample, wherein:
testing is performed at a pre-determined time following administration of the virus, wherein the predetermined time is a time sufficient for the virus to infect a tumor cell in the subject, but before efficacious therapy would shrink tumors or eliminate any circulating tumor cells (CTCs); and
detection of the reporter virus in tumor cells in the body fluid sample is indicative that the treatment with the oncolytic virus is or will be efficacious.

2. The method of claim 1, wherein the tumors comprise solid tumors, and the tumor cells in the body fluids comprise circulating tumor cells (CTCs) from the tumors.

3. The method of claim 1, further comprising:
if the reporter virus is detected in tumor cells in the body fluid sample indicating that treatment is efficacious, initiating or continuing treatment with the oncolytic reporter virus or with an oncolytic virus that is the same as the reporter virus, except that it does not contain the reporter gene or it encodes a therapeutic protein in addition to or in place of the reporter gene; or,
if the reporter virus is not detected in tumor cells in the body fluid sample, discontinuing treatment with the oncolytic reporter virus or with an oncolytic virus that is the same as reporter virus, except that it does not contain the reporter gene.

4. The method of claim 1, wherein the body fluid sample is tested in vitro after obtaining the body fluid sample from a subject.

5. The method of claim 1, wherein, prior to testing,
the oncolytic reporter virus was administered at a dosage sufficient to be detected but that is lower than a dosage for treatment; or
the oncolytic reporter virus was administered at a dosage for treatment of a tumor or cancer.

6. The method of claim 1, wherein the sample is from a subject having a solid tumor.

7. The method of claim 1, wherein prior to testing, the method comprises enriching tumor cells in the sample to produce an enriched sample.

8. The method of claim 1, wherein:
if the treatment is efficacious as evidenced by detection of reporter virus, continuing treatment of the subject by administering an oncolytic virus for treatment; and
the oncolytic virus for treatment is the same oncolytic reporter virus or is an oncolytic virus where the reporter gene is not present, or the oncolytic virus, with or without the reporter gene, comprises heterologous nucleic acid encoding a therapeutic protein.

9. The method of claim 1, wherein:
detecting of tumor cells is performed to monitor treatment of the subject; and
the body fluid sample is tested at a pre-determined time or pre-determined intervals following administration of the virus;
the predetermined time is at least sufficient for the virus to infect tumor cells; and
changes in the number of detected tumor cells compared to a control sample or the initial sample is an indicator of the progress of treatment.

10. The method of claim 9, wherein:
the samples are obtained prior to 24 days after first administering the virus;
if the number of infected tumor cells in the sample is substantially the same or increased compared to the control or initial sample, then the treatment is continued or accelerated;
if the number of infected tumor cells in the sample is reduced compared to the control, then the treatment is reduced or discontinued; and
if no infected tumor cells are detected, then the treatment is discontinued.

11. The method of claim 9, wherein the control is a sample is of the same bodily fluid from a healthy subject, is a baseline sample from the subject prior to treatment with the oncolytic virus, is a sample from a subject after a previous dose of oncolytic virus, or is a sample from a subject prior to the last dose of oncolytic virus, or is a sample from a subject prior to the last dose of oncolytic virus or is an initial sample from the subject prior to the first dose or immediately after the first dose.

12. The method of claim 9, wherein:
the samples are from a subject to whom a dosage or regiment for treatment of the tumor or cancer was administered;
the samples are obtained more than at least about 24 or at least about 30 days after first administering the virus;
if the number of infected tumor cells in the sample is substantially the same or increased compared to the control or initial sample, then treatment is discontinued;
if the number of infected tumor cells in the sample is reduced compared to the control, then the treatment continued.

13. The method of claim 1, wherein the predetermined time is no more than 6 hours, 12 hours, 18 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days or 24 days following administration of the virus, wherein detection of virus in tumor cells in the sample indicates that the virus has infected tumor cells, and, thus, is predicted to be an efficacious treatment.

14. The method of claim 13, wherein treatment is predicted to efficacious, and the method comprises administering an oncolytic reporter virus once or a plurality of times for treatment of the subject.

15. The method of claim 1, wherein the body fluid sample is a sample selected from blood, peripheral blood, lymph, bone marrow fluid, pleural fluid, peritoneal fluid, spinal fluid, abdominal fluid, pancreatic fluid, cerebrospinal fluid (CSF), brain fluid, ascites, urine, saliva, bronchial lavage, bile, sweat, tears, ear flow, sputum, semen, vaginal flow, milk, amniotic fluid, or secretions of the respiratory, intestinal or genitourinary tract, or is a sample that contains dissociated bone marrow cells from a bone marrow biopsy.

16. The method of claim 1 that is for monitoring therapy, wherein:
the sample is tested after 24 days as treatment progresses to assess whether there is decrease in reporter virus and, thus, tumor cells; and
a decrease in tumor cells indicates that treatment is effective.

17. The method of claim 1, wherein a body fluid sample is obtained a plurality of times at successive time points following administration of the virus, whereby a plurality of samples are obtained from the subject.

18. The method of claim 7, wherein enriching tumor cells from the sample comprises selecting tumor cells from the sample or removing non-tumor cells from the fluid sample.

19. The method of claim 7, wherein enriching tumor cells in the sample comprises capturing or selecting cells based upon larger size, shear modulus, increased stiffness, reduced deformability, increased density or expression of a surface moiety or moieties.

20. The method of claim 7, comprising enriching tumor cells in the sample by separating tumor cells from non-tumor cells in using one or more of microfluidic device, a microfilter, a density gradient, immunomagnetic separation and acoustophoresis.

21. The method of claim 7, wherein:
enriching tumor cells is effected with a microfluidic device through which the cells flow, wherein the device comprises an array of isolation wells; and
each isolation well comprises:
a cell trap that prevents the passage of tumor cells and permits the passage of non-tumor cells and other components of the fluid sample; or
a cell trap that prevents the passage of non-tumor cells and permits the passage of tumor cells in the fluid sample.

22. The method of claim 21, wherein:
the microfluidic device separates tumor cells based on deformability, size and/or stiffness; and
the microfluidic device comprises one or more linear channels, wherein each linear channel has a length and a cross-section of a height and a width defining an aspect ratio adapted to isolate tumor cells along at least one portion of the cross-section of the channel based on reduced deformability or larger size of tumor cells as compared to non-tumor cells, wherein tumor cells flow along a first portion of the channel to a first outlet and non-tumor cells flow along a second portion of the channel to a second outlet.

23. The method of claim 7, wherein enriching tumor cells comprises separating tumor cells from non-tumor cells based on expression of a moiety on the tumor cell surface.

24. The method of claim 23, wherein the tumor cells are separated by contacting the sample with a device, chip or bead, wherein the device, chip or bead contains an immobilized capturing agent that specifically binds to a moiety on the tumor cell surface to thereby effect capture of the tumor cell.

25. The method of claim 23, wherein the cell surface moiety is a cytokeratin or EpCam.

26. The method of claim 24, wherein the capturing agent is an antibody, an antibody fragment, a receptor or a ligand binding domain.

27. The method of claim 1, wherein detecting the oncolytic reporter virus in a sample is effected by a method selected from among flow cytometry, fluorescence microscopy, fluorescence spectroscopy, magnetic resonance spectroscopy and luminescence spectroscopy.

28. The method of claim 1, wherein the reporter virus encodes a reporter gene product that is inserted into or in place of a non-essential gene or region in the genome of the virus.

29. The method of claim 15, wherein:
the body fluid is CSF; and
leptomeningeal metastases (LM) are detected.

30. The method of claim 15, wherein:
the body fluid is peritoneal fluid; and
the method effects diagnosis of peritoneal carcinomatosis by detecting tumor cells in the peritoneal fluid.

31. The method of claim 1, wherein subject has a cancer of the lung, breast, colon, brain, prostate, liver, pancreas, esophagus, kidney, stomach, thyroid, bladder, uterus, cervix or ovary.

32. The method of claim 1, wherein the subject has metastatic cancer.

33. The method of claim 1, wherein the oncolytic virus or oncolytic reporter virus is a vaccinia virus.

34. The method of claim 33, wherein the oncolytic virus or oncolytic reporter virus is a Lister strain virus.

35. The method of claim 34 virus is an LIVP virus, a clonal strain of an LIVP virus, or a modified form thereof containing nucleic acid encoding a heterologous gene product.

36. The method of claim 1, wherein the virus comprises nucleic acid encoding a heterologous gene product that is a therapeutic or diagnostic agent.

37. The method of claim 1, wherein the reporter virus comprises a reporter gene that encodes a fluorescent protein, a bioluminescent protein, a receptor or an enzyme.

38. The method of claim 37, wherein the fluorescent protein is selected from among a green fluorescent protein, an enhanced green fluorescent protein, a blue fluorescent protein, a cyan fluorescent protein, a yellow fluorescent protein, a red fluorescent protein, or a far-red fluorescent protein.

39. The method of claim 38, where the fluorescent protein is designated TurboFP635.

40. The method of claim 37, wherein the reporter gene encodes an enzyme is selected from among a luciferase, β-glucuronidase, β-galactosidase, chloramphenicol acetyl transferase (CAT), alkaline phosphatase and horseradish peroxidase, or encodes a receptor that binds to a detectable moiety or a ligand attached to a detectable moiety.

41. The method of claim 1, wherein the oncolytic virus comprises nucleic acid that encodes a protein that is expressed on the surface of the infected cell; and detection of the virus is effected by detecting the protein expressed on the surface of the infected cell.

42. The method of claim 41, wherein the cell surface protein is a receptor or transporter protein.

43. The method of claim 41, wherein the cell surface protein is a sodium ion transporter.

44. The method of claim 43, wherein the sodium ion transporter is a norepinephrine transporter (NET) or the sodium iodide symporter (NIS).

45. The method of claim 44, wherein the NIS or NET is a human NIS or NET protein.

46. The method of claim 41, wherein detection is effected by contacting the cells with an antibody that specifically binds to an epitope on the extracellular domain of the protein expressed on the cell surface.

47. The method of claim 46, wherein the antibody comprises a polyclonal antibody preparation or is a monoclonal antibody.

48. The method of claim 46, wherein the antibody is linked to a magnetic bead for separating cells that express the cell surface protein to thereby isolate virus-infected cells.

49. The method of claim 43, wherein:
the antibody specifically binds to an epitope in the NIS protein.

50. The method of any of claim 50, wherein the antibody specifically binds to a polypeptide that comprises the sequence NDSSRAPSSGMDAS (SEQ ID NO: 53) or an epitope therein.

51. The method of claim 41, wherein the oncolytic virus is a lister strain vaccinia virus.

52. The method of claim 51, wherein the lister strain virus is the virus designated GLV-1h68 or a derivative thereof or is an LIVP clonal strain.

53. A method of detecting a viable tumor cells in a body fluid sample, comprising:
a) enriching tumor cells in a body fluid sample from a subject administered an oncolytic reporter virus to produce an enriched sample; and
b) detecting the reporter virus in tumor cells in the sample, thereby detecting viable tumor cells in the sample.

54. A method of detecting a tumor cell in a body fluid sample, comprising testing a body fluid sample from a subject, wherein the subject has not been treated with an oncolytic reporter virus, the method comprising:
a) enriching tumor cells from the sample to produce an enriched sample;
b) contacting tumor cells from the sample with an oncolytic reporter virus; and
c) detecting the oncolytic reporter virus, thereby detecting tumor cells in the sample.

55. The method of claim 54, wherein detecting tumor cells in the sample indicates that the oncolytic virus is a candidate for treatment of the tumor.

56. The method of claim 54, wherein detection of tumor cells indicates that the subject has a tumor.

57. A method for detecting viable circulating tumor cells, comprising:
a) detecting tumor cells in a body fluid sample that is infected with an oncolytic reporter virus, wherein:
the sample is obtained from a subject who has been administered an oncolytic reporter virus; and
the tumor cells are detected by detecting a tumor cell marker;
b) optionally enriching tumor cells in the sample to produce an enriched sample; and then
c) detecting tumor cells with the tumor cell marker that are infected with the virus by detecting the oncolytic reporter virus, whereby detection of infected tumor cells effects detection of viable circulating tumor cells.

58. The method of claim 57, wherein the tumor cell marker is an epithelial cell marker or cancer stem cell marker.

59. The method of claim 58, wherein the body fluid is cerebrospinal fluid or peritoneal fluid.

60. The method of claim 59, wherein:
the body fluid is cerebrospinal fluid, and detection of circulating tumor cells in the cerebrospinal fluid indicates the subject has leptomeningeal metastases; or
the body fluid is peritoneal fluid, and detection of circulating tumor cells in the peritoneal fluid indicates that the subject has peritoneal carcinomatosis.

61. A method of assessing prognosis of a cancer, comprising testing a body fluid sample from a subject by:
a) enriching tumor cells in the sample to produce an enriched sample;
b) contacting the sample or enriched sample or cells from the same with an oncolytic reporter virus; and
c) identifying cancer stem cells by:
i) detecting the oncolytic reporter virus to identify cells infected with the virus and from the identified cells identifying stem cells; or
ii) identifying stem cells and from among the identified stem cells identifying cells infected with virus, whereby the presence of cancer stem cells is indicative of the presence of an aggressive cancer.

62. The method of claim 61, wherein stem cells are identified based on expression of aldehyde dehydrogenase (ALDH1).

63. An antibody that specifically binds to the extracellular domain of NIS that is expressed in cell, wherein the NIS protein is encoded by an oncolytic virus that has infected the cells that express the NIS protein.

64. An isolated polypeptide, comprising the sequence NDSSRAPSSGMDAS (SEQ ID NO: 53), wherein the polypeptide does not comprise the complete extracellular domain of NIS.

65. An antibody that specifically binds to the polypeptide of claim 65, and also binds to an epitope on the extracellular domain of NIS when expressed on the surface of a cell.

66. An antibody of claim 64 that binds an epitope within a region corresponding to amino acids 502-515 of the NIS polypeptide of SEQ ID NO:46.
